(19) United States
(12) Patent Application Publication
(10) Pub. No.: US 2024/0043536 A1
(43) Pub. Date: Feb. 8, 2024

(54) PEPTIDE COMPOSITIONS AND METHODS FOR ANTI-CD3 BINDING DOMAINS

(71) Applicant: Janux Therapeutics, Inc., San Diego, CA (US)

(72) Inventors: David CAMPBELL, San Diego, CA (US); Thomas R. DIRAIMONDO, San Diego, CA (US)

(21) Appl. No.: 18/256,278

(22) PCT Filed: Dec. 7, 2021
(86) PCT No.: PCT/US2021/062233

§ 371 Date: Jun. 7, 2023

Related U.S. Application Data
(60) Provisional application No. 63/122,820, filed on Dec. 8, 2020.

(51) Int. Cl. C07K 16/28; C07K 7/08; C07K 16/30; A61P 35/00

(52) U.S. Cl. CPC C07K 16/2809; C07K 7/08; C07K 16/3007; A61P 35/00; C07K 2319/50; C07K 2317/622; C07K 2317/569; C07K 2317/92; C07K 2317/76; C07K 2317/73; C07K 2317/94; C07K 2317/22; C07K 2317/31

(57) ABSTRACT

Disclosed herein are polypeptide and polypeptide complex compositions that comprise a peptide that selectively impairs binding of an anti-CD3 binding domain to CD3 in healthy tissue but not in a disease state.

### CROSS-REFERENCE

The present application claims the benefit of U.S. Provisional Application No. 63/122,820, filed Dec. 8, 2020, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Dec. 2, 2021, is named 52426-732_601_SL.txt and is 280,999 bytes in size.

### SUMMARY

Disclosed herein are polypeptides or polypeptide complexes comprising a peptide that impairs binding of an anti-CD3 binding domain to CD3 wherein the peptide comprises an amino acid sequence according to Peptide-1 (GSQCLGPEWEVCPY) SEQ ID NO: 1 or Peptide-2 (VYCGPEFDESVGCM) SEQ ID NO: 2, or the peptide comprises 1, 2, or 3, amino acid substitutions, additions, or deletions relative to the amino acid sequence of Peptide-1 or Peptide-2. In some embodiments, the peptide comprises the amino acid sequence of Peptide-1. In some embodiments, the peptide comprises the amino acid sequence of Peptide-2. In some embodiments, the peptide is connected to the anti-CD3 binding domain in a configuration according to: A₁-L₁-P₁ (Formula I) wherein A₁ comprises the anti-CD3 binding domain; L₁ comprises a cleavable linker that is a substrate for a tumor specific protease; P₁ comprises the peptide that impairs binding of the anti-CD3 binding domain to CD3. In some embodiments, P₁ is connected N-terminal to the cleavable linker and A₁ is connected C-terminal to the cleavable linker. In some embodiments, P₁ is connected C-terminal to the cleavable linker and A₁ is connected N-terminal to the cleavable linker. In some embodiments, P₁ is bound to A₁ through ionic interactions, electrostatic interactions, hydrophobic interactions, Pi-stacking interactions, or H-bonding interactions, or a combination thereof. In some embodiments, A₁ is further linked to a tumor antigen binding domain (A₂). In some embodiments, the polypeptide or polypeptide complex is according to Formula Ia: P₂-L₂-A₂-A₁-L₁-P₁ wherein P₂ comprises a peptide that impairs binding of A₂ to a tumor antigen; and L₂ comprises a second cleavable linker that connects A₂ to P₂ and is a substrate for a tumor specific protease. In some embodiments, A₁ comprises an antibody or antibody fragment. In some embodiments, A₁ comprises an antibody or antibody fragment that is human or humanized. In some embodiments, L₁ is bound to N-terminus of the antibody or antibody fragment of A₁. In some embodiments, A₂ is bound to N-terminus of the antibody or antibody fragment of A₁. In some embodiments, L₁ is bound to the C-terminus of the antibody or antibody fragment of A₁. In some embodiments, A₂ is bound to the C-terminus of the antibody or antibody fragment of A₁. In some embodiments, the antibody or antibody fragment of A₁ comprises a single chain variable fragment, a single domain antibody, or a Fab fragment. In some embodiments, A₁ is the single chain variable fragment (scFv). In some embodiments, the scFv comprises a scFv heavy chain polypeptide and a scFv light chain polypeptide. In some embodiments, A₁ is the single domain antibody. In some embodiments, the antibody or antibody fragment thereof comprises a single chain variable fragment (scFv), a heavy chain variable domain (VH domain), a light chain variable domain (VL domain), or a variable domain (VHH) of a camelid derived single domain antibody. In some embodiments, A₁ comprises an anti-CD3e single chain variable fragment. In some embodiments, A₁ comprises an anti-CD3e single chain variable fragment that has a KD binding of 1 μM or less to CD3 on CD3 expressing cells. In some embodiments, A₁ comprises a variable light chain and variable heavy chain each of which is capable of specifically binding to human CD3. In some embodiments, A₁ comprises the scFv comprising a scFv heavy chain variable domain and an scFv light chain variable domain comprising complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 3; HC-CDR2: SEQ ID NO: 4; and HC-CDR3: SEQ ID NO: 5; and the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 6; LC-CDR2: SEQ ID NO: 7; and LC-CDR3: SEQ ID NO: 8. In some embodiments, the polypeptide or polypeptide complex of formula I binds to an effector cell when L₁ is cleaved by the tumor specific protease. In some embodiments, the polypeptide or polypeptide complex of formula I binds to an effector cell when L₁ is cleaved by the tumor specific protease and A₁ binds to the effector cell. In some embodiments, the effector cell is a T cell. In some embodiments, A₁ binds to a polypeptide that is part of a TCR-CD3 complex on the effector cell. In some embodiments, the polypeptide that is part of the TCR-CD3 complex is human CD3ε. In some embodiments, A₂ comprises an antibody or antibody fragment. In some embodiments, the antibody or antibody fragment thereof of A₂ comprises a single chain variable fragment, a single domain antibody, Fab′, or a Fab. In some embodiments, the antibody or antibody fragment thereof of A₂ comprises a single chain variable fragment (scFv), a heavy chain variable domain (VH domain), a light chain variable domain (VL domain), or a variable domain (VHH) of a camelid derived single domain antibody. In some embodiments, the antibody or antibody fragment thereof of A₂ is humanized or human. In some embodiments, A₂ is the Fab or Fab′. In some embodiments, the Fab or Fab′ comprises (a) a Fab light chain polypeptide and (b) a Fab heavy chain polypeptide. In some embodiments, A₂ comprises an epidermal growth factor receptor (EGFR) binding domain. In some embodiments, A₂ comprises a carcinoembryonic antigen-related cell adhesion molecule CEACAM5 binding domain. In some embodiments, P₁ is further linked to a half-life extending moiety (H₁). In some embodiments, the half-life extending moiety is a single-domain antibody. In some embodiments, H₁ comprises a polymer. In some embodiments, the polymer is polyethylene glycol (PEG). In some embodiments, H₁ comprises albumin. In some embodiments, H₁ comprises an Fc domain. In some embodiments, the albumin is serum albumin. In some embodiments, the albumin is human serum albumin. In some embodiments, H₁ comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin comprises IgGl, IgG2, IgG3, IgG4, slgA, IgM or IgD. In some embodiments, the serum protein is albumin. In some embodiments, the polypeptide is an antibody. In some embodiments, the antibody comprises a single domain antibody, a single chain variable fragment, or a Fab. In some embodiments, the single domain antibody comprises a single domain antibody that binds to albumin. In some embodiments, the single domain antibody is a human antibody or humanized antibody. In some embodiments, the single domain antibody is 645gH1gL1. In some embodiments, the single domain antibody is 645dsgH5gL4. In some embodiments, the single domain antibody is 23-13-A01-sc02. In some embodiments, the single domain antibody is A10m3 or a fragment thereof. In some embodiments, the single domain antibody is DOM7r-31. In some embodiments, the single domain antibody is Alb-1, Alb-8, or Alb-23. In some embodiments, the single domain antibody is 10G or 10GE. In some embodiments, the single domain antibody is SA21. In some embodiments, the polypeptide or polypeptide complex comprises a modified amino acid, a non-natural amino acid, a modified non-natural amino acid, or a combination thereof. In some embodiments, the modified amino acid or modified non-natural amino acid comprises a post-translational modification. In some embodiments, H₁ comprises a linking moiety (L₃) that connects H₁ to P₁. In some embodiments, L₃ is a peptide sequence having at least 5 to no more than 50 amino acids. In some embodiments, L₃ is a peptide sequence having at least 10 to no more than 30 amino acids. In some embodiments, L₃ is a peptide sequence having at least 10 amino acids. In some embodiments, L₃ is a peptide sequence having at least 18 amino acids. In some embodiments, L₃ is a peptide sequence having at least 26 amino acids. In some embodiments, L₃ has a formula selected from the group consisting of (G₂S)ₙ, (GS)ₙ, (GSGGS)ₙ (SEQ ID NO: 917), (GGGS)ₙ (SEQ ID NO: 918), (GGGGS)ₙ (SEQ ID NO: 919), and (GSSGGS)ₙ (SEQ ID NO: 920), wherein n is an integer of at least 1. In some embodiments, L₃ comprises an amino acid sequence according to (GGGGSGGGS (SEQ ID NO: 921)). In some embodiments, the Fab light chain polypeptide of A₂ is bound to a C-terminus of the single chain variable fragment (scFv) of A₁. In some embodiments, the Fab heavy chain polypeptide of A₂ is bound to a C-terminus of the single chain variable fragment (scFv) A₁. In some embodiments, the Fab light chain polypeptide of A₂ is bound to a N-terminus of the single chain variable fragment (scFv) of A₁. In some embodiments, the Fab heavy chain polypeptide of A₂ is bound to a N-terminus of the single chain variable fragment (scFv) A₁. In some embodiments, the Fab heavy chain polypeptide of A₂ is bound to the scFv heavy chain polypeptide of A₁. In some embodiments, the Fab light chain polypeptide of A₂ is bound to the scFv heavy chain polypeptide of A₁. In some embodiments, the Fab heavy chain polypeptide of A₂ is bound to the scFv light chain polypeptide of A₁. In some embodiments, the Fab light chain polypeptide of A₂ is bound to the scFv light chain polypeptide of A₁. In some embodiments, the Fab heavy chain polypeptide of A₂ is bound to the scFv heavy chain polypeptide of A₁ and L₂ is bound to the Fab light chain polypeptide of A₂. In some embodiments, the Fab light chain polypeptide of A₂ is bound to the scFv heavy chain polypeptide of A₁ and L₂ is bound to the Fab heavy chain polypeptide of A₂. In some embodiments, the Fab heavy chain polypeptide of A₂ is bound to the scFv light chain polypeptide of A₁ and L₂ is bound to the Fab light chain polypeptide of A₂. In some embodiments, the Fab light chain polypeptide of A₂ is bound to the scFv light chain polypeptide of A₁ and L₂ is bound to the Fab heavy chain polypeptide of A₂. In some embodiments, P₂ impairs binding of A₂ to the tumor antigen. In some embodiments, P₂ is bound to A₂ through ionic interactions, electrostatic interactions, hydrophobic interactions, Pi-stacking interactions, or H-bonding interactions, or a combination thereof. In some embodiments, P₂ is bound to A₂ at or near an antigen binding site. In some embodiments, P₂ has less than 70% sequence homology to the tumor antigen. In some embodiments, P₂ comprises a peptide sequence of at least 10 amino acids in length. In some embodiments, P₂ comprises a peptide sequence of at least 10 amino acids in length and no more than 20 amino acids in length. In some embodiments, P₂ comprises a peptide sequence of at least 16 amino acids in length. In some embodiments, P₂ comprises a peptide sequence of no more than 40 amino acids in length. In some embodiments, P₂ comprises at least two cysteine amino acid residues. In some embodiments, P₂ comprises a cyclic peptide or a linear peptide. In some embodiments, P₂ comprises a cyclic peptide. In some embodiments, P₂ comprises a linear peptide. In some embodiments, L₁ is bound to N-terminus of A₁. In some embodiments, L₁ is bound to C-terminus of A₁. In some embodiments, L₂ is bound to N-terminus of A₂. In some embodiments, L₂ is bound to C-terminus of A₂. In some embodiments, L₁ or L₂ is a peptide sequence having at least 5 to no more than 50 amino acids. In some embodiments, L₁ or L₂ is a peptide sequence having at least 10 to no more than 30 amino acids. In some embodiments, L₁ or L₂ is a peptide sequence having at least 10 amino acids. In some embodiments, L₁ or L₂ is a peptide sequence having at least 18 amino acids. In some embodiments, L₁ or L₂ is a peptide sequence having at least 26 amino acids. In some embodiments, L₁ or L₂ has a formula comprising (G₂S)ₙ (SEQ ID NO: 922), wherein n is an integer from 1 to 3. In some embodiments, L₁ has a formula selected from the group consisting of (G₂S)ₙ, (GS)ₙ, (GSGGS)ₙ (SEQ ID NO: 917), (GGGS)ₙ (SEQ ID NO: 918), (GGGGS)ₙ (SEQ ID NO: 919), and (GSSGGS)ₙ (SEQ ID NO: 920), wherein n is an integer of at least 1. In some embodiments, P₁ becomes unbound from A₁ when L₁ is cleaved by the tumor specific protease thereby exposing A₁ to CD3. In some embodiments, P₂ becomes unbound from A₂ when L₂ is cleaved by the tumor specific protease thereby exposing A₂ to the tumor antigen. In some embodiments, the tumor specific protease is selected from the group consisting of metalloprotease, serine protease, cysteine protease, threonine protease, and aspartic protease. In some embodiments, L₁ or L₂ comprises a urokinase cleavable amino acid sequence, a matriptase cleavable amino acid sequence, a matrix metalloprotease cleavable amino acid sequence, or a legumain cleavable amino acid sequence. In some embodiments, L₁ or L₂ comprises an amino acid sequence selected from the group consisting of GGGGSLSGRSDNHGSSGT (SEQ ID NO: 923), GGGGSSGGSGGSGLSGRSDNHGSSGT (SEQ ID NO: 924), ASGRSDNH (SEQ ID NO: 925), LAGRSDNH (SEQ ID NO: 926), ISSGLASGRSDNH (SEQ ID NO: 927), ISSGLLAGRSDNH (SEQ ID NO: 928), LSGRSDNH (SEQ ID NO: 929), ISSGLLSGRSDNP (SEQ ID NO: 930), ISSGLLSGRSDNH (SEQ ID NO: 931), LSGRSDNHSPLGLAGS (SEQ ID NO: 932), SPLGLAGSLSGRSDNH (SEQ ID NO: 933), SPLGLSGRSDNH (SEQ ID NO: 934), LAGRSDNHSPLGLAGS (SEQ ID NO: 935), LSGRSDNHVPLSLKMG (SEQ ID NO: 936), and LSGRSDNHVPLSLSMG (SEQ ID NO: 937). In some embodiments, L₁ or L₂ comprises an amino acid sequence selected from the group consisting of ASGRSDNH (SEQ ID NO: 925), LAGRSDNH (SEQ ID NO: 926), ISSGLASGRSDNH (SEQ ID NO: 927), and ISSGLLAGRSDNH (SEQ ID NO: 928).

Disclosed herein are pharmaceutical compositions comprising (i) the polypeptides or polypeptide complexes of any one of the previous embodiments; and (ii) a pharmaceutically acceptable excipient.

Disclosed herein are isolated recombinant nucleic acid molecules encoding the polypeptides or polypeptide complexes of any one of the previous embodiments.

Disclosed herein are isolated polypeptides or polypeptide complexes according to Formula II: L₁ₐ-P₁ₐ-H₁ₐ wherein: L₁ₐ comprises a tumor specific protease-cleaved linking moiety that when uncleaved connects P₁ₐ to an anti-CD3 binding domain that binds to CD3 and; P₁ₐ comprises a peptide that impairs binding of the anti-CD3 binding domain to CD3 when L₁ₐ is uncleaved wherein the peptide comprises an amino acid sequence according to Peptide-1 (GSQCLGPEWEVCPY) SEQ ID NO: 1 or Peptide-2 (VYCGPEFDESVGCM) SEQ ID NO: 2, or the peptide comprises 1, 2, or 3, amino acid substitutions, additions, or deletions relative to the amino acid sequence of Peptide-1 or Peptide-2; and H₁ₐ comprises a half-life extending molecule. In some embodiments, the peptide comprises the amino acid sequence of Peptide-1. In some embodiments, the peptide comprises the amino acid sequence of Peptide-2. In some embodiments, H₁ₐ comprises a polymer. In some embodiments, the polymer is polyethylene glycol (PEG). In some embodiments, H₁ₐ comprises albumin. In some embodiments, H₁ₐ comprises an Fc domain. In some embodiments, the albumin is serum albumin. In some embodiments, the albumin is human serum albumin. In some embodiments, H₁ₐ comprises a polypeptide, a ligand, or a small molecule. In some embodiments, the polypeptide, the ligand or the small molecule binds a serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgGl, IgG2, IgG3, IgG4, slgA, IgM or IgD. In some embodiments, the serum protein is albumin. In some embodiments, the polypeptide is an antibody. In some embodiments, the antibody comprises a single domain antibody, a single chain variable fragment or a Fab. In some embodiments, the antibody comprises a single domain antibody that binds to albumin. In some embodiments, the antibody is a human or humanized antibody. In some embodiments, the single domain antibody is 645gH1gL1. In some embodiments, the single domain antibody is 645dsgH5gL4. In some embodiments, the single domain antibody is 23-13-A01-sc02. In some embodiments, the single domain antibody is A10m3 or a fragment thereof. In some embodiments, the single domain antibody is DOM7r-31. In some embodiments, the single domain antibody is DOM7h-11-15. In some embodiments, the single domain antibody is Alb-1, Alb-8, or Alb-23. In some embodiments, the single domain antibody is 10G or 10GE. In some embodiments, the single domain antibody is SA21. In some embodiments, H₁ₐ comprises a linking moiety (L₃ₐ) that connects H₁ₐ to P₁ₐ. In some embodiments, L₃ₐ is a peptide sequence having at least 5 to no more than 50 amino acids.

In some embodiments, L₃ₐ is a peptide sequence having at least 10 to no more than 30 amino acids. In some embodiments, L₃ₐ is a peptide sequence having at least 10 amino acids. In some embodiments, L₃ₐ is a peptide sequence having at least 18 amino acids. In some embodiments, L₃ₐ is a peptide sequence having at least 26 amino acids. In some embodiments, L₃ₐ has a formula selected from the group consisting of (G₂S)ₙ, (GS)ₙ, (GSGGS)ₙ (SEQ ID NO: 917), (GGGS)ₙ (SEQ ID NO: 918), (GGGGS)ₙ (SEQ ID NO: 919), and (GSSGGS)ₙ (SEQ ID NO: 920), wherein n is an integer of at least 1. In some embodiments, L₃ₐ comprises an amino acid sequence according to (GGGGSGGGS (SEQ ID NO: 921)).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates Peptide-1 peptide binding data to SP34.185 scFv by ELISA.

FIG. 1B illustrates Peptide-1 alanine scanning peptides binding data to SP34.185 scFv by ELISA.

FIG. 1C illustrates Peptide-1 alanine scanning peptides binding data to SP34.185 scFv by ELISA.

FIG. 1D illustrates Peptide-2 peptide binding data to SP34.185 scFv by ELISA.

FIG. 1E illustrates Peptide-2 alanine scanning peptides binding data to SP34.185 scFv by ELISA.

FIG. 1F illustrates Peptide-2 alanine scanning peptides binding data to SP34.185 scFv by ELISA.

FIG. 2A illustrates Peptide-1 inhibitory data of SP34.185 binding to CD3e.

FIG. 2B illustrates Peptide-1 alanine scanning peptides inhibitory data of SP34.185 binding to CD3e.

FIG. 2C illustrates Peptide-1 alanine scanning peptides inhibitory data of SP34.185 binding to CD3e.

FIG. 2D illustrates Peptide-2 inhibitory data of SP34.185 binding to CD3e.

FIG. 2E illustrates Peptide-2 alanine scanning peptides inhibitory data of SP34.185 binding to CD3e.

FIG. 2F illustrates Peptide-2 alanine scanning peptides inhibitory data of SP34.185 binding to CD3e.

FIG. 3 illustrates titration data for CD3R binding for several polypeptide complexes of this disclosure.

FIG. 4 illustrates functional tumor cell killing of PC-1, PC-2, and PC-3.

FIG. 5A illustrates peptides (Peptide-29-Peptide-34) peptide binding data to SP34.185 scFv by ELISA. Peptide-2 is used as a positive control.

FIG. 5B illustrates peptides (Peptide-35-Peptide-40) peptide binding data to SP34.185 scFv by ELISA. Peptide-2 is used as a positive control.

FIG. 6A illustrates peptides (Peptide-29-Peptide-34) inhibiting binding of SP34.185 scFv to CD3 by ELISA. Peptide-2 is used as a positive control.

FIG. 6B illustrates peptides (Peptide-35-Peptide-40) inhibiting binding of SP34.185 scFv to CD3 by ELISA. Peptide-2 is used as a positive control.

FIG. 7 illustrates the core sequence motif of Peptide-2 generated by WebLogo 3.

### DETAILED DESCRIPTION

Protein-based therapies such as antibodies and bispecific or multispecific antibodies, such as T cell engagers, have proven effective for a variety diseases and disorders. As with any therapy, there is a need to minimize off-target effects of the protein-based therapy in healthy tissue while maintaining activity of the protein-based therapy in disease tissue. One such strategy is to create an inactive form of the protein-based therapy in which a necessary binding site on the protein-based therapy is blocked with a peptide linked to the protein-based therapy, thereby preventing the protein-based therapy from binding or interacting with its cognate receptor or target antigen when in healthy tissue. For activating the protein-based therapy in the desired disease-state microenvironment, the peptide is linked to the protein-based therapy with a linker that is cleavable by a protease that is specific to the disease-state microenvironment. The peptide is then released from the protein-based therapy when in the disease-state microenvironment.

Accordingly, disclosed herein, are peptides that impair binding of anti-CD3 binding domains to cluster of differentiation 3 (CD3). The peptides as disclosed herein can be applied to a variety of antibody formats that bind to anti-CD3 to reduce the off-target effects of the antibodies in healthy tissue, while maintaining activity of the anti-CD3 antibody in a disease tissue. The peptides are attached to the anti-CD3 binding domains via protease cleavable linkers. In some embodiments, the antibodies described herein are used in a method of treating cancer. In some instances, the cancer is a solid tumor cancer. In some embodiments, the cancer is lung, breast (e.g. HER2+; ER/PR+; TNBC), cervical, ovarian, colorectal, pancreatic or gastric.

Disclosed herein are isolated polypeptides or polypeptide complexes comprising a peptide that impairs binding of an anti-CD3 binding domain to CD3 wherein the peptide comprises an amino acid sequence according to Peptide-1 (GSQCLGPEWEVCPY) SEQ ID NO: 1 or Peptide-2 (VYCGPEFDESVGCM) SEQ ID NO: 2, or the peptide comprises 1, 2, or 3, amino acid substitutions, additions, or deletions relative to the amino acid sequence of Peptide-1 or Peptide-2.

Disclosed herein are isolated polypeptides or polypeptide complexes comprising a peptide that impairs binding of an anti-CD3 binding domain to CD3 wherein the peptide comprises an amino acid sequence selected from SEQ ID NOs: 1-2 and 19-55 or the peptide comprises 1, 2, or 3, amino acid substitutions, additions, or deletions relative to an amino acid sequence selected from SEQ ID NOs: 1-2 and 19-55. In some embodiments, the peptide comprises an amino acid sequence selected from SEQ ID NOs: 1-2, and 19-55. In some embodiments, the peptide comprises the amino acid sequence of SEQ ID NO: 1. In some embodiments, the peptide comprises the amino acid sequence of SEQ ID NO: 2. In some embodiments, the peptide comprises the amino acid sequence of SEQ ID NO: 54.

Disclosed herein are isolated polypeptides or polypeptide complexes comprising an anti-CD3 binding domain that is linked to a peptide that impairs binding of the anti-CD3 binding domain to CD3 wherein the peptide comprises an amino acid sequence according to Z₁-Z₂-C-Z₄-P-Z₆-Z₇-Z₈-Z₉-Z₁₀-Z₁₁-Z₁₂-C-Z₁₄ and Z₁ is selected from D, Y, F, I, N, V, H, L, A, T, S, and P; Z₂ is selected from D, Y, L, F, I, N, A, V, H, T, and S; Z₄ is selected from G and W; Z₆ is selected from E, D, V, and P; Z₇ is selected from W, L, F, V, G, M, I, and Y; Z₈ is selected from E, D, P, and Q; Z₉ is selected from E, D, Y, V, F, W, P, L, and Q; Z₁₀ is selected from S, D, Y, T, I, F, V, N, A, P, L, and H; Z₁₁ is selected from I, Y, F, V, L, T, N, S, D, A, and H; Z₁₂ is selected from F, D, Y, L, I, V, A, N, T, P, S, and H; and Z₁₄ is selected from D, Y, N, F, I, P, V, A, T, H, L and S. In some embodiments, Z₁ is selected from D, Y, F, I, and N; Z₂ is selected from D, Y, L, F, I, and N; Z₄ is selected from G and W; Z₆ is selected from E and D; Z₇ is selected from W, L, F, and V; Z₈ is selected from E and D; Z₉ is selected from E, D, Y, and V; Z₁₀ is selected from S, D, Y, T, and I; Z₁₁ is selected from I, Y, F, V, L, and T; Z₁₂ is selected from F, D, Y, L, I, V, A, and N; and Z₁₄ is selected from D, Y, N, F, I, and P. In some embodiments, Z₁ is selected from D, Y, and F; Z₂ is selected from D, Y, L, and F; Z₄ is selected from G and W; Z₆ is selected from E and D; Z₇ is selected from W, L, and F; Z₈ is selected from E and D; Z₉ is selected from E and D; Z₁₀ is selected from S, D, and Y; Z₁₁ is selected from I, Y, and F; Z₁₂ is selected from F, D, Y, and L; and Z₁₄ is selected from D, Y, and N. In some embodiments, the peptide comprises an amino acid sequence of Table 17. In some embodiments, the peptide comprises an amino acid sequence according to any one of SEQ ID NOs: 44-55. In some embodiments, the peptide comprises the amino acid sequence of SEQ ID NO: 2. In some embodiments, the peptide comprises the amino acid sequence of SEQ ID NO: 54.

Disclosed herein are isolated polypeptides or polypeptide complexes comprising an anti-CD3 binding domain that is linked to a peptide that impairs binding of the anti-CD3 binding domain to CD3 wherein the peptide comprises an amino acid sequence according to U₁-U₂-C-U₄-P-U₆-U₇-U₈-U₉-U₁₀-U₁₁-U₁₂-C-U₁₄ and U₁ is selected from D, Y, F, I, N, V, H, L, A, T, S, and P; U₂ is selected from D, Y, L, F, I, N, A, V, H, T, and S; U₄ is selected from G and W; U₆ is selected from E, D, V, and P; U₇ is selected from W, L, F, V, G, M, I, and Y; U₈ is selected from E, D, P, and Q; U₉ is selected from E, D, Y, V, F, W, P, L, and Q; U₁₀ is selected from S, D, Y, T, I, F, V, N, A, P, L, and H; U₁₁ is selected from I, Y, F, V, L, T, N, S, D, A, and H; U₁₂ is selected from F, D, Y, L, I, V, A, N, T, P, S, G, and H; and U₁₄ is selected from D, Y, N, F, I, P, V, A, T, H, L, M, and S. In some embodiments, U₁ is selected from D, Y, F, I, V, and N; U₂ is selected from D, Y, L, F, I, and N; U₄ is selected from G and W; U₆ is selected from E and D; U₇ is selected from W, L, F, G, and V; U₈ is selected from E and D; U₉ is selected from E, D, Y, and V; U₁₀ is selected from S, D, Y, T, and I; U₁₁ is selected from I, Y, F, V, L, and T; U₁₂ is selected from F, D, Y, L, I, V, A, G, and N; and U₁₄ is selected from D, Y, N, F, I, M, and P. In some embodiments, U₁ is selected from D, Y, V, and F; U₂ is selected from D, Y, L, and F; U₄ is selected from G and W; U₆ is selected from E and D; U₇ is selected from W, L, G, and F; U₈ is selected from E and D; U₉ is selected from E and D; U₁₀ is selected from S, D, T, and Y; U₁₁ is selected from I, Y, V, L, and F; U₁₂ is selected from F, D, Y, G, A, and L; and U₁₄ is selected from D, Y, M, and N. In some embodiments, the peptide comprises an amino acid sequence of Table 17. In some embodiments, the peptide comprises an amino acid sequence according to any one of SEQ ID NOs: 44-55. In some embodiments, the peptide comprises the amino acid sequence of SEQ ID NO: 2. In some embodiments, the peptide comprises the amino acid sequence of SEQ ID NO: 54.

Disclosed herein, are methods of treating cancer comprising administering to a subject in need thereof an isolated polypeptide or polypeptide complex comprising a peptide that impairs binding of an anti-CD3 binding domain to CD3 wherein the peptide comprises an amino acid sequence according to Peptide-1 (GSQCLGPEWEVCPY) SEQ ID NO: 1 or Peptide-2 (VYCGPEFDESVGCM) SEQ ID NO: 2, or the peptide comprises 1, 2, or 3, amino acid substitutions, additions, or deletions relative to the amino acid sequence of Peptide-1 or Peptide-2.

Disclosed herein, are methods of treating cancer comprising administering to a subject in need thereof an isolated polypeptide or polypeptide complex comprising a peptide that impairs binding of an anti-CD3 binding domain to CD3 wherein the peptide comprises an amino acid sequence selected from SEQ ID NOs: 1-2, and 19-55 or the peptide comprises 1, 2, or 3, amino acid substitutions, additions, or deletions relative to amino acid sequences of SEQ ID NOs: 1-2, and 19-55. In some embodiments, the peptide comprises an amino acid sequence selected from SEQ ID NOs: 1-2, and 19-55. In some embodiments, the peptide comprises the amino acid sequence of SEQ ID NO: 1. In some embodiments, the peptide comprises the amino acid sequence of SEQ ID NO: 2. In some embodiments, the peptide comprises the amino acid sequence of SEQ ID NO: 54.

Disclosed herein, are methods of treating cancer comprising administering to a subject in need thereof an isolated polypeptide or polypeptide complex comprising an anti-CD3 binding domain that is linked to a peptide that impairs binding of the anti-CD3 binding domain to CD3 wherein the peptide comprises an amino acid sequence according to Z₁-Z₂-C-Z₄-P-Z₆-Z₇-Z₈-Z₉-Z₁₀-Z₁₁-Z₁₂-C-Z₁₄ and Z₁ is selected from D, Y, F, I, N, V, H, L, A, T, S, and P; Z₂ is selected from D, Y, L, F, I, N, A, V, H, T, and S; Z₄ is selected from G and W; Z₆ is selected from E, D, V, and P; Z₇ is selected from W, L, F, V, G, M, I, and Y; Z₈ is selected from E, D, P, and Q; Z₉ is selected from E, D, Y, V, F, W, P, L, and Q; Z₁₀ is selected from S, D, Y, T, I, F, V, N, A, P, L, and H; Z₁₁ is selected from I, Y, F, V, L, T, N, S, D, A, and H; Z₁₂ is selected from F, D, Y, L, I, V, A, N, T, P, S, and H; and Z₁₄ is selected from D, Y, N, F, I, P, V, A, T, H, L and S. In some embodiments, Z₁ is selected from D, Y, F, I, and N; Z₂ is selected from D, Y, L, F, I, and N; Z₄ is selected from G and W; Z₆ is selected from E and D; Z₇ is selected from W, L, F, and V; Z₈ is selected from E and D; Z₉ is selected from E, D, Y, and V; Z₁₀ is selected from S, D, Y, T, and I; Z₁₁ is selected from I, Y, F, V, L, and T; Z₁₂ is selected from F, D, Y, L, I, V, A, and N; and Z₁₄ is selected from D, Y, N, F, I, and P. In some embodiments, Z₁ is selected from D, Y, and F; Z₂ is selected from D, Y, L, and F; Z₄ is selected from G and W; Z₆ is selected from E and D; Z₇ is selected from W, L, and F; Z₈ is selected from E and D; Z₉ is selected from E and D; Z₁₀ is selected from S, D, and Y; Z₁₁ is selected from I, Y, and F; Z₁₂ is selected from F, D, Y, and L; and Z₁₄ is selected from D, Y, and N. In some embodiments, the peptide comprises an amino acid sequence of Table 17. In some embodiments, the peptide comprises an amino acid sequence according to any one of SEQ ID NOs: 44-55. In some embodiments, the peptide comprises the amino acid sequence of SEQ ID NO: 2. In some embodiments, the peptide comprises the amino acid sequence of SEQ ID NO: 54.

Disclosed herein, are methods of treating cancer comprising administering to a subject in need thereof an isolated polypeptide or polypeptide complex comprising an anti-CD3 binding domain that is linked to a peptide that impairs binding of the anti-CD3 binding domain to CD3 wherein the peptide comprises an amino acid sequence according to U₁-U₂-C-U₄-P-U₆-U₇-U₈-U₉-U₁₀-U₁₁-U₁₂-C-U₁₄ and U₁ is selected from D, Y, F, I, N, V, H, L, A, T, S, and P; U₂ is selected from D, Y, L, F, I, N, A, V, H, T, and S; U₄ is selected from G and W; U₆ is selected from E, D, V, and P; U₇ is selected from W, L, F, V, G, M, I, and Y; U₈ is selected from E, D, P, and Q; U₉ is selected from E, D, Y, V, F, W, P, L, and Q; U₁₀ is selected from S, D, Y, T, I, F, V, N, A, P, L, and H; U₁₁ is selected from I, Y, F, V, L, T, N, S, D, A, and H; U₁₂ is selected from F, D, Y, L, I, V, A, N, T, P, S, G, and H; and U₁₄ is selected from D, Y, N, F, I, P, V, A, T, H, L, M, and S. In some embodiments, U₁ is selected from D, Y, F, I, V, and N; U₂ is selected from D, Y, L, F, I, and N; U₄ is selected from G and W; U₆ is selected from E and D; U₇ is selected from W, L, F, G, and V; U₈ is selected from E and D; U₉ is selected from E, D, Y, and V; U₁₀ is selected from S, D, Y, T, and I; U₁₁ is selected from I, Y, F, V, L, and T; U₁₂ is selected from F, D, Y, L, I, V, A, G, and N; and U₁₄ is selected from D, Y, N, F, I, M, and P. In some embodiments, U₁ is selected from D, Y, V, and F; U₂ is selected from D, Y, L, and F; U₄ is selected from G and W; U₆ is selected from E and D; U₇ is selected from W, L, G, and F; U₈ is selected from E and D; U₉ is selected from E and D; U₁₀ is selected from S, D, T, and Y; U₁₁ is selected from I, Y, V, L, and F; U₁₂ is selected from F, D, Y, G, A, and L; and U₁₄ is selected from D, Y, M, and N. In some embodiments, the peptide comprises an amino acid sequence of Table 17. In some embodiments, the peptide comprises an amino acid sequence according to any one of SEQ ID NOs: 44-55. In some embodiments, the peptide comprises the amino acid sequence of SEQ ID NO: 2. In some embodiments, the peptide comprises the amino acid sequence of SEQ ID NO: 54.

In some embodiments, the peptide is connected to the anti-CD3 binding domain in a configuration according to: A₁-L₁-P₁(Formula I) wherein A₁ comprises the anti-CD3 binding domain; L₁ comprises a cleavable linker that is a substrate for a tumor specific protease; P₁ comprises the peptide that impairs binding of the anti-CD3 binding domain to CD3. In some embodiments, the peptide is connected to the anti-CD3 binding domain in a configuration according to: A₁-L₁-P₁ (Formula I) wherein A₁ is the anti-CD3 binding domain; L₁ is a cleavable linker that is a substrate for a tumor specific protease; P₁ is the peptide that impairs binding of the anti-CD3 binding domain to CD3. In some embodiments, the peptide is connected to the anti-CD3 binding domain in a configuration comprising Formula I: A₁-L₁-P₁ (Formula I) wherein A₁ comprises the anti-CD3 binding domain; L₁ comprises a cleavable linker that is a substrate for a tumor specific protease; P₁ comprises the peptide that impairs binding of the anti-CD3 binding domain to CD3. In some embodiments, the peptide is connected to the anti-CD3 binding domain in a configuration comprising Formula I: A₁-L₁-P₁ (Formula I) wherein A₁ is the anti-CD3 binding domain; L₁ is a cleavable linker that is a substrate for a tumor specific protease; P₁ is the peptide that impairs binding of the anti-CD3 binding domain to CD3. In some embodiments, A₁ is further linked to a tumor antigen binding domain (A₂). In some embodiments, the polypeptide or polypeptide complex is according to Formula Ia: P₂-L₂-A₂-A₁-L₁-P₁ (Formula Ia) wherein P₂ comprises a peptide that impairs binding of A₂ to a tumor antigen; and L₂ comprises a second cleavable linker that connects A₂ to P₂ and is a substrate for a tumor specific protease.

In some embodiments, P₁ is connected N-terminal to the cleavable linker and A₁ is connected C-terminal to the cleavable linker. In some embodiments, P₁ is connected C-terminal to the cleavable linker and A₁ is connected N-terminal to the cleavable linker. In some embodiments, P₁ is bound to A₁ through ionic interactions, electrostatic interactions, hydrophobic interactions, Pi-stacking interactions, or H-bonding interactions, or a combination thereof.

Disclosed herein are isolated polypeptides or polypeptide complexes according to Formula II: L₁ₐ-P₁ₐ-H₁ₐ (Formula II) wherein: L₁ₐ comprises a tumor specific protease-cleaved linking moiety that when uncleaved connects P₁ₐ to an anti-CD3 binding domain that binds to CD3 and; P₁ₐ comprises a peptide that impairs binding of the anti-CD3 binding domain to CD3 when L₁ₐ is uncleaved wherein the peptide comprises an amino acid sequence selected from SEQ ID NOs: 1-2, 19-55 or the peptide comprises 1, 2, or 3, amino acid substitutions, additions, or deletions relative to amino acid sequences of SEQ ID NOs: 1-2, 19-55; and H₁ₐ comprises a half-life extending molecule. In some embodiments, P₁ₐ comprises an amino acid sequence selected from SEQ ID NOs: 1-2, and 19-55. In some embodiments, P₁ₐ comprises the amino acid sequence of SEQ ID NO: 1. In some embodiments, P₁ₐ comprises the amino acid sequence of SEQ ID NO: 2. In some embodiments, P₁ₐ comprises the amino acid sequence of SEQ ID NO: 54.

Disclosed herein are isolated polypeptides or polypeptide complexes according to Formula III: L_{1b}-P_{1b}-H_{1b} wherein: L_{1b} comprises a tumor specific protease-cleaved linking moiety that when uncleaved connects P_{1b} to an anti-CD3 binding domain that binds to CD3 and; P_{1b} comprises a peptide that impairs binding of the anti-CD3 binding domain to CD3 when L_{1b} is uncleaved wherein the peptide comprises the amino acid sequence according to Z₁-Z₂-C-Z₄-P-Z₆-Z₇-Z₈-Z₉-Z₁₀-Z₁₁-Z₁₂-C-Z₁₄ and Z₁ is selected from D, Y, F, I, N, V, H, L, A, T, S, and P; Z₂ is selected from D, Y, L, F, I, N, A, V, H, T, and S; Z₄ is selected from G and W; Z₆ is selected from E, D, V, and P; Z₇ is selected from W, L, F, V, G, M, I, and Y; Z₈ is selected from E, D, P, and Q; Z₉ is selected from E, D, Y, V, F, W, P, L, and Q; Z₁₀ is selected from S, D, Y, T, I, F, V, N, A, P, L, and H; Z₁₁ is selected from I, Y, F, V, L, T, N, S, D, A, and H; Z₁₂ is selected from F, D, Y, L, I, V, A, N, T, P, S, and H; and Z₁₄ is selected from D, Y, N, F, I, P, V, A, T, H, L and S; and H_{1b} comprises a half-life extending molecule. In some embodiments, Z₁ is selected from D, Y, F, I, and N; Z₂ is selected from D, Y, L, F, I, and N; Z₄ is selected from G and W; Z₆ is selected from E and D; Z₇ is selected from W, L, F, and V; Z₈ is selected from E and D; Z₉ is selected from E, D, Y, and V; Z₁₀ is selected from S, D, Y, T, and I; Z₁₁ is selected from I, Y, F, V, L, and T; Z₁₂ is selected from F, D, Y, L, I, V, A, and N; and Z₁₄ is selected from D, Y, N, F, I, and P. In some embodiments, Z₁ is selected D, Y, and F; Z₂ is selected from D, Y, L, and F; Z₄ is selected from G and W; Z₆ is selected from E and D; Z₇ is selected from W, L, and F; Z₈ is selected from E and D; Z₉ is selected from E and D; Z₁₀ is selected from S, D, and Y; Z₁₁ is selected from I, Y, and F; Z₁₂ is selected from F, D, Y, and L; Z₁₄ is selected from D, Y, and N. In some embodiments, P_{1b} comprises an amino acid sequence of Table 17. In some embodiments, P_{1b} comprises an amino acid sequences according to any one of SEQ ID NOs: 44-55. In some embodiments, P_{1b} comprises the amino acid sequences according to SEQ ID NO: 2. In some embodiments, P_{1b} comprises the amino acid sequences according to SEQ ID NO: 54.

Disclosed herein are isolated polypeptide or polypeptide complex according to Formula IV: L_{1c}-P_{1c}-H_{1c} wherein: L_{1c} comprises a tumor specific protease-cleaved linking moiety that when uncleaved connects P_{1c} to an anti-CD3 binding domain that binds to CD3 and; P_{1c} comprises a peptide that impairs binding of the anti-CD3 binding domain to CD3 when L_{1b} is uncleaved wherein the peptide comprises the amino acid sequence according to U₁-U₂-C-U₄-P-U₆-U₇-U₈-U₉-U₁₀-U₁₁-U₁₂-C-U₁₄ and U₁ is selected from D, Y, F, I, N, V, H, L, A, T, S, and P; U₂ is selected from D, Y, L, F, I, N, A, V, H, T, and S; U₄ is selected from G and W; U₆ is selected from E, D, V, and P; U₇ is selected from W, L, F, V, G, M, I, and Y; U₈ is selected from E, D, P, and Q; U₉ is selected from E, D, Y, V, F, W, P, L, and Q; U₁₀ is selected from S, D, Y, T, I, F, V, N, A, P, L, and H; U₁₁ is selected from I, Y, F, V, L, T, N, S, D, A, and H; U₁₂ is selected from F, D, Y, L, I, V, A, N, T, P, S, G, and H; U₁₄ is selected from D, Y, N, F, I, P, V, A, T, H, L, M, and S; and H_{1c} comprises a half-life extending molecule. In some embodiments, U₁ is selected from D, Y, F, I, V, and N; U₂ is selected from D, Y, L, F, I, and N; U₄ is selected from G and W; U₆ is selected from E and D; U₇ is selected from W, L, F, G, and V; U₈ is selected from E and D; U₉ is selected from E, D, Y, and V; U₁₀ is selected from S, D, Y, T, and I; U₁₁ is selected from I, Y, F, V, L, and T; U₁₂ is selected from F, D, Y, L, I, V, A, G, and N; U₁₄ is selected from D, Y, N, F, I, M, and P. In some embodiments, U₁ is selected from D, Y, V, and F; U₂ is selected from D, Y, L, and F; U₄ is selected from G and W; U₆ is selected from E and D; U₇ is selected from W, L, G, and F; U₈ is selected from E and D; U₉ is selected from E and D; U₁₀ is selected from S, D, T, and Y; U₁₁ is selected from I, Y, V, L, and F; U₁₂ is selected from F, D, Y, G, A, and L; U₁₄ is selected from D, Y, M, and N. In some embodiments, P_{1c} comprises comprises an amino acid sequence of Table 17. In some embodiments, P_{1c} comprises an amino acid sequences according to any one of SEQ ID NOs: 44-55. In some embodiments, P_{1c} comprises the amino acid sequences according to SEQ ID NO: 2. In some embodiments, P_{1c} comprises the amino acid sequences according to SEQ ID NO: 54.

In some embodiments, the peptide (P₁, P₁ₐ, P_{1b}, or P_{1c}) comprises the amino acid sequence of Peptide-1.

In some embodiments, the peptide (P₁, P₁ₐ, P_{1b}, or P_{1c}) comprises the amino acid sequence of Peptide-2.

In some embodiments, the peptide (P₁, P₁ₐ, P_{1b} or P_{1c}) comprises an amino acid sequence that has 1 amino acid substitution relative to Peptide-1. In some embodiments, the peptide (P₁, P₁ₐ, P_{1b} or P_{1c}) comprises an amino acid sequence that has 2 amino acid substitution relative to Peptide-1. In some embodiments, the peptide (P₁, P₁ₐ, P_{1b} or P_{1c}) comprises an amino acid sequence that has 3 amino acid substitution relative to Peptide-1.

In some embodiments, the peptide (P₁, P₁ₐ, P_{1b} or P_{1c}) comprises an amino acid sequence that has 1 amino acid substitution relative to Peptide-2. In some embodiments, the peptide (P₁, P₁ₐ, P_{1b} or P_{1c}) comprises an amino acid sequence that has 2 amino acid substitutions relative to Peptide-2. In some embodiments, the peptide (P₁, P₁ₐ, P_{1b} or P_{1c}) comprises an amino acid sequence that has 3 amino acid substitutions relative to Peptide-2.

In some embodiments, the peptide (P₁, P₁ₐ, P_{1b} or P_{1c}) comprises an amino acid sequence that has 1 amino acid deletion relative to Peptide-1. In some embodiments, the peptide (P₁, P₁ₐ, P_{1b} or P_{1c}) comprises an amino acid sequence that has 2 amino acid deletions relative to Peptide-1. In some embodiments, the peptide (P₁, P₁ₐ, P_{1b} or P_{1c}) comprises an amino acid sequence that has 3 amino acid deletions relative to Peptide-1.

In some embodiments, the peptide (P₁, P₁ₐ, P_{1b} or P_{1c}) comprises an amino acid sequence that has 1 amino acid deletions relative to Peptide-2. In some embodiments, the peptide (P₁, P₁ₐ, P_{1b} or P_{1c}) comprises an amino acid sequence that has 2 amino acid deletions relative to Peptide-2. In some embodiments, the peptide (P₁, P₁ₐ, P_{1b} or P_{1c}) comprises an amino acid sequence that has 3 amino acid deletions relative to Peptide-2.

In some embodiments, the peptide (P₁, P₁ₐ, P_{1b} or P_{1c}) comprises an amino acid sequence that has 1 amino acid additions relative to Peptide-1. In some embodiments, the peptide (P₁, P₁ₐ, P_{1b} or P_{1c}) comprises an amino acid sequence that has 2 amino acid additions relative to Peptide-1. In some embodiments, the peptide (P₁, P₁ₐ, P_{1b} or P_{1c}) comprises an amino acid sequence that has 3 amino acid additions relative to Peptide-1.

In some embodiments, the peptide (P₁, P₁ₐ, P_{1b} or P_{1c}) comprises an amino acid sequence that has 1 amino acid addition relative to Peptide-2. In some embodiments, the peptide (P₁, P₁ₐ, P_{1b} or P_{1c}) comprises an amino acid sequence that has 2 amino acid additions relative to Peptide-2. In some embodiments, the peptide (P₁, P₁ₐ, P_{1b} or P_{1c}) comprises an amino acid sequence that has 3 amino acid additions relative to Peptide-2.

In some embodiments, the peptide (P₁, P₁ₐ, P_{1b} or P_{1c}) comprises an amino acid sequence that has 1 amino acid addition relative to Peptide-39. In some embodiments, the peptide (P₁, P₁ₐ, P_{1b} or P_{1c}) comprises an amino acid sequence that has 2 amino acid additions relative to Peptide-39. In some embodiments, the peptide (P₁, P₁ₐ, P_{1b} or P_{1c}) comprises an amino acid sequence that has 3 amino acid additions relative to Peptide-39.

In some embodiments, P₁ is bound to A₁ through ionic interactions, electrostatic interactions, hydrophobic interactions, Pi-stacking interactions, or H-bonding interactions, or a combination thereof.

In some embodiments, P₁ₐ is bound to the anti-CD3 binding domain through ionic interactions, electrostatic interactions, hydrophobic interactions, Pi-stacking interactions, or H-bonding interactions, or a combination thereof when L₁ₐ is uncleaved.

In some embodiments, P₁, P₁ₐ, P_{1b} or P_{1c} comprise a modified amino acid or non-natural amino acid, or a modified non-natural amino acid, or a combination thereof. In some embodiments, the modified amino acid or a modified non-natural amino acid comprises a post-translational modification. In some embodiments P₁, P₁ₐ, P_{1b} or P_{1c} comprises a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to P₁, P₁ₐ, P_{1b} or P_{1c} including the peptide backbone, the amino acid side chains, and the terminus.

TABLE 1
Amino acid sequence of Peptide-1 and Peptide-2

| Construct | Amino Acid Sequence | |
|---|---|---|
| Description | (N to C) | SEQ ID NO: |
| Peptide-1 | GSQCLGPEWEVCPY | 1 |
| Peptide-2 | VYCGPEFDESVGCM | 2 |

### Anti-CD3 Binding Domain (A₁)

In some embodiments, A₁ comprises an antibody or antibody fragment. In some embodiments, A₁ comprises an antibody or antibody fragment that is human or humanized. In some embodiments, L₁ is bound to N-terminus of the antibody or antibody fragment of A₁. In some embodiments, A₂ is bound to N-terminus of the antibody or antibody fragment of A₁. In some embodiments, L₁ is bound to the C-terminus of the antibody or antibody fragment of A₁. In some embodiments, A₂ is bound to the C-terminus of the antibody or antibody fragment of A₁. In some embodiments, the antibody or antibody fragment comprises a single chain variable fragment, a single domain antibody, or a Fab fragment. In some embodiments, A₁ is the single chain variable fragment (scFv). In some embodiments, the scFv comprises a scFv heavy chain polypeptide and a scFv light chain polypeptide. In some embodiments, A₁ is the single domain antibody. In some embodiments, the antibody or antibody fragment thereof comprises a single chain variable fragment (scFv), a heavy chain variable domain (VH domain), a light chain variable domain (VL domain), or a variable domain (VHH) of a camelid derived single domain antibody.

In some embodiments, the first target antigen is CD3. In some embodiments, A₁ comprises an anti-CD3e single chain variable fragment. In some embodiments, A₁ comprises an anti-CD3e single chain variable fragment that has a KD binding of 1 μM or less to CD3 on CD3 expressing cells. In some embodiments, A₁ comprises a variable light chain and variable heavy chain each of which is capable of specifically binding to human CD3. In some embodiments, A₁ comprises complementary determining regions (CDRs) from sp34 scFv. In some embodiments, the scFv comprises a scFv heavy chain variable domain and an scFv light chain variable domain. In some embodiments, the polypeptide or polypeptide complex of formula I binds to an effector cell when L₁ is cleaved by the tumor specific protease. In some embodiments, the polypeptide or polypeptide complex of formula I binds to an effector cell when L₁ is cleaved by the tumor specific protease and A₁ binds to the effector cell. In some embodiments, the effector cell is a T cell. In some embodiments, A₁ binds to a polypeptide that is part of a TCR-CD3 complex on the effector cell. In some embodiments, the polypeptide that is part of the TCR-CD3 complex is human CD3ε.

In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 3; HC-CDR2: SEQ ID NO: 4; HC-CDR3: SEQ ID NO: 5, and wherein the CDRs comprise from 0-2 amino acid modifications in at least one of the HC-CDR1, HC-CDR2, or HC-CDR3.

In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 6; LC-CDR2: SEQ ID NO: 7; and LC-CDR3: SEQ ID NO: 8, and wherein the CDRs comprise from 0-2 amino acid modifications in at least one of the LC-CDR1, LC-CDR2, or LC-CDR3.

In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 3; HC-CDR2: SEQ ID NO: 4; HC-CDR3: SEQ ID NO: 5; and the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 6; LC-CDR2: SEQ ID NO: 7; and LC-CDR3: SEQ ID NO: 8.

In some embodiments, A₁ comprises complementarity determining regions (CDRs): HC-CDR1, HC-CDR2, and HC-CDR3, wherein the HC-CDR1, the HC-CDR2, and the HC-CDR3 of A₁ comprise: HC-CDR1: SEQ ID NO: 3; HC-CDR2: SEQ ID NO: 4; HC-CDR3: SEQ ID NO: 5, and wherein the CDRs comprise from 0-2 amino acid modifications in at least one of the HC-CDR1, HC-CDR2, or HC-CDR3.

In some embodiments, A₁ comprises complementarity determining regions (CDRs): LC-CDR1, LC-CDR2, and LC-CDR3, wherein the LC-CDR1, the LC-CDR2, and the LC-CDR3 of A₁ comprise: LC-CDR1: SEQ ID NO: 6; LC-CDR2: SEQ ID NO: 7; and LC-CDR3: SEQ ID NO: 8, and wherein the CDRs comprise from 0-2 amino acid modifications in at least one of the LC-CDR1, LC-CDR2, or LC-CDR3.

In some embodiments, A₁ comprises complementarity determining regions (CDRs): HC-CDR1, HC-CDR2, and HC-CDR3, wherein the HC-CDR1, the HC-CDR2, and the HC-CDR3 of A₁ comprise: HC-CDR1: SEQ ID NO: 3; HC-CDR2: SEQ ID NO: 4; HC-CDR3: SEQ ID NO: 5; and A₁ comprises complementarity determining regions (CDRs): LC-CDR1, LC-CDR2, and LC-CDR3, wherein the LC-CDR1, the LC-CDR2, and the LC-CDR3 of A₁ comprise: LC-CDR1: SEQ ID NO: 6; LC-CDR2: SEQ ID NO: 7; and LC-CDR3: SEQ ID NO: 8.

In some embodiments, the scFv heavy chain variable domain comprises an amino acid sequence that has at least 70% sequence identity to the amino acid sequence according to SEQ ID NO: 9. In some embodiments, the scFv heavy chain variable domain comprises an amino acid sequence that has at least 80% sequence identity to the amino acid sequence according to SEQ ID NO: 9. In some embodiments, the scFv heavy chain variable domain comprises an amino acid sequence that has at least 85% sequence identity to the amino acid sequence according to SEQ ID NO: 9. In some embodiments, the scFv heavy chain variable domain comprises an amino acid sequence that has at least 90% sequence identity to the amino acid sequence according to SEQ ID NO: 9. In some embodiments, the scFv heavy chain variable domain comprises an amino acid sequence that has at least 91% sequence identity to the amino acid sequence according to SEQ ID NO: 9. In some embodiments, the scFv heavy chain variable domain comprises an amino acid sequence that has at least 92% sequence identity to the amino acid sequence according to SEQ ID NO: 9. In some embodiments, the scFv heavy chain variable domain comprises an amino acid sequence that has at least 93% sequence identity to the amino acid sequence according to SEQ ID NO: 9. In some embodiments, the scFv heavy chain variable domain comprises an amino acid sequence that has at least 94% sequence identity to the amino acid sequence according to SEQ ID NO: 9. In some embodiments, the scFv heavy chain variable domain comprises an amino acid sequence that has at least 95% sequence identity to the amino acid sequence according to SEQ ID NO: 9. In some embodiments, the scFv heavy chain variable domain comprises an amino acid sequence that has at least 99% sequence identity to the amino acid sequence according to SEQ ID NO: 9. In some embodiments, the scFv heavy chain variable domain comprises an amino acid sequence according to SEQ ID NO: 9.

In some embodiments, the scFv heavy chain variable domain comprises an amino acid sequence of at least 50 consecutive amino acid residues of SEQ ID NO: 9, and has at least 95% sequence identity to the at least 50 consecutive amino acid residues of SEQ ID NO: 9. In some embodiments, the scFv heavy chain variable domain comprises an amino acid sequence of at least 60 consecutive amino acid residues of SEQ ID NO: 9, and has at least 95% sequence identity to the at least 60 consecutive amino acid residues of SEQ ID NO: 9. In some embodiments, the scFv heavy chain variable domain comprises an amino acid sequence of at least 70 consecutive amino acid residues of SEQ ID NO: 9, and has at least 95% sequence identity to the at least 70 consecutive amino acid residues of SEQ ID NO: 9. In some embodiments, the scFv heavy chain variable domain comprises an amino acid sequence of at least 80 consecutive amino acid residues of SEQ ID NO: 9, and has at least 95% sequence identity to the at least 80 consecutive amino acid residues of SEQ ID NO: 9. In some embodiments, the scFv heavy chain variable domain comprises an amino acid sequence of at least 90 consecutive amino acid residues of SEQ ID NO: 9, and has at least 95% sequence identity to the at least 90 consecutive amino acid residues of SEQ ID NO: 9. In some embodiments, the scFv heavy chain variable domain comprises an amino acid sequence of at least 100 consecutive amino acid residues of SEQ ID NO: 9, and has at least 95% sequence identity to the at least 100 consecutive amino acid residues of SEQ ID NO: v. In some embodiments, the scFv heavy chain variable domain comprises an amino acid sequence of at least 105 consecutive amino acid residues of SEQ ID NO: 9, and has at least 95% sequence identity to the at least 105 consecutive amino acid residues of SEQ ID NO: 9. In some embodiments, the scFv heavy chain variable domain comprises an amino acid sequence of at least 110 consecutive amino acid residues of SEQ ID NO: 9, and has at least 95% sequence identity to the at least 110 consecutive amino acid residues of SEQ ID NO: 9. In some embodiments, the scFv heavy chain variable domain comprises an amino acid sequence of at least 115 consecutive amino acid residues of SEQ ID NO: 9, and has at least 95% sequence identity to the at least 115 consecutive amino acid residues of SEQ ID NO: 9. In some embodiments, the scFv heavy chain variable domain comprises an amino acid sequence of at least 120 consecutive amino acid residues of SEQ ID NO: 9, and has at least 95% sequence identity to the at least 120 consecutive amino acid residues of SEQ ID NO: 9.

TABLE 2
Exemplary amino acid sequence of Anti-CD3 binding domain (A1) (CDRs
determined by IMGT definition)

| | Amino Acid Sequence | |
|---|---|---|
| Construct Description | (N to C) | SEQ ID NO: |
| CD3: HC: CDR1 | GFTFNKYA | 3 |
| CD3: HC: CDR2 | IRSKYNNYAT | 4 |
| CD3: HC: CDR3 | VRHGNFGNSYISYWAY | 5 |
| CD3: LC: CDR1 | TGAVTSGNY | 6 |
| CD3: LC: CDR2 | GTK | 7 |
| CD3: LC: CDR3 | VLWYSNRWV | 8 |
| Anti-CD3 SP34.185 scFv | EVQLVESGGGLVQPGGSLKLSCAASGFTFNK | 9 |
| | YAMNWVRQAPGKGLEWVARIRSKYNNYAT | |
| | YYADSVKDRFTISRDDSKNTAYLQMNNLKTE | |
| | DTAVYYCVRHGNFGNSYISYWAYWGQGTL | |
| | VTVSSGGGGSGGGGSGGGGSQTVVTQEPSLT | |
| | VSPGGTVTLTCGSSTGAVTSGNYPNWVQQKP | |
| | GQAPRGLIGGTKFLAPGTPARFSGSLLGGKAA | |
| | LTLSGVQPEDEAEYYCVLWYSNRWVFGGGT | |
| | KLTVL | |

### Tumor Antigen Binding Domain (A₂)

In some embodiments, A₂ comprises an antibody or antibody fragment. In some embodiments, the antibody or antibody fragment thereof of A₂ comprises a single chain variable fragment, a single domain antibody, Fab′, or a Fab. In some embodiments, the antibody or antibody fragment thereof of A₂ comprises a single chain variable fragment (scFv), a heavy chain variable domain (VH domain), a light chain variable domain (VL domain), or a variable domain (VHH) of a camelid derived single domain antibody. In some embodiments, the antibody or antibody fragment thereof of A₂ is humanized or human. In some embodiments, A₂ is the Fab or Fab′. In some embodiments, the Fab or Fab′ comprises (a) a Fab light chain polypeptide and (b) a Fab heavy chain polypeptide. In some embodiments, the second target antigen comprises a tumor antigen. In some embodiments, the antibody or antibody fragment thereof comprises an epidermal growth factor receptor (EGFR) binding domain. In some embodiments, the antibody or antibody fragment thereof comprises a mesothelin binding domain. In some embodiments, A₂ comprises an amino acid sequence disclosed in Table 3 or a sequence substantially identical thereto (e.g., a sequence that has at least 90%, 95%, 96%, 97%, 98%, or 99% sequence identity to an amino acid sequence disclosed in Table 3).

TABLE 3
Exemplary amino acid sequences of Anti-EGFR Fab

| | Amino Acid Sequence | |
|---|---|---|
| Construct Description | (N to C) | SEQ ID NO: |
| Anti-EGFR Fab | DILLTQSPVILSVSPGERVSFSCRASQSIGTNIH | 10 |
| light chain | WYQQRTNGSPRLLIKYASESISGIPSRFSGSGS | |
| | GTDFTLSINSVESEDIADYYCQQNNNWPTTF | |
| | GAGTKLELKRTVAAPSVFIFPPSDEQLKSGTA | |
| | SVVCLLNNFYPREAKVQWKVDNALQSGNSQ | |
| | ESVTEQDSKDSTYSLSSTLTLSKADYEKHKVY | |
| | ACEVTHQGLSSPVTKSFNRGEC | |
| Anti-EGFR Fab | QVQLKQSGPGLVQPSQSLSITCTVSGFSLTNY | 11 |
| heavy chain | GVHWVRQSPGKGLEWLGVIWSGGNTDYNT | |
| | PFTSRLSINKDNSKSQVFFKMNSLQSQDTAIY | |
| | YCARALTYYDYEFAYWGQGTLVTVSAASTK | |
| | GPSVFPLAPSSKSTSGGTAALGCLVKDYFPEP | |
| | VTVSWNSGALTSGVHTFPAVLQSSGLYSLSSV | |
| | VTVPSSSLGTQTYICNVNHKPSNTKVDKKVEP | |
| | KSC | |

Half-Life Extending Molecule (H₁, H₁ₐ, H_{1b}, H_{1c})

In some embodiments, P₁ is further linked to a half-life extending moiety (H₁).

In some embodiments, the half-life extending moiety is a single-domain antibody.

In some embodiments, H₁ does not block P₁ binding to CD3. In some embodiments, H₁ₐ does not block the anti-CD3 binding domain from binding to the CD3. In some embodiments, H_{1b} does not block the anti-CD3 binding domain from binding to the CD3. In some embodiments, H_{1c} does not block the anti-CD3 binding domain from binding to the CD3. In some embodiments, the half-life extending molecule (H₁, H₁ₐ, H_{1b}, or H_{1c}) does not have binding affinity to CD3. In some embodiments, the half-life extending molecule (H₁, H₁ₐ, H_{1b}, or H_{1c}) does not have binding affinity to CD3. In some embodiments, the half-life extending molecule (H₁, H₁ₐ, H_{1b}, or H_{1c}) does not shield the anti-CD3 binding domain from CD3.

In some embodiments, H₁, H₁ₐ, H_{1b}, or H_{1c} comprise an amino acid sequence that has repetitive sequence motifs. In some embodiments, H₁, H₁ₐ, H_{1b}, or H_{1c} comprises an amino acid sequence that has highly ordered secondary structure. “Highly ordered secondary structure,” as used in this context, means that at least about 50%, or about 70%, or about 80%, or about 90%, of amino acid residues of H₁ or H₁ₐ contribute to secondary structure, as measured or determined by means, including, but not limited to, spectrophotometry (e.g. by circular dichroism spectroscopy in the “far-UV” spectral region (190-250 nm), and computer programs or algorithms, such as the Chou-Fasman algorithm and the Garnier-Osguthorpe-Robson (“GOR”) algorithm.

In some embodiments, H₁, H₁ₐ, H_{1b}, or H_{1c} comprises a polymer. In some embodiments, the polymer is polyethylene glycol (PEG). In some embodiments, H₁ or H₁ₐ comprises albumin. In some embodiments, H₁ or H₁ₐ comprises a Fc domain. In some embodiments, the albumin is serum albumin. In some embodiments, the albumin is human serum albumin. In some embodiments, H₁, H₁ₐ, H_{1b}, or H_{1c} comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgGl, IgG2, IgG3, IgG4, slgA, IgM or IgD. In some embodiments, the serum protein is albumin. In some embodiments, the polypeptide is an antibody. In some embodiments, the antibody comprises a single domain antibody, a single chain variable fragment or a Fab. In some embodiments, the antibody comprises a single domain antibody. In some embodiments, the antibody comprises a single domain antibody that binds to albumin. In some embodiments, the antibody comprises a single domain antibody that binds to human serum albumin. In some embodiments, the antibody is a human or humanized antibody. In some embodiments, the antibody is selected from the group consisting of 645gH1gL1, 645dsgH5gL4, 23-13-A01-sc02, A10m3 or a fragment thereof, DOM7r-31, DOM7h-11-15, Alb-1, Alb-8, Alb-23, 10G, 10GE, and SA21.

In some embodiments, H₁ comprises a single domain antibody. In some embodiments, H₁ comprises a single domain antibody that binds to albumin. In some embodiments, H₁ comprises a single domain antibody that binds to human serum albumin. In some embodiments, H₁ₐ comprises a single domain antibody. In some embodiments, H₁ₐ comprises a single domain antibody that binds to albumin. In some embodiments, H₁ₐ comprises a single domain antibody that binds to human serum albumin. In some embodiments, H_{1b} comprises a single domain antibody. In some embodiments, H_{1b} comprises a single domain antibody that binds to albumin. In some embodiments, H_{1b} comprises a single domain antibody that binds to human serum albumin. In some embodiments, H_{1c} comprises a single domain antibody. In some embodiments, H_{1c} comprises a single domain antibody that binds to albumin. In some embodiments, H_{1c} comprises a single domain antibody that binds to human serum albumin.

In some embodiments, H₁, H₁ₐ, H_{1b}, or H_{1c} comprise a modified amino acid or non-natural amino acid, or a modified non-natural amino acid, or a combination thereof. In some embodiments, the modified amino acid or a modified non-natural amino acid comprises a post-translational modification. In some embodiments H₁, H₁ₐ, H_{1b}, or H_{1c} comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to H₁, H₁ₐ, H_{1b}, or H_{1c} including the peptide backbone, the amino acid side chains, and the terminus. In some embodiments, H₁, H₁ₐ, H_{1b}, or H_{1c} comprises an amino acid sequence disclosed in Table 4 or a sequence substantially identical thereto (e.g., a sequence that has at least 90%, 95%, 96%, 97%, 98%, or 99% sequence identity to an amino acid sequence disclosed in Table 4).

TABLE 4
Exemplary amino acid sequence of half-life extending molecule
(H₁, H₁ₐ, H_{1b}, H_{1c})

| | Amino Acid Sequence | |
|---|---|---|
| Construct Description | (N to C) | SEQ ID NO: |
| 10G single | EVQLVESGGGLVQPGNSLRLSCAASGFTFSK | 12 |
| domain antibody | FGMSWVRQAPGKGLEWVSSISGSGRDTLYA | |
| | DSVKGRFTISRDNAKTTLYLQMNSLRPEDTA | |
| | VYYCTIGGSLSVSSQGTLVTVSS | |

### Peptide (P₂)

In some embodiments, P₂ impairs binding of A₂ to the tumor antigen. In some embodiments, P₂ is bound to A₂ through ionic interactions, electrostatic interactions, hydrophobic interactions, Pi-stacking interactions, or H-bonding interactions, or a combination thereof. In some embodiments, P₂ is bound to A₂ at or near an antigen binding site. In some embodiments, P₂ has less than 70% sequence homology to the tumor antigen. In some embodiments, P₂ has less than 75% sequence homology to the tumor antigen. In some embodiments, P₂ has less than 80% sequence homology to the tumor antigen. In some embodiments, P₂ has less than 85% sequence homology to the tumor antigen. In some embodiments, P₂ has less than 90% sequence homology to the tumor antigen. In some embodiments, P₂ has less than 95% sequence homology to the tumor antigen. In some embodiments, P₂ has less than 98% sequence homology to the tumor antigen. In some embodiments, P₂ has less than 99% sequence homology to the tumor antigen.

In some embodiments, P₂ comprises a peptide sequence of at least 10 amino acids in length. In some embodiments, P₂ comprises a peptide sequence of at least 10 amino acids in length and no more than 20 amino acids in length. In some embodiments, P₂ comprises a peptide sequence of at least 16 amino acids in length. In some embodiments, P₂ comprises a peptide sequence of no more than 40 amino acids in length. In some embodiments, P₂ comprises at least two cysteine amino acid residues. In some embodiments, P₂ comprises a cyclic peptide or a linear peptide. In some embodiments, P₂ comprises a cyclic peptide. In some embodiments, P₂ comprises a linear peptide.

In some embodiments, P₂ comprise a modified amino acid or non-natural amino acid, or a modified non-natural amino acid, or a combination thereof. In some embodiments, the modified amino acid or a modified non-natural amino acid comprises a post-translational modification. In some embodiments P₂ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to P₂ including the peptide backbone, the amino acid side chains, and the terminus.

In some embodiments, P₂ does not comprise albumin or an albumin fragment. In some embodiments, P₂ does not comprise an albumin binding domain.

Cleavable Linker (L₁ or L₂)

In some embodiments, L₁ or L₂ is a peptide sequence having at least 5 to no more than 50 amino acids. In some embodiments, L₁ or L₂ is a peptide sequence having at least 10 to no more than 30 amino acids. In some embodiments, L₁ or L₂ is a peptide sequence having at least 10 amino acids. In some embodiments, L₁ or L₂ is a peptide sequence having at least 18 amino acids. In some embodiments, L₁ or L₂ is a peptide sequence having at least 26 amino acids.

In some embodiments, L₁ has a formula selected from the group consisting of (G₂S)ₙ, (GS)ₙ, (GSGGS)ₙ (SEQ ID NO: 917), (GGGS)ₙ (SEQ ID NO: 918), (GGGGS)ₙ (SEQ ID NO: 919), and (GSSGGS)ₙ (SEQ ID NO: 920), wherein n is an integer of at least 1. In some embodiments, L₁ has a formula selected from the group consisting of (G₂S)ₙ, (GS)ₙ, (GSGGS)ₙ (SEQ ID NO: 917), (GGGS)ₙ (SEQ ID NO: 918), (GGGGS)ₙ (SEQ ID NO: 919), and (GSSGGS)ₙ (SEQ ID NO: 920), wherein n is an integer of 1. In some embodiments, L₁ has a formula selected from the group consisting of (G₂S)ₙ (SEQ ID NO: 922), (GS)ₙ (SEQ ID NO: 938), (GSGGS)ₙ (SEQ ID NO: 939), (GGGS)ₙ (SEQ ID NO: 940), (GGGGS)ₙ (SEQ ID NO: 941), and (GSSGGS)ₙ (SEQ ID NO: 942), wherein n is an integer from 1 to 3.

In some embodiments, L₁ has a formula of (G₂S)ₙ, wherein n is an integer of least 1. In some embodiments, L₁ has a formula of (GS)ₙ, wherein n is an integer of least 1. In some embodiments, L₁ has a formula of (GSGGS)ₙ (SEQ ID NO: 917), wherein n is an integer of least 1. In some embodiments, L₁ has a formula of (GGGS)ₙ (SEQ ID NO: 918), wherein n is an integer of least 1. In some embodiments, L₁ has a formula of (GGGGS)ₙ (SEQ ID NO: 919), wherein n is an integer of least 1. In some embodiments, L₁ has a formula of (GSSGGS)ₙ (SEQ ID NO: 920), wherein n is an integer of least 1.

In some embodiments, L₁ has a formula of (G₂S)ₙ, wherein n is an integer of 1. In some embodiments, L₁ has a formula of (GS)ₙ, wherein n is an integer of 1. In some embodiments, L₁ has a formula of (GSGGS)ₙ (SEQ ID NO: 917), wherein n is an integer of 1. In some embodiments, L₁ has a formula of (GGGS)ₙ (SEQ ID NO: 918), wherein n is an integer of 1. In some embodiments, L₁ has a formula of (GGGGS)ₙ (SEQ ID NO: 919), wherein n is an integer of 1. In some embodiments, L₁ has a formula of (GSSGGS)ₙ (SEQ ID NO: 920), wherein n is an integer of 1.

In some embodiments, L₁ has a formula of (G₂S)ₙ (SEQ ID NO: 922), wherein n is an integer from 1 to 3. In some embodiments, L₁ has a formula of (GS)ₙ (SEQ ID NO: 938), wherein n is an integer from 1 to 3. In some embodiments, L₁ has a formula of (GSGGS)ₙ (SEQ ID NO: 939), wherein n is an integer from 1 to 3. In some embodiments, L₁ has a formula of (GGGS)ₙ (SEQ ID NO: 940), wherein n is an integer from 1 to 3. In some embodiments, L₁ has a formula of (GGGGS)ₙ (SEQ ID NO: 941), wherein n is an integer from 1 to 3. In some embodiments, L₁ has a formula of (GSSGGS)ₙ (SEQ ID NO: 942), wherein n is an integer from 1 to 3.

In some embodiments, L₁ is a substrate for a tumor specific protease. In some embodiments, the tumor specific protease is selected from the group consisting of metalloprotease, serine protease, cysteine protease, threonine protease, and aspartic protease. In some embodiments, L₁ comprises a urokinase cleavable amino acid sequence, a matriptase cleavable amino acid sequence, a matrix metalloprotease cleavable amino acid sequence, or a legumain cleavable amino acid sequence. In some embodiments, L₁ comprises an amino acid sequence selected from the group consisting of GGGGSLSGRSDNHGSSGT (SEQ ID NO: 923), GGGGSSGGSGGSGLSGRSDNHGSSGT (SEQ ID NO: 924), ASGRSDNH (SEQ ID NO: 925), LAGRSDNH (SEQ ID NO: 926), ISSGLASGRSDNH (SEQ ID NO: 927), ISSGLLAGRSDNH (SEQ ID NO: 928), LSGRSDNH (SEQ ID NO: 929), ISSGLLSGRSDNP (SEQ ID NO: 930), ISSGLLSGRSDNH (SEQ ID NO: 931), LSGRSDNHSPLGLAGS (SEQ ID NO: 932), SPLGLAGSLSGRSDNH (SEQ ID NO: 933), SPLGLSGRSDNH (SEQ ID NO: 934), LAGRSDNHSPLGLAGS (SEQ ID NO: 935), LSGRSDNHVPLSLKMG (SEQ ID NO: 936), and LSGRSDNHVPLSLSMG (SEQ ID NO: 937). In some embodiments, L₁ comprises an amino acid sequence selected from the group consisting of ASGRSDNH (SEQ ID NO: 925), LAGRSDNH (SEQ ID NO: 926), ISSGLASGRSDNH (SEQ ID NO: 927), and ISSGLLAGRSDNH (SEQ ID NO: 928).

In some embodiments, L₂ has a formula selected from the group consisting of (G₂S)ₙ, (GS)ₙ, (GSGGS)ₙ (SEQ ID NO: 917), (GGGS)ₙ (SEQ ID NO: 918), (GGGGS)ₙ (SEQ ID NO: 919), and (GSSGGS)ₙ (SEQ ID NO: 920), wherein n is an integer of at least 1. In some embodiments, L₂ has a formula selected from the group consisting of (G₂S)ₙ, (GS)ₙ, (GSGGS)ₙ (SEQ ID NO: 917), (GGGS)ₙ (SEQ ID NO: 918), (GGGGS)ₙ (SEQ ID NO: 919), and (GSSGGS)ₙ (SEQ ID NO: 920), wherein n is an integer of 1. In some embodiments, L₂ has a formula selected from the group consisting of (G₂S)ₙ (SEQ ID NO: 922), (GS)ₙ (SEQ ID NO: 938), (GSGGS)ₙ (SEQ ID NO: 939), (GGGS)ₙ (SEQ ID NO: 940), (GGGGS)ₙ (SEQ ID NO: 941), and (GSSGGS)ₙ (SEQ ID NO: 942), wherein n is an integer from 1 to 3.

In some embodiments, L₂ has a formula of (G₂S)ₙ, wherein n is an integer of least 1. In some embodiments, L₂ has a formula of (GS)ₙ, wherein n is an integer of least 1. In some embodiments, L₂ has a formula of (GSGGS)ₙ (SEQ ID NO: 917), wherein n is an integer of least 1. In some embodiments, L₂ has a formula of (GGGS)ₙ (SEQ ID NO: 918), wherein n is an integer of least 1. In some embodiments, L₂ has a formula of (GGGGS)ₙ (SEQ ID NO: 919), wherein n is an integer of least 1. In some embodiments, L₂ has a formula of (GSSGGS)ₙ (SEQ ID NO: 920), wherein n is an integer of least 1.

In some embodiments, L₂ has a formula of (G₂S)ₙ, wherein n is an integer of 1. In some embodiments, L₂ has a formula of (GS)ₙ, wherein n is an integer of 1. In some embodiments, L₂ has a formula of (GSGGS)ₙ (SEQ ID NO: 917), wherein n is an integer of 1. In some embodiments, L₂ has a formula of (GGGS)ₙ (SEQ ID NO: 918), wherein n is an integer of 1. In some embodiments, L₂ has a formula of (GGGGS)ₙ (SEQ ID NO: 919), wherein n is an integer of 1. In some embodiments, L₂ has a formula of (GSSGGS)ₙ (SEQ ID NO: 920), wherein n is an integer of 1.

In some embodiments, L₂ has a formula of (G₂S)ₙ (SEQ ID NO: 922), wherein n is an integer from 1 to 3. In some embodiments, L₂ has a formula of (GS)ₙ (SEQ ID NO: 938), wherein n is an integer from 1 to 3. In some embodiments, L₂ has a formula of (GSGGS)ₙ (SEQ ID NO: 939), wherein n is an integer from 1 to 3. In some embodiments, L₂ has a formula of (GGGS)ₙ (SEQ ID NO: 940), wherein n is an integer from 1 to 3. In some embodiments, L₂ has a formula of (GGGGS)ₙ (SEQ ID NO: 941), wherein n is an integer from 1 to 3. In some embodiments, L₂ has a formula of (GSSGGS)ₙ (SEQ ID NO: 942), wherein n is an integer from 1 to 3.

In some embodiments, L₂ is a substrate for a tumor specific protease. In some embodiments, the tumor specific protease is selected from the group consisting of metalloprotease, serine protease, cysteine protease, threonine protease, and aspartic protease. In some embodiments, L₂ comprises a urokinase cleavable amino acid sequence, a matriptase cleavable amino acid sequence, a matrix metalloprotease cleavable amino acid sequence, or a legumain cleavable amino acid sequence. In some embodiments, L₂ comprises an amino acid sequence selected from the group consisting of GGGGSLSGRSDNHGSSGT (SEQ ID NO: 923), GGGGSSGGSGGSGLSGRSDNHGSSGT (SEQ ID NO: 924), ASGRSDNH (SEQ ID NO: 925), LAGRSDNH (SEQ ID NO: 926), ISSGLASGRSDNH (SEQ ID NO: 927), ISSGLLAGRSDNH (SEQ ID NO: 928), LSGRSDNH (SEQ ID NO: 929), ISSGLLSGRSDNP (SEQ ID NO: 930), ISSGLLSGRSDNH (SEQ ID NO: 931), LSGRSDNHSPLGLAGS (SEQ ID NO: 932), SPLGLAGSLSGRSDNH (SEQ ID NO: 933), SPLGLSGRSDNH (SEQ ID NO: 934), LAGRSDNHSPLGLAGS (SEQ ID NO: 935), LSGRSDNHVPLSLKMG (SEQ ID NO: 936), and LSGRSDNHVPLSLSMG (SEQ ID NO: 937). In some embodiments, L₂ comprises an amino acid sequence selected from the group consisting of ASGRSDNH (SEQ ID NO: 925), LAGRSDNH (SEQ ID NO: 926), ISSGLASGRSDNH (SEQ ID NO: 927), ISSGLLAGRSDNH (SEQ ID NO: 928), or GGGGSGGGSGGISSGLLSGRSDNHGGGS (SEQ ID NO: 943).

In some embodiments, L₁ or L₂ comprises a modified amino acid or non-natural amino acid, or a modified non-natural amino acid, or a combination thereof. In some embodiments, the modified amino acid or a modified non-natural amino acid comprises a post-translational modification. In some embodiments, L₁ or L₂ comprises a modification including, but not limited, to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. In some embodiments, modifications are made anywhere to L₁ or L₂ including the peptide backbone, or the amino acid side chains.

Linking Moiety (L₃, L₃ₐ, L_{3b}, or L_{3c})

In some embodiments, H₁ comprises a linking moiety (L₃) that connects H₁ to P₁. In some embodiments, H₁ₐ comprises a linking moiety (L₃ₐ) that connects H₁ₐ to P₁ₐ. In some embodiments, H_{1b} comprises a linking moiety (L_{3b}) that connects H_{1b} to P_{1b}. In some embodiments, H_{1b} comprises a linking moiety (L_{3c}) that connects H_{1c} to P_{1c}.

In some embodiments, L₃, L₃ₐ, L_{3b}, or L_{3c} is a peptide sequence having at least 5 to no more than 50 amino acids. In some embodiments, L₃, L₃ₐ, L_{3b}, or L_{3c} is a peptide sequence having at least 10 to no more than 30 amino acids. In some embodiments, L₃, L₃ₐ, L_{3b}, or L_{3c} is a peptide sequence having at least 10 amino acids. In some embodiments, L₃, L₃ₐ, L_{3b}, or L_{3c} is a peptide sequence having at least 18 amino acids. In some embodiments, L₃, L₃ₐ, L_{3b}, or L_{3c} is a peptide sequence having at least 26 amino acids.

In some embodiments, L₃, L₃ₐ, L_{3b}, or L_{3c} has a formula selected from the group consisting of (G₂S)ₙ, (GS)ₙ, (GSGGS)ₙ (SEQ ID NO: 917), (GGGS)ₙ (SEQ ID NO: 918), (GGGGS)ₙ (SEQ ID NO: 919), and (GSSGGS)ₙ (SEQ ID NO: 920), wherein n is an integer of at least 1. In some embodiments, L₃, L₃ₐ, L_{3b}, or L_{3c} has a formula selected from the group consisting of (G₂S)ₙ, (GS)ₙ, (GSGGS)ₙ (SEQ ID NO: 917), (GGGS)ₙ (SEQ ID NO: 918), (GGGGS)ₙ (SEQ ID NO: 919), and (GSSGGS)ₙ (SEQ ID NO: 920), wherein n is an integer of 1. In some embodiments, L₃, L₃ₐ, L_{3b}, or L_{3c} has a formula selected from the group consisting of (G₂S)ₙ (SEQ ID NO: 922), (GS)ₙ (SEQ ID NO: 938), (GSGGS)ₙ (SEQ ID NO: 939), (GGGS)ₙ (SEQ ID NO: 940), (GGGGS)ₙ (SEQ ID NO: 941), and (GSSGGS)ₙ (SEQ ID NO: 942), wherein n is an integer from 1 to 3.

In some embodiments, L₃, L₃ₐ, L_{3b}, or L_{3c} has a formula of (G₂S)ₙ, wherein n is an integer of least 1. In some embodiments, L₃, L₃ₐ, L_{3b}, or L_{3c} has a formula of (GS)ₙ, wherein n is an integer of least 1. In some embodiments, L₃, L₃ₐ, L_{3b}, or L_{3c} has a formula of (GSGGS)ₙ (SEQ ID NO: 917), wherein n is an integer of least 1. In some embodiments, L₃, L₃ₐ, L_{3b}, or L_{3c} has a formula of (GGGS)ₙ (SEQ ID NO: 918), wherein n is an integer of least 1. In some embodiments, L₃, L₃ₐ, L_{3b}, or L_{3c} has a formula of (GGGGS)ₙ (SEQ ID NO: 919), wherein n is an integer of least 1. In some embodiments, L₃, L₃ₐ, L_{3b}, or L_{3c} has a formula of (GSSGGS)ₙ (SEQ ID NO: 920), wherein n is an integer of least 1.

In some embodiments, L₃ or L₃ₐ has a formula of (G₂S)ₙ, wherein n is an integer of 1. In some embodiments, L₃, L₃ₐ, L_{3b}, or L_{3c} has a formula of (GS)ₙ, wherein n is an integer of 1. In some embodiments, L₃, L₃ₐ, L_{3b}, or L_{3c} has a formula of (GSGGS)ₙ (SEQ ID NO: 917), wherein n is an integer of 1. In some embodiments, L₃, L₃ₐ, L_{3b}, or L_{3c} has a formula of (GGGS)ₙ (SEQ ID NO: 918), wherein n is an integer of 1. In some embodiments, L₃, L₃ₐ, L_{3b}, or L_{3c} has a formula of (GGGGS)ₙ (SEQ ID NO: 919), wherein n is an integer of 1. In some embodiments, L₃, L₃ₐ, L_{3b}, or L_{3c} has a formula of (GSSGGS)ₙ (SEQ ID NO: 920), wherein n is an integer of 1.

In some embodiments, L₃, L₃ₐ, L_{3b}, or L_{3c} has a formula of (G₂S)ₙ (SEQ ID NO: 922), wherein n is an integer from 1 to 3. In some embodiments, L₃, L₃ₐ, L_{3b}, or L_{3c} has a formula of (GS)ₙ (SEQ ID NO: 938), wherein n is an integer from 1 to 3. In some embodiments, L₃, L₃ₐ, L_{3b}, or L_{3c} has a formula of (GSGGS)ₙ (SEQ ID NO: 939), wherein n is an integer from 1 to 3. In some embodiments, L₃, L₃ₐ, L_{3b}, or L_{3c} has a formula of (GGGS)ₙ (SEQ ID NO: 940), wherein n is an integer from 1 to 3. In some embodiments, L₃, L₃ₐ, L_{3b}, or L_{3c} has a formula of (GGGGS)ₙ (SEQ ID NO: 941), wherein n is an integer from 1 to 3. I In some embodiments, L₃, L₃ₐ, L_{3b}, or L_{3c} has a formula of (GSSGGS)ₙ (SEQ ID NO: 942), wherein n is an integer from 1 to 3.

### Configurations

In some embodiments, the Fab light chain polypeptide of A₂ is bound to a C-terminus of the single chain variable fragment (scFv) of A₁. In some embodiments, the Fab heavy chain polypeptide of A₂ is bound to a C-terminus of the single chain variable fragment (scFv) A₁. In some embodiments, the Fab light chain polypeptide of A₂ is bound to a N-terminus of the single chain variable fragment (scFv) of A₁. In some embodiments, the Fab heavy chain polypeptide of A₂ is bound to a N-terminus of the single chain variable fragment (scFv) A₁. In some embodiments, the Fab heavy chain polypeptide of A₂ is bound to the scFv heavy chain polypeptide of A₁. In some embodiments, the Fab light chain polypeptide of A₂ is bound to the scFv heavy chain polypeptide of A₁. In some embodiments, the Fab heavy chain polypeptide of A₂ is bound to the scFv light chain polypeptide of A₁. In some embodiments, the Fab light chain polypeptide of A₂ is bound to the scFv light chain polypeptide of A₁. In some embodiments, the Fab heavy chain polypeptide of A₂ is bound to the scFv heavy chain polypeptide of A₁ and L₂ is bound to the Fab light chain polypeptide of A₂.

In some embodiments, the Fab light chain polypeptide of A₂ is bound to the scFv heavy chain polypeptide of A₁ and L₂ is bound to the Fab heavy chain polypeptide of A₂. In some embodiments, the Fab heavy chain polypeptide of A₂ is bound to the scFv light chain polypeptide of A₁ and L₂ is bound to the Fab light chain polypeptide of A₂. In some embodiments, the Fab light chain polypeptide of A₂ is bound to the scFv light chain polypeptide of A₁ and L₂ is bound to the Fab heavy chain polypeptide of A₂.

Disclosed herein, in some embodiments, are polypeptides or polypeptide complexes comprising a structural arrangement according to Configuration 1:

wherein the polypeptide or polypeptide complex comprises a single chain variable fragment (scFv) comprising a light chain variable domain and a heavy chain variable domain, wherein the scFv is linked to a peptide (P₁) wherein the peptide comprises an amino acid sequence according to Peptide-1 (GSQCLGPEWEVCPY) SEQ ID NO: 1 or Peptide-2 (VYCGPEFDESVGCM) SEQ ID NO: 2, or the peptide comprises 1, 2, or 3, amino acid substitutions, additions, or deletions relative to the amino acid sequence of Peptide-1 or Peptide-2, wherein P₁ impairs binding of the scFv to CD3 and P₁ is linked to a N-terminus of the light chain variable domain of the scFv with a linking moiety (L₁) that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab heavy chain polypeptide is linked to a C terminus of the heavy chain variable domain of the scFv, and wherein the Fab is linked to P₂ and L₂, wherein P₂ comprises a peptide that impairs binding of the Fab to the tumor cell antigen; and L₂ comprises a linking moiety that connects the Fab light chain polypeptide to P₂ and is a substrate for a tumor specific protease.

Disclosed herein, in some embodiments, are polypeptides or polypeptide complexes comprising a structural arrangement according to Configuration 2:

wherein the polypeptide or polypeptide complex comprises a single chain variable fragment (scFv) comprising a light chain variable domain and a heavy chain variable domain, wherein the scFv is linked to a peptide wherein the peptide comprises an amino acid sequence according to Peptide-1 (GSQCLGPEWEVCPY) SEQ ID NO: 1 or Peptide-2 (VYCGPEFDESVGCM) SEQ ID NO: 2, or the peptide comprises 1, 2, or 3, amino acid substitutions, additions, or deletions relative to the amino acid sequence of Peptide-1 or Peptide-2, wherein the peptide impairs binding of the scFv to CD3 and the peptide is linked to the light chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide chain and a Fab heavy chain polypeptide chain, and wherein the Fab heavy chain polypeptide chain is linked to a C terminus of the heavy chain variable domain of the scFv.

Disclosed herein, in some embodiments, are polypeptides or polypeptide complexes comprising a structural arrangement according to Configuration 3:

wherein the polypeptide or polypeptide complex comprises a single chain variable fragment (scFv) comprising a light chain variable domain and a heavy chain variable domain, wherein the scFv is linked to a peptide (P₁) wherein the peptide comprises an amino acid sequence according to Peptide-1 (GSQCLGPEWEVCPY) SEQ ID NO: 1 or Peptide-2 (VYCGPEFDESVGCM) SEQ ID NO: 2, or the peptide comprises 1, 2, or 3, amino acid substitutions, additions, or deletions relative to the amino acid sequence of Peptide-1 or Peptide-2, wherein P₁ impairs binding of the scFv to CD3 and P₁ is linked to a N-terminus of the light chain variable domain of the scFv with a linking moiety that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the heavy chain variable domain of the scFv, and wherein the Fab is linked to P₂ and L₂, wherein P₂ comprises a peptide that impairs binding to the tumor cell antigen; and L₂ comprises a linking moiety that connects the Fab heavy chain polypeptide to P₂ and is a substrate for a tumor specific protease.

Disclosed herein, in some embodiments, are polypeptides or polypeptide complexes comprising a structural arrangement according to Configuration 4:

wherein the polypeptide or polypeptide complex comprises a single chain variable fragment (scFv) comprising a light chain variable domain and a heavy chain variable domain, wherein the scFv is further linked to a peptide wherein the peptide comprises an amino acid sequence according to Peptide-1 (GSQCLGPEWEVCPY) SEQ ID NO: 1 or Peptide-2 (VYCGPEFDESVGCM) SEQ ID NO: 2, or the peptide comprises 1, 2, or 3, amino acid substitutions, additions, or deletions relative to the amino acid sequence of Peptide-1 or Peptide-2, wherein the peptide impairs binding of the scFv to CD3 and the peptide is linked to a N-terminus of the light chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the heavy chain variable domain of the scFv.

Disclosed herein, in some embodiments, are polypeptides or polypeptide complexes comprising a structural arrangement according to Configuration 5:

wherein the polypeptide or polypeptide complex comprises a single chain variable fragment (scFv) comprising a light chain variable domain and a heavy chain variable domain, wherein the scFv is linked to a peptide (P₁) wherein the peptide comprises an amino acid sequence according to Peptide-1 (GSQCLGPEWEVCPY) SEQ ID NO: 1 or Peptide-2 (VYCGPEFDESVGCM) SEQ ID NO: 2, or the peptide comprises 1, 2, or 3, amino acid substitutions, additions, or deletions relative to the amino acid sequence of Peptide-1 or Peptide-2, wherein P₁ impairs binding of the scFv to CD3 and P₁ is linked to a N-terminus of the heavy chain variable domain of the scFv with a linking moiety (L₁) that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab heavy chain polypeptide is linked to a C terminus of the light chain variable domain of the scFv, and wherein the Fab is linked to P₂ and L₂, wherein P₂ comprises a peptide that impairs binding of the Fab to the tumor cell antigen; and L₂ comprises a linking moiety that connects the Fab light chain polypeptide to P₂ and is a substrate for a tumor specific protease.

Disclosed herein, in some embodiments, are polypeptides or polypeptide complexes comprising a structural arrangement according to Configuration 6:

wherein the polypeptide or polypeptide complex comprises a single chain variable fragment (scFv) comprising a light chain variable domain and a heavy chain variable domain, wherein the scFv is linked to a peptide wherein the peptide comprises an amino acid sequence according to Peptide-1 (GSQCLGPEWEVCPY) SEQ ID NO: 1 or Peptide-2 (VYCGPEFDESVGCM) SEQ ID NO: 2, or the peptide comprises 1, 2, or 3, amino acid substitutions, additions, or deletions relative to the amino acid sequence of Peptide-1 or Peptide-2, wherein the peptide impairs binding of the scFv to CD3 and the peptide is linked to the heavy chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide chain and a Fab heavy chain polypeptide chain, and wherein the Fab heavy chain polypeptide chain is linked to a C terminus of the light chain variable domain of the scFv.

Disclosed herein, in some embodiments, are polypeptides or polypeptide complexes comprising a structural arrangement according to Configuration 7:

wherein the polypeptide or polypeptide complex comprises a single chain variable fragment (scFv) comprising a light chain variable domain and a heavy chain variable domain, wherein the scFv is linked to a peptide (P₁) wherein the peptide comprises an amino acid sequence according to Peptide-1 (GSQCLGPEWEVCPY) SEQ ID NO: 1 or Peptide-2 (VYCGPEFDESVGCM) SEQ ID NO: 2, or the peptide comprises 1, 2, or 3, amino acid substitutions, additions, or deletions relative to the amino acid sequence of Peptide-1 or Peptide-2, wherein P₁ impairs binding of the scFv to CD3 and P₁ is linked to a N-terminus of the heavy chain variable domain of the scFv with a linking moiety (L₁) that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the light chain variable domain of the scFv, and wherein the Fab is linked to P₂ and L₂, wherein P₂ comprises a peptide that impairs binding of the Fab to the tumor cell antigen; and L₂ comprises a linking moiety that connects the Fab heavy chain polypeptide to P₂ and is a substrate for a tumor specific protease.

Disclosed herein, in some embodiments, are polypeptides or polypeptide complexes comprising a structural arrangement according to Configuration 8:

wherein the polypeptide or polypeptide complex comprises a single chain variable fragment (scFv) comprising a light chain variable domain and a heavy chain variable domain, wherein the scFv is linked to a peptide wherein the peptide comprises an amino acid sequence according to Peptide-1 (GSQCLGPEWEVCPY) SEQ ID NO: 1 or Peptide-2 (VYCGPEFDESVGCM) SEQ ID NO: 2, or the peptide comprises 1, 2, or 3, amino acid substitutions, additions, or deletions relative to the amino acid sequence of Peptide-1 or Peptide-2, wherein the peptide impairs binding of the scFv to CD3 and the peptide is linked to a N-terminus of the heavy chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the light chain variable domain of the scFv.

Disclosed herein, in some embodiments, are polypeptides or polypeptide complexes comprising a structural arrangement according to Configuration 9:

wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab light chain polypeptide with a linking moiety (L₁) that is a substrate for a tumor specific protease, and the P₁ is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab heavy chain polypeptide, wherein the scFv is linked to P₂ and L₂, wherein P₂ comprises a peptide according to an amino acid sequence according to Peptide-1 (GSQCLGPEWEVCPY) SEQ ID NO: 1 or Peptide-2 (VYCGPEFDESVGCM) SEQ ID NO: 2, or P₂ comprises 1, 2, or 3, amino acid substitutions, additions, or deletions relative to the amino acid sequence of Peptide-1 or Peptide-2, wherein P₂ impairs binding of the scFv to CD3, and L₂ comprises a linking moiety that connects the light chain variable domain of the scFv to P₂ and is a substrate for a tumor specific protease.

Disclosed herein, in some embodiments, are polypeptides or polypeptide complexes comprising a structural arrangement according to Configuration 10:

wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab heavy chain polypeptide with a linking moiety (L₁) that is a substrate for a tumor specific protease, and P₁
is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab light chain polypeptide, wherein the scFv further is linked to P₂ and L₂, wherein P₂ comprises a peptide according to an amino acid sequence according to Peptide-1 (GSQCLGPEWEVCPY) SEQ ID NO: 1 or Peptide-2 (VYCGPEFDESVGCM) SEQ ID NO: 2, or P₂ comprises 1, 2, or 3, amino acid substitutions, additions, or deletions relative to the amino acid sequence of Peptide-1 or Peptide-2, wherein P₂ impairs binding of the scFv to CD3, and L₂ comprises a linking moiety that connects the light chain variable domain of the scFv to P₂ and is a substrate for a tumor specific protease.

Disclosed herein, in some embodiments, are polypeptides or polypeptide complexes comprising a structural arrangement according to Configuration 11:

wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab light chain polypeptide with a linking moiety (L₁) that is a substrate for a tumor specific protease, and P₁
is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab heavy chain polypeptide, wherein the scFv is linked to P₂ and L₂, wherein P₂ comprises a peptide according to an amino acid sequence according to Peptide-1 (GSQCLGPEWEVCPY) SEQ ID NO: 1 or Peptide-2 (VYCGPEFDESVGCM) SEQ ID NO: 2, or P₂ comprises 1, 2, or 3, amino acid substitutions, additions, or deletions relative to the amino acid sequence of Peptide-1 or Peptide-2, wherein P₂ impairs binding of the scFv to CD3, and L₂ comprises a linking moiety that connects the heavy chain variable domain of the scFv to P₂ and is a substrate for a tumor specific protease.

Disclosed herein, in some embodiments, are polypeptides or polypeptide complexes comprising a structural arrangement according to Configuration 12:

wherein the polypeptide or polypeptide complex comprises a Fab that binds to a tumor cell antigen, the Fab comprising a Fab light chain polypeptide and a Fab heavy chain polypeptide, wherein the Fab is linked to a (P₁) that impairs binding of the Fab to the tumor cell antigen and P₁ is linked to a N terminus of the Fab heavy chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab light chain polypeptide, wherein the scFv is linked to P₂ and L₂, wherein P₂ comprises a peptide according to an amino acid sequence according to Peptide-1 (GSQCLGPEWEVCPY) SEQ ID NO: 1 or Peptide-2 (VYCGPEFDESVGCM) SEQ ID NO: 2, or P₂ comprises 1, 2, or 3, amino acid substitutions, additions, or deletions relative to the amino acid sequence of Peptide-1 or Peptide-2, wherein P₂ impairs binding of the scFv to CD3, and L₂ comprises a linking moiety that connects the heavy chain variable domain of the scFv to P₂ and is a substrate for a tumor specific protease.
Polypeptides or polypeptide complexes, in some embodiments, comprise a sequence set forth in Table 5. In some embodiments, the sequence comprises at least or about 70%, 80%, 85%, 90%, 91%, 92%, 93%, 94%, 95%, 96%, 97%, 98%, 99%, or 100% sequence identity to any one of SEQ ID NOs: 13-18.

TABLE 5
Exemplary amino acid sequences of isolated polypeptides or polypeptide
complexes (PC) comprising Peptide-1 or Peptide-2

| | Amino Acid Sequence | |
|---|---|---|
| Construct Description | (N to C) | SEQ ID NO: |
| PC-1-light chain | QGQSGQLSCEGWAMNREQCRAGSSGGSGGS | 13 |
| | GGSGISSGLLSGRSDNHGSSGTDILLTQSPVILS | |
| | VSPGERVSFSCRASQSIGTNIHWYQQRTNGSP | |
| | RLLIKYASESISGIPSRFSGSGSGTDFTLSINSVE | |
| | SEDIADYYCQQNNNWPTTFGAGTKLELKRT | |
| | VAAPSVFIFPPSDEQLKSGTASVVCLLNNFYPR | |
| | EAKVQWKVDNALQSGNSQESVTEQDSKDST | |
| | YSLSSTLTLSKADYEKHKVYACEVTHQGLSSP | |
| | VTKSFNRGEC | |
| PC-1-heavy chain | EVQLVESGGGLQPGNSLRLSCAASGFTFSK | 14 |
| | FGMSWVRQAPGKGLEWVSSISGSGRDTLYA | |
| | DSVKGRFTISRDNAKTTLYLQMNSLRPEDTA | |
| | VYYCTIGGSLSVSSQGTLVTVSSGGGGSGGG | |
| | SGGGSQCLGPEWEVCPYGGGGSGGGSGGISS | |
| | GLLSGRSDNHGGGSEVQLVESGGGLVQPGGS | |
| | LKLSCAASGFTFNKYAMNWVRQAPGKGLE | |
| | WVARIRSKYNNYATYYADSVKDRFTISRDDS | |
| | KNTAYLQMNNLKTEDTAVYYCVRHGNFGN | |
| | SYISYWAYWGQGTLVTVSSGGGGSGGGGSG | |
| | GGGSQTVVTQEPSLTVSPGGTVTLTCGSSTGA | |
| | VTSGNYPNWVQQKPGQAPRGLIGGTKFLAP | |
| | GTPARFSGSLLGGKAALTLSGVQPEDEAEYY | |
| | CVLWYSNRWVFGGGTKLTVLGGGGSQVQL | |
| | KQSGPGLVQPSQSLSITCTVSGFSLTNYGVH | |
| | WVRQSPGKGLEWLGVIWSGGNTDYNTPFTS | |
| | RLSINKDNSKSQVFFKMNSLQSQDTAIYYCAR | |
| | ALTYYDYEFAYWGQGTLVTVSAASTKGPSV | |
| | FPLAPSSKSTSGGTAALGCLVKDYFPEPVTVS | |
| | WNSGALTSGVHTFPAVLQSSGLYSLSSVVTVP | |
| | SSSLGTQTYICNVNHKPSNTKVDKKVEPKSC | |
| PC-2-light chain | QGQSGQLSCEGWAMNREQCRAGSSGGSGGS | 15 |
| | GGSGISSGLLSGRSDNHGSSGTDILLTQSPVILS | |
| | VSPGERVSFSCRASQSIGTNIHWYQQRTNGSP | |
| | RLLIKYASESISGIPSRFSGSGSGTDFTLSINSVE | |
| | SEDIADYYCQQNNNWPTTFGAGTKLELKRT | |
| | VAAPSVFIFPPSDEQLKSGTASVVCLLNNFYPR | |
| | EAKVQWKVDNALQSGNSQESVTEQDSKDST | |
| | YSLSSTLTLSKADYEKHKVYACEVTHQGLSSP | |
| | VTKSFNRGEC | |
| PC-2-heavy chain | EVQLVESGGGLVQPGNSLRLSCAASGFTFSK | 16 |
| | FGMSWVRQAPGKGLEWVSSISGSGRDTLYA | |
| | DSVKGRFTISRDNAKTTLYLQMNSLRPEDTA | |
| | VYYCTIGGSLSVSSQGTLVTVSSGGGGSGGG | |
| | SGGVYCGPEFDESVGCMGGGGSGGGSGGISS | |
| | GLLSGRSDNHGGGSEVQLVESGGGLVQPGGS | |
| | LKLSCAASGFTFNKYAMNWVRQAPGKGLE | |
| | WVARIRSKYNNYATYYADSVKDRFTISRDDS | |
| | KNTAYLQMNNLKTEDTAVYYCVRHGNFGN | |
| | SYISYWAYWGQGTLVTVSSGGGGSGGGGSG | |
| | GGGSQTVVTQEPSLTVSPGGTVTLTCGSSTGA | |
| | VTSGNYPNWVQQKPGQAPRGLIGGTKFLAP | |
| | GTPARFSGSLLGGKAALTLSGVQPEDEAEYY | |
| | CVLWYSNRWVFGGGTKLTVLGGGGSQVQL | |
| | KQSGPGLVQPSQSLSITCTVSGFSLTNYGVH | |
| | WVRQSPGKGLEWLGVIWSGGNTDYNTPFTS | |
| | RLSINKDNSKSQVFFKMNSLQSQDTAIYYCAR | |
| | ALTYYDYEFAYWGQGTLVTVSAASTKGPSV | |
| | FPLAPSSKSTSGGTAALGCLVKDYFPEPVTVS | |
| | WNSGALTSGVHTFPAVLQSSGLYSLSSVVTVP | |
| | SSSLGTQTYICNVNHKPSNTKVDKKVEPKSC | |
| PC-3-light chain | QILLTQSPVILSVSPGERVSFSCRASQSIGTNIH | 17 |
| | WYQQRTNGSPRLLIKYASESISGIPSRFSGSGS | |
| | GTDFTLSINSVESEDIADYYCQQNNNWPTTF | |
| | GAGTKLELKRTVAAPSVFIFPPSDEQLKSGTA | |
| | SVVCLLNNFYPREAKVQWKVDNALQSGNSQ | |
| | ESVTEQDSKDSTYSLSSTLTLSKADYEKHKVY | |
| | ACEVTHQGLSSPVTKSFNRGEC | |
| PC-3-heavy chain | EVQLVESGGGLVQPGGSLKLSCAASGFTFNK | 18 |
| | YAMNWVRQAPGKGLEWVARIRSKYNNYAT | |
| | YYADSVKDRFTISRDDSKNTAYLQMNNLKTE | |
| | DTAVYYCVRHGNFGNSYISYWAYWGQGTL | |
| | VTVSSGGGGSGGGGSGGGGSQTVVTQEPSLT | |
| | VSPGGTVTLTCGSSTGAVTSGNYPNWVQQKP | |
| | GQAPRGLIGGTKFLAPGTPARFSGSLLGGKAA | |
| | LTLSGVQPEDEAEYYCVLWYSNRWVFGGGT | |
| | KLTVLGGGGSQVQLKQSGPGLVQPSQSLSITC | |
| | TVSGFSLTNYGVHWVRQSPGKGLEWLGVIW | |
| | SGGNTDYNTPFTSRLSINKDNSKSQVFFKMNS | |
| | LQSQDTAIYYCARALTYYDYEFAYWGQGTL | |
| | VTVSAASTKGPSVFPLAPSSKSTSGGTAALGC | |
| | LVKDYFPEPVTVSWNSGALTSGVHTFPAVLQ | |
| | SSGLYSLSSVVTVPSSSLGTQTYICNVNHKPSN | |
| | TKVDKKVEPKSC | |

### Polynucleotides Encoding Polypeptides or Polypeptide Complexes

Disclosed herein, in some embodiments, are isolated recombinant nucleic acid molecules encoding polypeptides or polypeptide complexes as disclosed herein. In some embodiments, the polypeptides or polypeptide complexes comprise an antibody or an antibody fragment.

Disclosed herein, in some embodiments, are isolated recombinant nucleic acid molecules encoding polypeptide or polypeptide complex comprising a peptide that impairs binding of an anti-CD3 binding domain to CD3 wherein the peptide comprises an amino acid sequence according to Peptide-1 (GSQCLGPEWEVCPY) SEQ ID NO: 1 or Peptide-2 (VYCGPEFDESVGCM) SEQ ID NO: 2, or the peptide comprises 1, 2, or 3, amino acid substitutions, additions, or deletions relative to the amino acid sequence of Peptide-1 or Peptide-2. Disclosed herein, in some embodiments, are isolated recombinant nucleic acid molecules encoding polypeptide or polypeptide complex comprising a peptide that impairs binding of an anti-CD3 binding domain to CD3 wherein the peptide comprises an amino acid sequence according to Peptide-1 (GSQCLGPEWEVCPY) SEQ ID NO: 1 or Peptide-2 (VYCGPEFDESVGCM) SEQ ID NO: 2.

Disclosed herein, in some embodiments, are isolated recombinant nucleic acid molecules encoding polypeptides or polypeptide complexes comprising a peptide that impairs binding of an anti-CD3 binding domain to CD3 wherein the peptide comprises an amino acid sequence selected from SEQ ID NOs: 1-2, 19-55 or the peptide comprises 1, 2, or 3, amino acid substitutions, additions, or deletions relative to an amino acid sequence selected from SEQ ID NOs: 1-2, 19-55. In some embodiments, the peptide comprises an amino acid sequence selected from SEQ ID NOs: 1-2, and 19-55. In some embodiments, the peptide comprises the amino acid sequence of SEQ ID NO: 1. In some embodiments, the peptide comprises the amino acid sequence of SEQ ID NO: 2. In some embodiments, the peptide comprises the amino acid sequence of SEQ ID NO: 54.

Disclosed herein, in some embodiments, are isolated recombinant nucleic acid molecules encoding polypeptides or polypeptide complexes comprising an anti-CD3 binding domain that is linked to a peptide that impairs binding of the anti-CD3 binding domain to CD3 wherein the peptide comprises an amino acid sequence according to Z₁-Z₂-C-Z₄-P-Z₆-Z₇-Z₈-Z₉-Z₁₀-Z₁₁-Z₁₂-C-Z₁₄ and Z₁ is selected from D, Y, F, I, N, V, H, L, A, T, S, and P; Z₂ is selected from D, Y, L, F, I, N, A, V, H, T, and S; Z₄ is selected from G and W; Z₆ is selected from E, D, V, and P; Z₇ is selected from W, L, F, V, G, M, I, and Y; Z₈ is selected from E, D, P, and Q; Z₉ is selected from E, D, Y, V, F, W, P, L, and Q; Z₁₀ is selected from S, D, Y, T, I, F, V, N, A, P, L, and H; Z₁₁ is selected from I, Y, F, V, L, T, N, S, D, A, and H; Z₁₂ is selected from F, D, Y, L, I, V, A, N, T, P, S, and H; and Z₁₄ is selected from D, Y, N, F, I, P, V, A, T, H, L and S. In some embodiments, Z₁ is selected from D, Y, F, I, and N; Z₂ is selected from D, Y, L, F, I, and N; Z₄ is selected from G and W; Z₆ is selected from E and D; Z₇ is selected from W, L, F, and V; Z₈ is selected from E and D; Z₉ is selected from E, D, Y, and V; Z₁₀ is selected from S, D, Y, T, and I; Z₁₁ is selected from I, Y, F, V, L, and T; Z₁₂ is selected from F, D, Y, L, I, V, A, and N; and Z₁₄ is selected from D, Y, N, F, I, and P. In some embodiments, Z₁ is selected from D, Y, and F; Z₂ is selected from D, Y, L, and F; Z₄ is selected from G and W; Z₆ is selected from E and D; Z₇ is selected from W, L, and F; Z₈ is selected from E and D; Z₉ is selected from E and D; Z₁₀ is selected from S, D, and Y; Z₁₁ is selected from I, Y, and F; Z₁₂ is selected from F, D, Y, and L; and Z₁₄ is selected from D, Y, and N. In some embodiments, the peptide comprises an amino acid sequence of Table 17. In some embodiments, the peptide comprises an amino acid sequence according to any one of SEQ ID NOs: 44-55. In some embodiments, the peptide comprises the amino acid sequence of SEQ ID NO: 2. In some embodiments, the peptide comprises the amino acid sequence of SEQ ID NO: 54.

Disclosed herein, in some embodiments, are isolated recombinant nucleic acid molecules encoding polypeptides or polypeptide complexes comprising an anti-CD3 binding domain that is linked to a peptide that impairs binding of the anti-CD3 binding domain to CD3 wherein the peptide comprises an amino acid sequence according to U₁-U₂-C-U₄-P-U₆-U₇-U₈-U₉-U₁₀-U₁₁-U₁₂-C-U₁₄ and U₁ is selected from D, Y, F, I, N, V, H, L, A, T, S, and P; U₂ is selected from D, Y, L, F, I, N, A, V, H, T, and S; U₄ is selected from G and W; U₆ is selected from E, D, V, and P; U₇ is selected from W, L, F, V, G, M, I, and Y; U₈ is selected from E, D, P, and Q; U₉ is selected from E, D, Y, V, F, W, P, L, and Q; U₁₀ is selected from S, D, Y, T, I, F, V, N, A, P, L, and H; U₁₁ is selected from I, Y, F, V, L, T, N, S, D, A, and H; U₁₂ is selected from F, D, Y, L, I, V, A, N, T, P, S, G, and H; and U₁₄ is selected from D, Y, N, F, I, P, V, A, T, H, L, M, and S. In some embodiments, U₁ is selected from D, Y, F, I, V, and N; U₂ is selected from D, Y, L, F, I, and N; U₄ is selected from G and W; U₆ is selected from E and D; U₇ is selected from W, L, F, G, and V; U₈ is selected from E and D; U₉ is selected from E, D, Y, and V; U₁₀ is selected from S, D, Y, T, and I; U₁₁ is selected from I, Y, F, V, L, and T; U₁₂ is selected from F, D, Y, L, I, V, A, G, and N; and U₁₄ is selected from D, Y, N, F, I, M, and P. In some embodiments, U₁ is selected from D, Y, V, and F; U₂ is selected from D, Y, L, and F; U₄ is selected from G and W; U₆ is selected from E and D; U₇ is selected from W, L, G, and F; U₈ is selected from E and D; U₉ is selected from E and D; U₁₀ is selected from S, D, T, and Y; U₁₁ is selected from I, Y, V, L, and F; U₁₂ is selected from F, D, Y, G, A, and L; and U₁₄ is selected from D, Y, M, and N. In some embodiments, the peptide comprises an amino acid sequence of Table 17. In some embodiments, the peptide comprises an amino acid sequence according to any one of SEQ ID NOs: 44-55. In some embodiments, the peptide comprises the amino acid sequence of SEQ ID NO: 2. In some embodiments, the peptide comprises the amino acid sequence of SEQ ID NO: 54.

In some embodiments, the peptide is connected to the anti-CD3 binding domain in a configuration according to: A₁-L₁-P₁ (Formula I) wherein A₁ comprises the anti-CD3 binding domain; L₁ comprises a cleavable linker that is a substrate for a tumor specific protease; P₁ comprises the peptide that impairs binding of the anti-CD3 binding domain to CD3. In some embodiments, the peptide is connected to the anti-CD3 binding domain in a configuration according to: A₁-L₁-P (Formula I) wherein A₁ is the anti-CD3 binding domain; L₁ is a cleavable linker that is a substrate for a tumor specific protease; P₁ is the peptide that impairs binding of the anti-CD3 binding domain to CD3. In some embodiments, the peptide is connected to the anti-CD3 binding domain in a configuration comprising Formula I: A₁-L₁-P₁ (Formula I) wherein A₁ comprises the anti-CD3 binding domain; L₁ comprises a cleavable linker that is a substrate for a tumor specific protease; P₁ comprises the peptide that impairs binding of the anti-CD3 binding domain to CD3. In some embodiments, the peptide is connected to the anti-CD3 binding domain in a configuration comprising Formula I: A₁-L₁-P₁ (Formula I) wherein A₁ is the anti-CD3 binding domain; L₁ is a cleavable linker that is a substrate for a tumor specific protease; P₁ is the peptide that impairs binding of the anti-CD3 binding domain to CD3. In some embodiments, A₁ is further linked to a tumor antigen binding domain (A₂). In some embodiments, the polypeptide or polypeptide complex is according to Formula Ia: P₂-L₂-A₂-A₁-L₁-P₁ (Formula Ia) wherein P₂ comprises a peptide that impairs binding of A₂ to a tumor antigen; and L₂ comprises a second cleavable linker that connects A₂ to P₂ and is a substrate for a tumor specific protease.

### Pharmaceutical Compositions

Disclosed herein, in some embodiments, are pharmaceutical compositions comprising: (a) the polypeptides or polypeptide complexes as disclosed herein; and (b) a pharmaceutically acceptable excipient.

In some embodiments, the polypeptide or polypeptide complex further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the polypeptide or polypeptide complex as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk−, hgprt− or aprt− cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

In some embodiments, vectors include any suitable vectors derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or coli bacilli.

Exemplary prokaryotic host cells include, but are not limited to, BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS 115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, FUT8 KO CHOK1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a bispecific antibody comprising a first antigen-binding site that specifically binds to CD3 and a second antigen-binding site that specifically binds to a tumor antigen defined herein before.

The label or package insert indicates that the composition is used for treating the condition of choice. Moreover, the article of manufacture may comprise (a) a first container with a composition contained therein, wherein the composition comprises the bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2: 106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

The term “Fab” refers to a protein that contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EMBODIMENTS

Embodiment 1 comprises an isolated polypeptide or polypeptide complex comprising a peptide that impairs binding of an anti-CD3 binding domain to CD3 wherein the peptide comprises an amino acid sequence selected from SEQ ID NOs: 1-2, 19-55 or the peptide comprises 1, 2, or 3, amino acid substitutions, additions, or deletions relative to an amino acid sequence selected from SEQ ID NOs: 1-2, 19-55.

Embodiment 2 comprises an isolated polypeptide or polypeptide complex of embodiment 1, wherein the peptide comprises an amino acid sequence selected from SEQ ID NOs: 1-2, and 19-55.

Embodiment 3 comprises an isolated polypeptide or polypeptide complex of any one of embodiments 1-2, wherein the peptide comprises the amino acid sequence of SEQ ID NO: 1.

Embodiment 4 comprises an isolated polypeptide or polypeptide complex of any one of embodiments 1-2, wherein the peptide comprises the amino acid sequence of SEQ ID NO: 2.

Embodiment 5 comprises an isolated polypeptide or polypeptide complex of any one of embodiments 1-2, wherein the peptide comprises the amino acid sequence of SEQ ID NO: 54.

Embodiment 6 comprises an isolated polypeptide or polypeptide complex comprising an anti-CD3 binding domain that is linked to a peptide that impairs binding of the anti-CD3 binding domain to CD3 wherein the peptide comprises an amino acid sequence according to Z₁-Z₂-C-Z₄-P-Z₆-Z₇-Z₈-Z₉-Z₁₀-Z₁₁-Z₁₂-C-Z₁₄ and Z₁ is selected from D, Y, F, I, N, V, H, L, A, T, S, and P; Z₂ is selected from D, Y, L, F, I, N, A, V, H, T, and S; Z₄ is selected from G and W; Z₆ is selected from E, D, V, and P; Z₇ is selected from W, L, F, V, G, M, I, and Y; Z₈ is selected from E, D, P, and Q; Z₉ is selected from E, D, Y, V, F, W, P, L, and Q; Z₁₀ is selected from S, D, Y, T, I, F, V, N, A, P, L, and H; Z₁₁ is selected from I, Y, F, V, L, T, N, S, D, A, and H; Z₁₂ is selected from F, D, Y, L, I, V, A, N, T, P, S, and H; and Z₁₄ is selected from D, Y, N, F, I, P, V, A, T, H, L and S.

Embodiment 7 comprises an isolated polypeptide or polypeptide complex of embodiment 6, wherein Z₁ is selected from D, Y, F, I, and N; Z₂ is selected from D, Y, L, F, I, and N; Z₄ is selected from G and W; Z₆ is selected from E and D; Z₇ is selected from W, L, F, and V; Z₈ is selected from E and D; Z₉ is selected from E, D, Y, and V; Z₁₀ is selected from S, D, Y, T, and I; Z₁₁ is selected from I, Y, F, V, L, and T; Z₁₂ is selected from F, D, Y, L, I, V, A, and N; and Z₁₄ is selected from D, Y, N, F, I, and P.

Embodiment 8 comprises an isolated polypeptide or polypeptide complex of any one of embodiments 6-7, wherein Z₁ is selected from D, Y, and F; Z₂ is selected from D, Y, L, and F; Z₄ is selected from G and W; Z₆ is selected from E and D; Z₇ is selected from W, L, and F; Z₈ is selected from E and D; Z₉ is selected from E and D; Z₁₀ is selected from S, D, and Y; Z₁₁ is selected from I, Y, and F; Z₁₂ is selected from F, D, Y, and L; and Z₁₄ is selected from D, Y, and N.

Embodiment 9 comprises an isolated polypeptide or polypeptide complex comprising an anti-CD3 binding domain that is linked to a peptide that impairs binding of the anti-CD3 binding domain to CD3 wherein the peptide comprises an amino acid sequence according to U₁-U₂-C-U₄-P-U₆-U₇-U₈-U₉-U₁₀-U₁₁-U₁₂-C-U₁₄ and U₁ is selected from D, Y, F, I, N, V, H, L, A, T, S, and P; U₂ is selected from D, Y, L, F, I, N, A, V, H, T, and S; U₄ is selected from G and W; U₆ is selected from E, D, V, and P; U₇ is selected from W, L, F, V, G, M, I, and Y; U₈ is selected from E, D, P, and Q; U₉ is selected from E, D, Y, V, F, W, P, L, and Q; U₁₀ is selected from S, D, Y, T, I, F, V, N, A, P, L, and H; U₁₁ is selected from I, Y, F, V, L, T, N, S, D, A, and H; U₁₂ is selected from F, D, Y, L, I, V, A, N, T, P, S, G, and H; and U₁₄ is selected from D, Y, N, F, I, P, V, A, T, H, L, M, and S.

Embodiment 10 comprises an isolated polypeptide or polypeptide complex of embodiment 9, wherein U₁ is selected from D, Y, F, I, V, and N; U₂ is selected from D, Y, L, F, I, and N; U₄ is selected from G and W; U₆ is selected from E and D; U₇ is selected from W, L, F, G, and V; U₈ is selected from E and D; U₉ is selected from E, D, Y, and V; U₁₀ is selected from S, D, Y, T, and I; U₁₁ is selected from I, Y, F, V, L, and T; U₁₂ is selected from F, D, Y, L, I, V, A, G, and N; and U₁₄ is selected from D, Y, N, F, I, M, and P.

Embodiment 11 comprises an isolated polypeptide or polypeptide complex of any one of embodiments 9-10, wherein U₁ is selected from D, Y, V, and F; U₂ is selected from D, Y, L, and F; U₄ is selected from G and W; U₆ is selected from E and D; U₇ is selected from W, L, G, and F; U₈ is selected from E and D; U₉ is selected from E and D; U₁₀ is selected from S, D, T, and Y; U₁₁ is selected from I, Y, V, L, and F; U₁₂ is selected from F, D, Y, G, A, and L; and U₁₄ is selected from D, Y, M, and N.

Embodiment 12 comprises an isolated polypeptide or polypeptide complex of any one of embodiments 6-11, wherein the peptide comprises an amino acid sequence of Table 17.

Embodiment 13 comprises an isolated polypeptide or polypeptide complex of any one of embodiments 6-11, wherein the peptide comprises an amino acid sequence according to any one of SEQ ID NOs: 44-55.

Embodiment 14 comprises an isolated polypeptide or polypeptide complex of any one of embodiments 6 and 9-12, wherein the peptide comprises the amino acid sequence of SEQ ID NO: 2.

Embodiment 15 comprises an isolated polypeptide or polypeptide complex of any one of embodiments 6-7 and 9-13, wherein the peptide comprises the amino acid sequence of SEQ ID NO: 54.

Embodiment 16 comprises an isolated polypeptide or polypeptide complex of any one of embodiments 1-15, wherein the peptide is connected to the anti-CD3 binding domain in a configuration according to Formula I: A₁-L₁-P₁ wherein A₁ comprises the anti-CD3 binding domain; L₁ comprises a cleavable linker that is a substrate for a tumor specific protease; P₁ comprises the peptide that impairs binding of the anti-CD3 binding domain to CD3.

Embodiment 17 comprises an isolated polypeptide or polypeptide complex of embodiment 16, wherein P₁ is connected N-terminal to the cleavable linker and A₁ is connected C-terminal to the cleavable linker.

Embodiment 18 comprises an isolated polypeptide or polypeptide complex of embodiment 16, wherein P₁ is connected C-terminal to the cleavable linker and A₁ is connected N-terminal to the cleavable linker.

Embodiment 19 comprises an isolated polypeptide or polypeptide complex of any one of embodiments 16-18, wherein P₁ is bound to A₁ through ionic interactions, electrostatic interactions, hydrophobic interactions, P₁-stacking interactions, or H-bonding interactions, or a combination thereof.

Embodiment 20 comprises an isolated polypeptide or polypeptide complex of any one of embodiments 16-19, wherein A₁ is further linked to a tumor antigen binding domain (A₂).

Embodiment 21 comprises an isolated polypeptide or polypeptide complex of any one of embodiments 16-20, wherein the polypeptide or polypeptide complex is according to Formula Ia: P₂-L₂-A₂-A₁-L₁-P₁ wherein P₂ comprises a peptide that impairs binding of A₂ to a tumor antigen; and L₂ comprises a second cleavable linker that connects A₂ to P₂ and is a substrate for a tumor specific protease.

Embodiment 22 comprises an isolated polypeptide or polypeptide complex of any one of embodiments 16-21, wherein A₁ comprises an antibody or antibody fragment.

Embodiment 23 comprises an isolated polypeptide or polypeptide complex of any one of embodiments 16-22, wherein A₁ comprises an antibody or antibody fragment that is human or humanized.

Embodiment 24 comprises an isolated polypeptide or polypeptide complex of any one of embodiments 16-17 and 19-23, wherein L₁ is bound to N-terminus of the antibody or antibody fragment of A₁.

Embodiment 25 comprises an isolated polypeptide or polypeptide complex of any one of embodiments 16 and 18-23, wherein A₂ is bound to N-terminus of the antibody or antibody fragment of A₁.

Embodiment 26 comprises an isolated polypeptide or polypeptide complex of any one of embodiments 16, 18-23, and 25, wherein L₁ is bound to the C-terminus of the antibody or antibody fragment of A₁.

Embodiment 27 comprises an isolated polypeptide or polypeptide complex of any one of embodiments 16-17 and 19-24, wherein A₂ is bound to the C-terminus of the antibody or antibody fragment of A₁.

Embodiment 28 comprises an isolated polypeptide or polypeptide complex of any one of embodiments 22-27, wherein the antibody or antibody fragment of A₁ comprises a single chain variable fragment, a single domain antibody, or a Fab fragment.

Embodiment 29 comprises an isolated polypeptide or polypeptide complex of embodiment 28, wherein A₁ is the single chain variable fragment (scFv).

Embodiment 30 comprises an isolated polypeptide or polypeptide complex of embodiment 29, wherein the scFv comprises a scFv heavy chain polypeptide and a scFv light chain polypeptide.

Embodiment 31 comprises an isolated polypeptide or polypeptide complex of embodiment 28, wherein A₁ is the single domain antibody.

Embodiment 32 comprises an isolated polypeptide or polypeptide complex of any one of embodiments 22-27, wherein the antibody or antibody fragment thereof comprises a single chain variable fragment (scFv), a heavy chain variable domain (VH domain), a light chain variable domain (VL domain), or a variable domain (VHH) of a camelid derived single domain antibody.

Embodiment 33 comprises an isolated polypeptide or polypeptide complex of any one of embodiments 16-30 and 32, wherein A₁ comprises an anti-CD3e single chain variable fragment.

Embodiment 34 comprises an isolated polypeptide or polypeptide complex of any one of embodiments 16-32, wherein A₁ comprises an anti-CD3e single chain variable fragment that has a KD binding of 1 μM or less to CD3 on CD3 expressing cells.

Embodiment 35 comprises an isolated polypeptide or polypeptide complex of any one of embodiments 16-30 and 32-34, wherein A₁ comprises a variable light chain and variable heavy chain each of which is capable of specifically binding to human CD3.

Embodiment 36 comprises an isolated polypeptide or polypeptide complex of any one of embodiments 28-30 and 32-35, wherein A₁ comprises the scFv comprising a scFv heavy chain variable domain and an scFv light chain variable domain comprising complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 3; HC-CDR2: SEQ ID NO: 4; HC-CDR3: SEQ ID NO: 5; and the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 6; LC-CDR2: SEQ ID NO: 7; and LC-CDR3: SEQ ID NO: 8.

Embodiment 37 comprises an isolated polypeptide or polypeptide complex of any one of embodiments 16-30 and 32-35, wherein A₁ comprises a heavy chain variable domain and a light chain variable domain comprising complementarity determining regions (CDRs): HC-CDR1, HC-CDR2, and HC-CDR3, wherein the HC-CDR1, the HC-CDR2, and the HC-CDR3 of the heavy chain variable domain comprise: HC-CDR1: SEQ ID NO: 3; HC-CDR2: SEQ ID NO: 4; HC-CDR3: SEQ ID NO: 5; and the light chain variable domain comprises complementarity determining regions (CDRs): LC-CDR1, LC-CDR2, and LC-CDR3, wherein the LC-CDR1, the LC-CDR2, and the LC-CDR3 of the light chain variable domain comprise: LC-CDR1: SEQ ID NO: 6; LC-CDR2: SEQ ID NO: 7; and LC-CDR3: SEQ ID NO: 8.

Embodiment 38 comprises an isolated polypeptide or polypeptide complex of any one of embodiments 16-37, wherein the polypeptide or polypeptide complex of formula I binds to an effector cell when L₁ is cleaved by the tumor specific protease.

Embodiment 39 comprises an isolated polypeptide or polypeptide complex of any one of embodiments 16-38, wherein the polypeptide or polypeptide complex of formula I binds to an effector cell when L₁ is cleaved by the tumor specific protease and A₁ binds to the effector cell.

Embodiment 40 comprises an isolated polypeptide or polypeptide complex of any one of embodiments 38-39, wherein the effector cell is a T cell.

Embodiment 41 comprises an isolated polypeptide or polypeptide complex of any one of embodiments 38-40, wherein A₁ binds to a polypeptide that is part of a TCR-CD3 complex on the effector cell.

Embodiment 42 comprises an isolated polypeptide or polypeptide complex of embodiment 41, wherein the polypeptide that is part of the TCR-CD3 complex is human CD3E.

Embodiment 43 comprises an isolated polypeptide or polypeptide complex of any one of embodiments 20-42, wherein A₂ comprises an antibody or antibody fragment.

Embodiment 44 comprises an isolated polypeptide or polypeptide complex of embodiment 43, wherein the antibody or antibody fragment thereof of A₂ comprises a single chain variable fragment, a single domain antibody, Fab′, or a Fab.

Embodiment 45 comprises an isolated polypeptide or polypeptide complex of embodiment 43, wherein the antibody or antibody fragment thereof of A₂ comprises a single chain variable fragment (scFv), a heavy chain variable domain (VH domain), a light chain variable domain (VL domain), or a variable domain (VHH) of a camelid derived single domain antibody.

Embodiment 46 comprises an isolated polypeptide or polypeptide complex of any one of embodiments 43-45, wherein the antibody or antibody fragment thereof of A₂ is humanized or human.

Embodiment 47 comprises an isolated polypeptide or polypeptide complex of embodiment 44, wherein A₂ is the Fab or Fab′.

Embodiment 48 comprises an isolated polypeptide or polypeptide complex of embodiment 47, wherein the Fab or Fab′ comprises (a) a Fab light chain polypeptide and (b) a Fab heavy chain polypeptide.

Embodiment 49 comprises an isolated polypeptide or polypeptide complex of any one of embodiments 20-48, wherein A₂ comprises an epidermal growth factor receptor (EGFR) binding domain.

Embodiment 50 comprises an isolated polypeptide or polypeptide complex of any one of embodiments 20-48, wherein A₂ comprises a carcinoembryonic antigen-related cell adhesion molecule CEACAM5 binding domain.

Embodiment 51 comprises an isolated polypeptide or polypeptide complex of any one of embodiments 16-50, wherein P₁ is further linked to a half-life extending moiety (H₁).

Embodiment 52 comprises an isolated polypeptide or polypeptide complex of embodiment 51, wherein the half-life extending moiety is a single-domain antibody.

Embodiment 53 comprises an isolated polypeptide or polypeptide complex of embodiment 51, wherein H₁ comprises a polymer.

Embodiment 54 comprises an isolated polypeptide or polypeptide complex of embodiment 53, wherein the polymer is polyethylene glycol (PEG).

Embodiment 55 comprises an isolated polypeptide or polypeptide complex of embodiment 51, wherein H₁ comprises albumin.

Embodiment 56 comprises an isolated polypeptide or polypeptide complex of embodiment 51, wherein H₁ comprises an Fc domain.

Embodiment 57 comprises an isolated polypeptide or polypeptide complex of embodiment 55, wherein the albumin is serum albumin.

Embodiment 58 comprises an isolated polypeptide or polypeptide complex of any one of embodiments 55 and 57, wherein the albumin is human serum albumin.

Embodiment 59 comprises an isolated polypeptide or polypeptide complex of any one of embodiments 51-58, wherein H₁ comprises a polypeptide, a ligand, or a small molecule.

Embodiment 60 comprises an isolated polypeptide or polypeptide complex of embodiment 59, wherein the polypeptide, the ligand or the small molecule binds serum protein or a fragment thereof, a circulating immunoglobulin or a fragment thereof, or CD35/CR1.

Embodiment 61 comprises an isolated polypeptide or polypeptide complex of embodiment 60, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 62 comprises an isolated polypeptide or polypeptide complex of embodiment 60, wherein the circulating immunoglobulin molecule comprises IgGl, IgG2, IgG3, IgG4, slgA, IgM or IgD.

Embodiment 63 comprises an isolated polypeptide or polypeptide complex of embodiment 60 or 61, wherein the serum protein is albumin.

Embodiment 64 comprises an isolated polypeptide or polypeptide complex of any one of embodiments 59-63, wherein the polypeptide is an antibody.

Embodiment 65 comprises an isolated polypeptide or polypeptide complex of embodiment 64, wherein the antibody comprises a single domain antibody, a single chain variable fragment, or a Fab.

Embodiment 66 comprises an isolated polypeptide or polypeptide complex of embodiment 65, wherein the single domain antibody comprises a single domain antibody that binds to albumin.

Embodiment 67 comprises an isolated polypeptide or polypeptide complex of any one of embodiments 65-66, wherein the single domain antibody is a human or humanized antibody.

Embodiment 68 comprises an isolated polypeptide or polypeptide complex of any one of embodiments 65-67, wherein the single domain antibody is 645gH1gL1.

Embodiment 69 comprises an isolated polypeptide or polypeptide complex of any one of embodiments 65-67, wherein the single domain antibody is 645dsgH5gL4.

Embodiment 70 comprises an isolated polypeptide or polypeptide complex of embodiment 65, wherein the single domain antibody is 23-13-A01-sc02.

Embodiment 71 comprises an isolated polypeptide or polypeptide complex of any one of embodiments 65-66, wherein the single domain antibody is A10m3 or a fragment thereof.

Embodiment 72 comprises an isolated polypeptide or polypeptide complex of any one of embodiments 65-66, wherein the single domain antibody is DOM7r-31.

Embodiment 73 comprises an isolated polypeptide or polypeptide complex of any one of embodiments 65-66, wherein the single domain antibody is DOM7h-11-15.

Embodiment 74 comprises an isolated polypeptide or polypeptide complex of any one of embodiments 65-66, wherein the single domain antibody is Alb-1, Alb-8, or Alb-23.

Embodiment 75 comprises an isolated polypeptide or polypeptide complex of embodiment 65, wherein the single domain antibody is 10G or 10GE.

Embodiment 76 comprises an isolated polypeptide or polypeptide complex of any one of embodiments 65-66, wherein the single domain antibody is SA21.

Embodiment 77 comprises an isolated polypeptide or polypeptide complex of any one of embodiments 1-76, wherein the polypeptide or polypeptide complex comprises a modified amino acid, a non-natural amino acid, a modified non-natural amino acid, or a combination thereof.

Embodiment 78 comprises an isolated polypeptide or polypeptide complex of embodiment 77, wherein the modified amino acid or modified non-natural amino acid comprises a post-translational modification.

Embodiment 79 comprises an isolated polypeptide or polypeptide complex of any one of embodiments 51-78, wherein H₁ comprises a linking moiety (L₃) that connects H₁ to P₁.

Embodiment 80 comprises an isolated polypeptide or polypeptide complex of embodiment 79, wherein L₃ is a peptide sequence having at least 5 to no more than 50 amino acids.

Embodiment 81 comprises an isolated polypeptide or polypeptide complex of any one of embodiments 79-80, wherein L₃ is a peptide sequence having at least 10 to no more than 30 amino acids.

Embodiment 82 comprises an isolated polypeptide or polypeptide complex of any one of embodiments 79-81, wherein L₃ is a peptide sequence having at least 10 amino acids.

Embodiment 83 comprises an isolated polypeptide or polypeptide complex of any one of embodiments 79-82, wherein L₃ is a peptide sequence having at least 18 amino acids.

Embodiment 84 comprises an isolated polypeptide or polypeptide complex of any one of embodiments 79-83, wherein L₃ is a peptide sequence having at least 26 amino acids.

Embodiment 85 comprises an isolated polypeptide or polypeptide complex of any one of embodiments 79-84, wherein L₃ has a formula selected from the group consisting of (G₂S)ₙ, (GS)ₙ, (GSGGS)ₙ (SEQ ID NO: 917), (GGGS)ₙ (SEQ ID NO: 918), (GGGGS)ₙ (SEQ ID NO: 919), and (GSSGGS)ₙ (SEQ ID NO: 920), wherein n is an integer of at least 1.

Embodiment 86 comprises an isolated polypeptide or polypeptide complex of any one of embodiments 79-84, wherein L₃ comprises an amino acid sequence according to (GGGGSGGGS (SEQ ID NO: 921)).

Embodiment 87 comprises an isolated polypeptide or polypeptide complex of any one of embodiments 48-86, wherein the Fab light chain polypeptide of A₂ is bound to a C-terminus of the single chain variable fragment (scFv) of A₁.

Embodiment 88 comprises an isolated polypeptide or polypeptide complex of any one of embodiments 48-86, wherein the Fab heavy chain polypeptide of A₂ is bound to a C-terminus of the single chain variable fragment (scFv) A₁.

Embodiment 89 comprises an isolated polypeptide or polypeptide complex of any one of embodiments 48-86, wherein the Fab light chain polypeptide of A₂ is bound to a N-terminus of the single chain variable fragment (scFv) of A₁.

Embodiment 90 comprises an isolated polypeptide or polypeptide complex of any one of embodiments 48-86, wherein the Fab heavy chain polypeptide of A₂ is bound to a N-terminus of the single chain variable fragment (scFv) A₁.

Embodiment 91 comprises an isolated polypeptide or polypeptide complex of any one of embodiments 48-86, 88, and 90, wherein the Fab heavy chain polypeptide of A₂ is bound to the scFv heavy chain polypeptide of A₁.

Embodiment 92 comprises an isolated polypeptide or polypeptide complex of any one of embodiments 48-86, 87, and 89, wherein the Fab light chain polypeptide of A₂ is bound to the scFv heavy chain polypeptide of A₁.

Embodiment 93 comprises an isolated polypeptide or polypeptide complex of any one of embodiments 48-86, 88, and 90, wherein the Fab heavy chain polypeptide of A₂ is bound to the scFv light chain polypeptide of A₁.

Embodiment 94 comprises an isolated polypeptide or polypeptide complex of any one of embodiments 48-86, 87, and 89, wherein the Fab light chain polypeptide of A₂ is bound to the scFv light chain polypeptide of A₁.

Embodiment 95 comprises an isolated polypeptide or polypeptide complex of any one of embodiments 48-86, 88, 90, and 91, wherein the Fab heavy chain polypeptide of A₂ is bound to the scFv heavy chain polypeptide of A₁ and L₂ is bound to the Fab light chain polypeptide of A₂.

Embodiment 96 comprises an isolated polypeptide or polypeptide complex of any one of embodiments 48-87, 89, and 92, wherein the Fab light chain polypeptide of A₂ is bound to the scFv heavy chain polypeptide of A₁ and L₂ is bound to the Fab heavy chain polypeptide of A₂.

Embodiment 97 comprises an isolated polypeptide or polypeptide complex of any one of embodiments 1-86, 88, 90, and 93, wherein the Fab heavy chain polypeptide of A₂ is bound to the scFv light chain polypeptide of A₁ and L₂ is bound to the Fab light chain polypeptide of A₂.

Embodiment 98 comprises an isolated polypeptide or polypeptide complex of any one of embodiments 1-87, 89, and 94, wherein the Fab light chain polypeptide of A₂ is bound to the scFv light chain polypeptide of A₁ and L₂ is bound to the Fab heavy chain polypeptide of A₂.

Embodiment 99 comprises an isolated polypeptide or polypeptide complex of any one of embodiments 21-98, wherein P₂ impairs binding of A₂ to the tumor antigen.

Embodiment 100 comprises an isolated polypeptide or polypeptide complex of any one of embodiments 21-99, wherein P₂ is bound to A₂ through ionic interactions, electrostatic interactions, hydrophobic interactions, Pi-stacking interactions, or H-bonding interactions, or a combination thereof.

Embodiment 101 comprises an isolated polypeptide or polypeptide complex of any one of embodiments 21-100, wherein P₂ is bound to A₂ at or near an antigen binding site.

Embodiment 102 comprises an isolated polypeptide or polypeptide complex of any one of embodiments 21-101, wherein P₂ has less than 70% sequence homology to the tumor antigen.

Embodiment 103 comprises an isolated polypeptide or polypeptide complex of any one of embodiments 21-102, wherein P₂ comprises a peptide sequence of at least 10 amino acids in length.

Embodiment 104 comprises an isolated polypeptide or polypeptide complex of any one of embodiments 21-103, wherein P₂ comprises a peptide sequence of at least 10 amino acids in length and no more than 20 amino acids in length.

Embodiment 105 comprises an isolated polypeptide or polypeptide complex of any one of embodiments 21-104, wherein P₂ comprises a peptide sequence of at least 16 amino acids in length.

Embodiment 106 comprises an isolated polypeptide or polypeptide complex of any one of embodiments 21-103 and 105, wherein P₂ comprises a peptide sequence of no more than 40 amino acids in length.

Embodiment 107 comprises an isolated polypeptide or polypeptide complex of any one of embodiments 21-106, wherein P₂ comprises at least two cysteine amino acid residues.

Embodiment 108 comprises an isolated polypeptide or polypeptide complex of any one of embodiments 21-107, wherein P₂ comprises a cyclic peptide or a linear peptide.

Embodiment 109 comprises an isolated polypeptide or polypeptide complex of any one of embodiments 21-108, wherein P₂ comprises a cyclic peptide.

Embodiment 110 comprises an isolated polypeptide or polypeptide complex of any one of embodiments 21-108, wherein P₂ comprises a linear peptide.

Embodiment 111 comprises an isolated polypeptide or polypeptide complex of any one of embodiments 16-17, 19-24, 27-88, and 91-110, wherein L₁ is bound to N-terminus of A₁.

Embodiment 112 comprises an isolated polypeptide or polypeptide complex of any one of embodiments 16, 18-23, 25-26, 28-86, 89-110, wherein L₁ is bound to C-terminus of A₁.

Embodiment 113 comprises an isolated polypeptide or polypeptide complex of any one of embodiments 21-23, 25-26, 28-86, 89-110, and 112, wherein L₂ is bound to N-terminus of A₂.

Embodiment 114 comprises an isolated polypeptide or polypeptide complex of any one of embodiments 21, 24, 27-88, and 91-111, wherein L₂ is bound to C-terminus of A₂.

Embodiment 115 comprises an isolated polypeptide or polypeptide complex of any one of embodiments 16-114, wherein L₁ or L₂ is a peptide sequence having at least 5 to no more than 50 amino acids.

Embodiment 116 comprises an isolated polypeptide or polypeptide complex of any one of embodiments 16-115, wherein L₁ or L₂ is a peptide sequence having at least 10 to no more than 30 amino acids.

Embodiment 117 comprises an isolated polypeptide or polypeptide complex of any one of embodiments 16-116, wherein L₁ or L₂ is a peptide sequence having at least 10 amino acids.

Embodiment 118 comprises an isolated polypeptide or polypeptide complex of any one of embodiments 16-117, wherein L₁ or L₂ is a peptide sequence having at least 18 amino acids.

Embodiment 119 comprises an isolated polypeptide or polypeptide complex of any one of embodiments 16-118, wherein L₁ or L₂ is a peptide sequence having at least 26 amino acids.

Embodiment 120 comprises an isolated polypeptide or polypeptide complex of any one of embodiments 16-115, wherein L₁ or L₂ has a formula comprising (G₂S)ₙ (SEQ ID NO: 922), wherein n is an integer from 1 to 3.

Embodiment 121 comprises an isolated polypeptide or polypeptide complex of any one of embodiments 16-120, wherein L₁ has a formula selected from the group consisting of (G₂S)ₙ, (GS)ₙ, (GSGGS)ₙ (SEQ ID NO: 917), (GGGS)ₙ (SEQ ID NO: 918), (GGGGS)ₙ (SEQ ID NO: 919), and (GSSGGS)ₙ (SEQ ID NO: 920), wherein n is an integer of at least 1.

Embodiment 122 comprises an isolated polypeptide or polypeptide complex of any one of embodiments 16-121, wherein P₁ becomes unbound from A₁ when L₁ is cleaved by the tumor specific protease thereby exposing A₁ to CD3.

Embodiment 123 comprises an isolated polypeptide or polypeptide complex of any one of embodiments 21-122, wherein P₂ becomes unbound from A₂ when L₂ is cleaved by the tumor specific protease thereby exposing A₂ to the tumor antigen.

Embodiment 124 comprises an isolated polypeptide or polypeptide complex of any one of embodiments 16-123, wherein the tumor specific protease is selected from the group consisting of metalloprotease, serine protease, cysteine protease, threonine protease, and aspartic protease.

Embodiment 125 comprises an isolated polypeptide or polypeptide complex of any one of embodiments 16-124, wherein L₁ or L₂ comprises a urokinase cleavable amino acid sequence, a matriptase cleavable amino acid sequence, a matrix metalloprotease cleavable amino acid sequence, or a legumain cleavable amino acid sequence.

Embodiment 126 comprises an isolated polypeptide or polypeptide complex of any one of embodiments 16-119 and 122-125, wherein L₁ or L₂ comprises an amino acid sequence selected from the group consisting of GGGGSLSGRSDNHGSSGT (SEQ ID NO: 923), GGGGSSGGSGGSGLSGRSDNHGSSGT (SEQ ID NO: 924), ASGRSDNH (SEQ ID NO: 925), LAGRSDNH (SEQ ID NO: 926), ISSGLASGRSDNH (SEQ ID NO: 927), ISSGLLAGRSDNH (SEQ ID NO: 928), LSGRSDNH (SEQ ID NO: 929), ISSGLLSGRSDNP (SEQ ID NO: 930), ISSGLLSGRSDNH (SEQ ID NO: 931), LSGRSDNHSPLGLAGS (SEQ ID NO: 932), SPLGLAGSLSGRSDNH (SEQ ID NO: 933), SPLGLSGRSDNH (SEQ ID NO: 934), LAGRSDNHSPLGLAGS (SEQ ID NO: 935), LSGRSDNHVPLSLKMG (SEQ ID NO: 936), and LSGRSDNHVPLSLSMG (SEQ ID NO: 937).

Embodiment 127 comprises an isolated polypeptide or polypeptide complex of any one of embodiments 16-119 and 122-126, wherein L₁ or L₂ comprises an amino acid sequence selected from the group consisting of ASGRSDNH (SEQ ID NO: 925), LAGRSDNH (SEQ ID NO: 926), ISSGLASGRSDNH (SEQ ID NO: 927), and ISSGLLAGRSDNH (SEQ ID NO: 928).

Embodiment 128 comprises a pharmaceutical composition comprising: (i) the polypeptide or polypeptide complex of any one of embodiments 1-127; and (ii) a pharmaceutically acceptable excipient.

Embodiment 129 comprises an isolated recombinant nucleic acid molecule encoding the polypeptide or polypeptide complex of any one of embodiments 1-127.

Embodiment 130 comprises an isolated polypeptide or polypeptide complex according to Formula II: L₁ₐ-P₁ₐ-H₁ₐ wherein: L₁ₐ comprises a tumor specific protease-cleaved linking moiety that when uncleaved connects P₁ₐ to an anti-CD3 binding domain that binds to CD3 and; P₁ₐ comprises a peptide that impairs binding of the anti-CD3 binding domain to CD3 when L₁ₐ is uncleaved wherein the peptide comprises an amino acid sequence selected from SEQ ID NOs: 1-2, and 19-55 or the peptide comprises 1, 2, or 3, amino acid substitutions, additions, or deletions relative to amino acid sequences of SEQ ID NOs: 1-2, and 19-55; and H₁ₐ comprises a half-life extending molecule.

Embodiment 131 comprises an isolated polypeptide or polypeptide complex of embodiment 130, wherein the peptide comprises an amino acid sequence selected from SEQ ID NOs: 1-2, and 19-55.

Embodiment 132 comprises an isolated polypeptide or polypeptide complex of any one of embodiments 130-131, wherein the peptide comprises the amino acid sequence of SEQ ID NO: 1.

Embodiment 133 comprises an isolated polypeptide or polypeptide complex of any one of embodiments 130-131, wherein the peptide comprises the amino acid sequence of SEQ ID NO: 2.

Embodiment 134 comprises an isolated polypeptide or polypeptide complex of any one of embodiments 130-131, wherein the peptide comprises the amino acid sequence of SEQ ID NO: 54.

Embodiment 135 comprises an isolated polypeptide or polypeptide complex of any one of embodiments 130-134, wherein H₁ₐ comprises a polymer.

Embodiment 136 comprises an isolated polypeptide or polypeptide complex of embodiment 135, wherein the polymer is polyethylene glycol (PEG).

Embodiment 137 comprises an isolated polypeptide or polypeptide complex of any one of embodiments 130-134, wherein H₁ₐ comprises albumin.

Embodiment 138 comprises an isolated polypeptide or polypeptide complex of any one of embodiments 130-134, wherein H₁ₐ comprises an Fc domain.

Embodiment 139 comprises an isolated polypeptide or polypeptide complex of embodiment 137, wherein the albumin is serum albumin.

Embodiment 140 comprises an isolated polypeptide or polypeptide complex of any one of embodiments 137 and 139, wherein the albumin is human serum albumin.

Embodiment 141 comprises an isolated polypeptide or polypeptide complex of any one of embodiments 130-140, wherein H₁ₐ comprises a polypeptide, a ligand, or a small molecule.

Embodiment 142 comprises an isolated polypeptide or polypeptide complex of embodiment 141, wherein the polypeptide, the ligand or the small molecule binds a serum protein or a fragment thereof, a circulating immunoglobulin or a fragment thereof, or CD35/CR1.

Embodiment 143 comprises an isolated polypeptide or polypeptide complex of embodiment 142, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 144 comprises an isolated polypeptide or polypeptide complex of embodiment 142, wherein the circulating immunoglobulin molecule comprises IgGl, IgG2, IgG3, IgG4, slgA, IgM or IgD.

Embodiment 145 comprises an isolated polypeptide or polypeptide complex of any one of embodiments 142-143, wherein the serum protein is albumin.

Embodiment 146 comprises an isolated polypeptide or polypeptide complex of any one of embodiments 141-145, wherein the polypeptide is an antibody.

Embodiment 147 comprises an isolated polypeptide or polypeptide complex of embodiment 146, wherein the antibody comprises a single domain antibody, a single chain variable fragment or a Fab.

Embodiment 148 comprises an isolated polypeptide or polypeptide complex of any one of embodiments 146-147, wherein the antibody comprises a single domain antibody that binds to albumin.

Embodiment 149 comprises an isolated polypeptide or polypeptide complex of any one of embodiments 146-148, wherein the antibody is a human or humanized antibody.

Embodiment 150 comprises an isolated polypeptide or polypeptide complex of any one of embodiments 147-149, wherein the single domain antibody is 645gH1gL1.

Embodiment 151 comprises an isolated polypeptide or polypeptide complex of any one of embodiments 147-149, wherein the single domain antibody is 645dsgH5gL4.

Embodiment 152 comprises an isolated polypeptide or polypeptide complex of embodiment 147, wherein the single domain antibody is 23-13-A01-sc02.

Embodiment 153 comprises an isolated polypeptide or polypeptide complex of any one of embodiments 147-148, wherein the single domain antibody is A10m3 or a fragment thereof.

Embodiment 154 comprises an isolated polypeptide or polypeptide complex of any one of embodiments 147-148, wherein the single domain antibody is DOM7r-31.

Embodiment 155 comprises an isolated polypeptide or polypeptide complex of any one of embodiments 147-148, wherein the single domain antibody is DOM7h-11-15.

Embodiment 156 comprises an isolated polypeptide or polypeptide complex of any one of embodiments 147-148, wherein the single domain antibody is Alb-1, Alb-8, or Alb-23.

Embodiment 157 comprises an isolated polypeptide or polypeptide complex of embodiment 147, wherein the single domain antibody is 10G or 10GE.

Embodiment 158 comprises an isolated polypeptide or polypeptide complex of any one of embodiments 147-148, wherein the single domain antibody is SA21.

Embodiment 159 comprises an isolated polypeptide or polypeptide complex of any one of embodiments 130-158, wherein H₁ₐ comprises a linking moiety (L₃ₐ) that connects H₁ₐ to P₁ₐ.

Embodiment 160 comprises an isolated polypeptide or polypeptide complex of embodiment 159, wherein L₃ₐ is a peptide sequence having at least 5 to no more than 50 amino acids.

Embodiment 161 comprises an isolated polypeptide or polypeptide complex of any one of embodiments 159-160, wherein L₃ₐ is a peptide sequence having at least 10 to no more than 30 amino acids.

Embodiment 162 comprises an isolated polypeptide or polypeptide complex of any one of embodiments 159-161, wherein L₃ₐ is a peptide sequence having at least 10 amino acids.

Embodiment 163 comprises an isolated polypeptide or polypeptide complex of any one of embodiments 159-162, wherein L₃ₐ is a peptide sequence having at least 18 amino acids.

Embodiment 164 comprises an isolated polypeptide or polypeptide complex of any one of embodiments 159-163, wherein L₃ₐ is a peptide sequence having at least 26 amino acids.

Embodiment 165 comprises an isolated polypeptide or polypeptide complex of any one of embodiments 159-164, wherein L₃ₐ has a formula selected from the group consisting of (G₂S)ₙ, (GS)ₙ, (GSGGS)ₙ (SEQ ID NO: 917), (GGGS)ₙ (SEQ ID NO: 918), (GGGGS)ₙ (SEQ ID NO: 919), and (GSSGGS)ₙ (SEQ ID NO: 920), wherein n is an integer of at least 1.

Embodiment 166 comprises an isolated polypeptide or polypeptide complex of any one of embodiments 159-164, wherein L₃ₐ comprises an amino acid sequence according to (GGGGSGGGS (SEQ ID NO: 921)).

Embodiment 167 comprises an isolated polypeptide or polypeptide complex according to Formula III: L_{1b}-P_{1b}-H_{1b} wherein: L_{1b} comprises a tumor specific protease-cleaved linking moiety that when uncleaved connects P_{1b} to an anti-CD3 binding domain that binds to CD3 and; P_{1b} comprises a peptide that impairs binding of the anti-CD3 binding domain to CD3 when L_{1b} is uncleaved wherein the peptide comprises the amino acid sequence according to Z₁-Z₂-C-Z₄-P-Z₆-Z₇-Z₈-Z₉-Z₁₀-Z₁₁-Z₁₂-C-Z₁₄ and Z₁ is selected from D, Y, F, I, N, V, H, L, A, T, S, and P; Z₂ is selected from D, Y, L, F, I, N, A, V, H, T, and S; Z₄ is selected from G and W; Z₆ is selected from E, D, V, and P; Z₇ is selected from W, L, F, V, G, M, I, and Y; Z₈ is selected from E, D, P, and Q; Z₉ is selected from E, D, Y, V, F, W, P, L, and Q; Z₁₀ is selected from S, D, Y, T, I, F, V, N, A, P, L, and H; Z₁₁ is selected from I, Y, F, V, L, T, N, S, D, A, and H; Z₁₂ is selected from F, D, Y, L, I, V, A, N, T, P, S, and H; and Z₁₄ is selected from D, Y, N, F, I, P, V, A, T, H, L, and S; and H_{1b} comprises a half-life extending molecule.

Embodiment 168 comprises an isolated polypeptide or polypeptide complex of embodiment 167, wherein Z₁ is selected from D, Y, F, I, and N; Z₂ is selected from D, Y, L, F, I, and N; Z₄ is selected from G and W; Z₆ is selected from E and D; Z₇ is selected from W, L, F, and V; Z₈ is selected from E and D; Z₉ is selected from E, D, Y, and V; Z₁₀ is selected from S, D, Y, T, and I; Z₁₁ is selected from I, Y, F, V, L, and T; Z₁₂ is selected from F, D, Y, L, I, V, A, and N; and Z₁₄ is selected from D, Y, N, F, I, and P.

Embodiment 169 comprises an isolated polypeptide or polypeptide complex of any one of embodiments 167-168, wherein Z₁ is selected from D, Y, and F; Z₂ is selected from D, Y, L, and F; Z₄ is selected from G and W; Z₆ is selected from E and D; Z₇ is selected from W, L, and F; Z₈ is selected from E and D; Z₉ is selected from E and D; Z₁₀ is selected from S, D, and Y; Z₁₁ is selected from I, Y, and F; Z₁₂ is selected from F, D, Y, and L; and Z₁₄ is selected from D, Y, and N.

Embodiment 170 comprises an isolated polypeptide or polypeptide complex of any one of embodiments 167-169, wherein P_{1b} comprises comprises an amino acid sequence of Table 17.

Embodiment 171 comprises an isolated polypeptide or polypeptide complex of any one of embodiments 167-170, wherein P_{1b} comprises an amino acid sequences according to any one of SEQ ID NOs: 44-55.

Embodiment 172 comprises an isolated polypeptide or polypeptide complex of embodiment 167, wherein P_{1b} comprises the amino acid sequences according to SEQ ID NO: 2.

Embodiment 173 comprises an isolated polypeptide or polypeptide complex of embodiment 167-168, wherein P_{1b} comprises the amino acid sequences according to SEQ ID NO: 54.

Embodiment 174 comprises an isolated polypeptide or polypeptide complex of any one of embodiments 167-173, wherein H_{1b} comprises a polymer.

Embodiment 175 comprises an isolated polypeptide or polypeptide complex of embodiment 174, wherein the polymer is polyethylene glycol (PEG).

Embodiment 176 comprises an isolated polypeptide or polypeptide complex of any one of embodiments 167-173, wherein H_{1b} comprises albumin.

Embodiment 177 comprises an isolated polypeptide or polypeptide complex of any one of embodiments 167-173, wherein H_{1b} comprises an Fc domain.

Embodiment 178 comprises an isolated polypeptide or polypeptide complex of embodiment 176, wherein the albumin is serum albumin.

Embodiment 179 comprises an isolated polypeptide or polypeptide complex of any one of embodiments 176 and 178, wherein the albumin is human serum albumin.

Embodiment 180 comprises an isolated polypeptide or polypeptide complex of any one of embodiments 167-179, wherein H_{1b} comprises a polypeptide, a ligand, or a small molecule.

Embodiment 181 comprises an isolated polypeptide or polypeptide complex of embodiment 180, wherein the polypeptide, the ligand or the small molecule binds a serum protein or a fragment thereof, a circulating immunoglobulin or a fragment thereof, or CD35/CR1.

Embodiment 182 comprises an isolated polypeptide or polypeptide complex of embodiment 181, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 183 comprises an isolated polypeptide or polypeptide complex of embodiment 181, wherein the circulating immunoglobulin molecule comprises IgGl, IgG2, IgG3, IgG4, slgA, IgM or IgD.

Embodiment 184 comprises an isolated polypeptide or polypeptide complex of any one of embodiments 181-182, wherein the serum protein is albumin.

Embodiment 185 comprises an isolated polypeptide or polypeptide complex of any one of embodiments 180-184, wherein the polypeptide is an antibody.

Embodiment 186 comprises an isolated polypeptide or polypeptide complex of embodiment 185, wherein the antibody comprises a single domain antibody, a single chain variable fragment or a Fab.

Embodiment 187 comprises an isolated polypeptide or polypeptide complex of any one of embodiments 185-186, wherein the antibody comprises a single domain antibody that binds to albumin.

Embodiment 188 comprises an isolated polypeptide or polypeptide complex of any one of embodiments 185-187, wherein the antibody is a human or humanized antibody.

Embodiment 189 comprises an isolated polypeptide or polypeptide complex of any one of embodiments 186-188, wherein the single domain antibody is 645gH1gL1.

Embodiment 190 comprises an isolated polypeptide or polypeptide complex of any one of embodiments 186-188, wherein the single domain antibody is 645dsgH5gL4.

Embodiment 191 comprises an isolated polypeptide or polypeptide complex of any one of embodiments 186, wherein the single domain antibody is 23-13-A01-sc02.

Embodiment 192 comprises an isolated polypeptide or polypeptide complex of any one of embodiments 186-187, wherein the single domain antibody is A10m3 or a fragment thereof.

Embodiment 193 comprises an isolated polypeptide or polypeptide complex of any one of embodiments 186-187, wherein the single domain antibody is DOM7r-31.

Embodiment 194 comprises an isolated polypeptide or polypeptide complex of any one of embodiments 186-187, wherein the single domain antibody is DOM7h-11-15.

Embodiment 195 comprises an isolated polypeptide or polypeptide complex of any one of embodiments 186-187, wherein the single domain antibody is Alb-1, Alb-8, or Alb-23.

Embodiment 196 comprises an isolated polypeptide or polypeptide complex of embodiment 186, wherein the single domain antibody is 10G or 10GE.

Embodiment 197 comprises an isolated polypeptide or polypeptide complex of any one of embodiments 186-187, wherein the single domain antibody is SA21.

Embodiment 198 comprises an isolated polypeptide or polypeptide complex of any one of embodiments 167-197, wherein H_{1b} comprises a linking moiety (L_{3b}) that connects H_{1b} to P_{1b}.

Embodiment 199 comprises an isolated polypeptide or polypeptide complex of embodiment 198, wherein L_{3b} is a peptide sequence having at least 5 to no more than 50 amino acids.

Embodiment 200 comprises an isolated polypeptide or polypeptide complex of any one of embodiments 198-199, wherein L_{3b} is a peptide sequence having at least 10 to no more than 30 amino acids.

Embodiment 201 comprises an isolated polypeptide or polypeptide complex of any one of embodiments 198-200, wherein L_{3b} is a peptide sequence having at least 10 amino acids.

Embodiment 202 comprises an isolated polypeptide or polypeptide complex of any one of embodiments 198-201, wherein L_{3b} is a peptide sequence having at least 18 amino acids.

Embodiment 203 comprises an isolated polypeptide or polypeptide complex of any one of embodiments 198-202, wherein L_{3b} is a peptide sequence having at least 26 amino acids.

Embodiment 204 comprises an isolated polypeptide or polypeptide complex of any one of embodiments 198-203, wherein L_{3b} has a formula selected from the group consisting of (G₂S)ₙ, (GS)ₙ, (GSGGS)ₙ (SEQ ID NO: 917), (GGGS)ₙ (SEQ ID NO: 918), (GGGGS)ₙ (SEQ ID NO: 919), and (GSSGGS)ₙ (SEQ ID NO: 920), wherein n is an integer of at least 1.

Embodiment 205 comprises an isolated polypeptide or polypeptide complex of any one of embodiments 198-203, wherein L_{3b} comprises an amino acid sequence according to (GGGGSGGGS (SEQ ID NO: 921)).

Embodiment 206 comprises an isolated polypeptide or polypeptide complex according to Formula IV: L_{1c}-P_{1c}-H_{1c} wherein: L_{1c} comprises a tumor specific protease-cleaved linking moiety that when uncleaved connects P_{1c} to an anti-CD3 binding domain that binds to CD3 and; P_{1c} comprises a peptide that impairs binding of the anti-CD3 binding domain to CD3 when L_{1b} is uncleaved wherein the peptide comprises the amino acid sequence according to U₁-U₂-C-U₄-P-U₆-U₇-U₈-U₉-U₁₀-U₁₁-U₁₂-C-U₁₄ and U₁ is selected from D, Y, F, I, N, V, H, L, A, T, S, and P; U₂ is selected from D, Y, L, F, I, N, A, V, H, T, and S; U₄ is selected from G and W; U₆ is selected from E, D, V, and P; U₇ is selected from W, L, F, V, G, M, I, and Y; U₈ is selected from E, D, P, and Q; U₉ is selected from E, D, Y, V, F, W, P, L, and Q; U₁₀ is selected from S, D, Y, T, I, F, V, N, A, P, L, and H; U₁₁ is selected from I, Y, F, V, L, T, N, S, D, A, and H; U₁₂ is selected from F, D, Y, L, I, V, A, N, T, P, S, G, and H; and U₁₄ is selected from D, Y, N, F, I, P, V, A, T, H, L, M, and S; and H_{1c} comprises a half-life extending molecule.

Embodiment 207 comprises an isolated polypeptide or polypeptide complex of embodiment 206, wherein U₁ is selected from D, Y, F, I, V, and N; U₂ is selected from D, Y, L, F, I, and N; U₄ is selected from G and W; U₆ is selected from E and D; U₇ is selected from W, L, F, G, and V; U₈ is selected from E and D; U₉ is selected from E, D, Y, and V; U₁₀ is selected from S, D, Y, T, and I; U₁₁ is selected from I, Y, F, V, L, and T; U₁₂ is selected from F, D, Y, L, I, V, A, G, and N; and U₁₄ is selected from D, Y, N, F, I, M, and P.

Embodiment 208 comprises an isolated polypeptide or polypeptide complex of any one of embodiments 206-207, wherein U₁ is selected from D, Y, V, and F; U₂ is selected from D, Y, L, and F; U₄ is selected from G and W; U₆ is selected from E and D; U₇ is selected from W, L, G, and F; U₈ is selected from E and D; U₉ is selected from E and D; U₁₀ is selected from S, D, T, and Y; U₁₁ is selected from I, Y, V, L, and F; U₁₂ is selected from F, D, Y, G, A, and L; and U₁₄ is selected from D, Y, M, and N.

Embodiment 209 comprises an isolated polypeptide or polypeptide complex of any one of embodiments 206-208, wherein P_{1c} comprises comprises an amino acid sequence of Table 17.

Embodiment 210 comprises an isolated polypeptide or polypeptide complex of any one of embodiments 206-209, wherein P_{1c} comprises an amino acid sequences according to any one of SEQ ID NOs: 44-55.

Embodiment 211 comprises an isolated polypeptide or polypeptide complex of any one of embodiments 206-209, wherein P_{1c} comprises the amino acid sequences according to SEQ ID NO: 2.

Embodiment 212 comprises an isolated polypeptide or polypeptide complex of any one of embodiments 206-210, wherein P_{1c} comprises the amino acid sequences according to SEQ ID NO: 54.

Embodiment 213 comprises an isolated polypeptide or polypeptide complex of any one of embodiments 206-212, wherein H_{1c} comprises a polymer.

Embodiment 214 comprises an isolated polypeptide or polypeptide complex of embodiment 213, wherein the polymer is polyethylene glycol (PEG).

Embodiment 215 comprises an isolated polypeptide or polypeptide complex of any one of embodiments 206-212, wherein H_{1c} comprises albumin.

Embodiment 216 comprises an isolated polypeptide or polypeptide complex of any one of embodiments 206-212, wherein H_{1c} comprises an Fc domain.

Embodiment 217 comprises an isolated polypeptide or polypeptide complex of embodiment 215, wherein the albumin is serum albumin.

Embodiment 218 comprises an isolated polypeptide or polypeptide complex of any one of embodiments 215 and 217, wherein the albumin is human serum albumin.

Embodiment 219 comprises an isolated polypeptide or polypeptide complex of any one of embodiments 206-218, wherein H_{1c} comprises a polypeptide, a ligand, or a small molecule.

Embodiment 220 comprises an isolated polypeptide or polypeptide complex of embodiment 219, wherein the polypeptide, the ligand or the small molecule binds a serum protein or a fragment thereof, a circulating immunoglobulin or a fragment thereof, or CD35/CR1.

Embodiment 221 comprises an isolated polypeptide or polypeptide complex of embodiment 220, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 222 comprises an isolated polypeptide or polypeptide complex of embodiment 220, wherein the circulating immunoglobulin molecule comprises IgGl, IgG2, IgG3, IgG4, slgA, IgM or IgD.

Embodiment 223 comprises an isolated polypeptide or polypeptide complex of any one of embodiments 220-221, wherein the serum protein is albumin.

Embodiment 224 comprises an isolated polypeptide or polypeptide complex of any one of embodiments 219-223, wherein the polypeptide is an antibody.

Embodiment 225 comprises an isolated polypeptide or polypeptide complex of embodiment 224, wherein the antibody comprises a single domain antibody, a single chain variable fragment or a Fab.

Embodiment 226 comprises an isolated polypeptide or polypeptide complex of any one of embodiments 224-225, wherein the antibody comprises a single domain antibody that binds to albumin.

Embodiment 227 comprises an isolated polypeptide or polypeptide complex of any one of embodiments 224-226, wherein the antibody is a human or humanized antibody.

Embodiment 228 comprises an isolated polypeptide or polypeptide complex of any one of embodiments 225-227, wherein the single domain antibody is 645gH1gL1.

Embodiment 229 comprises an isolated polypeptide or polypeptide complex of any one of embodiments 225-227, wherein the single domain antibody is 645dsgH5gL4.

Embodiment 230 comprises an isolated polypeptide or polypeptide complex of embodiment 225, wherein the single domain antibody is 23-13-A01-sc02.

Embodiment 231 comprises an isolated polypeptide or polypeptide complex of any one of embodiments 225-226, wherein the single domain antibody is A10m3 or a fragment thereof.

Embodiment 232 comprises an isolated polypeptide or polypeptide complex of any one of embodiments 225-226, wherein the single domain antibody is DOM7r-31.

Embodiment 233 comprises an isolated polypeptide or polypeptide complex of any one of embodiments 225-226, wherein the single domain antibody is DOM7h-11-15.

Embodiment 234 comprises an isolated polypeptide or polypeptide complex of any one of embodiments 225-226, wherein the single domain antibody is Alb-1, Alb-8, or Alb-23.

Embodiment 235 comprises an isolated polypeptide or polypeptide complex of embodiment 225, wherein the single domain antibody is 10G or 10GE.

Embodiment 236 comprises an isolated polypeptide or polypeptide complex of any one of embodiments 225-226, wherein the single domain antibody is SA21.

Embodiment 237 comprises an isolated polypeptide or polypeptide complex of any one of embodiments 206-236, wherein H_{1c} comprises a linking moiety (L_{3c}) that connects H_{1c} to Pic.

Embodiment 238 comprises an isolated polypeptide or polypeptide complex of embodiment 237, wherein L_{3c} is a peptide sequence having at least 5 to no more than 50 amino acids.

Embodiment 239 comprises an isolated polypeptide or polypeptide complex of any one of embodiments 237-238, wherein L_{3c} is a peptide sequence having at least 10 to no more than 30 amino acids.

Embodiment 240 comprises an isolated polypeptide or polypeptide complex of any one of embodiments 237-239, wherein L_{3c} is a peptide sequence having at least 10 amino acids.

Embodiment 241 comprises an isolated polypeptide or polypeptide complex of any one of embodiments 237-240, wherein L_{3c} is a peptide sequence having at least 18 amino acids.

Embodiment 242 comprises an isolated polypeptide or polypeptide complex of any one of embodiments 237-241, wherein L_{3c} is a peptide sequence having at least 26 amino acids.

Embodiment 243 comprises an isolated polypeptide or polypeptide complex of any one of embodiments 237-242, wherein L_{3c} has a formula selected from the group consisting of (G₂S)ₙ, (GS)ₙ, (GSGGS)ₙ (SEQ ID NO: 917), (GGGS)ₙ (SEQ ID NO: 918), (GGGGS)ₙ (SEQ ID NO: 919), and (GSSGGS)ₙ (SEQ ID NO: 920), wherein n is an integer of at least 1.

Embodiment 244 comprises an isolated polypeptide or polypeptide complex of any one of embodiments 237-242, wherein L_{3c} comprises an amino acid sequence according to (GGGGSGGGS (SEQ ID NO: 921)).

### EXAMPLES

### Example 1: Structure Activity Relationships for Peptide-1 and Peptide 2 by Ala Scanning

Sequence activity relationships were established for Peptide-1 and Peptide-2 by mutating each individual residue within the peptide to alanine and measuring binding and inhibition against SP34.185 scFv. Peptide residues whose alanine mutations significantly weakened binding and inhibition were considered key residues where mutations were not tolerated. Peptide residues whose alanine mutations performed similarly to the non-mutated sequence were considered non-critical sites where mutations were indeed tolerated.

SP34.185 scFv Binding to Peptides by ELISA

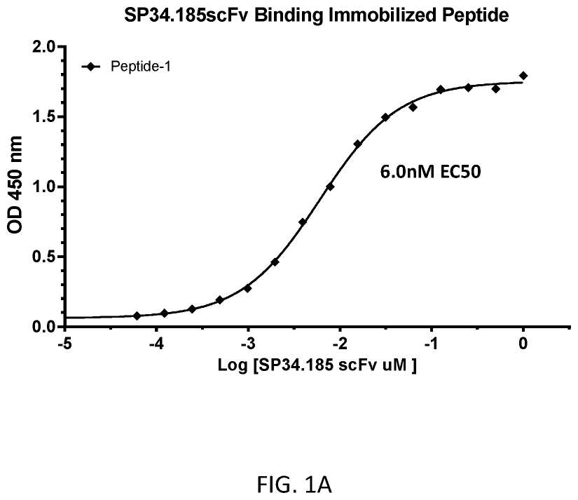

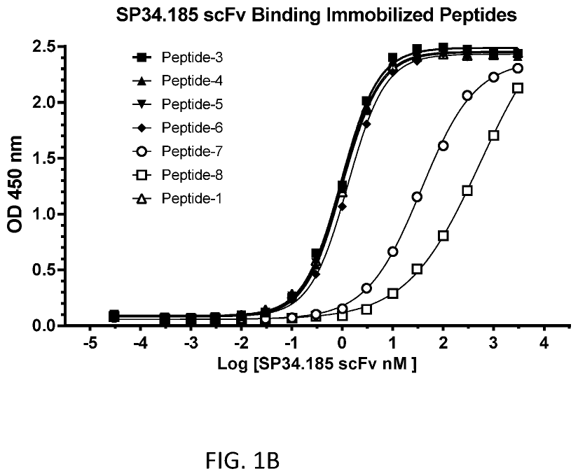

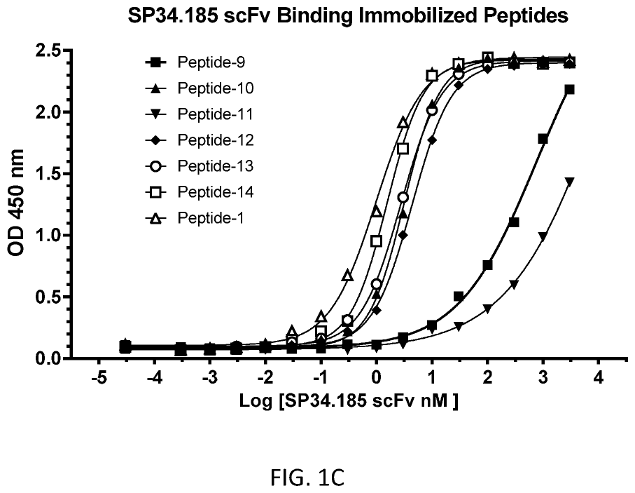

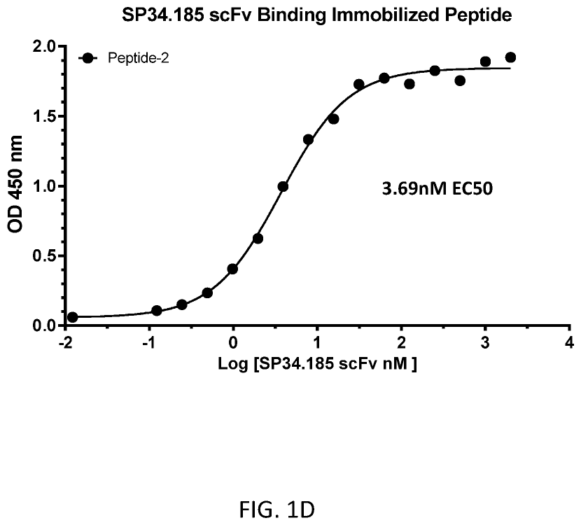

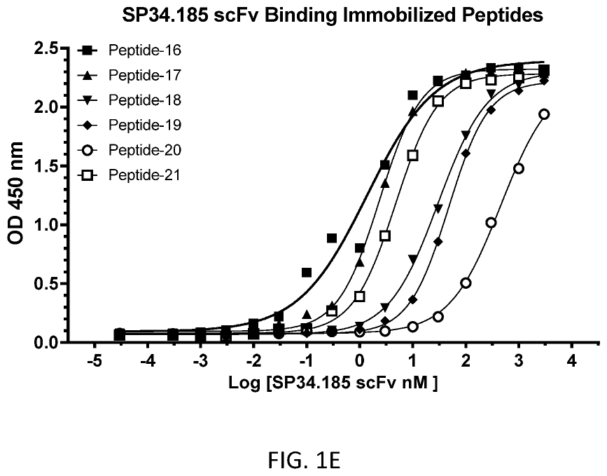

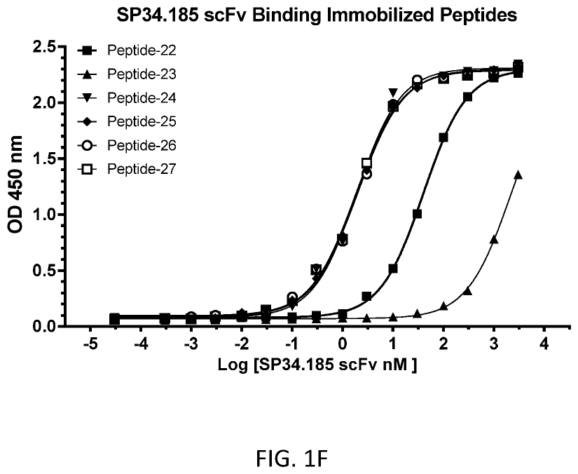

Peptides were evaluated for their ability to bind SP34.185 scFv by standard enzyme linked immunosorbent assays (ELISAs). Briefly, biotinylated peptides were captured on neutravidin coated plates, quench with biocytin followed by a washing step. SP34.185 scFv was then titrated onto the peptide captured plates. Plates were then washed and bound SP34.185 scFv was detected using a secondary horse radish peroxidase antibody conjugate. After washing again, plates were developed using standard ELISA techniques and stopped using acid. The concentration of SP34.185 scFv required to achieve 50% maximal signal or EC50 was calculated using Graphpad prism. Data is shown in FIGS. 1A-1F.

TABLE 6
Summary of FIG. 1B

| | Pep- | Pep- | Pep- | Pep- | Pep- | Pep- | Pep- |
|---|---|---|---|---|---|---|---|
| | tide- | tide- | tide- | tide- | tide- | tide- | tide- |
| ELISA | 1 | 3 | 4 | 5 | 6 | 7 | 8 |
| EC50 | 1.013 | 0.9429 | 1.018 | 0.9738 | 1.27 | 47.5 | 346.2 |
| nM | | | | | | | |

TABLE 7
Summary of FIG. 1C

| | Pep- | Pep- | Pep- | Pep- | Pep- | Pep- | Pep- |
|---|---|---|---|---|---|---|---|
| | tide- | tide- | tide- | tide- | tide- | tide- | tide- |
| ELISA | 1 | 9 | 10 | 11 | 12 | 13 | 14 |
| EC50 | 0.986 | 310.8 | 3.134 | 1.960 | 4.363 | 2.76 | 1.546 |
| nM | | | | | | | |

TABLE 8
Summary of FIG. 1E

| | Peptide- | Peptide- | Peptide- | Peptide- | Peptide- | Peptide- |
|---|---|---|---|---|---|---|
| ELISA | 16 | 17 | 18 | 19 | 20 | 21 |
| EC50 nM | 1.356 | 2.359 | 30.04 | 47.50 | 457.1 | 4.762 |

TABLE 9
Summary of FIG. 1F

| | Peptide- | Peptide- | Peptide- | Peptide- | Peptide- | Peptide- |
|---|---|---|---|---|---|---|
| ELISA | 22 | 23 | 24 | 25 | 26 | 27 |
| EC50 nM | 39.90 | 2168 | 1.916 | 1.948 | 2.012 | 1.833 |

Peptide Inhibition of SP34.185 scFv by ELISA

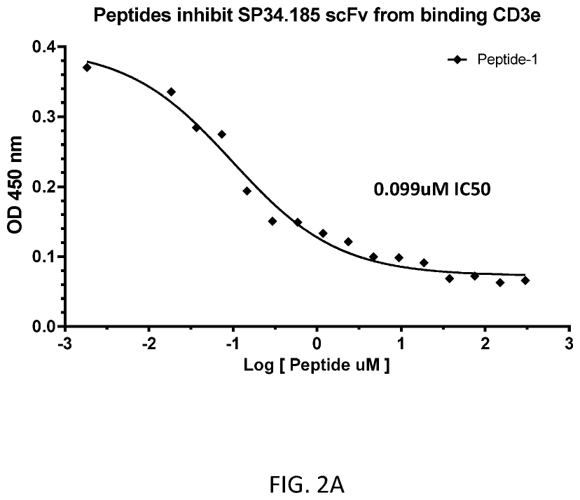

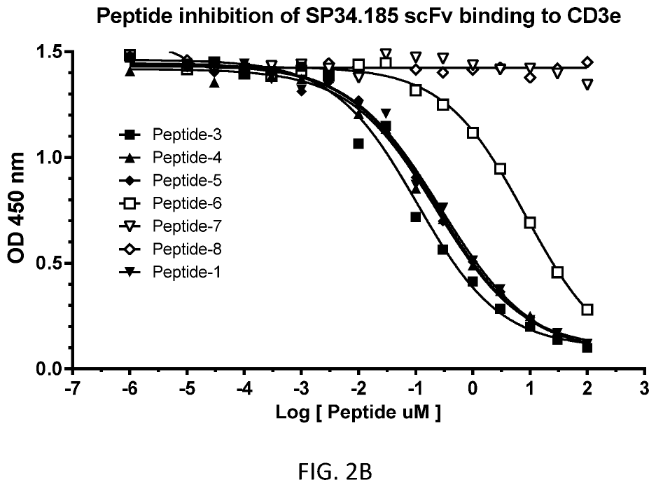

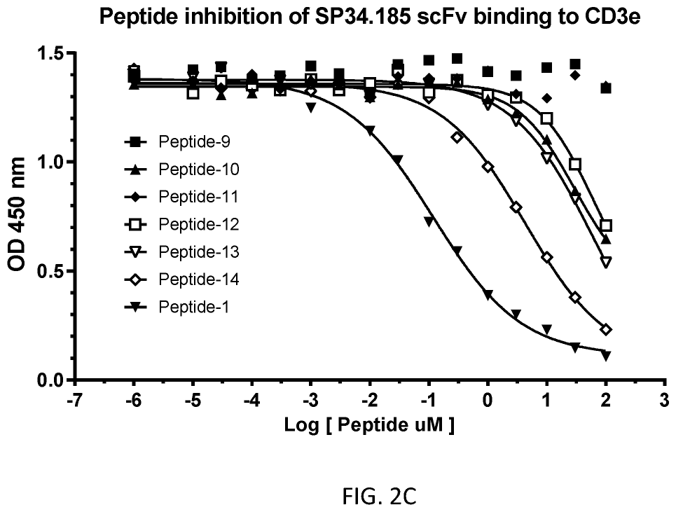

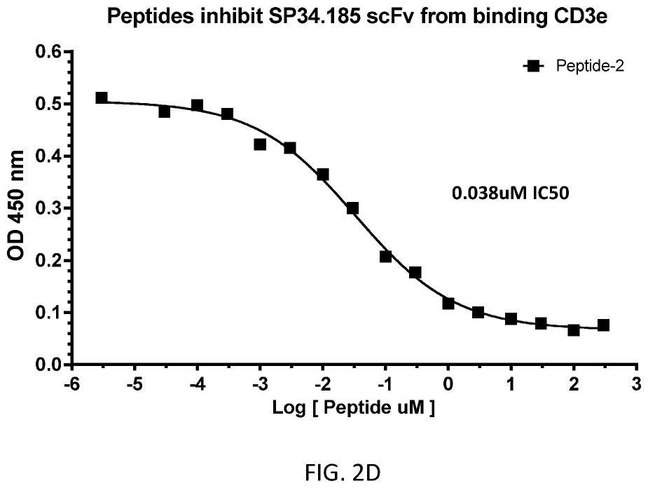

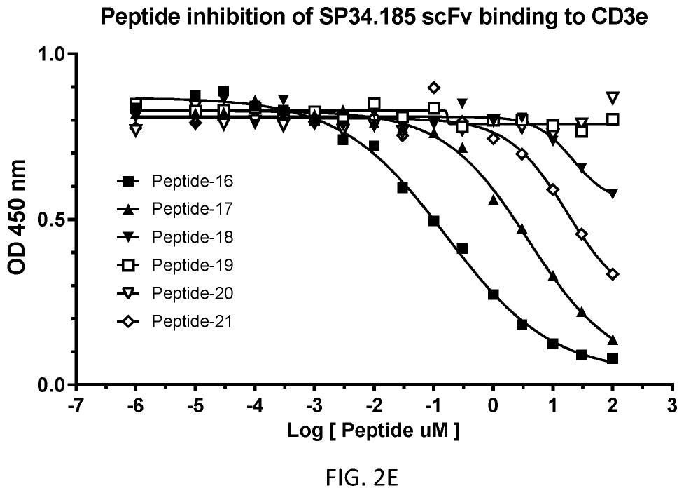

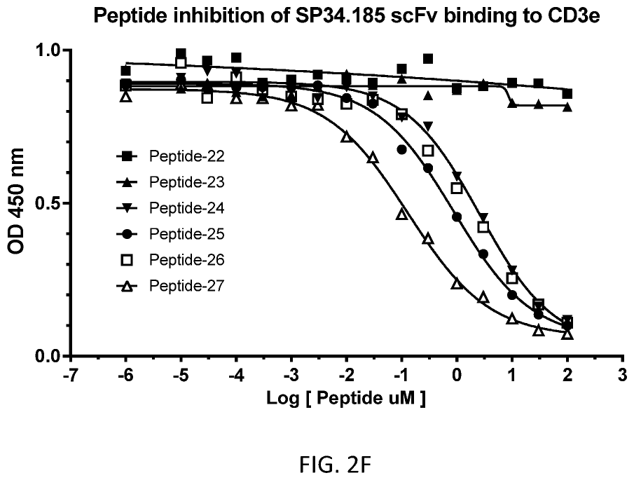

Peptides were evaluated for their ability to inhibit SP34.185 scFv from binding CD3e by standard enzyme linked immunosorbent assays (ELISAs). Briefly, a fixed concentration of SP34.185 scFv was incubated with varying concentrations of peptides in solution. SP34.185scFv and peptide solutions were incubated for 1 hr prior to addition to CD3 coated plates. Binding was allowed to proceed for 30 min prior to washing. After washing, bound SP34.185 scFv was measured using a secondary horse radish peroxidase antibody conjugate. After washing again, plates were developed using standard ELISA techniques and stopped using acid. The concentration of peptide required to inhibit 50% of the SP34.185 scFv CD3 binding signal (IC50) was calculated using Graphpad prism. Data is shown in FIGS. 2A-2F.

TABLE 10
Summary of FIG. 2B

| | Pep- | Pep- | Pep- | Pep- | Pep- | Pep- | Pep- |
|---|---|---|---|---|---|---|---|
| | tide- | tide- | tide- | tide- | tide- | tide- | tide- |
| ELISA | 1 | 3 | 4 | 5 | 6 | 7 | 8 |
| IC50 | 0.1926 | 0.1025 | 0.2318 | 0.1905 | 5.484 | >100 | >100 |
| uM | | | | | | | |

TABLE 11
Summary of FIG. 2C

| | Pep- | Pep- | Pep- | Pep- | Pep- | Pep- | Pep- |
|---|---|---|---|---|---|---|---|
| | tide- | tide- | tide- | tide- | tide- | tide- | tide- |
| ELISA | 1 | 9 | 10 | 11 | 12 | 13 | 14 |
| IC50 | 0.1138 | >100 | 63.18 | >100 | 86.78 | 36.66 | 3.009 |
| uM | | | | | | | |

TABLE 12
Summary of FIG. 2E

| | Peptide- | Peptide- | Peptide- | Peptide- | Peptide- | Peptide- |
|---|---|---|---|---|---|---|
| ELISA | 16 | 17 | 18 | 19 | 20 | 21 |
| IC50 uM | 0.1473 | 3.333 | >100 | >100 | >100 | 41.46 |

TABLE 13
Summary of FIG. 2F

| | Peptide- | Peptide- | Peptide- | Peptide- | Peptide- | Peptide- |
|---|---|---|---|---|---|---|
| ELISA | 22 | 23 | 24 | 25 | 26 | 27 |
| IC50 uM | >100 | >100 | 1.912 | 0.6992 | 1.456 | 0.1180 |

TABLE 14
Summary of Peptide Sequences

| | anti-CD3 | | SEQ |
|---|---|---|---|
| Peptide-ID | Panned target | Sequence | ID NO: |
| Peptide-1 | SP34.185 | GSQCLGPEWEVCPY | 1 |
| Peptide-3 | SP34.185 | ASQCLGPEWEVCPY | 19 |
| Peptide-4 | SP34.185 | GAQCLGPEWEVCPY | 20 |
| Peptide-5 | SP34.185 | GSACLGPEWEVCPY | 21 |
| Peptide-6 | SP34.185 | GSQCAGPEWEVCPY | 22 |
| Peptide-7 | SP34.185 | GSQCLAPEWEVCPY | 23 |
| Peptide-8 | SP34.185 | GSQCLGAEWEVCPY | 24 |
| Peptide-9 | SP34.185 | GSQCLGPAWEVCPY | 25 |
| Peptide-10 | SP34.185 | GSQCLGPEAEVCPY | 26 |
| Peptide-11 | SP34.185 | GSQCLGPEWAVCPY | 27 |
| Peptide-12 | SP34.185 | GSQCLGPEWEACPY | 28 |
| Peptide-13 | SP34.185 | GSQCLGPEWEVCAY | 29 |
| Peptide-14 | SP34.185 | GSQCLGPEWEVCPA | 30 |
| Peptide-15 | SP34.185 | GSQCLGPEWEVCPY | 31 |
| Peptide-2 | SP34.185 | VYCGPEFDESVGCM | 2 |
| Peptide-16 | SP34.185 | AYCGPEFDESVGCM | 32 |
| Peptide-17 | SP34.185 | VACGPEFDESVGCM | 33 |
| Peptide-18 | SP34.185 | VYCAPEFDESVGCM | 34 |
| Peptide-19 | SP34.185 | VYCGAEFDESVGCM | 35 |
| Peptide-20 | SP34.185 | VYCGPAFDESVGCM | 36 |
| Peptide-21 | SP34.185 | VYCGPEADESVGCM | 37 |
| Peptide-22 | SP34.185 | VYCGPEFAESVGCM | 38 |
| Peptide-23 | SP34.185 | VYCGPEFDASVGCM | 39 |
| Peptide-24 | SP34.185 | VYCGPEFDEAVGCM | 40 |
| Peptide-25 | SP34.185 | VYCGPEFDESAGCM | 41 |
| Peptide-26 | SP34.185 | VYCGPEFDESVACM | 42 |
| Peptide-27 | SP34.185 | VYCGPEFDESVGCA | 43 |

### Example 2: Polypeptide Binding to CD3 by ELISA

Peptide masks were incorporated into bispecific polypeptide molecules via tethering to SP34.185 scFv using a cleavable linker. Polypeptide molecules were then evaluated for their ability to bind CD3 before and after protease treatment via standard enzyme linked immunosorbent assays (ELISAs). Briefly, biotinylated CD3 was captured on neutravidin coated plates, quenched with biocytin, and washed. Polypeptide molecules were treated with protease when indicated. Polypeptide molecules were then titrated in buffer and added to the CD3 captured ELISA plate. After a brief incubation, plates were washed, and bound polypeptide molecules were detected using a standard secondary horse radish peroxidase conjugate antibody. After another wash, ELISA plates were developed and stopped in acid. The concentration of polypeptide required to achieve a 50% maximal signal (EC50) was calculated in Graphpad prism. Tethering of inhibitory peptides to SP34.185 scFv significantly hindered the ability to bind CD3 relative to the control polypeptide that lacks inhibitory peptide masking.

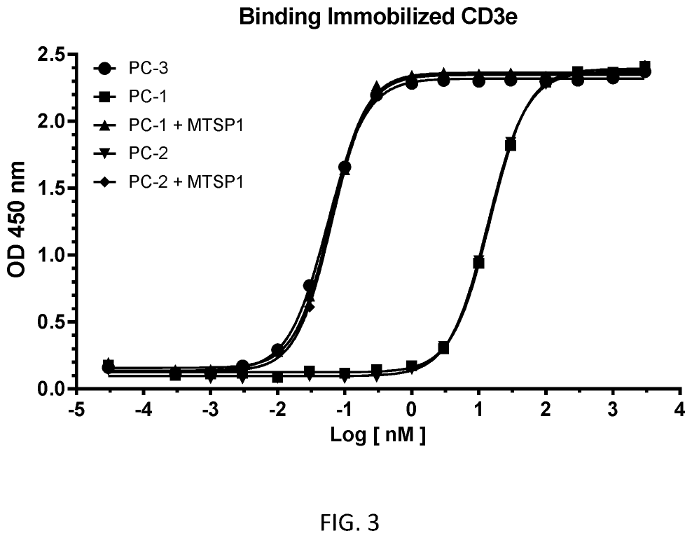

TABLE 15
Summary of FIG. 3

| | | PC-1 + | | PC-2 + | |
|---|---|---|---|---|---|
| | PC-1 | MTSP1 | PC-2 | MTSP1 | PC-3 |
| EC50 nM | 13.66 | 0.05722 | 13.28 | 0.06099 | 0.05401 |
| Fold shift | 252.9× | 1.1× | 245.9× | 1.1× | 1× |

### Example 3: Polypeptide Mediated Tumor Cell Killing

Peptide masks were incorporated into bispecific polypeptide molecules via tethering to SP34.185 scFv using a cleavable linker. Bispecific polypeptide molecules' functional activities were then evaluated before and after protease treatment via tumor cell line killing assays. Tumor cell killing was measured using an xCelligence real time cell analyzer (RTCA from Agilent) that relies on surface impedance to monitor cell growth kinetics. Briefly, tumor cell lines were seeded on E-plates and allowed to adhere overnight in a humidified incubator at 37 C with 5% carbon dioxide atmosphere. After the overnight incubation, bispecific polypeptide molecules were titrated in cell culture medium and added to the plates. In addition, effector cells such as PBMCs or CD8+ T cells were added to the plate at the indicated effector cell to tumor cell ratio. Bispecific polypeptides were treated with protease when indicated. Plates were then placed back on the RTCA inside the humidified incubator and impedance measurements (cell index) collected every 10 minutes for multiple days. After multiple days, the area under the tumor growth curves (cell index times hours) were plotted against the logarithmic concentration of polypeptide molecule. The concentration of polypeptide molecule required to inhibit tumor cell growth by 50% (IC50) was calculated in Graphpad Prism. Masking of the polypeptide binding domains significantly inhibited functional tumor killing while treatment of masked polypeptides with protease restored functional tumor killing.

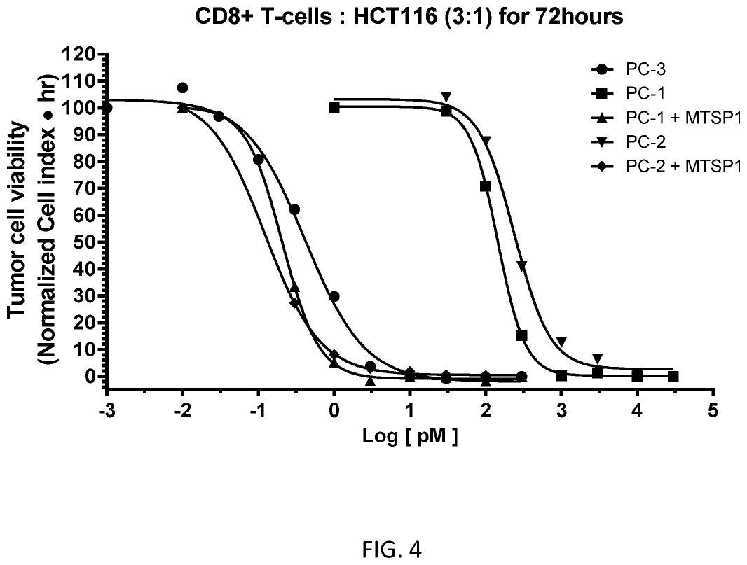

TABLE 16
Summary of FIG. 4

| | | PC-1 + | | PC-2 + | |
|---|---|---|---|---|---|
| | PC-1 | MTSP1 | PC-2 | MTSP1 | PC-3 |
| IC50 pM | 0.2463 | 144.3 | 0.1234 | 239.5 | 0.4171 |
| Fold shift | 1× | 585.9× | 1× | 1940.8× | control |

### Example 4: Optimized Phage Library Construction

Using the peptide sequence activity relationships (SAR) of Example 1, DNA oligo libraries were constructed where codons encoding critical residues within each peptide sequence were minimally mutated and codons encoding non-critical residues were heavily mutated. The resulting oligos were cloned into bacteriophage vectors used to display the SAR guided peptides via fusion to the pIII filament of the bacteriophage. The relevant vectors were then used to produce the phage optimization libraries via amplification in bacteria using standard techniques in the field.

### Example 5: Panning of the Optimized Phage Library

Once the phage optimization libraries were completed, phage libraries were bio-panned using SP34.185 scFv loaded beads. Multiple rounds of panning were performed where bacteriophage was allowed to bind to SP34.185 scFv loaded beads, washed, eluted, and amplified. Additional selective pressure was included during each round of panning using a fixed concentration of CD3, peptide-1, or peptide-2. After panning, phage infected bacteria were plated out and colonies picked into 96 well blocks. Clonal phage was then amplified and separated from bacterial cells via centrifugation. Phage containing supernatants were tested in binding ELISAs against SP34.185 scFv coated plates in the presence or absence of saturating concentration of CD3. Phage able to bind SP34.185 scFv were selected for sequence analysis if the binding signal was reduced in the presence of CD3.

### Example 6: Panning ELISAs

Clonal phage were harvested as crude supernatants and screened via standard enzyme linked immunosorbent assays (ELISAs). Briefly, biotinylated SP34.185 scFv was captured on neutravidin coated plates. Prior to the addition of clonal phage, wells were incubated with blocking buffer and CD3 or blocking buffer alone. Without washing or aspirating, clonal phage supernatants were then added to the wells and incubated for a short time. Wells were then washed followed by detection of bound phage using a horse radish peroxidase conjugated anti-M13 antibody. Clonal phage of interest were then sent for sequence analysis.

Phage panning results of Peptide-2 library sequences are shown in Table 17.

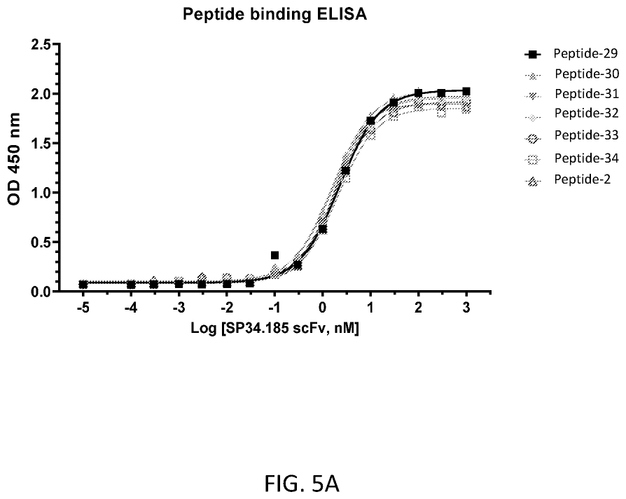

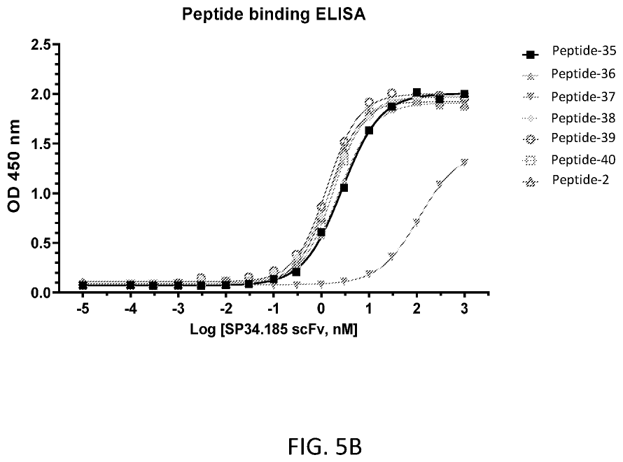

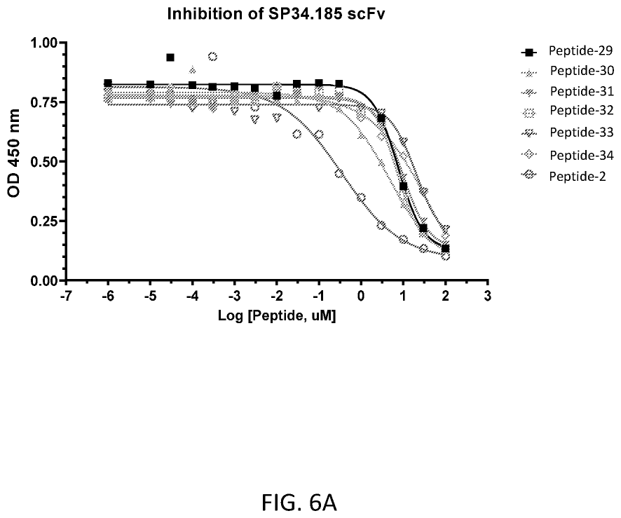

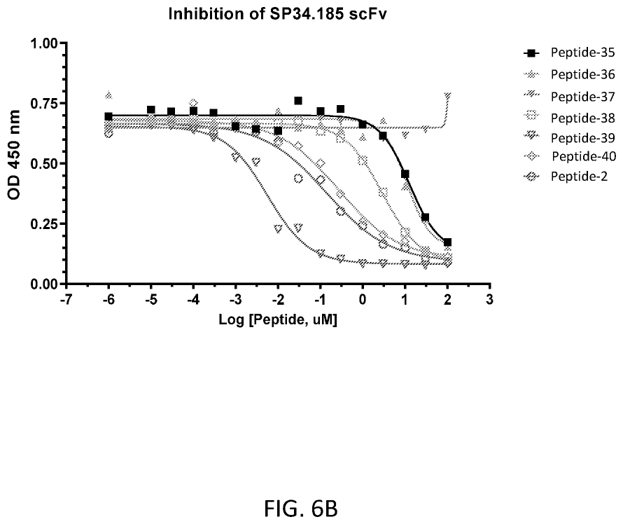

Peptide-29 to Peptide-40 were selected for sequence analysis and sequences are shown in Table 18. Peptide binding to SP34.185 scFv to CD3 is shown in FIGS. 5A-5B and summarized in Tables 19-20, respectively. Peptide inhibition of SP34.185 scFv binding to CD3 by ELISA is shown in FIGS. 6A-6B and summarized in Tables 21-22, respectively.

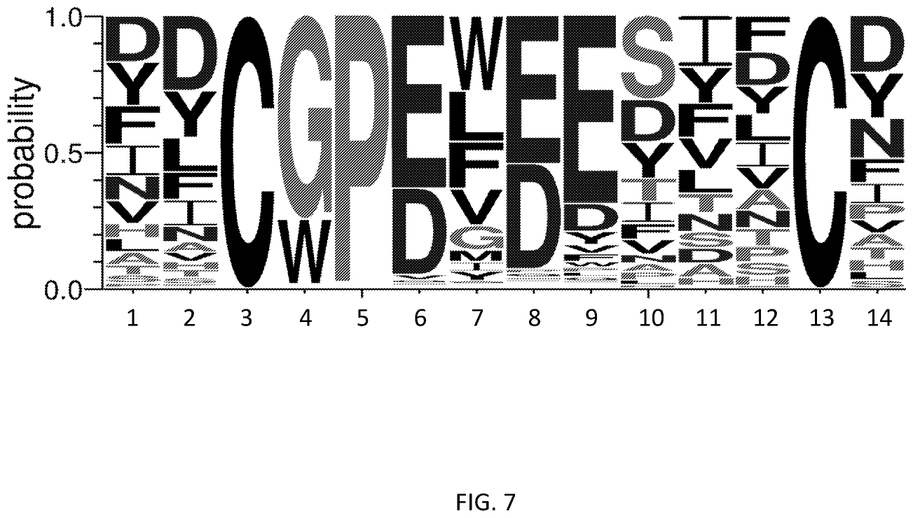

The core sequence motif for Peptide-2 is shown in FIG. 7.

TABLE 17
Clonal Phage Peptide Sequences from the Peptide-2 Optimization Library
Panning (—) indicates same amino acid as in Peptide-2
corresponding position (e.g. Phage-1 position).

| | | | | | | | | | | | | | | | Phage binding ELISA | | | |
|---|---|---|---|---|---|---|---|---|---|---|---|---|---|---|---|---|---|---|
| | | | | | | | | | | | | | | | | | SP34.185 | |
| | Amino acid position | | | | | | | | | | | | | | Back- | SP34.185 | scFv signal | SEQ |
| Phage | sequence | | | | | | | | | | | | | | ground | scFv | in presence | ID |
| ID | 1 | 2 | 3 | 4 | 5 | 6 | 7 | 8 | 9 | 10 | 11 | 12 | 13 | 14 | signal | signal | of CD3 | NO: |
| Phage-1/ | V | Y | C | G | P | E | F | D | E | S | V | G | C | M | 0.06 | 2.79 | 0.09 | 56 |
| Peptide 2 | | | | | | | | | | | | | | | | | | |
| Phage-2 | D | D | — | W | — | D | W | E | F | D | F | A | — | A | 0.08 | 2.75 | 0.09 | 57 |
| Phage-3 | Y | I | — | — | L | D | — | P | D | F | L | Y | — | D | 0.08 | 2.88 | 0.10 | 58 |
| Phage-4 | F | D | — | W | — | D | W | E | — | Y | F | V | — | D | 0.08 | 2.79 | 0.09 | 59 |
| Phage-5 | Y | I | — | W | — | D | W | E | — | Y | F | D | — | D | 0.08 | 2.74 | 0.09 | 60 |
| Phage-6 | N | I | — | W | — | D | W | E | D | D | Y | F | — | F | 0.09 | 2.54 | 0.09 | 61 |
| Phage-7 | N | F | — | W | — | D | W | E | Y | I | Y | P | — | I | 0.07 | 2.77 | 0.09 | 62 |
| Phage-8 | — | D | — | W | — | D | W | E | — | D | F | L | — | I | 0.08 | 2.54 | 0.08 | 63 |
| Phage-9 | H | A | — | W | — | D | W | E | — | Y | F | P | — | N | 0.08 | 2.85 | 0.09 | 64 |
| Phage- | Y | D | — | — | — | D | V | — | — | — | Y | V | — | V | 0.09 | 2.63 | 0.10 | 65 |
| 10 | | | | | | | | | | | | | | | | | | |
| Phage- | I | D | — | W | — | D | W | E | D | D | T | F | — | Y | 0.09 | 2.73 | 0.08 | 66 |
| 11 | | | | | | | | | | | | | | | | | | |
| Phage- | Y | L | — | — | — | D | G | — | — | T | L | A | — | Y | 0.08 | 2.66 | 0.15 | 67 |
| 12 | | | | | | | | | | | | | | | | | | |
| Phage- | — | D | — | — | — | D | G | — | — | — | I | L | — | Y | 0.11 | 2.13 | 0.08 | 68 |
| 13 | | | | | | | | | | | | | | | | | | |
| Phage- | F | I | — | W | — | D | W | E | — | D | Y | F | — | A | 0.07 | 2.44 | 0.09 | 69 |
| 14 | | | | | | | | | | | | | | | | | | |
| Phage- | G | D | — | W | — | D | W | E | W | D | F | Y | — | D | 0.07 | 2.71 | 0.07 | 70 |
| 15 | | | | | | | | | | | | | | | | | | |
| Phage- | Y | L | — | W | — | D | W | E | Y | I | D | L | — | D | 0.12 | 2.67 | 0.08 | 71 |
| 16 | | | | | | | | | | | | | | | | | | |
| Phage- | S | F | — | W | — | D | W | E | — | Y | F | D | — | D | 0.10 | 2.60 | 0.07 | 72 |
| 17 | | | | | | | | | | | | | | | | | | |
| Phage- | D | D | — | W | — | D | W | E | — | Y | A | S | — | D | 0.09 | 2.57 | 0.07 | 73 |
| 18 | | | | | | | | | | | | | | | | | | |
| Phage- | N | L | — | W | — | D | W | E | Y | P | F | F | — | D | 0.09 | 2.52 | 0.09 | 74 |
| 19 | | | | | | | | | | | | | | | | | | |
| Phage- | F | D | — | W | — | D | W | E | — | — | F | V | — | D | 0.08 | 2.34 | 0.09 | 75 |
| 20 | | | | | | | | | | | | | | | | | | |
| Phage- | D | I | — | — | — | D | G | — | — | T | I | I | — | D | 0.13 | 2.30 | 0.10 | 76 |
| 21 | | | | | | | | | | | | | | | | | | |
| Phage- | D | D | — | W | — | D | W | E | Y | Y | A | V | — | D | 0.09 | 2.28 | 0.09 | 77 |
| 22 | | | | | | | | | | | | | | | | | | |
| Phage- | Y | D | — | W | — | D | W | E | — | Y | S | N | — | D | 0.10 | 2.17 | 0.08 | 78 |
| 23 | | | | | | | | | | | | | | | | | | |
| Phage- | I | N | — | W | — | D | W | E | D | Y | F | F | — | D | 0.07 | 2.16 | 0.07 | 79 |
| 24 | | | | | | | | | | | | | | | | | | |
| Phage- | N | I | — | W | — | D | W | E | D | D | T | F | — | F | 0.06 | 2.87 | 0.07 | 80 |
| 25 | | | | | | | | | | | | | | | | | | |
| Phage- | N | I | — | W | — | D | W | E | P | N | S | F | — | F | 0.09 | 2.87 | 0.08 | 81 |
| 26 | | | | | | | | | | | | | | | | | | |
| Phage- | Y | D | — | — | — | — | M | — | — | — | T | D | — | F | 0.09 | 2.39 | 0.08 | 82 |
| 27 | | | | | | | | | | | | | | | | | | |
| Phage- | D | F | — | W | — | D | W | E | F | P | F | I | — | H | 0.11 | 2.73 | 0.12 | 83 |
| 28 | | | | | | | | | | | | | | | | | | |
| Phage- | D | F | — | — | — | — | M | — | — | — | I | T | — | I | 0.07 | 2.36 | 0.08 | 84 |
| 29 | | | | | | | | | | | | | | | | | | |
| Phage- | Y | D | — | — | — | — | — | — | — | — | T | V | — | I | 0.10 | 2.32 | 0.08 | 85 |
| 30 | | | | | | | | | | | | | | | | | | |
| Phage- | H | D | — | W | — | D | W | E | W | D | I | F | — | I | 0.07 | 2.26 | 0.08 | 86 |
| 31 | | | | | | | | | | | | | | | | | | |
| Phage- | H | A | — | W | — | D | W | E | — | Y | N | P | — | N | 0.11 | 2.71 | 0.11 | 87 |
| 32 | | | | | | | | | | | | | | | | | | |
| Phage- | D | V | — | W | — | D | W | E | W | D | F | F | — | N | 0.08 | 2.65 | 0.08 | 88 |
| 33 | | | | | | | | | | | | | | | | | | |
| Phage- | N | — | — | W | — | D | W | E | Y | Y | — | P | — | N | 0.10 | 2.57 | 0.08 | 89 |
| 34 | | | | | | | | | | | | | | | | | | |
| Phage- | I | I | — | W | — | D | W | E | F | I | D | Y | — | N | 0.08 | 2.10 | 0.07 | 90 |
| 35 | | | | | | | | | | | | | | | | | | |
| Phage- | S | L | — | W | — | D | W | E | Y | D | I | A | — | P | 0.07 | 2.53 | 0.08 | 91 |
| 36 | | | | | | | | | | | | | | | | | | |
| Phage- | D | L | — | — | — | — | L | — | — | — | I | F | — | P | 0.08 | 2.49 | 0.09 | 92 |
| 37 | | | | | | | | | | | | | | | | | | |
| Phage- | T | N | — | W | — | D | W | E | W | V | L | P | — | P | 0.14 | 2.47 | 0.10 | 93 |
| 38 | | | | | | | | | | | | | | | | | | |
| Phage- | T | E | — | W | — | D | W | E | P | N | Y | F | — | P | 0.13 | 2.29 | 0.09 | 94 |
| 39 | | | | | | | | | | | | | | | | | | |
| Phage- | I | F | — | W | — | D | W | E | D | Y | I | D | — | P | 0.07 | 2.28 | 0.07 | 95 |
| 40 | | | | | | | | | | | | | | | | | | |
| Phage- | T | D | — | W | — | D | W | E | Y | D | F | F | — | P | 0.07 | 2.26 | 0.08 | 96 |
| 41 | | | | | | | | | | | | | | | | | | |
| Phage- | L | F | — | W | — | D | W | E | D | — | F | F | — | P | 0.18 | 2.11 | 0.13 | 97 |
| 42 | | | | | | | | | | | | | | | | | | |
| Phage- | — | D | — | W | — | D | W | E | D | Y | A | D | — | T | 0.11 | 2.20 | 0.10 | 98 |
| 43 | | | | | | | | | | | | | | | | | | |
| Phage- | — | I | — | W | — | D | W | E | D | Y | F | P | — | V | 0.11 | 2.34 | 0.09 | 99 |
| 44 | | | | | | | | | | | | | | | | | | |
| Phage- | I | E | — | W | — | D | W | E | P | I | Y | P | — | Y | 0.09 | 2.85 | 0.09 | 100 |
| 45 | | | | | | | | | | | | | | | | | | |
| Phage- | I | T | — | W | — | D | W | E | V | Y | F | P | — | Y | 0.07 | 2.55 | 0.08 | 101 |
| 46 | | | | | | | | | | | | | | | | | | |
| Phage- | I | D | — | W | — | D | W | E | Y | I | H | P | — | Y | 0.06 | 2.51 | 0.09 | 102 |
| 47 | | | | | | | | | | | | | | | | | | |
| Phage- | I | D | — | W | — | D | W | E | Y | I | N | P | — | Y | 0.12 | 2.50 | 0.12 | 103 |
| 48 | | | | | | | | | | | | | | | | | | |
| Phage- | A | D | — | W | — | D | W | E | — | A | F | P | I | Y | 0.09 | 2.44 | 0.09 | 104 |
| 49 | | | | | | | | | | | | | | | | | | |
| Phage- | I | D | — | W | — | D | W | E | Y | I | Y | P | — | Y | 0.09 | 2.31 | 0.07 | 105 |
| 50 | | | | | | | | | | | | | | | | | | |
| Phage- | N | I | — | W | — | D | W | E | D | D | N | F | — | F | 0.09 | 2.08 | 0.09 | 106 |
| 51 | | | | | | | | | | | | | | | | | | |
| Phage- | Y | D | — | W | — | D | W | E | Y | V | D | A | — | Y | 0.09 | 2.06 | 0.09 | 107 |
| 52 | | | | | | | | | | | | | | | | | | |
| Phage- | F | — | — | — | — | D | G | — | — | — | Y | V | — | D | 0.09 | 2.03 | 0.11 | 108 |
| 53 | | | | | | | | | | | | | | | | | | |
| Phage- | D | I | — | W | — | D | W | E | Y | I | N | I | — | S | 0.11 | 2.02 | 0.11 | 109 |
| 54 | | | | | | | | | | | | | | | | | | |
| Phage- | F | V | — | W | — | D | W | E | D | F | N | F | — | D | 0.07 | 2.01 | 0.08 | 110 |
| 55 | | | | | | | | | | | | | | | | | | |
| Phage- | F | A | — | W | — | D | W | E | D | Y | — | A | — | D | 0.07 | 2.01 | 0.09 | 111 |
| 56 | | | | | | | | | | | | | | | | | | |
| Phage- | D | N | — | W | — | D | W | E | Y | D | F | F | — | V | 0.08 | 1.99 | 0.09 | 112 |
| 57 | | | | | | | | | | | | | | | | | | |
| Phage- | Y | D | — | W | — | D | W | E | — | Y | N | D | — | A | 0.09 | 1.96 | 0.11 | 113 |
| 58 | | | | | | | | | | | | | | | | | | |
| Phage- | D | D | — | — | — | D | G | — | — | T | I | I | — | V | 0.07 | 1.91 | 0.09 | 114 |
| 59 | | | | | | | | | | | | | | | | | | |
| Phage- | F | P | — | W | — | D | W | E | — | Y | A | I | — | D | 0.10 | 1.89 | 0.10 | 115 |
| 60 | | | | | | | | | | | | | | | | | | |
| Phage- | P | D | — | — | — | D | G | — | — | — | L | F | — | T | 0.12 | 1.86 | 0.07 | 116 |
| 61 | | | | | | | | | | | | | | | | | | |
| Phage- | D | N | — | W | — | D | W | E | Y | D | Y | F | — | V | 0.07 | 1.83 | 0.07 | 117 |
| 62 | | | | | | | | | | | | | | | | | | |
| Phage- | I | F | — | W | — | D | W | E | — | F | Y | D | — | Y | 0.12 | 1.82 | 0.08 | 118 |
| 63 | | | | | | | | | | | | | | | | | | |
| Phage- | A | D | — | W | — | D | W | E | — | Y | F | P | — | N | 0.08 | 1.82 | 0.08 | 119 |
| 64 | | | | | | | | | | | | | | | | | | |
| Phage- | H | T | — | W | — | D | W | E | D | D | I | F | — | N | 0.12 | 1.81 | 0.10 | 120 |
| 65 | | | | | | | | | | | | | | | | | | |
| Phage- | F | A | — | W | — | D | W | E | — | A | F | L | — | L | 0.09 | 1.80 | 0.09 | 121 |
| 66 | | | | | | | | | | | | | | | | | | |
| Phage- | Y | D | — | — | — | — | L | — | — | — | I | A | — | D | 0.08 | 1.77 | 0.08 | 122 |
| 67 | | | | | | | | | | | | | | | | | | |
| Phage- | N | S | — | W | — | D | W | E | Y | D | I | I | — | D | 0.08 | 1.77 | 0.10 | 123 |
| 68 | | | | | | | | | | | | | | | | | | |
| Phage- | F | A | — | W | — | D | W | E | — | V | A | P | — | Y | 0.07 | 1.75 | 0.07 | 124 |
| 69 | | | | | | | | | | | | | | | | | | |
| Phage- | L | D | — | — | — | D | G | — | — | T | L | T | — | Y | 0.10 | 1.75 | 0.12 | 125 |
| 70 | | | | | | | | | | | | | | | | | | |
| Phage- | — | L | — | W | — | D | W | E | — | F | Y | D | — | P | 0.07 | 1.74 | 0.09 | 126 |
| 71 | | | | | | | | | | | | | | | | | | |
| Phage- | H | A | — | W | — | V | W | E | — | Y | F | P | — | N | 0.07 | 1.72 | 0.08 | 127 |
| 72 | | | | | | | | | | | | | | | | | | |
| Phage- | N | E | — | W | — | N | G | E | P | T | F | P | — | T | 0.08 | 1.71 | 0.07 | 128 |
| 73 | | | | | | | | | | | | | | | | | | |
| Phage- | L | T | — | — | — | D | G | — | — | T | L | Y | — | D | 0.08 | 1.70 | 0.07 | 129 |
| 74 | | | | | | | | | | | | | | | | | | |
| Phage- | Y | D | — | — | — | — | Y | — | — | — | — | P | I | I | 0.13 | 1.67 | 0.09 | 130 |
| 75 | | | | | | | | | | | | | | | | | | |
| Phage- | I | E | — | W | — | D | W | E | P | N | S | F | — | D | 0.09 | 1.66 | 0.08 | 131 |
| 76 | | | | | | | | | | | | | | | | | | |
| Phage- | Y | D | — | — | — | — | L | — | — | — | I | H | — | Y | 0.12 | 1.66 | 0.09 | 132 |
| 77 | | | | | | | | | | | | | | | | | | |
| Phage- | I | — | — | — | — | — | — | — | — | — | T | I | — | N | 0.08 | 1.63 | 0.08 | 133 |
| 78 | | | | | | | | | | | | | | | | | | |
| Phage- | I | — | — | — | — | — | V | E | — | A | Y | L | — | Y | 0.09 | 1.62 | 0.10 | 134 |
| 79 | | | | | | | | | | | | | | | | | | |
| Phage- | F | D | — | — | — | D | G | — | — | T | — | Y | — | D | 0.09 | 1.61 | 0.08 | 135 |
| 80 | | | | | | | | | | | | | | | | | | |
| Phage- | I | D | — | — | — | D | G | — | — | T | I | S | — | Y | 0.08 | 1.57 | 0.11 | 136 |
| 81 | | | | | | | | | | | | | | | | | | |
| Phage- | N | — | — | — | — | — | — | — | — | — | S | T | — | L | 0.10 | 1.55 | 0.11 | 137 |
| 82 | | | | | | | | | | | | | | | | | | |
| Phage- | Y | D | — | — | — | D | G | — | — | — | Y | F | — | D | 0.08 | 1.53 | 0.08 | 138 |
| 83 | | | | | | | | | | | | | | | | | | |
| Phage- | N | F | — | W | — | D | W | E | Y | F | N | D | — | N | 0.09 | 1.53 | 0.09 | 139 |
| 84 | | | | | | | | | | | | | | | | | | |
| Phage- | — | L | — | W | — | D | W | E | A | F | F | D | — | D | 0.07 | 1.47 | 0.07 | 140 |
| 85 | | | | | | | | | | | | | | | | | | |
| Phage- | I | — | — | — | — | — | W | E | W | P | — | A | — | N | 0.16 | 1.47 | 0.10 | 141 |
| 86 | | | | | | | | | | | | | | | | | | |
| Phage- | — | F | I | W | — | D | W | E | D | N | F | F | — | N | 0.08 | 1.46 | 0.10 | 142 |
| 87 | | | | | | | | | | | | | | | | | | |
| Phage- | — | V | — | W | — | D | W | E | T | F | F | P | — | D | 0.08 | 1.46 | 0.08 | 143 |
| 88 | | | | | | | | | | | | | | | | | | |
| Phage- | D | N | — | — | — | D | G | — | — | T | Y | I | — | N | 0.10 | 1.45 | 0.09 | 144 |
| 89 | | | | | | | | | | | | | | | | | | |
| Phage- | D | N | — | W | — | D | W | E | Y | N | F | F | — | V | 0.07 | 1.45 | 0.08 | 145 |
| 90 | | | | | | | | | | | | | | | | | | |
| Phage- | F | — | — | — | — | — | V | E | — | D | Y | L | — | I | 0.10 | 1.43 | 0.10 | 146 |
| 91 | | | | | | | | | | | | | | | | | | |
| Phage- | D | N | — | W | — | D | W | E | Y | D | T | F | — | V | 0.07 | 1.43 | 0.07 | 147 |
| 92 | | | | | | | | | | | | | | | | | | |
| Phage- | I | D | — | — | — | — | — | — | — | — | I | A | — | P | 0.08 | 1.42 | 0.08 | 148 |
| 93 | | | | | | | | | | | | | | | | | | |
| Phage- | Y | F | — | — | — | — | V | E | I | Y | T | L | I | F | 0.10 | 1.42 | 0.10 | 149 |
| 94 | | | | | | | | | | | | | | | | | | |
| Phage- | F | — | — | — | — | — | — | — | — | — | A | P | — | N | 0.06 | 1.37 | 0.08 | 150 |
| 95 | | | | | | | | | | | | | | | | | | |
| Phage- | F | D | — | — | — | — | V | E | — | Y | F | Y | — | A | 0.11 | 1.36 | 0.08 | 151 |
| 96 | | | | | | | | | | | | | | | | | | |
| Phage- | D | F | — | W | — | D | W | E | D | F | F | F | — | A | 0.18 | 1.35 | 0.12 | 152 |
| 97 | | | | | | | | | | | | | | | | | | |
| Phage- | F | F | — | — | — | D | G | — | — | T | L | S | — | N | 0.08 | 1.35 | 0.09 | 153 |
| 98 | | | | | | | | | | | | | | | | | | |
| Phage- | F | I | — | — | — | — | — | — | — | — | — | A | — | L | 0.14 | 1.35 | 0.09 | 154 |
| 99 | | | | | | | | | | | | | | | | | | |
| Phage- | Y | — | — | — | — | — | — | — | — | A | I | — | — | Y | 0.09 | 1.32 | 0.10 | 155 |
| 100 | | | | | | | | | | | | | | | | | | |
| Phage- | Y | T | — | W | — | D | W | E | — | Y | L | Y | — | P | 0.10 | 1.32 | 0.15 | 156 |
| 101 | | | | | | | | | | | | | | | | | | |
| Phage- | F | D | — | W | — | D | W | E | — | P | T | T | — | H | 0.08 | 1.31 | 0.08 | 157 |
| 102 | | | | | | | | | | | | | | | | | | |
| Phage- | Y | D | — | W | — | D | W | E | D | F | P | I | — | D | 0.14 | 1.31 | 0.10 | 158 |
| 103 | | | | | | | | | | | | | | | | | | |
| Phage- | — | V | — | W | — | D | W | E | Y | I | D | D | — | S | 0.08 | 1.30 | 0.07 | 159 |
| 104 | | | | | | | | | | | | | | | | | | |
| Phage- | I | N | — | W | — | D | W | E | V | T | S | F | — | D | 0.12 | 1.30 | 0.08 | 160 |
| 105 | | | | | | | | | | | | | | | | | | |
| Phage- | L | S | — | W | — | D | W | E | — | V | T | P | — | L | 0.10 | 1.29 | 0.10 | 161 |
| 106 | | | | | | | | | | | | | | | | | | |
| Phage- | F | A | — | W | — | D | W | E | — | V | D | I | — | Y | 0.09 | 1.28 | 0.08 | 162 |
| 107 | | | | | | | | | | | | | | | | | | |
| Phage- | Y | D | — | — | — | — | M | — | — | — | I | V | — | D | 0.10 | 1.25 | 0.08 | 163 |
| 108 | | | | | | | | | | | | | | | | | | |
| Phage- | Y | D | — | W | — | D | W | E | V | F | I | V | — | D | 0.06 | 1.25 | 0.07 | 164 |
| 109 | | | | | | | | | | | | | | | | | | |
| Phage- | D | N | — | W | — | D | W | E | H | N | F | F | — | V | 0.10 | 1.25 | 0.08 | 165 |
| 110 | | | | | | | | | | | | | | | | | | |
| Phage- | Y | D | — | — | — | D | G | — | — | — | T | Y | — | P | 0.07 | 1.23 | 0.08 | 166 |
| 111 | | | | | | | | | | | | | | | | | | |
| Phage- | Y | D | — | — | — | — | — | E | F | P | Y | Y | — | F | 0.12 | 1.23 | 0.12 | 167 |
| 112 | | | | | | | | | | | | | | | | | | |
| Phage- | A | D | — | — | — | — | Y | — | — | — | — | P | — | V | 0.11 | 1.22 | 0.09 | 168 |
| 113 | | | | | | | | | | | | | | | | | | |
| Phage- | F | L | — | — | — | — | V | E | — | V | H | Y | — | S | 0.08 | 1.22 | 0.10 | 169 |
| 114 | | | | | | | | | | | | | | | | | | |
| Phage- | T | D | — | W | — | D | W | E | Y | T | T | S | — | S | 0.08 | 1.22 | 0.08 | 170 |
| 115 | | | | | | | | | | | | | | | | | | |
| Phage- | A | F | — | — | — | — | L | — | — | — | I | T | — | D | 0.09 | 1.21 | 0.09 | 171 |
| 116 | | | | | | | | | | | | | | | | | | |
| Phage- | N | D | — | W | — | D | W | E | — | Y | F | S | — | Y | 0.09 | 1.19 | 0.09 | 172 |
| 117 | | | | | | | | | | | | | | | | | | |
| Phage- | F | D | — | — | — | — | W | E | I | V | T | D | — | Y | 0.08 | 1.19 | 0.09 | 173 |
| 118 | | | | | | | | | | | | | | | | | | |
| Phage- | N | L | — | — | — | — | M | — | — | — | I | I | — | P | 0.13 | 1.19 | 0.11 | 174 |
| 119 | | | | | | | | | | | | | | | | | | |
| Phage- | D | L | — | — | — | — | M | — | — | — | I | Y | — | D | 0.10 | 1.19 | 0.14 | 175 |
| 120 | | | | | | | | | | | | | | | | | | |
| Phage- | F | D | — | — | — | D | G | V | — | D | Y | I | — | D | 0.09 | 1.18 | 0.09 | 176 |
| 121 | | | | | | | | | | | | | | | | | | |
| Phage- | Y | A | — | W | — | D | W | E | — | D | F | A | — | Y | 0.11 | 1.18 | 0.08 | 177 |
| 122 | | | | | | | | | | | | | | | | | | |
| Phage- | H | D | — | — | — | — | M | — | — | — | I | V | — | V | 0.10 | 1.17 | 0.10 | 178 |
| 123 | | | | | | | | | | | | | | | | | | |
| Phage- | — | F | — | — | — | — | — | E | F | I | F | L | — | A | 0.07 | 1.17 | 0.08 | 179 |
| 124 | | | | | | | | | | | | | | | | | | |
| Phage- | Y | D | — | — | — | — | L | — | — | — | I | L | — | D | 0.08 | 1.16 | 0.09 | 180 |
| 125 | | | | | | | | | | | | | | | | | | |
| Phage- | S | V | — | W | — | D | W | E | — | F | Y | S | — | D | 0.11 | 1.16 | 0.10 | 181 |
| 126 | | | | | | | | | | | | | | | | | | |
| Phage- | P | — | — | — | — | D | G | — | — | T | A | I | — | T | 0.13 | 1.16 | 0.10 | 182 |
| 127 | | | | | | | | | | | | | | | | | | |
| Phage- | D | D | — | — | — | — | L | E | W | Y | Y | P | — | Y | 0.09 | 1.16 | 0.08 | 183 |
| 128 | | | | | | | | | | | | | | | | | | |
| Phage- | F | I | — | — | — | — | — | — | — | — | L | P | — | N | 0.08 | 1.14 | 0.09 | 184 |
| 129 | | | | | | | | | | | | | | | | | | |
| Phage- | I | D | — | — | — | — | — | — | — | — | L | P | — | D | 0.11 | 1.14 | 0.33 | 185 |
| 130 | | | | | | | | | | | | | | | | | | |
| Phage- | F | L | — | — | — | — | — | E | I | D | A | P | — | Y | 0.08 | 1.13 | 0.08 | 186 |
| 131 | | | | | | | | | | | | | | | | | | |
| Phage- | I | F | — | — | — | D | G | — | — | T | H | I | — | H | 0.10 | 1.13 | 0.07 | 187 |
| 132 | | | | | | | | | | | | | | | | | | |
| Phage- | — | F | — | W | — | D | W | E | Y | I | D | F | — | N | 0.10 | 1.11 | 0.21 | 188 |
| 133 | | | | | | | | | | | | | | | | | | |
| Phage- | I | F | — | — | — | — | Y | — | — | — | L | H | — | I | 0.12 | 1.11 | 0.11 | 189 |
| 134 | | | | | | | | | | | | | | | | | | |
| Phage- | H | L | — | W | — | D | W | E | W | Y | — | D | — | P | 0.08 | 1.11 | 0.10 | 190 |
| 135 | | | | | | | | | | | | | | | | | | |
| Phage- | F | I | — | — | — | — | M | — | — | — | I | A | — | N | 0.08 | 1.11 | 0.09 | 191 |
| 136 | | | | | | | | | | | | | | | | | | |
| Phage- | I | F | — | — | — | — | V | E | M | I | F | L | — | N | 0.09 | 1.10 | 0.08 | 192 |
| 137 | | | | | | | | | | | | | | | | | | |
| Phage- | Y | D | — | — | — | — | W | E | F | P | — | D | — | I | 0.11 | 1.09 | 0.11 | 193 |
| 138 | | | | | | | | | | | | | | | | | | |
| Phage- | N | L | — | — | — | — | L | — | — | — | I | T | — | F | 0.10 | 1.09 | 0.08 | 194 |
| 139 | | | | | | | | | | | | | | | | | | |
| Phage- | F | — | — | — | — | — | V | E | D | F | Y | F | — | Y | 0.08 | 1.09 | 0.08 | 195 |
| 140 | | | | | | | | | | | | | | | | | | |
| Phage- | D | — | — | — | — | — | — | — | — | — | L | I | — | N | 0.11 | 1.07 | 0.11 | 196 |
| 141 | | | | | | | | | | | | | | | | | | |
| Phage- | D | — | — | — | — | — | — | — | — | — | L | P | — | D | 0.08 | 1.07 | 0.08 | 197 |
| 142 | | | | | | | | | | | | | | | | | | |
| Phage- | A | I | — | — | — | — | L | — | — | — | I | A | — | P | 0.09 | 1.07 | 0.09 | 198 |
| 143 | | | | | | | | | | | | | | | | | | |
| Phage- | — | I | — | — | — | — | V | E | D | Y | N | L | — | Y | 0.08 | 1.07 | 0.09 | 199 |
| 144 | | | | | | | | | | | | | | | | | | |
| Phage- | H | T | — | W | — | D | W | E | D | Y | T | V | — | P | 0.10 | 1.06 | 0.09 | 200 |
| 145 | | | | | | | | | | | | | | | | | | |
| Phage- | S | D | — | W | — | D | W | E | Y | F | Y | D | — | N | 0.10 | 1.06 | 0.08 | 201 |
| 146 | | | | | | | | | | | | | | | | | | |
| Phage- | — | F | — | — | — | D | G | — | — | T | — | H | — | D | 0.09 | 1.05 | 0.08 | 202 |
| 147 | | | | | | | | | | | | | | | | | | |
| Phage- | D | — | — | — | — | I | Y | — | — | — | — | H | — | I | 0.09 | 1.05 | 0.08 | 203 |
| 148 | | | | | | | | | | | | | | | | | | |
| Phage- | A | D | — | — | — | D | G | — | — | — | I | I | — | H | 0.07 | 1.05 | 0.08 | 204 |
| 149 | | | | | | | | | | | | | | | | | | |
| Phage- | F | — | — | — | — | — | L | — | — | — | L | T | — | V | 0.10 | 1.05 | 0.08 | 205 |
| 150 | | | | | | | | | | | | | | | | | | |
| Phage- | I | L | — | — | — | — | V | E | — | D | Y | Y | — | Y | 0.11 | 1.04 | 0.09 | 206 |
| 151 | | | | | | | | | | | | | | | | | | |
| Phage- | H | L | 1 | W | — | D | W | E | — | Y | H | S | — | D | 0.09 | 1.04 | 0.09 | 207 |
| 152 | | | | | | | | | | | | | | | | | | |
| Phage- | I | F | — | W | — | D | W | E | D | Y | N | F | — | T | 0.08 | 1.04 | 0.11 | 208 |
| 153 | | | | | | | | | | | | | | | | | | |
| Phage- | I | V | — | — | — | D | G | — | — | T | L | I | — | H | 0.12 | 1.04 | 0.11 | 209 |
| 154 | | | | | | | | | | | | | | | | | | |
| Phage- | A | D | — | W | — | D | W | E | W | D | Y | T | — | D | 0.12 | 1.03 | 0.11 | 210 |
| 155 | | | | | | | | | | | | | | | | | | |
| Phage- | I | T | — | — | — | — | — | — | — | — | T | T | — | N | 0.20 | 1.02 | 0.21 | 211 |
| 156 | | | | | | | | | | | | | | | | | | |
| Phage- | Y | H | — | W | — | D | W | E | — | Y | T | S | — | D | 0.20 | 1.02 | 0.09 | 212 |
| 157 | | | | | | | | | | | | | | | | | | |
| Phage- | N | — | — | — | — | — | V | E | — | Y | A | L | — | T | 0.11 | 1.01 | 0.10 | 213 |
| 158 | | | | | | | | | | | | | | | | | | |
| Phage- | F | I | — | — | — | — | M | — | — | — | I | H | — | D | 0.15 | 1.00 | 0.19 | 214 |
| 159 | | | | | | | | | | | | | | | | | | |
| Phage- | D | N | — | W | — | D | W | E | — | F | A | V | — | P | 0.14 | 1.00 | 0.10 | 215 |
| 160 | | | | | | | | | | | | | | | | | | |
| Phage- | Y | D | — | — | — | — | L | — | — | T | — | V | — | D | 0.10 | 1.00 | 0.09 | 216 |
| 161 | | | | | | | | | | | | | | | | | | |
| Phage- | Y | D | — | — | — | — | — | — | — | — | I | A | — | Y | 0.08 | 0.99 | 0.08 | 217 |
| 162 | | | | | | | | | | | | | | | | | | |
| Phage- | I | D | — | W | — | D | W | E | Y | T | — | H | — | D | 0.07 | 0.97 | 0.09 | 218 |
| 163 | | | | | | | | | | | | | | | | | | |
| Phage- | D | D | — | — | — | — | L | — | — | — | I | I | — | I | 0.09 | 0.96 | 0.09 | 219 |
| 164 | | | | | | | | | | | | | | | | | | |
| Phage- | — | — | — | — | — | — | Y | — | — | — | S | F | — | F | 0.09 | 0.91 | 0.08 | 220 |
| 165 | | | | | | | | | | | | | | | | | | |
| Phage- | F | N | — | W | — | D | W | E | D | P | Y | F | — | V | 0.09 | 0.86 | 0.07 | 221 |
| 166 | | | | | | | | | | | | | | | | | | |
| Phage- | Y | D | — | — | — | — | Y | — | — | — | S | Y | — | S | 0.08 | 0.82 | 0.07 | 222 |
| 167 | | | | | | | | | | | | | | | | | | |
| Phage- | — | A | — | W | — | — | W | E | Y | T | D | S | — | F | 0.13 | 0.79 | 0.09 | 223 |
| 168 | | | | | | | | | | | | | | | | | | |
| Phage- | T | D | — | — | — | — | — | — | — | — | — | A | — | Y | 0.10 | 0.77 | 0.09 | 224 |
| 169 | | | | | | | | | | | | | | | | | | |
| Phage- | T | D | — | W | — | D | W | E | F | Y | A | D | — | D | 0.07 | 0.75 | 0.08 | 225 |
| 170 | | | | | | | | | | | | | | | | | | |
| Phage- | Y | D | — | — | — | — | L | — | — | — | — | — | — | H | 0.09 | 0.69 | 0.09 | 226 |
| 171 | | | | | | | | | | | | | | | | | | |
| Phage- | S | D | — | — | — | D | G | — | — | — | I | I | — | T | 0.07 | 0.69 | 0.07 | 227 |
| 172 | | | | | | | | | | | | | | | | | | |
| Phage- | Y | I | — | — | — | — | — | — | — | — | I | D | — | D | 0.08 | 0.67 | 0.09 | 228 |
| 173 | | | | | | | | | | | | | | | | | | |
| Phage- | F | F | — | — | — | — | — | I | — | — | T | A | — | V | 0.08 | 0.62 | 0.09 | 229 |
| 174 | | | | | | | | | | | | | | | | | | |
| Phage- | D | — | — | — | — | — | — | — | — | — | T | F | — | D | 0.16 | 0.60 | 0.10 | 230 |
| 175 | | | | | | | | | | | | | | | | | | |
| Phage- | Y | D | — | — | — | — | W | E | W | P | I | D | — | V | 0.10 | 0.59 | 0.10 | 231 |
| 176 | | | | | | | | | | | | | | | | | | |
| Phage- | F | — | — | — | — | — | I | E | L | F | S | F | — | Y | 0.13 | 0.59 | 0.11 | 232 |
| 177 | | | | | | | | | | | | | | | | | | |
| Phage- | Y | — | — | — | — | — | V | — | — | — | I | T | — | P | 0.15 | 0.42 | 0.11 | 233 |
| 178 | | | | | | | | | | | | | | | | | | |
| Phage- | I | L | — | — | — | — | — | — | — | — | I | N | — | N | 0.09 | 0.37 | 0.25 | 234 |
| 179 | | | | | | | | | | | | | | | | | | |
| Phage- | — | V | — | — | — | A | M | G | Q | H | Y | L | — | D | 0.08 | 0.09 | 0.08 | 235 |
| 180 | | | | | | | | | | | | | | | | | | |
| Phage- | — | V | — | I | T | K | M | G | — | H | Y | L | — | S | 0.08 | 0.08 | 0.08 | 236 |
| 181 | | | | | | | | | | | | | | | | | | |
| Phage- | Y | D | — | W | — | D | W | E | Y | V | Y | A | — | Y | 0.08 | 0.98 | 0.08 | 237 |
| 182 | | | | | | | | | | | | | | | | | | |
| Phage- | D | L | — | — | — | — | L | — | — | — | — | N | — | D | 0.09 | 0.98 | 0.08 | 238 |
| 183 | | | | | | | | | | | | | | | | | | |
| Phage- | Y | — | — | — | — | — | — | — | — | — | T | V | — | Y | 0.14 | 0.97 | 0.16 | 239 |
| 184 | | | | | | | | | | | | | | | | | | |
| Phage- | L | D | — | W | — | D | W | E | W | P | Y | S | — | N | 0.08 | 0.96 | 0.09 | 240 |
| 185 | | | | | | | | | | | | | | | | | | |
| Phage- | F | I | — | W | — | D | W | E | D | D | F | F | I | Y | 0.08 | 0.96 | 0.09 | 241 |
| 186 | | | | | | | | | | | | | | | | | | |
| Phage- | D | L | — | — | — | — | V | E | W | Y | F | F | — | N | 0.11 | 0.95 | 0.10 | 242 |
| 187 | | | | | | | | | | | | | | | | | | |
| Phage- | Y | D | — | — | — | — | L | — | — | — | I | V | — | F | 0.07 | 0.94 | 0,08 | 243 |
| 188 | | | | | | | | | | | | | | | | | | |
| Phage- | L | N | — | W | — | V | W | E | D | D | — | F | — | Y | 0.09 | 0.92 | 0.09 | 244 |
| 189 | | | | | | | | | | | | | | | | | | |
| Phage- | F | N | — | W | — | D | W | E | D | P | N | F | — | V | 0.09 | 0.91 | 0.09 | 245 |
| 190 | | | | | | | | | | | | | | | | | | |
| Phage- | — | T | — | W | — | D | W | E | D | D | Y | F | — | P | 0.10 | 0.91 | 0.13 | 246 |
| 191 | | | | | | | | | | | | | | | | | | |
| Phage- | F | L | — | — | — | — | — | — | — | — | S | V | — | Y | 0.10 | 0.91 | 0.08 | 247 |
| 192 | | | | | | | | | | | | | | | | | | |
| Phage- | Y | D | — | — | — | — | L | — | — | — | I | F | — | Y | 0.10 | 0.91 | 0.09 | 248 |
| 193 | | | | | | | | | | | | | | | | | | |
| Phage- | H | L | — | — | — | D | G | — | — | — | F | T | — | F | 0.11 | 0.90 | 0.10 | 249 |
| 194 | | | | | | | | | | | | | | | | | | |
| Phage- | Y | F | — | — | — | — | M | — | — | — | L | Y | — | I | 0.08 | 0.90 | 0.08 | 250 |
| 195 | | | | | | | | | | | | | | | | | | |
| Phage- | Y | — | — | — | — | — | V | E | — | Y | A | N | — | Y | 0.07 | 0.90 | 0.07 | 251 |
| 196 | | | | | | | | | | | | | | | | | | |
| Phage- | N | T | — | — | — | — | — | — | — | — | T | A | — | Y | 0.16 | 0.90 | 0.58 | 252 |
| 197 | | | | | | | | | | | | | | | | | | |
| Phage- | I | D | — | W | — | D | W | E | — | A | F | N | — | Y | 0.09 | 0.90 | 0.08 | 253 |
| 198 | | | | | | | | | | | | | | | | | | |
| Phage- | A | — | — | — | — | — | L | E | — | F | F | L | — | T | 0.09 | 0.89 | 0.08 | 254 |
| 199 | | | | | | | | | | | | | | | | | | |
| Phage | I | — | — | — | — | — | V | E | — | V | H | H | — | Y | 0.08 | 0.89 | 0.08 | 255 |
| 200 | | | | | | | | | | | | | | | | | | |
| Phage- | F | F | — | — | — | — | — | — | — | — | — | A | — | D | 0.10 | 0.89 | 0.11 | 256 |
| 201 | | | | | | | | | | | | | | | | | | |
| Phage- | Y | D | — | — | — | — | L | — | — | T | I | I | — | A | 0.08 | 0.89 | 0.08 | 257 |
| 202 | | | | | | | | | | | | | | | | | | |
| Phage- | I | L | — | — | — | — | W | E | Y | P | L | D | — | S | 0.09 | 0.89 | 0.10 | 258 |
| 203 | | | | | | | | | | | | | | | | | | |
| Phage- | F | I | — | — | — | — | — | — | — | — | T | T | — | N | 0.09 | 0.88 | 0.10 | 259 |
| 204 | | | | | | | | | | | | | | | | | | |
| Phage- | F | I | — | — | — | — | L | — | — | — | — | S | — | D | 0.17 | 0.88 | 0.15 | 260 |
| 205 | | | | | | | | | | | | | | | | | | |
| Phage- | H | L | — | — | — | — | L | — | — | — | — | T | — | F | 0.10 | 0.87 | 0.10 | 261 |
| 206 | | | | | | | | | | | | | | | | | | |
| Phage- | L | I | — | — | — | — | V | E | D | Y | S | L | — | H | 0.09 | 0.87 | 0.09 | 262 |
| 207 | | | | | | | | | | | | | | | | | | |
| Phage- | Y | F | — | — | — | — | M | — | — | — | — | Y | — | D | 0.08 | 0.87 | 0.08 | 263 |
| 208 | | | | | | | | | | | | | | | | | | |
| Phage- | H | — | — | — | — | — | M | — | — | — | I | Y | — | I | 0.13 | 0.87 | 0.09 | 264 |
| 209 | | | | | | | | | | | | | | | | | | |
| Phage- | F | D | — | — | — | — | L | — | — | — | I | N | — | D | 0.08 | 0.87 | 0.09 | 265 |
| 210 | | | | | | | | | | | | | | | | | | |
| Phage- | Y | — | — | — | — | — | V | E | — | Y | I | Y | — | T | 0.07 | 0.87 | 0.08 | 266 |
| 211 | | | | | | | | | | | | | | | | | | |
| Phage- | L | A | — | W | — | V | R | E | — | I | N | A | — | I | 0.08 | 0.85 | 0.07 | 267 |
| 212 | | | | | | | | | | | | | | | | | | |
| Phage- | I | D | — | W | — | D | W | — | ED | I | T | F | — | D | 0.08 | 0.85 | 0.08 | 268 |
| 213 | | | | | | | | | | | | | | | | | | |
| Phage- | I | V | — | — | — | — | L | — | — | — | I | T | — | P | 0.11 | 0.85 | 0.15 | 269 |
| 214 | | | | | | | | | | | | | | | | | | |
| Phage- | F | — | — | — | — | — | — | E | L | P | A | D | — | D | 0.08 | 0.85 | 0.09 | 270 |
| 215 | | | | | | | | | | | | | | | | | | |
| Phage- | F | D | — | — | — | — | — | — | — | — | N | P | — | F | 0.10 | 0.85 | 0.09 | 271 |
| 216 | | | | | | | | | | | | | | | | | | |
| Phage- | D | A | — | W | — | D | W | E | — | Y | S | S | — | D | 0.10 | 0.83 | 0.10 | 272 |
| 217 | | | | | | | | | | | | | | | | | | |
| Phage- | D | H | — | W | — | D | W | E | P | N | Y | F | — | V | 0.08 | 0.83 | 0.09 | 273 |
| 218 | | | | | | | | | | | | | | | | | | |
| Phage- | D | — | — | W | — | D | W | E | I | N | Y | I | — | F | 0.09 | 0.83 | 0.10 | 274 |
| 219 | | | | | | | | | | | | | | | | | | |
| Phage- | I | — | — | W | — | D | W | E | Y | V | Y | A | — | N | 0.10 | 0.82 | 0.09 | 275 |
| 220 | | | | | | | | | | | | | | | | | | |
| Phage- | D | F | — | — | — | — | V | E | — | D | Y | L | — | D | 0.07 | 0.82 | 0.08 | 276 |
| 221 | | | | | | | | | | | | | | | | | | |
| Phage- | H | D | — | — | — | D | G | R | — | D | Y | D | — | A | 0.11 | 0.82 | 0.09 | 277 |
| 222 | | | | | | | | | | | | | | | | | | |
| Phage- | L | A | — | W | — | D | W | E | D | D | Y | F | — | V | 0.08 | 0.82 | 0.09 | 278 |
| 223 | | | | | | | | | | | | | | | | | | |
| Phage- | D | I | — | W | — | D | W | E | D | Y | L | P | — | V | 0.10 | 0.82 | 0.10 | 279 |
| 224 | | | | | | | | | | | | | | | | | | |
| Phage- | I | L | — | — | — | — | I | E | V | Y | A | L | — | P | 0.08 | 0.81 | 0.10 | 280 |
| 225 | | | | | | | | | | | | | | | | | | |
| Phage- | I | F | — | — | — | — | W | E | F | — | — | L | — | N | 0.10 | 0.81 | 0.11 | 281 |
| 226 | | | | | | | | | | | | | | | | | | |
| Phage- | T | — | — | — | — | — | V | E | D | F | S | L | — | V | 0.07 | 0.80 | 0.08 | 282 |
| 227 | | | | | | | | | | | | | | | | | | |
| Phage- | F | I | — | — | — | — | W | E | F | V | D | A | — | F | 0.11 | 0.80 | 0.09 | 283 |
| 228 | | | | | | | | | | | | | | | | | | |
| Phage- | F | A | — | W | — | D | W | E | — | D | S | P | — | D | 0.06 | 0.80 | 0.07 | 284 |
| 229 | | | | | | | | | | | | | | | | | | |
| Phage- | I | L | — | — | — | — | V | E | — | L | I | F | — | P | 0.12 | 0.80 | 0.08 | 285 |
| 230 | | | | | | | | | | | | | | | | | | |
| Phage- | F | — | — | — | — | — | V | E | — | Y | I | Y | — | Y | 0.08 | 0.80 | 0.08 | 286 |
| 231 | | | | | | | | | | | | | | | | | | |
| Phage- | D | S | — | — | — | — | L | — | — | — | I | I | — | D | 0.10 | 0.79 | 0.09 | 287 |
| 232 | | | | | | | | | | | | | | | | | | |
| Phage- | F | L | — | — | — | D | G | — | — | T | S | V | — | D | 0.11 | 0.79 | 0.08 | 288 |
| 233 | | | | | | | | | | | | | | | | | | |
| Phage- | F | N | — | W | — | N | G | E | P | T | Y | F | — | V | 0.11 | 0.79 | 0.08 | 289 |
| 234 | | | | | | | | | | | | | | | | | | |
| Phage- | L | A | — | W | — | V | W | E | Y | P | — | T | — | I | 0.09 | 0.78 | 0.09 | 290 |
| 235 | | | | | | | | | | | | | | | | | | |
| Phage- | D | — | — | — | — | I | V | E | — | D | — | Y | — | Y | 0.09 | 0.78 | 0.09 | 291 |
| 236 | | | | | | | | | | | | | | | | | | |
| Phage- | I | T | — | W | — | D | W | E | — | Y | A | N | — | T | 0.08 | 0.77 | 0.07 | 292 |
| 237 | | | | | | | | | | | | | | | | | | |
| Phage- | F | F | — | — | — | D | G | — | — | T | Y | S | — | I | 0.15 | 0.77 | 0.13 | 293 |
| 238 | | | | | | | | | | | | | | | | | | |
| Phage- | T | D | I | W | — | D | W | E | Y | A | H | S | — | D | 0.09 | 0.76 | 0.09 | 294 |
| 239 | | | | | | | | | | | | | | | | | | |
| Phage- | F | N | — | — | — | D | G | Y | — | D | Y | L | — | D | 0.10 | 0.76 | 0.11 | 295 |
| 240 | | | | | | | | | | | | | | | | | | |
| Phage- | Y | D | — | W | — | D | W | E | V | D | F | H | — | P | 0.11 | 0.76 | 0.08 | 296 |
| 241 | | | | | | | | | | | | | | | | | | |
| Phage- | N | I | — | W | — | D | W | E | D | D | S | F | — | F | 0.08 | 0.76 | 0.08 | 297 |
| 242 | | | | | | | | | | | | | | | | | | |
| Phage- | A | T | I | — | — | — | — | — | — | — | I | — | — | S | 0.13 | 0.75 | 0.09 | 298 |
| 243 | | | | | | | | | | | | | | | | | | |
| Phage- | S | I | — | — | — | — | — | — | — | — | T | F | — | D | 0.10 | 0.74 | 0.09 | 299 |
| 244 | | | | | | | | | | | | | | | | | | |
| Phage- | P | I | — | — | — | — | Y | — | — | I | D | V | — | A | 0.08 | 0.74 | 0.08 | 300 |
| 245 | | | | | | | | | | | | | | | | | | |
| Phage- | Y | I | — | — | — | D | G | — | — | Y | N | S | — | I | 0.11 | 0.74 | 0.11 | 301 |
| 246 | | | | | | | | | | | | | | | | | | |
| Phage- | — | D | — | W | — | D | W | — | E | F | I | A | — | D | 0.11 | 0.74 | 0.10 | 302 |
| 247 | | | | | | | | | | | | | | | | | | |
| Phage- | D | L | — | — | — | — | V | E | — | V | N | L | — | L | 0.12 | 0.74 | 0.10 | 303 |
| 248 | | | | | | | | | | | | | | | | | | |
| Phage- | F | D | — | — | — | — | M | — | — | — | T | T | — | F | 0.09 | 0.74 | 0.09 | 304 |
| 249 | | | | | | | | | | | | | | | | | | |
| Phage- | N | F | — | W | — | D | W | E | P | I | Y | F | — | T | 0.13 | 0.74 | 0.14 | 305 |
| 250 | | | | | | | | | | | | | | | | | | |
| Phage- | — | D | — | — | — | D | G | — | — | — | F | F | — | L | 0.08 | 0.73 | 0.08 | 306 |
| 251 | | | | | | | | | | | | | | | | | | |
| Phage- | — | D | — | — | — | D | G | — | — | H | A | F | — | I | 0.10 | 0.73 | 0.09 | 307 |
| 252 | | | | | | | | | | | | | | | | | | |
| Phage- | N | I | — | — | — | — | M | — | — | — | L | V | — | I | 0.10 | 0.73 | 0.09 | 308 |
| 253 | | | | | | | | | | | | | | | | | | |
| Phage- | I | I | — | — | — | — | — | — | — | — | F | F | — | F | 0.08 | 0.73 | 0.09 | 309 |
| 254 | | | | | | | | | | | | | | | | | | |
| Phage- | N | F | — | — | — | — | Y | — | — | — | I | S | — | I | 0.10 | 0.73 | 0.38 | 310 |
| 255 | | | | | | | | | | | | | | | | | | |
| Phage- | H | L | — | — | — | — | I | E | — | A | D | I | — | N | 0.11 | 0.73 | 0.51 | 311 |
| 256 | | | | | | | | | | | | | | | | | | |
| Phage- | D | — | I | — | — | — | V | E | I | D | Y | L | — | D | 0.08 | 0.72 | 0.09 | 312 |
| 257 | | | | | | | | | | | | | | | | | | |
| Phage- | D | — | I | — | — | — | L | — | — | — | I | N | — | D | 0.11 | 0.72 | 0.10 | 313 |
| 258 | | | | | | | | | | | | | | | | | | |
| Phage- | F | — | — | — | — | — | L | — | — | — | L | F | — | V | 0.09 | 0.72 | 0.08 | 314 |
| 259 | | | | | | | | | | | | | | | | | | |
| Phage- | P | D | — | — | — | — | W | E | F | Y | — | T | — | N | 0.12 | 0.72 | 0.08 | 315 |
| 260 | | | | | | | | | | | | | | | | | | |
| Phage- | F | D | — | — | — | — | — | E | Y | T | Y | A | — | T | 0.09 | 0.72 | 0.08 | 316 |
| 261 | | | | | | | | | | | | | | | | | | |
| Phage- | D | I | — | — | — | — | — | — | — | — | S | I | — | N | 0.12 | 0.72 | 0.11 | 317 |
| 262 | | | | | | | | | | | | | | | | | | |
| Phage- | D | F | — | — | — | — | V | E | — | Y | I | F | — | F | 0.08 | 0.72 | 0.07 | 318 |
| 263 | | | | | | | | | | | | | | | | | | |
| Phage- | P | V | — | W | — | D | W | E | Y | V | S | S | — | D | 0.08 | 0.71 | 0.08 | 319 |
| 264 | | | | | | | | | | | | | | | | | | |
| Phage- | Y | I | — | — | — | — | R | — | — | — | N | L | — | L | 0.09 | 0.71 | 0.09 | 320 |
| 265 | | | | | | | | | | | | | | | | | | |
| Phage- | Y | D | — | — | — | — | L | — | — | — | I | V | — | D | 0.11 | 0.71 | 0.11 | 321 |
| 266 | | | | | | | | | | | | | | | | | | |
| Phage- | H | D | — | W | — | D | W | E | D | F | Y | F | — | V | 0.09 | 0.71 | 0.08 | 322 |
| 267 | | | | | | | | | | | | | | | | | | |
| Phage- | H | — | — | — | — | — | Y | — | — | — | I | D | — | Y | 0.12 | 0.71 | 0.10 | 323 |
| 268 | | | | | | | | | | | | | | | | | | |
| Phage- | L | F | — | — | — | — | M | P | — | D | — | F | — | N | 0.08 | 0.71 | 0.08 | 324 |
| 269 | | | | | | | | | | | | | | | | | | |
| Phage- | H | D | — | — | — | — | L | E | F | H | Y | A | — | Y | 0.10 | 0.71 | 0.12 | 325 |
| 270 | | | | | | | | | | | | | | | | | | |
| Phage- | D | F | — | — | — | — | L | — | — | — | I | N | — | F | 0.08 | 0.70 | 0.08 | 326 |
| 271 | | | | | | | | | | | | | | | | | | |
| Phage- | Y | F | — | — | — | — | L | — | — | — | I | A | — | N | 0.10 | 0.70 | 0.10 | 327 |
| 272 | | | | | | | | | | | | | | | | | | |
| Phage- | T | D | — | W | — | D | W | E | D | D | I | T | — | D | 0.10 | 0.70 | 0.08 | 328 |
| 273 | | | | | | | | | | | | | | | | | | |
| Phage- | Y | D | — | — | — | — | L | — | — | — | I | Y | — | F | 0.09 | 0.70 | 0.08 | 329 |
| 274 | | | | | | | | | | | | | | | | | | |
| Phage- | Y | — | — | W | — | D | W | W | — | Y | — | T | — | D | 0.10 | 0.70 | 0.11 | 330 |
| 275 | | | | | | | | | | | | | | | | | | |
| Phage- | P | I | — | — | — | — | L | E | — | — | Y | L | — | N | 0.13 | 0.69 | 0.52 | 331 |
| 276 | | | | | | | | | | | | | | | | | | |
| Phage- | F | D | — | — | — | — | — | — | — | — | I | V | — | Y | 0.12 | 0.69 | 0.11 | 332 |
| 277 | | | | | | | | | | | | | | | | | | |
| Phage- | — | L | — | — | — | D | G | — | — | F | F | D | — | P | 0.09 | 0.68 | 0.07 | 333 |
| 278 | | | | | | | | | | | | | | | | | | |
| Phage- | A | — | — | — | — | — | Y | — | — | — | L | T | — | V | 0.07 | 0.68 | 0.07 | 334 |
| 279 | | | | | | | | | | | | | | | | | | |
| Phage- | D | F | — | — | — | — | L | — | — | — | I | I | — | A | 0.14 | 0.68 | 0.14 | 335 |
| 280 | | | | | | | | | | | | | | | | | | |
| Phage- | Y | D | — | — | — | — | — | — | — | — | L | D | — | N | 0.08 | 0.68 | 0.08 | 336 |
| 281 | | | | | | | | | | | | | | | | | | |
| Phage- | A | I | — | — | — | — | — | — | — | — | — | A | — | D | 0.14 | 0.67 | 0.08 | 337 |
| 282 | | | | | | | | | | | | | | | | | | |
| Phage- | L | L | — | — | — | D | G | V | — | D | F | F | — | D | 0.10 | 0.67 | 0.10 | 338 |
| 283 | | | | | | | | | | | | | | | | | | |
| Phage- | N | F | — | — | — | — | — | L | P | D | I | F | — | F | 0.12 | 0.66 | 0.13 | 339 |
| 284 | | | | | | | | | | | | | | | | | | |
| Phage- | F | — | — | — | — | — | V | E | — | V | S | L | — | N | 0.08 | 0.66 | 0.08 | 340 |
| 285 | | | | | | | | | | | | | | | | | | |
| Phage- | Y | D | — | — | — | D | G | Y | — | A | F | Y | — | H | 0.12 | 0.66 | 0.10 | 341 |
| 286 | | | | | | | | | | | | | | | | | | |
| Phage- | N | F | — | — | — | — | I | E | F | D | Y | L | — | D | 0.08 | 0.65 | 0.08 | 342 |
| 287 | | | | | | | | | | | | | | | | | | |
| Phage- | D | — | — | — | — | D | G | V | — | D | F | I | — | N | 0.06 | 0.65 | 0.08 | 343 |
| 288 | | | | | | | | | | | | | | | | | | |
| Phage- | T | D | — | W | — | D | W | E | Y | I | Y | S | — | S | 0.08 | 0.65 | 0.07 | 344 |
| 289 | | | | | | | | | | | | | | | | | | |
| Phage- | F | — | — | — | — | — | — | E | — | I | T | N | — | I | 0.14 | 0.65 | 0.11 | 345 |
| 290 | | | | | | | | | | | | | | | | | | |
| Phage- | F | D | — | W | — | D | W | E | — | — | F | F | — | H | 0.07 | 0.64 | 0.08 | 346 |
| 291 | | | | | | | | | | | | | | | | | | |
| Phage- | D | F | — | — | — | D | G | — | — | — | — | F | — | P | 0.08 | 0.63 | 0.08 | 347 |
| 292 | | | | | | | | | | | | | | | | | | |
| Phage- | H | N | — | — | — | — | L | — | — | — | L | V | — | D | 0.13 | 0.63 | 0.09 | 348 |
| 293 | | | | | | | | | | | | | | | | | | |
| Phage- | I | — | — | — | — | D | G | A | — | D | Y | T | — | D | 0.07 | 0.63 | 0.07 | 349 |
| 294 | | | | | | | | | | | | | | | | | | |
| Phage- | F | D | — | — | — | — | — | E | F | P | — | — | — | F | 0.08 | 0.62 | 0.08 | 350 |
| 295 | | | | | | | | | | | | | | | | | | |
| Phage- | Y | N | — | — | — | — | L | — | — | — | — | T | — | D | 0.09 | 0.62 | 0.08 | 351 |
| 296 | | | | | | | | | | | | | | | | | | |
| Phage- | F | D | — | — | — | — | L | — | — | — | I | H | — | A | 0.07 | 0.62 | 0.08 | 352 |
| 297 | | | | | | | | | | | | | | | | | | |
| Phage- | D | I | — | — | — | — | V | E | — | Y | F | L | — | F | 0.15 | 0.61 | 0.10 | 353 |
| 298 | | | | | | | | | | | | | | | | | | |
| Phage- | F | D | — | — | — | — | V | — | — | — | L | T | — | F | 0.10 | 0.61 | 0.09 | 354 |
| 299 | | | | | | | | | | | | | | | | | | |
| Phage- | F | D | — | — | — | — | — | E | — | F | H | L | — | — | 0.08 | 0.61 | 0.08 | 355 |
| 300 | | | | | | | | | | | | | | | | | | |
| Phage- | A | — | — | — | — | — | L | — | — | — | I | I | — | D | 0.12 | 0.61 | 0.10 | 356 |
| 301 | | | | | | | | | | | | | | | | | | |
| Phage- | Y | N | — | — | — | — | L | — | — | — | I | T | — | N | 0.09 | 0.61 | 0.10 | 357 |
| 302 | | | | | | | | | | | | | | | | | | |
| Phage- | F | D | — | W | — | D | W | E | — | P | — | D | — | L | 0.08 | 0.61 | 0.07 | 358 |
| 303 | | | | | | | | | | | | | | | | | | |
| Phage- | D | V | — | — | — | — | L | I | — | — | — | L | — | P | 0.08 | 0.60 | 0.08 | 359 |
| 304 | | | | | | | | | | | | | | | | | | |
| Phage- | N | — | — | — | — | — | L | — | — | — | L | P | — | P | 0.09 | 0.60 | 0.08 | 360 |
| 305 | | | | | | | | | | | | | | | | | | |
| Phage- | Y | — | — | W | — | D | W | E | Y | D | I | F | — | S | 0.08 | 0.60 | 0.10 | 361 |
| 306 | | | | | | | | | | | | | | | | | | |
| Phage- | D | D | — | — | — | — | — | — | — | — | T | Y | — | N | 0.08 | 0.60 | 0.09 | 362 |
| 307 | | | | | | | | | | | | | | | | | | |
| Phage- | F | — | — | — | — | — | — | E | — | V | F | H | — | Y | 0.09 | 0.60 | 0.09 | 363 |
| 308 | | | | | | | | | | | | | | | | | | |
| Phage- | T | D | — | W | — | D | W | E | — | Y | F | L | — | D | 0.07 | 0.60 | 0.09 | 364 |
| 309 | | | | | | | | | | | | | | | | | | |
| Phage- | — | — | — | — | — | — | W | E | — | — | Y | L | — | P | 0.09 | 0.60 | 0.09 | 365 |
| 310 | | | | | | | | | | | | | | | | | | |
| Phage- | D | D | — | — | — | N | G | Y | A | T | F | I | — | Y | 0.06 | 0.59 | 0.08 | 366 |
| 311 | | | | | | | | | | | | | | | | | | |
| Phage- | F | L | — | — | — | — | I | E | D | D | T | H | — | Y | 0.16 | 0.59 | 0.38 | 367 |
| 312 | | | | | | | | | | | | | | | | | | |
| Phage- | F | A | — | W | — | D | W | E | — | T | T | P | — | H | 0.08 | 0.59 | 0.08 | 368 |
| 313 | | | | | | | | | | | | | | | | | | |
| Phage- | — | L | — | — | — | — | — | — | — | — | Y | N | — | Y | 0.10 | 0.58 | 0.08 | 369 |
| 314 | | | | | | | | | | | | | | | | | | |
| Phage- | Y | D | — | — | — | — | — | — | — | — | I | S | — | I | 0.09 | 0.58 | 0.09 | 370 |
| 315 | | | | | | | | | | | | | | | | | | |
| Phage- | Y | D | — | — | — | — | — | — | — | — | — | N | — | Y | 0.12 | 0.57 | 0.10 | 371 |
| 316 | | | | | | | | | | | | | | | | | | |
| Phage- | A | I | — | W | — | D | W | E | — | F | — | D | — | Y | 0.10 | 0.57 | 0.09 | 372 |
| 317 | | | | | | | | | | | | | | | | | | |
| Phage- | L | T | — | W | — | V | R | E | — | I | F | A | — | D | 0.07 | 0.57 | 0.08 | 373 |
| 318 | | | | | | | | | | | | | | | | | | |
| Phage- | — | L | — | — | — | — | — | — | I | — | Y | Y | — | N | 0.09 | 0.57 | 0.09 | 374 |
| 319 | | | | | | | | | | | | | | | | | | |
| Phage- | N | V | — | — | — | — | Y | — | — | — | A | P | — | N | 0.07 | 0.56 | 0.08 | 375 |
| 320 | | | | | | | | | | | | | | | | | | |
| Phage- | H | D | — | — | — | — | — | — | — | — | I | S | — | V | 0.11 | 0.55 | 0.09 | 376 |
| 321 | | | | | | | | | | | | | | | | | | |
| Phage- | F | D | — | — | — | — | L | — | — | H | — | D | — | N | 0.12 | 0.55 | 0.41 | 377 |
| 322 | | | | | | | | | | | | | | | | | | |
| Phage- | Y | F | — | — | — | — | V | E | 1 | H | F | Y | — | Y | 0.09 | 0.55 | 0.08 | 378 |
| 323 | | | | | | | | | | | | | | | | | | |
| Phage- | D | — | — | — | — | — | L | — | — | — | I | I | — | H | 0.09 | 0.54 | 0.09 | 379 |
| 324 | | | | | | | | | | | | | | | | | | |
| Phage- | D | D | — | — | — | — | V | P | — | D | I | T | — | Y | 0.11 | 0.54 | 0.08 | 380 |
| 325 | | | | | | | | | | | | | | | | | | |
| Phage- | D | N | — | — | — | — | L | — | — | — | — | V | — | D | 0.10 | 0.54 | 0.08 | 381 |
| 326 | | | | | | | | | | | | | | | | | | |
| Phage- | — | H | — | W | — | D | W | E | P | N | Y | V | — | D | 0.10 | 0.54 | 0.08 | 382 |
| 327 | | | | | | | | | | | | | | | | | | |
| Phage- | D | — | — | — | — | — | L | — | — | — | L | F | — | L | 0.09 | 0.53 | 0.09 | 383 |
| 328 | | | | | | | | | | | | | | | | | | |
| Phage- | D | D | — | — | — | — | L | — | — | — | — | V | — | A | 0.08 | 0.53 | 0.11 | 384 |
| 329 | | | | | | | | | | | | | | | | | | |
| Phage- | A | A | — | — | — | — | L | — | — | — | T | V | — | D | 0.13 | 0.53 | 0.09 | 385 |
| 330 | | | | | | | | | | | | | | | | | | |
| Phage- | D | F | — | — | — | — | — | E | — | — | N | N | — | F | 0.14 | 0.52 | 0.48 | 386 |
| 331 | | | | | | | | | | | | | | | | | | |
| Phage- | Y | — | — | — | — | — | — | — | — | — | N | A | — | Y | 0.08 | 0.52 | 0.07 | 387 |
| 332 | | | | | | | | | | | | | | | | | | |
| Phage- | I | L | — | — | — | — | — | — | — | — | N | S | — | Y | 0.10 | 0.52 | 0.11 | 388 |
| 333 | | | | | | | | | | | | | | | | | | |
| Phage- | Y | D | — | — | — | — | — | — | — | — | I | D | — | D | 0.09 | 0.52 | 0.09 | 389 |
| 334 | | | | | | | | | | | | | | | | | | |
| Phage- | D | S | — | — | — | — | — | E | F | Y | Y | V | — | F | 0.11 | 0.52 | 0.14 | 390 |
| 335 | | | | | | | | | | | | | | | | | | |
| Phage- | Y | I | — | — | — | — | L | — | — | — | L | I | — | H | 0.10 | 0.52 | 0.08 | 391 |
| 336 | | | | | | | | | | | | | | | | | | |
| Phage- | F | D | — | — | — | — | V | E | — | D | Y | F | — | Y | 0.11 | 0.52 | 0.10 | 392 |
| 337 | | | | | | | | | | | | | | | | | | |
| Phage- | F | D | — | — | — | I | Y | — | — | — | L | Y | — | F | 0.08 | 0.52 | 0.07 | 393 |
| 338 | | | | | | | | | | | | | | | | | | |
| Phage- | — | L | I | — | — | D | G | — | — | Y | S | F | — | H | 0.10 | 0.51 | 0.10 | 394 |
| 339 | | | | | | | | | | | | | | | | | | |
| Phage- | I | p | — | — | — | — | M | — | — | — | — | V | — | N | 0.12 | 0.51 | 0.09 | 395 |
| 340 | | | | | | | | | | | | | | | | | | |
| Phage- | I | T | — | — | — | D | G | Y | — | D | F | T | — | D | 0.12 | 0.51 | 0.09 | 396 |
| 341 | | | | | | | | | | | | | | | | | | |
| Phage- | I | F | — | — | — | — | L | — | — | — | I | I | — | Y | 0.11 | 0.51 | 0.08 | 397 |
| 342 | | | | | | | | | | | | | | | | | | |
| Phage- | — | D | — | — | — | — | I | — | — | — | Y | D | — | Y | 0.18 | 0.51 | 0.18 | 398 |
| 343 | | | | | | | | | | | | | | | | | | |
| Phage- | L | S | — | — | — | — | M | — | — | — | L | Y | — | D | 0.12 | 0.51 | 0.08 | 399 |
| 344 | | | | | | | | | | | | | | | | | | |
| Phage- | Y | D | — | W | — | D | W | E | Y | N | T | D | — | T | 0.08 | 0.51 | 0.09 | 400 |
| 345 | | | | | | | | | | | | | | | | | | |
| Phage- | N | H | — | — | — | D | G | — | — | T | T | V | — | F | 0.09 | 0.51 | 0.08 | 401 |
| 346 | | | | | | | | | | | | | | | | | | |
| Phage- | N | F | — | — | — | — | L | — | — | — | I | P | — | H | 0.11 | 0.50 | 0.12 | 402 |
| 347 | | | | | | | | | | | | | | | | | | |
| Phage- | F | H | — | — | — | — | I | E | — | Y | A | L | — | D | 0.08 | 0.50 | 0.09 | 403 |
| 348 | | | | | | | | | | | | | | | | | | |
| Phage- | — | — | — | — | — | — | V | E | D | Y | N | L | — | Y | 0.07 | 0.50 | 0.08 | 404 |
| 349 | | | | | | | | | | | | | | | | | | |
| Phage- | — | — | — | — | — | D | G | — | — | L | A | N | — | Y | 0.09 | 0.50 | 0.08 | 405 |
| 350 | | | | | | | | | | | | | | | | | | |
| Phage- | L | I | — | — | — | V | I | A | — | D | L | P | — | N | 0.17 | 0.50 | 0.26 | 406 |
| 351 | | | | | | | | | | | | | | | | | | |
| Phage- | D | I | — | — | — | — | I | P | — | D | — | S | I | D | 0.10 | 0.50 | 0.08 | 407 |
| 352 | | | | | | | | | | | | | | | | | | |
| Phage- | I | — | — | — | — | — | W | E | — | A | D | Y | — | D | 0.11 | 0.50 | 0.45 | 408 |
| 353 | | | | | | | | | | | | | | | | | | |
| Phage- | I | D | — | W | — | D | W | E | D | D | S | I | — | Y | 0.10 | 0.50 | 0.10 | 409 |
| 354 | | | | | | | | | | | | | | | | | | |
| Phage- | — | L | — | — | — | — | V | E | D | F | T | L | — | D | 0.09 | 0.50 | 0.11 | 410 |
| 355 | | | | | | | | | | | | | | | | | | |
| Phage- | L | — | — | — | — | V | I | E | — | — | Y | Y | — | Y | 0.09 | 0.49 | 0.09 | 411 |
| 356 | | | | | | | | | | | | | | | | | | |
| Phage- | F | F | — | — | — | — | — | E | V | H | S | D | — | N | 0.14 | 0.49 | 0.38 | 412 |
| 357 | | | | | | | | | | | | | | | | | | |
| Phage- | N | D | — | — | — | — | V | E | L | V | S | D | — | N | 0.10 | 0.49 | 0.08 | 413 |
| 358 | | | | | | | | | | | | | | | | | | |
| Phage- | D | L | — | — | — | — | L | — | — | — | T | V | — | D | 0.09 | 0.49 | 0.08 | 414 |
| 359 | | | | | | | | | | | | | | | | | | |
| Phage- | I | P | — | — | — | — | V | E | D | Y | N | L | — | N | 0.08 | 0.49 | 0.08 | 415 |
| 360 | | | | | | | | | | | | | | | | | | |
| Phage- | Y | — | I | — | — | — | L | E | W | P | — | V | — | N | 0.10 | 0.49 | 0.10 | 416 |
| 361 | | | | | | | | | | | | | | | | | | |
| Phage- | Y | D | — | — | — | — | L | — | — | — | — | I | — | N | 0.08 | 0.49 | 0.10 | 417 |
| 362 | | | | | | | | | | | | | | | | | | |
| Phage- | — | — | — | — | — | D | G | — | — | — | F | D | — | A | 0.08 | 0.49 | 0.08 | 418 |
| 363 | | | | | | | | | | | | | | | | | | |
| Phage- | N | D | — | — | — | — | W | E | D | T | Y | F | — | L | 0.08 | 0.49 | 0.10 | 419 |
| 364 | | | | | | | | | | | | | | | | | | |
| Phage- | P | — | — | — | — | — | M | E | — | L | S | N | — | S | 0.13 | 0.48 | 0.16 | 420 |
| 365 | | | | | | | | | | | | | | | | | | |
| Phage- | D | D | — | — | — | — | — | E | V | I | S | D | I | Y | 0.15 | 0.48 | 0.10 | 421 |
| 366 | | | | | | | | | | | | | | | | | | |
| Phage- | D | L | — | — | — | — | — | P | — | D | — | P | — | D | 0.08 | 0.48 | 0.08 | 422 |
| 367 | | | | | | | | | | | | | | | | | | |
| Phage- | I | — | — | — | — | — | — | — | — | — | F | V | — | Y | 0.11 | 0.48 | 0.10 | 423 |
| 368 | | | | | | | | | | | | | | | | | | |
| Phage- | A | — | — | — | — | — | Y | E | V | F | A | D | — | N | 0.10 | 0.48 | 0.11 | 424 |
| 369 | | | | | | | | | | | | | | | | | | |
| Phage- | I | D | — | — | — | — | Y | — | — | — | — | D | — | L | 0.09 | 0.48 | 0.09 | 425 |
| 370 | | | | | | | | | | | | | | | | | | |
| Phage- | H | I | — | W | — | D | W | E | — | F | H | D | — | N | 0.07 | 0.48 | 0.08 | 426 |
| 371 | | | | | | | | | | | | | | | | | | |
| Phage- | Y | D | — | — | — | — | L | — | — | T | I | T | — | L | 0.08 | 0.48 | 0.08 | 427 |
| 372 | | | | | | | | | | | | | | | | | | |
| Phage- | Y | L | — | — | — | — | L | — | — | T | I | L | — | N | 0.09 | 0.48 | 0.08 | 428 |
| 373 | | | | | | | | | | | | | | | | | | |
| Phage- | F | F | — | — | — | — | — | E | — | A | F | L | — | F | 0.10 | 0.48 | 0.14 | 429 |
| 374 | | | | | | | | | | | | | | | | | | |
| Phage- | I | L | — | — | — | — | L | — | — | — | F | T | — | A | 0.07 | 0.47 | 0.08 | 430 |
| 375 | | | | | | | | | | | | | | | | | | |
| Phage- | F | H | — | — | — | — | V | E | L | Y | T | D | — | N | 0.09 | 0.47 | 0.08 | 431 |
| 376 | | | | | | | | | | | | | | | | | | |
| Phage- | N | L | — | — | — | — | V | E | — | Y | N | F | — | Y | 0.08 | 0.47 | 0.08 | 432 |
| 377 | | | | | | | | | | | | | | | | | | |
| Phage- | F | D | — | — | — | — | V | E | — | T | Y | Y | — | F | 0.21 | 0.47 | 0.09 | 433 |
| 378 | | | | | | | | | | | | | | | | | | |
| Phage- | — | F | — | — | — | — | — | E | — | D | H | Y | — | Y | 0.12 | 0.47 | 0.37 | 434 |
| 379 | | | | | | | | | | | | | | | | | | |
| Phage- | A | — | — | — | — | — | W | E | V | V | A | D | — | N | 0.11 | 0.47 | 0.11 | 435 |
| 380 | | | | | | | | | | | | | | | | | | |
| Phage- | F | I | — | W | — | D | W | E | — | D | N | Y | — | N | 0.12 | 0.47 | 0.25 | 436 |
| 381 | | | | | | | | | | | | | | | | | | |
| Phage- | I | — | — | — | — | — | — | — | — | — | F | I | — | D | 0.08 | 0.47 | 0.10 | 437 |
| 382 | | | | | | | | | | | | | | | | | | |
| Phage- | N | L | — | — | — | — | V | E | D | V | Y | D | — | H | 0.12 | 0.47 | 0.43 | 438 |
| 383 | | | | | | | | | | | | | | | | | | |
| Phage- | H | — | — | — | — | — | V | E | — | Y | H | N | — | N | 0.09 | 0.47 | 0.09 | 439 |
| 384 | | | | | | | | | | | | | | | | | | |
| Phage- | D | I | — | — | — | — | Y | — | — | — | Y | S | — | T | 0.09 | 0.47 | 0.09 | 440 |
| 385 | | | | | | | | | | | | | | | | | | |
| Phage- | D | — | — | — | — | — | L | — | — | T | L | I | — | A | 0.13 | 0.46 | 0.10 | 441 |
| 386 | | | | | | | | | | | | | | | | | | |
| Phage- | T | A | — | — | — | — | M | P | — | D | — | D | — | Y | 0.12 | 0.46 | 0.09 | 442 |
| 387 | | | | | | | | | | | | | | | | | | |
| Phage- | I | D | — | — | — | — | L | — | — | — | I | F | — | D | 0.10 | 0.46 | 0.10 | 443 |
| 388 | | | | | | | | | | | | | | | | | | |
| Phage- | Y | F | — | — | — | D | V | E | — | D | F | A | — | D | 0.11 | 0.46 | 0.11 | 444 |
| 389 | | | | | | | | | | | | | | | | | | |
| Phage- | Y | N | — | — | — | — | W | E | Y | A | I | L | — | D | 0.12 | 0.46 | 0.34 | 445 |
| 390 | | | | | | | | | | | | | | | | | | |
| Phage- | I | — | — | — | — | — | V | E | D | Y | I | V | — | N | 0.14 | 0.46 | 0.22 | 446 |
| 391 | | | | | | | | | | | | | | | | | | |
| Phage- | Y | D | — | — | — | — | I | — | — | — | T | P | — | A | 0.07 | 0.46 | 0.07 | 447 |
| 392 | | | | | | | | | | | | | | | | | | |
| Phage- | D | T | I | W | — | D | W | E | H | I | Y | A | — | D | 0.09 | 0.46 | 0.09 | 448 |
| 393 | | | | | | | | | | | | | | | | | | |
| Phage- | D | I | — | — | — | — | M | — | — | — | — | T | — | N | 0.13 | 0.45 | 0.12 | 449 |
| 394 | | | | | | | | | | | | | | | | | | |
| Phage- | Y | D | — | W | — | D | W | E | R | Y | — | P | — | I | 0.10 | 0.45 | 0.09 | 450 |
| 395 | | | | | | | | | | | | | | | | | | |
| Phage- | H | L | — | — | — | — | L | — | — | — | — | A | — | S | 0.13 | 0.45 | 0.11 | 451 |
| 396 | | | | | | | | | | | | | | | | | | |
| Phage- | Y | D | — | — | — | D | G | — | — | T | T | I | — | A | 0.09 | 0.45 | 0.09 | 452 |
| 397 | | | | | | | | | | | | | | | | | | |
| Phage- | Y | — | — | — | — | — | Y | E | D | V | L | D | — | F | 0.07 | 0.45 | 0.08 | 453 |
| 398 | | | | | | | | | | | | | | | | | | |
| Phage- | D | F | — | — | — | — | M | — | — | T | I | S | — | D | 0.14 | 0.45 | 0.10 | 454 |
| 399 | | | | | | | | | | | | | | | | | | |
| Phage- | I | L | — | — | — | — | L | — | — | — | L | V | — | D | 0.08 | 0.44 | 0.07 | 455 |
| 400 | | | | | | | | | | | | | | | | | | |
| Phage- | L | I | — | — | — | — | W | E | V | I | T | N | — | D | 0.12 | 0.44 | 0.40 | 456 |
| 401 | | | | | | | | | | | | | | | | | | |
| Phage- | Y | D | — | — | — | — | — | I | — | Y | F | — | — | P | 0.09 | 0.44 | 0.09 | 457 |
| 402 | | | | | | | | | | | | | | | | | | |
| Phage- | A | L | — | — | — | — | V | E | V | Y | D | V | — | V | 0.08 | 0.44 | 0.08 | 458 |
| 403 | | | | | | | | | | | | | | | | | | |
| Phage- | Y | H | — | W | — | D | W | E | D | V | N | F | — | Y | 0.10 | 0.44 | 0.09 | 459 |
| 404 | | | | | | | | | | | | | | | | | | |
| Phage- | F | L | — | — | — | M | G | G | L | T | F | Y | — | Y | 0.09 | 0.44 | 0.08 | 460 |
| 405 | | | | | | | | | | | | | | | | | | |
| Phage- | I | I | — | — | — | — | — | — | — | Y | — | — | — | F | 0.09 | 0.43 | 0.19 | 461 |
| 406 | | | | | | | | | | | | | | | | | | |
| Phage- | F | F | — | — | — | — | M | — | — | — | — | H | — | F | 0.11 | 0.43 | 0.09 | 462 |
| 407 | | | | | | | | | | | | | | | | | | |
| Phage- | A | F | — | — | — | — | — | — | — | — | L | F | — | A | 0.09 | 0.43 | 0.09 | 463 |
| 408 | | | | | | | | | | | | | | | | | | |
| Phage- | N | — | — | — | — | D | G | — | — | T | N | I | — | D | 0.08 | 0.43 | 0.08 | 464 |
| 409 | | | | | | | | | | | | | | | | | | |
| Phage- | Y | L | — | — | — | — | W | E | W | V | H | N | — | L | 0.13 | 0.43 | 0.09 | 465 |
| 410 | | | | | | | | | | | | | | | | | | |
| Phage- | A | T | — | — | — | D | G | — | — | — | H | I | — | A | 0.08 | 0.43 | 0.09 | 466 |
| 411 | | | | | | | | | | | | | | | | | | |
| Phage- | — | — | — | — | — | — | V | E | V | L | D | Y | — | D | 0.07 | 0.42 | 0.08 | 467 |
| 412 | | | | | | | | | | | | | | | | | | |
| Phage- | I | H | — | — | — | — | W | E | F | Y | T | D | — | D | 0.08 | 0.42 | 0.08 | 468 |
| 413 | | | | | | | | | | | | | | | | | | |
| Phage- | D | D | — | — | — | — | L | — | — | T | — | A | — | D | 0.13 | 0.42 | 0.32 | 469 |
| 414 | | | | | | | | | | | | | | | | | | |
| Phage- | Y | L | — | — | — | — | — | — | — | — | I | D | — | N | 0.11 | 0.42 | 0.17 | 470 |
| 415 | | | | | | | | | | | | | | | | | | |
| Phage- | L | L | — | — | — | — | V | E | D | V | F | A | — | Y | 0.09 | 0.42 | 0.09 | 471 |
| 416 | | | | | | | | | | | | | | | | | | |
| Phage- | Y | D | — | — | — | — | L | — | — | — | L | T | — | D | 0.08 | 0.42 | 0.08 | 472 |
| 417 | | | | | | | | | | | | | | | | | | |
| Phage- | F | D | — | — | — | — | L | — | — | T | — | N | — | Y | 0.09 | 0.41 | 0.09 | 473 |
| 418 | | | | | | | | | | | | | | | | | | |
| Phage- | F | A | — | W | — | D | W | E | — | I | N | D | — | H | 0.08 | 0.41 | 0.09 | 474 |
| 419 | | | | | | | | | | | | | | | | | | |
| Phage- | Y | — | — | — | — | — | Y | F | — | D | I | Y | — | N | 0.09 | 0.41 | 0.09 | 475 |
| 420 | | | | | | | | | | | | | | | | | | |
| Phage- | N | V | — | — | — | — | V | E | D | Y | T | F | — | Y | 0.09 | 0.40 | 0.08 | 476 |
| 421 | | | | | | | | | | | | | | | | | | |
| Phage- | A | — | — | — | — | — | L | E | — | Y | D | F | — | T | 0.12 | 0.40 | 0.08 | 477 |
| 422 | | | | | | | | | | | | | | | | | | |
| Phage- | F | D | — | — | — | — | I | — | — | — | T | I | — | T | 0.08 | 0.40 | 0.09 | 478 |
| 423 | | | | | | | | | | | | | | | | | | |
| Phage- | N | L | — | — | — | — | L | — | — | T | L | V | — | A | 0.10 | 0.40 | 0.09 | 479 |
| 424 | | | | | | | | | | | | | | | | | | |
| Phage- | Y | S | — | W | — | D | W | E | — | Y | L | A | — | N | 0.08 | 0.40 | 0.08 | 480 |
| 425 | | | | | | | | | | | | | | | | | | |
| Phage- | G | I | — | — | — | — | V | E | D | Y | N | Y | — | D | 0.09 | 0.40 | 0.10 | 481 |
| 426 | | | | | | | | | | | | | | | | | | |
| Phage- | F | F | — | — | — | — | L | — | — | — | — | N | — | H | 0.07 | 0.40 | 0.07 | 482 |
| 427 | | | | | | | | | | | | | | | | | | |
| Phage- | Y | — | — | — | — | — | Y | E | — | D | F | Y | — | F | 0.11 | 0.40 | 0.09 | 483 |
| 428 | | | | | | | | | | | | | | | | | | |
| Phage- | L | — | — | — | — | — | Y | — | — | — | T | D | — | Y | 0.11 | 0.40 | 0.10 | 484 |
| 429 | | | | | | | | | | | | | | | | | | |
| Phage- | D | — | — | — | — | — | V | E | — | D | F | L | — | Y | 0.10 | 0.40 | 0.08 | 485 |
| 430 | | | | | | | | | | | | | | | | | | |
| Phage- | T | L | — | — | — | — | V | E | L | Y | — | F | — | D | 0.08 | 0.40 | 0.09 | 486 |
| 431 | | | | | | | | | | | | | | | | | | |
| Phage- | F | — | — | — | — | — | — | E | Q | I | A | D | — | Y | 0.11 | 0.40 | 0.09 | 487 |
| 432 | | | | | | | | | | | | | | | | | | |
| Phage- | D | D | — | — | — | — | V | E | — | Y | H | L | — | D | 0.11 | 0.40 | 0.18 | 488 |
| 433 | | | | | | | | | | | | | | | | | | |
| Phage- | D | — | — | — | — | — | L | E | D | V | T | L | — | H | 0.13 | 0.39 | 0.09 | 489 |
| 434 | | | | | | | | | | | | | | | | | | |
| Phage- | — | L | — | — | — | — | V | E | D | V | N | L | — | Y | 0.09 | 0.39 | 0.08 | 490 |
| 435 | | | | | | | | | | | | | | | | | | |
| Phage- | D | — | — | — | — | — | L | — | — | T | I | D | — | Y | 0.09 | 0.39 | 0.09 | 491 |
| 436 | | | | | | | | | | | | | | | | | | |
| Phage- | T | — | — | — | — | — | V | E | — | D | I | N | — | Y | 0.08 | 0.39 | 0.08 | 492 |
| 437 | | | | | | | | | | | | | | | | | | |
| Phage- | I | V | — | W | — | D | W | E | — | Y | P | N | — | D | 0.08 | 0.39 | 0.08 | 493 |
| 438 | | | | | | | | | | | | | | | | | | |
| Phage- | S | D | I | — | — | — | L | — | — | — | I | I | — | T | 0.11 | 0.39 | 0.10 | 494 |
| 439 | | | | | | | | | | | | | | | | | | |
| Phage- | Y | D | — | — | — | — | L | P | — | D | Y | D | — | N | 0.15 | 0.39 | 0.10 | 495 |
| 440 | | | | | | | | | | | | | | | | | | |
| Phage- | N | L | — | W | — | D | W | E | — | Y | Y | A | — | D | 0.12 | 0.39 | 0.17 | 496 |
| 441 | | | | | | | | | | | | | | | | | | |
| Phage- | D | D | — | — | — | — | L | — | — | — | L | P | — | H | 0.10 | 0.39 | 0.08 | 497 |
| 442 | | | | | | | | | | | | | | | | | | |
| Phage- | S | L | — | — | — | D | G | Q | — | D | Y | T | — | F | 0.08 | 0.39 | 0.08 | 498 |
| 443 | | | | | | | | | | | | | | | | | | |
| Phage- | L | I | — | W | — | D | W | E | — | Y | N | F | — | T | 0.13 | 0.39 | 0.11 | 499 |
| 444 | | | | | | | | | | | | | | | | | | |
| Phage- | F | H | — | — | — | D | G | — | — | T | — | P | — | I | 0.08 | 0.39 | 0.08 | 500 |
| 445 | | | | | | | | | | | | | | | | | | |
| Phage- | F | D | — | — | — | — | W | E | W | I | Y | D | — | F | 0.08 | 0.38 | 0.08 | 501 |
| 446 | | | | | | | | | | | | | | | | | | |
| Phage- | I | — | — | — | — | — | W | — | — | — | L | D | — | D | 0.08 | 0.38 | 0.09 | 502 |
| 447 | | | | | | | | | | | | | | | | | | |
| Phage- | L | I | — | — | — | — | — | — | — | — | A | S | — | N | 0.10 | 0.38 | 0.11 | 503 |
| 448 | | | | | | | | | | | | | | | | | | |
| Phage- | T | S | — | W | V | D | W | E | — | F | S | D | — | I | 0.11 | 0.38 | 0.34 | 504 |
| 449 | | | | | | | | | | | | | | | | | | |
| Phage- | Y | — | — | — | — | — | V | E | — | D | Y | V | — | D | 0.10 | 0.38 | 0.08 | 505 |
| 450 | | | | | | | | | | | | | | | | | | |
| Phage- | D | D | — | — | — | — | Q | E | F | I | Y | A | — | I | 0.09 | 0.38 | 0.08 | 506 |
| 451 | | | | | | | | | | | | | | | | | | |
| Phage- | A | D | — | W | — | D | W | E | — | Y | A | D | — | Y | 0.11 | 0.38 | 0.10 | 507 |
| 452 | | | | | | | | | | | | | | | | | | |
| Phage- | Y | — | — | — | — | — | — | — | — | — | I | H | — | I | 0.08 | 0.38 | 0.07 | 508 |
| 453 | | | | | | | | | | | | | | | | | | |
| Phage- | S | E | — | W | — | D | W | E | P | F | F | D | — | N | 0.08 | 0.37 | 0.09 | 509 |
| 454 | | | | | | | | | | | | | | | | | | |
| Phage- | Y | H | — | — | — | — | M | — | — | — | L | I | — | T | 0.07 | 0.37 | 0.07 | 510 |
| 455 | | | | | | | | | | | | | | | | | | |
| Phage- | S | D | — | W | — | D | W | E | D | A | Y | F | — | I | 0.09 | 0.37 | 0.07 | 511 |
| 456 | | | | | | | | | | | | | | | | | | |
| Phage- | — | D | — | — | — | — | V | E | — | Y | Y | H | — | D | 0.08 | 0.37 | 0.08 | 512 |
| 457 | | | | | | | | | | | | | | | | | | |
| Phage- | D | N | — | — | — | — | Y | — | — | — | I | A | — | N | 0.10 | 0.37 | 0.10 | 513 |
| 458 | | | | | | | | | | | | | | | | | | |
| Phage- | L | — | — | — | — | V | — | E | F | Y | D | Y | — | Y | 0.08 | 0.37 | 0.10 | 514 |
| 459 | | | | | | | | | | | | | | | | | | |
| Phage- | Y | T | — | — | — | — | M | — | — | — | — | T | — | I | 0.09 | 0.37 | 0.08 | 515 |
| 460 | | | | | | | | | | | | | | | | | | |
| Phage- | N | F | — | W | — | D | W | E | V | N | S | F | — | D | 0.09 | 0.37 | 0.08 | 516 |
| 461 | | | | | | | | | | | | | | | | | | |
| Phage- | N | A | — | W | — | D | W | E | Y | I | D | F | — | N | 0.12 | 0.36 | 0.09 | 517 |
| 462 | | | | | | | | | | | | | | | | | | |
| Phage- | Y | N | — | — | — | — | M | — | — | — | T | F | — | S | 0.09 | 0.36 | 0.07 | 518 |
| 463 | | | | | | | | | | | | | | | | | | |
| Phage- | L | D | — | — | — | — | L | — | — | — | I | T | — | Y | 0.08 | 0.36 | 0.09 | 519 |
| 464 | | | | | | | | | | | | | | | | | | |
| Phage- | H | — | — | — | — | — | — | — | — | — | T | N | — | D | 0.11 | 0.36 | 0.08 | 520 |
| 465 | | | | | | | | | | | | | | | | | | |
| Phage- | I | I | — | — | — | — | L | P | — | D | Y | V | — | T | 0.08 | 0.36 | 0.08 | 521 |
| 466 | | | | | | | | | | | | | | | | | | |
| Phage- | D | I | — | — | — | — | — | — | — | — | T | D | — | S | 0.08 | 0.36 | 0.08 | 522 |
| 467 | | | | | | | | | | | | | | | | | | |
| Phage- | P | — | — | — | — | — | — | — | — | — | — | L | — | F | 0.10 | 0.36 | 0.09 | 523 |
| 468 | | | | | | | | | | | | | | | | | | |
| Phage- | — | F | — | — | — | — | — | — | — | — | — | D | — | Y | 0.07 | 0.36 | 0.08 | 524 |
| 469 | | | | | | | | | | | | | | | | | | |
| Phage- | Y | D | — | W | — | D | W | E | — | A | L | P | — | A | 0.08 | 0.36 | 0.08 | 525 |
| 470 | | | | | | | | | | | | | | | | | | |
| Phage- | D | D | — | W | — | D | W | E | D | Y | — | F | — | F | 0.10 | 0.36 | 0.10 | 526 |
| 471 | | | | | | | | | | | | | | | | | | |
| Phage- | H | F | — | — | — | — | W | E | L | F | S | D | — | Y | 0.11 | 0.36 | 0.11 | 527 |
| 472 | | | | | | | | | | | | | | | | | | |
| Phage- | I | T | — | W | — | D | W | E | V | N | F | P | — | Y | 0.07 | 0.35 | 0.07 | 528 |
| 473 | | | | | | | | | | | | | | | | | | |
| Phage- | P | D | — | — | — | — | L | — | — | — | I | T | — | N | 0.20 | 0.35 | 0.16 | 529 |
| 474 | | | | | | | | | | | | | | | | | | |
| Phage- | N | L | — | W | — | D | W | E | A | F | F | P | L | Y | 0.08 | 0.35 | 0.07 | 530 |
| 475 | | | | | | | | | | | | | | | | | | |
| Phage- | F | — | — | — | — | — | — | E | Y | I | R | D | — | Y | 0.08 | 0.35 | 0.07 | 531 |
| 476 | | | | | | | | | | | | | | | | | | |
| Phage- | F | F | — | — | — | — | — | — | — | — | I | I | — | D | 0.09 | 0.35 | 0.10 | 532 |
| 477 | | | | | | | | | | | | | | | | | | |
| Phage- | — | L | — | — | — | K | G | G | P | T | Y | N | — | S | 0.08 | 0.35 | 0.10 | 533 |
| 478 | | | | | | | | | | | | | | | | | | |
| Phage- | L | A | — | W | — | V | W | E | — | P | G | H | — | D | 0.11 | 0.35 | 0.10 | 534 |
| 479 | | | | | | | | | | | | | | | | | | |
| Phage- | D | — | — | — | — | — | V | E | D | V | N | D | — | Y | 0.07 | 0.35 | 0.08 | 535 |
| 480 | | | | | | | | | | | | | | | | | | |
| Phage- | D | — | — | — | — | — | — | E | — | A | H | Y | — | N | 0.08 | 0.35 | 0.07 | 536 |
| 481 | | | | | | | | | | | | | | | | | | |
| Phage- | — | L | — | — | — | — | L | — | — | T | L | T | — | I | 0.08 | 0.35 | 0.07 | 537 |
| 482 | | | | | | | | | | | | | | | | | | |
| Phage- | Y | — | — | — | — | — | I | E | D | Y | N | L | — | N | 0.10 | 0.34 | 0.09 | 538 |
| 483 | | | | | | | | | | | | | | | | | | |
| Phage- | Y | I | — | — | — | — | V | E | — | Y | Y | N | — | F | 0.13 | 0.34 | 0.14 | 539 |
| 484 | | | | | | | | | | | | | | | | | | |
| Phage- | D | I | — | — | — | — | L | — | — | — | I | F | — | F | 0.08 | 0.34 | 0.09 | 540 |
| 485 | | | | | | | | | | | | | | | | | | |
| Phage- | D | I | — | — | — | — | V | E | — | D | Y | L | — | Y | 0.07 | 0.34 | 0.08 | 541 |
| 486 | | | | | | | | | | | | | | | | | | |
| Phage- | T | L | — | — | — | — | — | E | — | D | A | P | — | I | 0.10 | 0.34 | 0.08 | 542 |
| 487 | | | | | | | | | | | | | | | | | | |
| Phage- | N | I | — | W | — | D | W | E | Y | I | N | S | — | V | 0.14 | 0.34 | 0.09 | 543 |
| 488 | | | | | | | | | | | | | | | | | | |
| Phage- | N | D | — | — | — | — | V | E | — | Y | Y | Y | — | T | 0.07 | 0.34 | 0.09 | 544 |
| 489 | | | | | | | | | | | | | | | | | | |
| Phage- | I | T | — | — | — | — | M | — | — | — | T | D | — | N | 0.08 | 0.34 | 0.08 | 545 |
| 490 | | | | | | | | | | | | | | | | | | |
| Phage- | Y | — | — | — | — | — | M | A | — | D | L | I | — | D | 0.10 | 0.34 | 0.29 | 546 |
| 491 | | | | | | | | | | | | | | | | | | |
| Phage- | I | D | — | — | — | — | L | — | — | — | I | V | — | T | 0.11 | 0.33 | 0.09 | 547 |
| 492 | | | | | | | | | | | | | | | | | | |
| Phage- | H | T | — | W | — | D | W | E | W | D | — | Y | — | D | 0.08 | 0.33 | 0.07 | 548 |
| 493 | | | | | | | | | | | | | | | | | | |
| Phage- | I | H | — | — | — | — | W | E | L | I | D | D | — | L | 0.08 | 0.33 | 0.10 | 549 |
| 494 | | | | | | | | | | | | | | | | | | |
| Phage- | Y | T | — | — | — | — | L | — | — | — | I | T | — | T | 0.08 | 0.33 | 0.08 | 550 |
| 495 | | | | | | | | | | | | | | | | | | |
| Phage- | F | H | — | — | — | — | V | E | — | T | — | Y | — | F | 0.11 | 0.33 | 0.09 | 551 |
| 496 | | | | | | | | | | | | | | | | | | |
| Phage- | D | — | — | — | — | — | L | — | — | — | L | I | — | N | 0.07 | 0.33 | 0.08 | 552 |
| 497 | | | | | | | | | | | | | | | | | | |
| Phage- | I | — | — | — | — | — | — | — | — | — | D | Y | — | T | 0.08 | 0.33 | 0.10 | 553 |
| 498 | | | | | | | | | | | | | | | | | | |
| Phage- | D | L | — | — | — | — | I | E | — | D | L | V | — | T | 0.09 | 0.33 | 0.09 | 554 |
| 499 | | | | | | | | | | | | | | | | | | |
| Phage- | N | I | — | — | — | — | L | Q | — | D | I | V | — | P | 0.09 | 0.33 | 0.09 | 555 |
| 500 | | | | | | | | | | | | | | | | | | |
| Phage- | N | N | — | — | — | — | M | — | — | — | I | T | — | Y | 0.08 | 0.33 | 0.08 | 556 |
| 501 | | | | | | | | | | | | | | | | | | |
| Phage- | H | T | — | — | — | — | L | — | — | — | I | V | — | V | 0.08 | 0.33 | 0.08 | 557 |
| 502 | | | | | | | | | | | | | | | | | | |
| Phage- | Y | — | — | — | — | — | I | E | D | I | L | V | — | T | 0.12 | 0.33 | 0.23 | 558 |
| 503 | | | | | | | | | | | | | | | | | | |
| Phage- | N | T | — | — | — | — | — | E | F | V | H | L | — | P | 0.07 | 0.33 | 0.11 | 559 |
| 504 | | | | | | | | | | | | | | | | | | |
| Phage- | D | I | — | — | — | — | M | — | — | — | T | V | — | D | 0.10 | 0.33 | 0.09 | 560 |
| 505 | | | | | | | | | | | | | | | | | | |
| Phage- | A | I | — | — | — | — | V | E | I | V | N | Y | — | Y | 0.09 | 0.32 | 0.07 | 561 |
| 506 | | | | | | | | | | | | | | | | | | |
| Phage- | H | L | — | — | — | — | V | E | D | P | T | A | — | V | 0.10 | 0.32 | 0.28 | 562 |
| 507 | | | | | | | | | | | | | | | | | | |
| Phage- | A | D | — | — | — | — | L | — | — | — | I | S | — | T | 0.10 | 0.32 | 0.09 | 563 |
| 508 | | | | | | | | | | | | | | | | | | |
| Phage- | F | D | — | — | — | — | L | — | — | — | — | I | — | D | 0.07 | 0.32 | 0.09 | 564 |
| 509 | | | | | | | | | | | | | | | | | | |
| Phage- | D | V | — | — | — | — | — | — | — | — | — | D | — | N | 0.10 | 0.32 | 0.08 | 565 |
| 510 | | | | | | | | | | | | | | | | | | |
| Phage- | Y | L | — | — | — | — | V | E | — | I | S | I | — | F | 0.08 | 0.32 | 0.07 | 566 |
| 511 | | | | | | | | | | | | | | | | | | |
| Phage- | S | A | — | — | — | — | — | — | — | — | L | H | — | V | 0.10 | 0.31 | 0.30 | 567 |
| 512 | | | | | | | | | | | | | | | | | | |
| Phage- | H | L | — | W | — | D | W | E | — | D | S | A | — | N | 0.08 | 0.31 | 0.08 | 568 |
| 513 | | | | | | | | | | | | | | | | | | |
| Phage- | H | T | — | W | — | D | W | E | Y | D | Y | D | — | F | 0.10 | 0.31 | 0.08 | 569 |
| 514 | | | | | | | | | | | | | | | | | | |
| Phage- | Y | D | — | — | — | — | W | E | — | V | A | L | — | N | 0.10 | 0.31 | 0.10 | 570 |
| 515 | | | | | | | | | | | | | | | | | | |
| Phage- | T | L | — | — | — | — | I | E | — | Y | I | V | — | Y | 0.09 | 0.31 | 0.23 | 571 |
| 516 | | | | | | | | | | | | | | | | | | |
| Phage- | S | — | — | — | — | — | — | — | — | — | I | F | — | T | 0.09 | 0.31 | 0.09 | 572 |
| 517 | | | | | | | | | | | | | | | | | | |
| Phage- | D | I | — | — | — | — | — | — | — | — | L | H | — | Y | 0.08 | 0.31 | 0.08 | 573 |
| 518 | | | | | | | | | | | | | | | | | | |
| Phage- | L | F | — | — | — | — | — | E | — | A | Y | L | — | I | 0.08 | 0.31 | 0.08 | 574 |
| 519 | | | | | | | | | | | | | | | | | | |
| Phage- | I | F | — | — | — | — | I | E | — | D | F | V | — | T | 0.10 | 0.31 | 0.10 | 575 |
| 520 | | | | | | | | | | | | | | | | | | |
| Phage- | D | D | — | — | — | — | W | E | Y | Y | — | A | — | V | 0.08 | 0.31 | 0.08 | 576 |
| 521 | | | | | | | | | | | | | | | | | | |
| Phage- | D | D | — | — | — | — | L | — | — | T | T | I | — | Y | 0.10 | 0.31 | 0.09 | 577 |
| 522 | | | | | | | | | | | | | | | | | | |
| Phage- | A | S | — | — | — | — | L | — | — | — | I | A | — | D | 0.10 | 0.31 | 0.09 | 578 |
| 523 | | | | | | | | | | | | | | | | | | |
| Phage- | Y | L | — | — | — | — | V | E | D | Y | D | Y | — | Y | 0.08 | 0.31 | 0.09 | 579 |
| 524 | | | | | | | | | | | | | | | | | | |
| Phage- | D | — | — | — | — | — | W | E | — | T | I | F | — | A | 0.12 | 0.30 | 0.09 | 580 |
| 525 | | | | | | | | | | | | | | | | | | |
| Phage- | D | F | — | — | — | D | G | E | — | F | Y | I | — | P | 0.12 | 0.30 | 0.11 | 581 |
| 526 | | | | | | | | | | | | | | | | | | |
| Phage- | — | — | — | — | — | — | V | E | — | N | I | L | — | H | 0.15 | 0.30 | 0.17 | 582 |
| 527 | | | | | | | | | | | | | | | | | | |
| Phage- | D | — | — | — | — | — | V | E | — | N | Y | F | — | F | 0.12 | 0.30 | 0.21 | 583 |
| 528 | | | | | | | | | | | | | | | | | | |
| Phage- | N | D | — | W | — | D | W | Y | — | F | L | S | — | D | 0.10 | 0.30 | 0.10 | 584 |
| 529 | | | | | | | | | | | | | | | | | | |
| Phage- | R | D | — | W | — | D | W | E | V | P | Y | F | — | D | 0.08 | 0.30 | 0.09 | 585 |
| 530 | | | | | | | | | | | | | | | | | | |
| Phage- | N | L | — | — | — | — | V | E | — | A | — | Y | — | Y | 0.10 | 0.30 | 0.13 | 586 |
| 531 | | | | | | | | | | | | | | | | | | |
| Phage- | F | D | — | W | — | D | G | E | L | N | Y | L | — | T | 0.23 | 0.30 | 0.08 | 587 |
| 532 | | | | | | | | | | | | | | | | | | |
| Phage- | Y | — | — | — | — | — | V | E | D | V | N | L | — | I | 0.18 | 0.30 | 0.10 | 588 |
| 533 | | | | | | | | | | | | | | | | | | |
| Phage- | D | N | — | — | — | — | Y | — | — | — | I | T | — | L | 0.11 | 0.29 | 0.09 | 589 |
| 534 | | | | | | | | | | | | | | | | | | |
| Phage- | L | N | — | W | — | D | W | E | — | D | Y | S | — | N | 0.09 | 0.29 | 0.09 | 590 |
| 535 | | | | | | | | | | | | | | | | | | |
| Phage- | P | T | — | — | — | — | V | E | — | L | L | S | — | N | 0.12 | 0.29 | 0.26 | 591 |
| 536 | | | | | | | | | | | | | | | | | | |
| Phage- | D | H | — | — | — | — | V | E | L | I | F | Y | — | H | 0.11 | 0.29 | 0.08 | 592 |
| 537 | | | | | | | | | | | | | | | | | | |
| Phage- | F | H | — | — | — | — | L | — | — | — | I | F | — | Y | 0.08 | 0.29 | 0.08 | 593 |
| 538 | | | | | | | | | | | | | | | | | | |
| Phage- | F | D | — | — | — | — | L | E | — | T | — | V | — | P | 0.16 | 0.29 | 0.16 | 594 |
| 539 | | | | | | | | | | | | | | | | | | |
| Phage- | D | F | — | — | — | — | L | — | — | — | L | P | — | A | 0.08 | 0.29 | 0.08 | 595 |
| 540 | | | | | | | | | | | | | | | | | | |
| Phage- | D | S | — | — | — | — | L | — | — | — | I | Y | — | D | 0.09 | 0.29 | 0.09 | 596 |
| 541 | | | | | | | | | | | | | | | | | | |
| Phage- | A | D | — | W | — | D | W | E | — | F | L | L | — | F | 0.12 | 0.29 | 0.10 | 597 |
| 542 | | | | | | | | | | | | | | | | | | |
| Phage- | I | L | — | — | — | — | V | E | — | L | D | F | — | N | 0.10 | 0.28 | 0.08 | 598 |
| 543 | | | | | | | | | | | | | | | | | | |
| Phage- | F | F | — | — | — | — | — | E | — | I | F | L | — | Y | 0.09 | 0.28 | 0.07 | 599 |
| 544 | | | | | | | | | | | | | | | | | | |
| Phage- | Y | N | — | — | — | D | G | — | — | — | Y | D | — | H | 0.07 | 0.28 | 0.07 | 600 |
| 545 | | | | | | | | | | | | | | | | | | |
| Phage- | D | N | — | — | — | — | L | — | — | T | I | T | — | F | 0.11 | 0.28 | 0.11 | 601 |
| 546 | | | | | | | | | | | | | | | | | | |
| Phage- | H | N | — | — | — | D | G | — | — | A | F | I | — | N | 0.09 | 0.28 | 0.23 | 602 |
| 547 | | | | | | | | | | | | | | | | | | |
| Phage- | D | — | — | — | — | — | V | E | — | D | L | V | — | P | 0.10 | 0.28 | 0.23 | 603 |
| 548 | | | | | | | | | | | | | | | | | | |
| Phage- | D | — | — | — | — | — | L | — | — | — | L | N | — | F | 0.08 | 0,28 | 0.07 | 604 |
| 549 | | | | | | | | | | | | | | | | | | |
| Phage- | D | — | — | — | — | — | — | Q | — | D | F | H | — | H | 0.11 | 0.28 | 0.27 | 605 |
| 550 | | | | | | | | | | | | | | | | | | |
| Phage- | Y | D | — | — | — | — | W | E | F | T | D | D | — | I | 0.10 | 0.28 | 0.18 | 606 |
| 551 | | | | | | | | | | | | | | | | | | |
| Phage- | D | I | — | — | — | — | Y | E | — | D | I | I | — | Y | 0.14 | 0.28 | 0.19 | 607 |
| 552 | | | | | | | | | | | | | | | | | | |
| Phage- | F | D | — | — | — | — | L | — | — | T | — | P | — | P | 0.11 | 0.28 | 0.08 | 608 |
| 553 | | | | | | | | | | | | | | | | | | |
| Phage- | N | — | — | — | — | — | L | — | — | T | S | V | — | D | 0.09 | 0.28 | 0.26 | 609 |
| 554 | | | | | | | | | | | | | | | | | | |
| Phage- | Y | — | — | — | — | — | W | E | F | — | F | D | — | D | 0.17 | 0.28 | 0.11 | 610 |
| 555 | | | | | | | | | | | | | | | | | | |
| Phage- | Y | A | — | — | — | — | L | — | — | — | — | H | — | D | 0.09 | 0.28 | 0.08 | 611 |
| 556 | | | | | | | | | | | | | | | | | | |
| Phage- | F | L | — | — | — | — | V | E | Q | D | Y | F | — | V | 0.08 | 0.28 | 0.10 | 612 |
| 557 | | | | | | | | | | | | | | | | | | |
| Phage- | D | N | — | — | — | — | — | — | — | — | — | R | — | D | 0.09 | 0.27 | 0.26 | 613 |
| 558 | | | | | | | | | | | | | | | | | | |
| Phage- | N | D | — | — | — | D | G | I | — | H | — | D | — | Y | 0.10 | 0.27 | 0.24 | 614 |
| 559 | | | | | | | | | | | | | | | | | | |
| Phage- | Y | F | — | — | — | — | V | E | D | Y | N | D | — | F | 0.08 | 0.27 | 0.09 | 615 |
| 560 | | | | | | | | | | | | | | | | | | |
| Phage- | N | L | — | — | — | — | — | — | — | — | I | F | — | Y | 0.10 | 0.27 | 0.07 | 616 |
| 561 | | | | | | | | | | | | | | | | | | |
| Phage- | I | D | — | W | — | D | W | E | — | Y | I | P | — | T | 0.10 | 0.27 | 0.08 | 617 |
| 562 | | | | | | | | | | | | | | | | | | |
| Phage- | I | — | — | — | — | D | G | — | — | — | F | I | — | A | 0.07 | 0.27 | 0.08 | 618 |
| 563 | | | | | | | | | | | | | | | | | | |
| Phage- | D | — | — | — | — | — | — | — | — | L | — | V | — | Y | 0.08 | 0.27 | 0.07 | 619 |
| 564 | | | | | | | | | | | | | | | | | | |
| Phage- | — | F | — | — | — | — | W | E | D | I | T | D | — | D | 0.13 | 0.27 | 0.09 | 620 |
| 565 | | | | | | | | | | | | | | | | | | |
| Phage- | L | D | — | — | — | — | V | — | — | T | F | T | — | H | 0.08 | 0.26 | 0.08 | 621 |
| 566 | | | | | | | | | | | | | | | | | | |
| Phage- | D | D | — | — | — | — | Y | — | — | — | F | A | — | H | 0.13 | 0.26 | 0.10 | 622 |
| 567 | | | | | | | | | | | | | | | | | | |
| Phage- | I | — | — | — | — | — | Y | Q | — | D | L | P | — | N | 0.12 | 0.26 | 0.11 | 623 |
| 568 | | | | | | | | | | | | | | | | | | |
| Phage- | L | D | — | — | — | — | V | E | — | Y | N | Y | — | V | 0.09 | 0.26 | 0.08 | 624 |
| 569 | | | | | | | | | | | | | | | | | | |
| Phage- | Y | V | — | — | — | — | — | — | — | — | S | A | — | N | 0.14 | 0.26 | 0.08 | 625 |
| 570 | | | | | | | | | | | | | | | | | | |
| Phage- | T | P | — | — | — | — | L | E | — | A | I | — | — | Y | 0.10 | 0.26 | 0.10 | 626 |
| 571 | | | | | | | | | | | | | | | | | | |
| Phage- | Y | F | — | — | — | — | — | — | — | — | A | D | — | N | 0.08 | 0.26 | 0.08 | 627 |
| 572 | | | | | | | | | | | | | | | | | | |
| Phage- | D | D | — | — | — | — | — | E | — | D | I | I | — | D | 0.12 | 0.26 | 0.25 | 628 |
| 573 | | | | | | | | | | | | | | | | | | |
| Phage- | L | — | — | — | — | V | V | E | — | L | N | H | — | N | 0.08 | 0.26 | 0.09 | 629 |
| 574 | | | | | | | | | | | | | | | | | | |
| Phage- | — | I | — | — | — | D | G | E | — | L | I | A | — | A | 0.09 | 0.26 | 0.27 | 630 |
| 575 | | | | | | | | | | | | | | | | | | |
| Phage- | F | A | — | W | — | D | W | Q | — | T | Y | V | — | N | 0.09 | 0.25 | 0.08 | 631 |
| 576 | | | | | | | | | | | | | | | | | | |
| Phage- | Y | I | — | — | — | — | V | E | F | L | F | F | — | N | 0.08 | 0.25 | 0.08 | 632 |
| 577 | | | | | | | | | | | | | | | | | | |
| Phage- | T | Q | — | — | — | K | G | E | P | T | Y | H | — | Y | 0.12 | 0.25 | 0.12 | 633 |
| 578 | | | | | | | | | | | | | | | | | | |
| Phage- | N | — | — | — | — | — | V | E | — | Y | H | N | — | D | 0.10 | 0.25 | 0.17 | 634 |
| 579 | | | | | | | | | | | | | | | | | | |
| Phage- | — | — | — | — | — | — | W | E | F | F | S | D | — | A | 0.08 | 0.25 | 0.07 | 635 |
| 580 | | | | | | | | | | | | | | | | | | |
| Phage- | F | — | — | — | — | — | L | E | — | — | F | F | — | Y | 0.10 | 0.25 | 0.22 | 636 |
| 581 | | | | | | | | | | | | | | | | | | |
| Phage- | D | — | — | — | — | — | I | E | — | N | F | Y | — | Y | 0.13 | 0.25 | 0.09 | 637 |
| 582 | | | | | | | | | | | | | | | | | | |
| Phage- | Y | A | — | — | — | — | V | E | — | Y | — | Y | — | A | 0.08 | 0.25 | 0.09 | 638 |
| 583 | | | | | | | | | | | | | | | | | | |
| Phage- | — | D | — | — | — | — | L | — | — | — | I | I | — | D | 0.08 | 0.25 | 0.07 | 639 |
| 584 | | | | | | | | | | | | | | | | | | |
| Phage- | N | D | — | — | — | — | M | — | — | — | I | A | — | Y | 0.07 | 0.25 | 0.07 | 640 |
| 585 | | | | | | | | | | | | | | | | | | |
| Phage- | — | — | — | — | — | — | — | — | — | Y | L | A | — | A | 0.09 | 0.25 | 0.21 | 641 |
| 586 | | | | | | | | | | | | | | | | | | |
| Phage- | — | I | — | — | — | — | — | — | — | — | A | N | — | D | 0.08 | 0.25 | 0.09 | 642 |
| 587 | | | | | | | | | | | | | | | | | | |
| Phage- | L | T | — | W | — | D | W | E | — | D | F | F | — | N | 0.07 | 0.24 | 0.07 | 643 |
| 588 | | | | | | | | | | | | | | | | | | |
| Phage- | F | — | — | — | — | — | Q | E | — | I | N | Y | — | Y | 0.10 | 0.24 | 0.23 | 644 |
| 589 | | | | | | | | | | | | | | | | | | |
| Phage- | T | — | — | — | — | — | L | E | — | F | F | L | — | Y | 0.13 | 0.24 | 0.08 | 645 |
| 590 | | | | | | | | | | | | | | | | | | |
| Phage- | P | D | — | — | — | — | L | — | — | — | — | A | — | H | 0.12 | 0.24 | 0.09 | 646 |
| 591 | | | | | | | | | | | | | | | | | | |
| Phage- | N | D | — | — | — | — | — | E | — | I | I | F | — | V | 0.09 | 0.24 | 0.24 | 647 |
| 592 | | | | | | | | | | | | | | | | | | |
| Phage- | Y | I | — | — | — | — | — | — | — | — | F | Y | — | N | 0.25 | 0.24 | 0.08 | 648 |
| 593 | | | | | | | | | | | | | | | | | | |
| Phage- | H | A | — | — | — | — | L | — | — | — | L | L | — | N | 0.20 | 0.24 | 0.07 | 649 |
| 594 | | | | | | | | | | | | | | | | | | |
| Phage- | Y | — | — | — | — | — | W | E | — | A | — | L | — | A | 0.09 | 0.24 | 0.21 | 650 |
| 595 | | | | | | | | | | | | | | | | | | |
| Phage- | A | F | — | — | — | — | V | E | — | Y | D | L | — | N | 0.10 | 0.24 | 0.16 | 651 |
| 596 | | | | | | | | | | | | | | | | | | |
| Phage- | Y | N | — | — | — | — | — | — | — | — | — | A | — | S | 0.15 | 0.23 | 0.09 | 652 |
| 597 | | | | | | | | | | | | | | | | | | |
| Phage- | Y | L | — | — | — | — | V | E | D | D | T | L | — | A | 0.08 | 0.23 | 0.09 | 653 |
| 598 | | | | | | | | | | | | | | | | | | |
| Phage- | A | V | — | — | — | — | — | — | — | — | — | N | — | D | 0.08 | 0.23 | 0.08 | 654 |
| 599 | | | | | | | | | | | | | | | | | | |
| Phage- | N | — | — | — | — | — | W | E | V | Y | S | L | — | P | 0.13 | 0.23 | 0.08 | 655 |
| 600 | | | | | | | | | | | | | | | | | | |
| Phage- | D | F | — | — | — | — | V | E | — | D | T | Y | — | H | 0.07 | 0.23 | 0.07 | 656 |
| 601 | | | | | | | | | | | | | | | | | | |
| Phage- | I | S | — | — | — | — | Y | E | W | D | Y | A | — | N | 0.08 | 0.23 | 0.10 | 657 |
| 602 | | | | | | | | | | | | | | | | | | |
| Phage- | — | N | — | — | — | — | L | — | — | — | I | I | — | Y | 0.08 | 0.23 | 0.08 | 658 |
| 603 | | | | | | | | | | | | | | | | | | |
| Phage- | Y | D | — | — | — | — | — | — | — | T | A | P | — | Y | 0.07 | 0.23 | 0.08 | 659 |
| 604 | | | | | | | | | | | | | | | | | | |
| Phage- | Y | L | — | — | — | — | — | E | — | N | F | L | — | T | 0.09 | 0.23 | 0.22 | 660 |
| 605 | | | | | | | | | | | | | | | | | | |
| Phage- | F | D | — | — | — | — | V | — | — | — | — | D | — | A | 0.08 | 0.22 | 0.09 | 661 |
| 606 | | | | | | | | | | | | | | | | | | |
| Phage- | Y | D | — | — | — | — | Q | E | — | I | S | F | — | N | 0.09 | 0.22 | 0.09 | 662 |
| 607 | | | | | | | | | | | | | | | | | | |
| Phage- | I | D | — | — | — | — | I | E | L | Y | D | D | — | F | 0.09 | 0.22 | 0.09 | 663 |
| 608 | | | | | | | | | | | | | | | | | | |
| Phage- | T | F | — | — | — | — | L | — | — | — | I | Y | — | Y | 0.08 | 0.22 | 0.07 | 664 |
| 609 | | | | | | | | | | | | | | | | | | |
| Phage- | F | F | — | — | — | — | I | — | — | — | N | A | — | V | 0.09 | 0.22 | 0.07 | 665 |
| 610 | | | | | | | | | | | | | | | | | | |
| Phage- | Y | H | — | W | — | D | W | E | P | I | Y | I | — | I | 0.12 | 0.22 | 0.10 | 666 |
| 611 | | | | | | | | | | | | | | | | | | |
| Phage- | A | I | — | — | — | — | Y | E | — | D | H | Y | — | Y | 0.08 | 0.22 | 0.08 | 667 |
| 612 | | | | | | | | | | | | | | | | | | |
| Phage- | P | L | — | — | — | D | G | F | — | N | Y | N | — | F | 0.12 | 0.22 | 0.08 | 668 |
| 613 | | | | | | | | | | | | | | | | | | |
| Phage- | F | P | — | W | — | D | W | E | W | D | N | N | — | H | 0.09 | 0.22 | 0.09 | 669 |
| 614 | | | | | | | | | | | | | | | | | | |
| Phage- | — | D | — | — | — | D | G | — | — | L | A | A | — | H | 0.10 | 0.22 | 0.11 | 670 |
| 615 | | | | | | | | | | | | | | | | | | |
| Phage- | — | D | — | W | — | D | W | E | — | Y | Y | S | — | D | 0.08 | 0.22 | 0.07 | 671 |
| 616 | | | | | | | | | | | | | | | | | | |
| Phage- | — | — | — | — | — | — | Y | — | — | — | Y | D | — | T | 0.07 | 0.21 | 0.10 | 672 |
| 617 | | | | | | | | | | | | | | | | | | |
| Phage- | N | L | — | — | — | — | W | E | N | F | A | D | — | F | 0.08 | 0.21 | 0.08 | 673 |
| 618 | | | | | | | | | | | | | | | | | | |
| Phage- | Y | L | — | — | — | — | L | E | V | F | F | V | — | D | 0.12 | 0.21 | 0.10 | 674 |
| 619 | | | | | | | | | | | | | | | | | | |
| Phage- | I | F | — | — | — | — | L | E | D | Y | S | I | — | F | 0.09 | 0.21 | 0.08 | 675 |
| 620 | | | | | | | | | | | | | | | | | | |
| Phage- | D | — | — | — | — | — | L | E | Q | Y | D | L | — | F | 0.09 | 0.21 | 0.08 | 676 |
| 621 | | | | | | | | | | | | | | | | | | |
| Phage- | L | L | — | — | — | V | N | E | D | P | L | D | — | Y | 0.11 | 0.21 | 0.13 | 677 |
| 622 | | | | | | | | | | | | | | | | | | |
| Phage- | I | D | — | — | — | — | — | — | — | — | — | F | — | Y | 0.08 | 0.21 | 0.08 | 678 |
| 623 | | | | | | | | | | | | | | | | | | |
| Phage- | I | I | — | — | — | — | V | E | — | I | D | I | — | S | 0.08 | 0.21 | 0.08 | 679 |
| 624 | | | | | | | | | | | | | | | | | | |
| Phage- | I | A | — | W | — | D | W | E | D | Y | S | S | — | P | 0.08 | 0.21 | 0.11 | 680 |
| 625 | | | | | | | | | | | | | | | | | | |
| Phage- | Y | — | — | — | — | — | V | E | D | — | N | D | — | I | 0.09 | 0.21 | 0.07 | 681 |
| 626 | | | | | | | | | | | | | | | | | | |
| Phage- | N | I | — | — | — | — | M | — | — | — | I | D | — | I | 0.08 | 0.21 | 0.07 | 682 |
| 627 | | | | | | | | | | | | | | | | | | |
| Phage- | F | D | — | W | — | D | W | E | — | L | — | S | — | Y | 0.07 | 0.21 | 0.08 | 683 |
| 628 | | | | | | | | | | | | | | | | | | |
| Phage- | Y | F | — | — | — | — | W | E | D | H | F | F | — | D | 0.09 | 0.21 | 0.19 | 684 |
| 629 | | | | | | | | | | | | | | | | | | |
| Phage- | T | — | — | — | — | — | — | E | — | D | S | Y | — | D | 0.12 | 0.20 | 0.09 | 685 |
| 630 | | | | | | | | | | | | | | | | | | |
| Phage- | N | L | — | — | — | — | V | E | L | I | D | I | — | S | 0.11 | 0.20 | 0.09 | 686 |
| 631 | | | | | | | | | | | | | | | | | | |
| Phage- | D | N | — | — | — | — | W | E | — | V | Y | L | — | N | 0.08 | 0.20 | 0.08 | 687 |
| 632 | | | | | | | | | | | | | | | | | | |
| Phage- | F | L | — | — | — | — | — | — | — | — | D | L | — | F | 0.08 | 0.20 | 0.09 | 688 |
| 633 | | | | | | | | | | | | | | | | | | |
| Phage- | H | I | — | — | — | — | Q | — | — | — | I | — | — | T | 0.09 | 0.20 | 0.19 | 689 |
| 634 | | | | | | | | | | | | | | | | | | |
| Phage- | F | D | — | W | — | D | W | E | D | N | S | Y | — | D | 0.10 | 0.20 | 0.09 | 690 |
| 635 | | | | | | | | | | | | | | | | | | |
| Phage- | T | A | — | — | — | — | W | E | F | D | F | N | — | D | 0.08 | 0.20 | 0.07 | 691 |
| 636 | | | | | | | | | | | | | | | | | | |
| Phage- | H | H | — | W | — | D | W | E | D | Y | S | T | — | P | 0.10 | 0.20 | 0.11 | 692 |
| 637 | | | | | | | | | | | | | | | | | | |
| Phage- | Y | — | — | — | — | — | — | — | — | — | — | N | — | F | 0.07 | 0.20 | 0.08 | 693 |
| 638 | | | | | | | | | | | | | | | | | | |
| Phage- | L | H | — | W | — | D | W | E | — | I | D | I | — | D | 0.08 | 0.20 | 0.09 | 694 |
| 639 | | | | | | | | | | | | | | | | | | |
| Phage- | D | I | I | — | — | D | G | Q | — | D | F | V | — | S | 0.08 | 0.20 | 0.09 | 695 |
| 640 | | | | | | | | | | | | | | | | | | |
| Phage- | D | V | — | W | — | D | W | E | V | N | Y | F | — | D | 0.09 | 0.20 | 0.07 | 696 |
| 641 | | | | | | | | | | | | | | | | | | |
| Phage- | — | N | — | — | — | — | M | — | — | — | T | D | — | A | 0.12 | 0.20 | 0.10 | 697 |
| 642 | | | | | | | | | | | | | | | | | | |
| Phage- | D | N | — | — | — | — | — | — | L | A | T | V | — | N | 0.11 | 0.19 | 0.19 | 698 |
| 643 | | | | | | | | | | | | | | | | | | |
| Phage- | D | L | — | — | — | — | — | E | I | V | H | N | — | N | 0.08 | 0.19 | 0.08 | 699 |
| 644 | | | | | | | | | | | | | | | | | | |
| Phage- | — | N | — | — | — | — | — | — | — | — | S | Y | — | F | 0.13 | 0.19 | 0.09 | 700 |
| 645 | | | | | | | | | | | | | | | | | | |
| Phage- | N | I | — | W | — | D | W | E | — | D | N | F | — | S | 0.08 | 0.19 | 0.08 | 701 |
| 646 | | | | | | | | | | | | | | | | | | |
| Phage- | F | V | — | — | — | — | W | E | V | Y | D | D | — | D | 0.08 | 0.19 | 0.08 | 702 |
| 647 | | | | | | | | | | | | | | | | | | |
| Phage- | A | — | — | — | — | — | L | E | V | V | H | L | — | V | 0.10 | 0.19 | 0.17 | 703 |
| 648 | | | | | | | | | | | | | | | | | | |
| Phage- | P | F | — | — | — | — | M | — | — | T | I | D | — | Y | 0.07 | 0.19 | 0.09 | 704 |
| 649 | | | | | | | | | | | | | | | | | | |
| Phage- | L | L | — | — | — | V | M | E | D | V | F | A | — | Y | 0.08 | 0.19 | 0.08 | 705 |
| 650 | | | | | | | | | | | | | | | | | | |
| Phage- | D | L | — | — | — | — | — | — | — | — | T | N | — | Y | 0.07 | 0.19 | 0.08 | 706 |
| 651 | | | | | | | | | | | | | | | | | | |
| Phage- | H | D | — | — | — | — | M | E | — | Y | Y | L | — | P | 0.10 | 0.18 | 0.10 | 707 |
| 652 | | | | | | | | | | | | | | | | | | |
| Phage- | T | D | — | — | — | — | Y | — | — | — | I | I | — | P | 0.08 | 0.18 | 0.09 | 708 |
| 653 | | | | | | | | | | | | | | | | | | |
| Phage- | — | L | — | W | — | D | W | E | D | Y | A | D | — | N | 0.09 | 0.18 | 0.08 | 709 |
| 654 | | | | | | | | | | | | | | | | | | |
| Phage- | N | D | — | — | — | — | L | — | — | — | L | T | — | D | 0.07 | 0.18 | 0.09 | 710 |
| 655 | | | | | | | | | | | | | | | | | | |
| Phage- | I | — | — | — | — | — | L | — | — | — | I | A | — | Y | 0.11 | 0.18 | 0.08 | 711 |
| 656 | | | | | | | | | | | | | | | | | | |
| Phage- | N | — | — | — | — | — | V | E | — | F | N | F | — | H | 0.11 | 0.18 | 0.14 | 712 |
| 657 | | | | | | | | | | | | | | | | | | |
| Phage- | D | V | — | — | — | — | I | E | — | Y | S | F | — | I | 0.08 | 0.18 | 0.09 | 713 |
| 658 | | | | | | | | | | | | | | | | | | |
| Phage- | D | L | — | — | — | — | V | E | — | I | T | D | — | A | 0.10 | 0.18 | 0.12 | 714 |
| 659 | | | | | | | | | | | | | | | | | | |
| Phage- | H | D | — | — | — | — | — | — | — | — | — | F | — | I | 0.12 | 0.18 | 0.12 | 715 |
| 660 | | | | | | | | | | | | | | | | | | |
| Phage- | P | L | — | — | — | V | L | E | — | D | I | Y | — | Y | 0.10 | 0.18 | 0.13 | 716 |
| 661 | | | | | | | | | | | | | | | | | | |
| Phage- | D | L | — | — | — | — | — | E | D | I | I | D | — | N | 0.10 | 0.18 | 0.11 | 717 |
| 662 | | | | | | | | | | | | | | | | | | |
| Phage- | D | — | — | — | — | — | V | E | V | P | S | N | — | N | 0.10 | 0.18 | 0.18 | 718 |
| 663 | | | | | | | | | | | | | | | | | | |
| Phage- | I | I | — | = | — | — | L | — | — | — | T | A | — | D | 0.10 | 0.18 | 0.09 | 719 |
| 664 | | | | | | | | | | | | | | | | | | |
| Phage- | D | H | — | — | — | — | — | — | — | — | — | N | — | D | 0.10 | 0.18 | 0.14 | 720 |
| 665 | | | | | | | | | | | | | | | | | | |
| Phage- | F | D | — | — | — | — | — | — | — | — | L | Y | — | S | 0.07 | 0.18 | 0.07 | 721 |
| 666 | | | | | | | | | | | | | | | | | | |
| Phage- | F | A | — | W | — | D | W | E | — | V | Y | I | — | Y | 0.08 | 0.18 | 0.08 | 722 |
| 667 | | | | | | | | | | | | | | | | | | |
| Phage- | L | — | — | — | — | — | — | — | — | — | L | D | — | S | 0.08 | 0.18 | 0.09 | 723 |
| 668 | | | | | | | | | | | | | | | | | | |
| Phage- | D | L | — | — | — | — | L | E | — | A | F | L | — | A | 0.09 | 0.18 | 0.08 | 724 |
| 669 | | | | | | | | | | | | | | | | | | |
| Phage- | F | A | — | — | — | — | L | — | — | T | L | T | — | L | 0.10 | 0.18 | 0.08 | 725 |
| 670 | | | | | | | | | | | | | | | | | | |
| Phage- | F | D | — | — | — | — | V | E | — | I | S | N | — | D | 0.17 | 0.18 | 0.10 | 726 |
| 671 | | | | | | | | | | | | | | | | | | |
| Phage- | — | H | — | — | — | — | L | E | Y | p | F | D | — | N | 0.09 | 0.17 | 0.16 | 727 |
| 672 | | | | | | | | | | | | | | | | | | |
| Phage- | A | — | — | — | — | — | — | E | — | H | T | T | — | N | 0.10 | 0.17 | 0.15 | 728 |
| 673 | | | | | | | | | | | | | | | | | | |
| Phage- | L | — | — | — | — | V | S | E | Q | F | T | F | — | I | 0.08 | 0.17 | 0.08 | 729 |
| 674 | | | | | | | | | | | | | | | | | | |
| Phage- | D | — | — | — | — | — | L | — | — | — | Y | D | — | N | 0.10 | 0.17 | 0.09 | 730 |
| 675 | | | | | | | | | | | | | | | | | | |
| Phage- | F | — | — | — | — | — | W | E | — | F | D | V | — | I | 0.13 | 0.17 | 0.15 | 731 |
| 676 | | | | | | | | | | | | | | | | | | |
| Phage- | F | T | — | — | — | — | V | E | — | Y | D | H | — | I | 0.08 | 0.17 | 0.09 | 732 |
| 677 | | | | | | | | | | | | | | | | | | |
| Phage- | Y | N | — | — | — | — | — | — | — | — | — | T | — | F | 0.12 | 0.17 | 0.11 | 733 |
| 678 | | | | | | | | | | | | | | | | | | |
| Phage- | A | V | — | — | — | — | N | — | — | — | N | S | — | A | 0.08 | 0.17 | 0.08 | 734 |
| 679 | | | | | | | | | | | | | | | | | | |
| Phage- | I | — | — | W | — | D | W | E | V | P | N | D | — | A | 0.10 | 0.17 | 0.09 | 735 |
| 680 | | | | | | | | | | | | | | | | | | |
| Phage- | Y | F | — | — | — | — | — | E | — | F | F | H | — | Y | 0.12 | 0.17 | 0.12 | 736 |
| 681 | | | | | | | | | | | | | | | | | | |
| Phage- | Y | V | — | — | — | D | G | — | — | — | S | F | — | D | 0.12 | 0.17 | 0.12 | 737 |
| 682 | | | | | | | | | | | | | | | | | | |
| Phage- | I | S | — | — | — | — | V | E | — | F | F | Y | — | Y | 0.10 | 0.17 | 0.08 | 738 |
| 683 | | | | | | | | | | | | | | | | | | |
| Phage- | L | I | — | — | — | V | — | E | — | D | — | Y | — | D | 0.17 | 0.17 | 0.15 | 739 |
| 684 | | | | | | | | | | | | | | | | | | |
| Phage- | — | — | — | — | — | — | V | E | D | H | N | Y | — | A | 0.14 | 0.17 | 0.16 | 740 |
| 685 | | | | | | | | | | | | | | | | | | |
| Phage- | L | D | — | — | — | — | — | E | F | V | Y | I | — | A | 0.08 | 0.17 | 0.10 | 741 |
| 686 | | | | | | | | | | | | | | | | | | |
| Phage- | Y | D | — | — | — | — | — | E | — | D | L | P | — | I | 0.17 | 0.17 | 0.11 | 742 |
| 687 | | | | | | | | | | | | | | | | | | |
| Phage- | D | V | — | — | — | — | V | E | — | D | Y | Y | — | D | 0.10 | 0.17 | 0.14 | 743 |
| 688 | | | | | | | | | | | | | | | | | | |
| Phage- | N | D | — | W | — | D | W | E | Y | D | N | V | — | V | 0.08 | 0.17 | 0.10 | 744 |
| 689 | | | | | | | | | | | | | | | | | | |
| Phage- | D | L | — | — | — | — | — | E | V | A | N | D | — | N | 0.10 | 0.16 | 0.16 | 745 |
| 690 | | | | | | | | | | | | | | | | | | |
| Phage- | H | D | — | — | — | — | L | — | — | — | I | S | — | N | 0.09 | 0.16 | 0.07 | 746 |
| 691 | | | | | | | | | | | | | | | | | | |
| Phage- | L | D | — | W | — | D | W | E | — | T | T | H | — | D | 0.08 | 0.16 | 0.08 | 747 |
| 692 | | | | | | | | | | | | | | | | | | |
| Phage- | I | I | — | — | — | — | V | E | — | D | D | Y | — | L | 0.09 | 0.16 | 0.09 | 748 |
| 693 | | | | | | | | | | | | | | | | | | |
| Phage- | Y | — | — | W | — | D | W | E | — | V | I | I | — | D | 0.09 | 0.16 | 0.09 | 749 |
| 694 | | | | | | | | | | | | | | | | | | |
| Phage- | F | D | — | — | — | — | I | — | — | Y | T | N | — | N | 0.12 | 0.16 | 0.09 | 750 |
| 695 | | | | | | | | | | | | | | | | | | |
| Phage- | I | T | — | — | — | — | L | — | — | T | I | N | — | D | 0.08 | 0.16 | 0.11 | 751 |
| 696 | | | | | | | | | | | | | | | | | | |
| Phage- | D | S | — | — | — | — | V | E | — | D | I | Y | — | I | 0.07 | 0.16 | 0.08 | 752 |
| 697 | | | | | | | | | | | | | | | | | | |
| Phage- | Y | L | — | — | — | — | I | — | — | — | G | N | — | H | 0.07 | 0.16 | 0.08 | 753 |
| 698 | | | | | | | | | | | | | | | | | | |
| Phage- | D | N | — | — | — | — | L | P | — | D | Y | F | — | D | 0.08 | 0.16 | 0.10 | 754 |
| 699 | | | | | | | | | | | | | | | | | | |
| Phage- | — | L | — | — | — | — | — | E | I | V | S | N | — | N | 0.11 | 0.16 | 0.08 | 755 |
| 700 | | | | | | | | | | | | | | | | | | |
| Phage- | — | D | — | W | — | D | W | E | — | D | I | V | — | D | 0.10 | 0.16 | 0.09 | 756 |
| 701 | | | | | | | | | | | | | | | | | | |
| Phage- | — | — | — | W | — | D | W | E | D | N | F | P | — | Y | 0.07 | 0.16 | 0.07 | 757 |
| 702 | | | | | | | | | | | | | | | | | | |
| Phage- | D | — | — | — | — | — | V | E | — | H | F | N | — | H | 0.08 | 0.16 | 0.08 | 758 |
| 703 | | | | | | | | | | | | | | | | | | |
| Phage- | A | D | — | — | — | — | — | E | — | D | A | Y | — | Y | 0.12 | 0.16 | 0.09 | 759 |
| 704 | | | | | | | | | | | | | | | | | | |
| Phage- | I | L | — | W | — | D | W | E | D | A | T | F | — | Y | 0.09 | 0.16 | 0.07 | 760 |
| 705 | | | | | | | | | | | | | | | | | | |
| Phage- | I | H | — | W | — | D | W | E | D | F | N | I | — | P | 0.09 | 0.16 | 0.08 | 761 |
| 706 | | | | | | | | | | | | | | | | | | |
| Phage- | T | I | — | — | — | — | V | E | D | Y | N | D | — | I | 0.07 | 0.16 | 0.07 | 762 |
| 707 | | | | | | | | | | | | | | | | | | |
| Phage- | D | D | — | — | — | — | L | — | — | — | — | A | — | T | 0.08 | 0.16 | 0.08 | 763 |
| 708 | | | | | | | | | | | | | | | | | | |
| Phage- | D | D | — | W | — | D | W | E | D | H | I | F | — | F | 0.13 | 0.16 | 0.08 | 764 |
| 709 | | | | | | | | | | | | | | | | | | |
| Phage- | I | N | — | — | — | — | V | E | — | I | I | F | — | D | 0.12 | 0.15 | 0.12 | 765 |
| 710 | | | | | | | | | | | | | | | | | | |
| Phage- | I | F | — | W | — | D | W | E | D | D | H | V | — | I | 0.08 | 0.15 | 0.09 | 766 |
| 711 | | | | | | | | | | | | | | | | | | |
| Phage- | I | I | — | — | — | — | — | E | — | I | S | D | — | L | 0.12 | 0.15 | 0.15 | 767 |
| 712 | | | | | | | | | | | | | | | | | | |
| Phage- | F | D | 1 | — | — | — | V | E | — | Y | N | D | — | D | 0.11 | 0.15 | 0.09 | 768 |
| 713 | | | | | | | | | | | | | | | | | | |
| Phage- | N | D | — | — | — | — | L | — | — | T | L | Y | — | I | 0.08 | 0.15 | 0.10 | 769 |
| 714 | | | | | | | | | | | | | | | | | | |
| Phage- | A | I | — | — | — | — | L | E | — | D | I | S | — | N | 0.12 | 0.15 | 0.17 | 770 |
| 715 | | | | | | | | | | | | | | | | | | |
| Phage- | H | L | — | — | — | — | — | — | — | — | T | N | — | Y | 0.07 | 0.15 | 0.07 | 771 |
| 716 | | | | | | | | | | | | | | | | | | |
| Phage- | S | — | — | — | — | — | L | — | — | — | — | A | — | I | 0.10 | 0.15 | 0.11 | 772 |
| 717 | | | | | | | | | | | | | | | | | | |
| Phage- | L | — | — | — | — | — | — | E | Q | L | A | D | — | T | 0.08 | 0.15 | 0.08 | 773 |
| 718 | | | | | | | | | | | | | | | | | | |
| Phage- | I | D | — | — | — | — | L | — | — | — | T | A | — | N | 0.07 | 0.15 | 0.08 | 774 |
| 719 | | | | | | | | | | | | | | | | | | |
| Phage- | F | D | — | — | — | D | G | Q | — | D | L | V | — | N | 0.10 | 0.15 | 0.08 | 775 |
| 720 | | | | | | | | | | | | | | | | | | |
| Phage- | N | L | — | — | — | — | — | E | — | F | F | D | — | Y | 0.09 | 0.15 | 0.15 | 776 |
| 721 | | | | | | | | | | | | | | | | | | |
| Phage- | S | I | — | — | — | — | L | Q | — | D | I | V | — | P | 0.09 | 0.14 | 0.14 | 777 |
| 722 | | | | | | | | | | | | | | | | | | |
| Phage- | N | P | — | — | — | — | Y | — | — | — | A | H | — | D | 0.08 | 0.14 | 0.08 | 778 |
| 723 | | | | | | | | | | | | | | | | | | |
| Phage- | Y | D | — | — | — | — | L | — | — | Y | Y | N | — | N | 0.12 | 0.14 | 0.11 | 779 |
| 724 | | | | | | | | | | | | | | | | | | |
| Phage- | I | D | — | — | — | — | L | — | — | T | I | F | — | D | 0.07 | 0.14 | 0.08 | 780 |
| 725 | | | | | | | | | | | | | | | | | | |
| Phage- | D | N | — | — | — | — | L | — | — | — | — | T | — | T | 0.09 | 0.14 | 0.10 | 781 |
| 726 | | | | | | | | | | | | | | | | | | |
| Phage- | — | D | — | — | — | — | L | — | — | — | S | Y | — | D | 0.10 | 0.14 | 0.13 | 782 |
| 727 | | | | | | | | | | | | | | | | | | |
| Phage- | Y | — | — | — | — | — | — | E | F | I | D | F | — | F | 0.07 | 0.14 | 0.07 | 783 |
| 728 | | | | | | | | | | | | | | | | | | |
| Phage- | Y | D | — | W | — | D | W | E | V | I | T | Y | — | N | 0.08 | 0.14 | 0.09 | 784 |
| 729 | | | | | | | | | | | | | | | | | | |
| Phage- | F | D | — | — | — | — | I | E | — | D | F | F | — | V | 0.06 | 0.14 | 0.07 | 785 |
| 730 | | | | | | | | | | | | | | | | | | |
| Phage- | I | F | — | W | — | D | W | — | D | I | N | F | — | D | 0.10 | 0.14 | 0.14 | 786 |
| 731 | | | | | | | | | | | | | | | | | | |
| Phage- | N | F | — | — | — | — | L | P | — | D | I | T | — | Y | 0.37 | 0.14 | 0.09 | 787 |
| 732 | | | | | | | | | | | | | | | | | | |
| Phage- | S | L | — | — | — | — | — | E | — | Y | Y | H | — | L | 0.09 | 0.14 | 0.07 | 788 |
| 733 | | | | | | | | | | | | | | | | | | |
| Phage- | A | S | — | — | — | — | L | — | — | — | L | D | — | L | 0.12 | 0.14 | 0.13 | 789 |
| 734 | | | | | | | | | | | | | | | | | | |
| Phage- | S | F | — | — | — | — | R | E | W | D | L | A | — | Y | 0.09 | 0.14 | 0.08 | 790 |
| 735 | | | | | | | | | | | | | | | | | | |
| Phage- | H | L | — | — | — | — | — | E | D | V | L | D | — | I | 0.08 | 0.14 | 0.12 | 791 |
| 736 | | | | | | | | | | | | | | | | | | |
| Phage- | L | D | — | — | — | D | G | — | — | F | Y | Y | — | L | 0.20 | 0.14 | 0.09 | 792 |
| 737 | | | | | | | | | | | | | | | | | | |
| Phage- | D | N | — | W | — | D | W | E | — | D | I | A | — | T | 0.16 | 0.14 | 0.11 | 793 |
| 738 | | | | | | | | | | | | | | | | | | |
| Phage- | S | D | — | — | — | — | L | — | — | T | I | H | — | I | 0.09 | 0.14 | 0.08 | 794 |
| 739 | | | | | | | | | | | | | | | | | | |
| Phage- | N | S | — | — | — | D | G | I | — | — | — | D | — | L | 0.08 | 0.14 | 0.08 | 795 |
| 740 | | | | | | | | | | | | | | | | | | |
| Phage- | D | L | — | — | — | — | L | — | — | — | T | L | — | I | 0.07 | 0.14 | 0.08 | 796 |
| 741 | | | | | | | | | | | | | | | | | | |
| Phage- | F | D | — | — | — | — | S | — | — | — | F | N | — | Y | 0.09 | 0.14 | 0.11 | 797 |
| 742 | | | | | | | | | | | | | | | | | | |
| Phage- | D | L | — | — | — | — | — | E | — | D | D | I | — | Y | 0.12 | 0.14 | 0.13 | 798 |
| 743 | | | | | | | | | | | | | | | | | | |
| Phage- | H | A | — | — | — | — | — | E | — | D | T | Y | — | F | 0.10 | 0.14 | 0.14 | 799 |
| 744 | | | | | | | | | | | | | | | | | | |
| Phage- | S | D | — | — | — | — | L | — | — | — | — | A | — | I | 0.11 | 0.14 | 0.08 | 800 |
| 745 | | | | | | | | | | | | | | | | | | |
| Phage- | I | V | — | — | — | — | L | P | — | D | Y | N | — | Y | 0.09 | 0.13 | 0.11 | 801 |
| 746 | | | | | | | | | | | | | | | | | | |
| Phage- | D | L | — | — | — | — | — | — | — | — | F | I | — | F | 0.09 | 0.13 | 0.08 | 802 |
| 747 | | | | | | | | | | | | | | | | | | |
| Phage- | Y | D | — | — | — | — | — | — | — | T | L | T | — | N | 0.13 | 0.13 | 0.08 | 803 |
| 748 | | | | | | | | | | | | | | | | | | |
| Phage- | Y | D | — | — | — | — | V | E | — | I | — | N | — | D | 0.11 | 0.13 | 0.12 | 804 |
| 749 | | | | | | | | | | | | | | | | | | |
| Phage- | — | F | — | — | — | — | I | E | D | D | H | V | — | I | 0.07 | 0.13 | 0.07 | 805 |
| 750 | | | | | | | | | | | | | | | | | | |
| Phage- | F | I | — | — | — | — | W | E | D | D | Y | A | — | S | 0.12 | 0.13 | 0.12 | 806 |
| 751 | | | | | | | | | | | | | | | | | | |
| Phage- | N | F | — | — | — | — | — | — | — | — | — | — | — | N | 0.10 | 0.13 | 0.09 | 807 |
| 752 | | | | | | | | | | | | | | | | | | |
| Phage- | N | L | — | — | — | — | V | E | — | I | L | I | — | D | 0.07 | 0.13 | 0.07 | 808 |
| 753 | | | | | | | | | | | | | | | | | | |
| Phage- | D | S | — | — | — | — | V | E | — | Y | D | L | — | N | 0.11 | 0.13 | 0.08 | 809 |
| 754 | | | | | | | | | | | | | | | | | | |
| Phage- | T | L | — | — | — | — | — | E | — | I | T | D | — | N | 0.09 | 0.13 | 0.08 | 810 |
| 755 | | | | | | | | | | | | | | | | | | |
| Phage- | T | V | — | — | — | K | M | E | M | N | S | T | — | D | 0.09 | 0.13 | 0,09 | 811 |
| 756 | | | | | | | | | | | | | | | | | | |
| Phage- | — | H | — | W | — | D | W | E | D | A | — | S | — | N | 0.10 | 0.12 | 0.11 | 812 |
| 757 | | | | | | | | | | | | | | | | | | |
| Phage- | D | L | — | — | — | D | G | N | — | L | D | F | — | F | 0.08 | 0.12 | 0.08 | 813 |
| 758 | | | | | | | | | | | | | | | | | | |
| Phage- | D | L | — | — | — | D | G | E | — | H | Y | Y | — | D | 0.09 | 0.12 | 0.07 | 814 |
| 759 | | | | | | | | | | | | | | | | | | |
| Phage- | F | N | — | — | — | — | V | E | — | I | L | L | — | T | 0.11 | 0.12 | 0.12 | 815 |
| 760 | | | | | | | | | | | | | | | | | | |
| Phage- | H | — | — | — | — | — | V | E | N | I | N | D | — | I | 0.09 | 0.12 | 0.07 | 816 |
| 761 | | | | | | | | | | | | | | | | | | |
| Phage- | I | D | — | — | — | — | L | — | — | — | — | I | — | D | 0.09 | 0.12 | 0.08 | 817 |
| 762 | | | | | | | | | | | | | | | | | | |
| Phage- | I | F | — | — | — | — | — | E | Q | P | A | L | — | Y | 0.08 | 0.12 | 0.09 | 818 |
| 763 | | | | | | | | | | | | | | | | | | |
| Phage- | Y | D | — | — | — | — | — | Q | — | D | L | V | — | P | 0.10 | 0.12 | 0.08 | 819 |
| 764 | | | | | | | | | | | | | | | | | | |
| Phage- | H | A | | W | | D | W | E | | P | N | Y | | D | 0.13 | 0.12 | 0.09 | 820 |
| 765 | | | | | | | | | | | | | | | | | | |
| Phage- | N | V | — | W | — | D | W | E | — | D | Y | N | — | Y | 0.11 | 0.12 | 0.10 | 821 |
| 766 | | | | | | | | | | | | | | | | | | |
| Phage- | S | F | — | — | Q | — | L | G | D | N | Y | D | — | I | 0.09 | 0.12 | 0.12 | 822 |
| 767 | | | | | | | | | | | | | | | | | | |
| Phage- | D | D | — | — | — | — | L | — | — | T | T | V | — | Y | 0.08 | 0.12 | 0.09 | 823 |
| 768 | | | | | | | | | | | | | | | | | | |
| Phage- | N | F | — | — | — | — | W | E | V | A | T | L | — | L | 0.09 | 0.12 | 0.14 | 824 |
| 769 | | | | | | | | | | | | | | | | | | |
| Phage- | D | L | — | — | — | — | V | E | — | D | T | Y | — | N | 0.09 | 0.11 | 0.07 | 825 |
| 770 | | | | | | | | | | | | | | | | | | |
| Phage- | A | L | — | — | — | — | V | E | Q | V | D | L | — | T | 0.08 | 0.11 | 0.08 | 826 |
| 771 | | | | | | | | | | | | | | | | | | |
| Phage- | D | D | — | — | — | — | L | — | — | — | — | N | — | N | 0.08 | 0.11 | 0.08 | 827 |
| 772 | | | | | | | | | | | | | | | | | | |
| Phage- | I | F | — | — | — | — | — | E | Q | I | I | Y | — | D | 0.09 | 0.11 | 0.11 | 828 |
| 773 | | | | | | | | | | | | | | | | | | |
| Phage- | S | D | — | W | — | D | W | E | — | V | Y | Y | — | S | 0.09 | 0.11 | 0.10 | 829 |
| 774 | | | | | | | | | | | | | | | | | | |
| Phage- | F | F | — | — | — | D | G | — | — | V | A | I | — | D | 0.09 | 0.11 | 0.07 | 830 |
| 775 | | | | | | | | | | | | | | | | | | |
| Phage- | D | D | — | W | — | D | W | E | D | D | — | Y | — | Y | 0.11 | 0.11 | 0.07 | 831 |
| 776 | | | | | | | | | | | | | | | | | | |
| Phage- | Y | D | — | — | — | — | — | — | — | T | — | V | — | P | 0.08 | 0.11 | 0.08 | 832 |
| 777 | | | | | | | | | | | | | | | | | | |
| Phage- | S | — | — | — | — | — | L | — | — | — | — | N | — | V | 0.10 | 0.11 | 0.08 | 833 |
| 778 | | | | | | | | | | | | | | | | | | |
| Phage- | A | F | — | V | S | F | Q | Q | S | L | P | H | — | D | 0.09 | 0.11 | 0.10 | 834 |
| 779 | | | | | | | | | | | | | | | | | | |
| Phage- | — | N | — | — | — | — | — | E | — | Y | F | V | — | F | 0.09 | 0.11 | 0.09 | 835 |
| 780 | | | | | | | | | | | | | | | | | | |
| Phage- | T | N | — | W | — | D | W | E | 1 | D | F | A | — | V | 0.07 | 0.11 | 0.08 | 836 |
| 781 | | | | | | | | | | | | | | | | | | |
| Phage- | D | D | — | — | — | — | — | E | — | I | I | L | — | F | 0.08 | 0.10 | 0.08 | 837 |
| 782 | | | | | | | | | | | | | | | | | | |
| Phage- | N | S | — | — | — | — | L | E | D | Y | H | L | — | P | 0.08 | 0.10 | 0.10 | 838 |
| 783 | | | | | | | | | | | | | | | | | | |
| Phage- | I | H | — | — | — | D | S | — | G | F | D | F | — | D | 0.09 | 0.10 | 0.08 | 839 |
| 784 | | | | | | | | | | | | | | | | | | |
| Phage- | F | D | — | — | — | — | L | E | — | D | H | L | — | F | 0.08 | 0.10 | 0.08 | 840 |
| 785 | | | | | | | | | | | | | | | | | | |
| Phage- | T | V | — | W | — | D | — | E | — | Y | A | D | — | D | 0.10 | 0.10 | 0.08 | 841 |
| 786 | | | | | | | | | | | | | | | | | | |
| Phage- | Y | F | — | W | — | D | W | E | — | A | A | D | — | L | 0.09 | 0.10 | 0.09 | 842 |
| 787 | | | | | | | | | | | | | | | | | | |
| Phage- | — | S | — | — | — | — | — | — | — | — | — | D | — | — | 0.08 | 0.10 | 0.07 | 843 |
| 788 | | | | | | | | | | | | | | | | | | |
| Phage- | A | V | — | — | — | — | L | P | — | D | I | V | — | Y | 0.08 | 0.10 | 0.08 | 844 |
| 789 | | | | | | | | | | | | | | | | | | |
| Phage- | — | L | — | — | — | — | V | E | — | Y | H | L | — | A | 0.08 | 0.10 | 0.30 | 845 |
| 790 | | | | | | | | | | | | | | | | | | |
| Phage- | S | L | — | W | — | D | W | E | — | V | D | N | — | F | 0.12 | 0.10 | 0.11 | 846 |
| 791 | | | | | | | | | | | | | | | | | | |
| Phage- | T | I | — | — | — | D | G | Q | — | D | Y | N | — | H | 0.07 | 0.10 | 0.07 | 847 |
| 792 | | | | | | | | | | | | | | | | | | |
| Phage- | F | L | — | — | — | — | G | E | P | T | Y | L | — | T | 0.12 | 0.10 | 0.09 | 848 |
| 793 | | | | | | | | | | | | | | | | | | |
| Phage- | D | D | — | W | L | — | 0 | H | D | I | Y | V | — | A | 0.10 | 0.10 | 0.08 | 849 |
| 794 | | | | | | | | | | | | | | | | | | |
| Phage- | I | F | — | — | — | — | V | E | — | V | A | F | — | F | 0.07 | 0.10 | 0.08 | 850 |
| 795 | | | | | | | | | | | | | | | | | | |
| Phage- | A | L | — | — | — | — | V | E | D | D | Y | D | — | L | 0.09 | 0.10 | 0.09 | 851 |
| 796 | | | | | | | | | | | | | | | | | | |
| Phage- | D | D | — | — | — | — | I | E | L | Y | L | T | — | A | 0.08 | 0.10 | 0.08 | 852 |
| 797 | | | | | | | | | | | | | | | | | | |
| Phage- | T | D | — | W | — | D | W | E | D | D | S | I | — | D | 0.07 | 0.10 | 0.07 | 853 |
| 798 | | | | | | | | | | | | | | | | | | |
| Phage- | S | I | — | — | — | — | L | E | — | I | F | L | — | N | 0.08 | 0.10 | 0.08 | 854 |
| 799 | | | | | | | | | | | | | | | | | | |
| Phage- | N | D | — | — | — | — | L | E | — | D | I | L | — | F | 0.07 | 0.10 | 0.09 | 855 |
| 800 | | | | | | | | | | | | | | | | | | |
| Phage- | D | D | — | — | — | — | L | — | — | H | Y | S | — | Y | 0.09 | 0.09 | 0.08 | 856 |
| 801 | | | | | | | | | | | | | | | | | | |
| Phage- | F | V | — | — | — | — | W | E | — | I | D | L | — | I | 0.10 | 0.09 | 0.09 | 857 |
| 802 | | | | | | | | | | | | | | | | | | |
| Phage- | Y | D | — | — | — | — | L | — | — | — | L | S | — | P | 0.07 | 0.09 | 0.07 | 858 |
| 803 | | | | | | | | | | | | | | | | | | |
| Phage- | N | L | — | — | — | — | L | — | — | — | I | I | — | P | 0.08 | 0.09 | 0.08 | 859 |
| 804 | | | | | | | | | | | | | | | | | | |
| Phage- | I | D | — | W | — | D | W | E | — | F | N | N | — | F | 0.08 | 0.09 | 0.09 | 860 |
| 805 | | | | | | | | | | | | | | | | | | |
| Phage- | A | — | — | — | — | — | L | E | — | H | D | Y | — | Y | 0.08 | 0.09 | 0.09 | 861 |
| 806 | | | | | | | | | | | | | | | | | | |
| Phage- | D | D | — | S | — | Q | — | — | Q | I | D | L | — | D | 0.14 | 0.09 | 0.09 | 862 |
| 807 | | | | | | | | | | | | | | | | | | |
| Phage- | S | — | — | — | — | — | S | — | — | — | — | Y | — | Y | 0.08 | 0.09 | 0.07 | 863 |
| 808 | | | | | | | | | | | | | | | | | | |
| Phage- | S | L | — | — | — | — | — | Q | — | D | A | P | — | N | 0.07 | 0.09 | 0.07 | 864 |
| 809 | | | | | | | | | | | | | | | | | | |
| Phage- | D | A | — | — | — | — | L | — | — | T | T | H | — | D | 0.09 | 0.09 | 0.08 | 865 |
| 810 | | | | | | | | | | | | | | | | | | |
| Phage- | N | D | — | — | — | — | V | E | — | V | A | D | — | F | 0.07 | 0.09 | 0.08 | 866 |
| 811 | | | | | | | | | | | | | | | | | | |
| Phage- | Y | I | — | — | — | — | — | E | Q | D | Y | F | — | F | 0.08 | 0.09 | 0.08 | 867 |
| 812 | | | | | | | | | | | | | | | | | | |
| Phage- | T | D | — | — | — | D | G | — | — | T | N | Y | — | F | 0.11 | 0.09 | 0.08 | 868 |
| 813 | | | | | | | | | | | | | | | | | | |
| Phage- | Y | D | — | — | — | — | V | — | — | — | — | L | — | P | 0.08 | 0.09 | 0.08 | 869 |
| 814 | | | | | | | | | | | | | | | | | | |
| Phage- | I | D | — | — | — | — | — | — | — | — | A | Y | — | T | 0.08 | 0.09 | 0.08 | 870 |
| 815 | | | | | | | | | | | | | | | | | | |
| Phage- | F | D | — | — | — | — | — | E | — | F | F | H | — | Y | 0.09 | 0.09 | 0.09 | 871 |
| 816 | | | | | | | | | | | | | | | | | | |
| Phage- | F | P | — | — | — | — | I | E | — | Y | D | Y | — | V | 0.09 | 0.09 | 0.08 | 872 |
| 817 | | | | | | | | | | | | | | | | | | |
| Phage- | A | D | — | — | — | I | — | E | S | I | D | I | — | V | 0.09 | 0.09 | 0.07 | 873 |
| 818 | | | | | | | | | | | | | | | | | | |
| Phage- | I | S | — | — | — | D | L | W | P | T | D | I | — | T | 0.10 | 0.09 | 0.10 | 874 |
| 819 | | | | | | | | | | | | | | | | | | |
| Phage- | D | D | — | — | — | D | G | — | — | V | H | T | I | N | 0.09 | 0.09 | 0.07 | 875 |
| 820 | | | | | | | | | | | | | | | | | | |
| Phage- | I | D | — | W | — | D | W | E | G | — | F | A | — | N | 0.09 | 0.09 | 0.08 | 876 |
| 821 | | | | | | | | | | | | | | | | | | |
| Phage- | L | N | — | — | — | D | G | — | — | T | F | Y | — | D | 0.08 | 0.08 | 0.07 | 877 |
| 822 | | | | | | | | | | | | | | | | | | |
| Phage- | I | H | — | — | — | — | L | G | A | Y | I | S | — | S | 0.08 | 0.08 | 0.09 | 878 |
| 823 | | | | | | | | | | | | | | | | | | |
| Phage- | T | I | — | W | — | D | W | E | — | D | Y | F | — | Y | 0.08 | 0.08 | 0.08 | 879 |
| 824 | | | | | | | | | | | | | | | | | | |
| Phage- | H | S | — | L | A | Q | — | — | Q | D | L | V | — | I | 0.07 | 0.08 | 0.07 | 880 |
| 825 | | | | | | | | | | | | | | | | | | |
| Phage- | N | N | — | A | S | D | L | S | — | D | N | S | — | I | 0.07 | 0.08 | 0.08 | 881 |
| 826 | | | | | | | | | | | | | | | | | | |
| Phage- | — | N | — | W | — | D | W | E | — | D | — | A | — | N | 0.09 | 0.08 | 0.07 | 882 |
| 827 | | | | | | | | | | | | | | | | | | |
| Phage- | — | — | — | — | — | — | L | — | — | — | F | Y | — | F | 0.08 | 0.08 | 0.07 | 883 |
| 828 | | | | | | | | | | | | | | | | | | |
| Phage- | L | F | — | — | T | V | L | — | — | F | F | D | — | D | 0.07 | 0.08 | 0.07 | 884 |
| 829 | | | | | | | | | | | | | | | | | | |
| Phage- | F | D | — | — | — | — | L | — | — | — | I | S | — | A | 0.08 | 0.08 | 0.07 | 885 |
| 830 | | | | | | | | | | | | | | | | | | |
| Phage- | Y | — | — | — | — | — | V | E | — | N | — | Y | — | I | 0.07 | 0.08 | 0.08 | 886 |
| 831 | | | | | | | | | | | | | | | | | | |
| Phage- | D | — | — | — | — | — | L | — | D | — | T | I | — | H | 0.12 | 0.08 | 0.10 | 887 |
| 832 | | | | | | | | | | | | | | | | | | |
| Phage- | — | F | — | — | — | Q | W | A | — | A | N | A | — | F | 0.09 | 0.08 | 0.07 | 888 |
| 833 | | | | | | | | | | | | | | | | | | |
| Phage- | F | T | — | — | — | — | Y | — | — | — | I | T | — | P | 0.09 | 0.08 | 0.09 | 889 |
| 834 | | | | | | | | | | | | | | | | | | |
| Phage- | L | N | — | — | — | V | N | — | — | — | S | V | — | I | 0.07 | 0.08 | 0.08 | 890 |
| 835 | | | | | | | | | | | | | | | | | | |
| Phage- | A | I | — | W | — | D | W | E | — | F | S | D | — | H | 0.07 | 0.08 | 0.07 | 891 |
| 836 | | | | | | | | | | | | | | | | | | |
| Phage- | Y | — | — | — | V | D | L | G | A | N | — | Y | — | Y | 0.10 | 0.08 | 0.09 | 892 |
| 837 | | | | | | | | | | | | | | | | | | |
| Phage- | H | — | — | — | — | — | V | E | — | D | Y | H | — | D | 0.07 | 0.08 | 0.07 | 893 |
| 838 | | | | | | | | | | | | | | | | | | |
| Phage- | N | D | — | — | S | L | Q | Y | D | I | P | T | — | V | 0.08 | 0.08 | 0.0 | 894 |
| 839 | | | | | | | | | | | | | | | | | | |
| Phage- | Y | V | — | R | — | Q | L | — | V | Y | H | Y | — | N | 0.15 | 0.08 | 0.07 | 895 |
| 840 | | | | | | | | | | | | | | | | | | |
| Phage- | H | D | — | — | — | D | G | — | — | — | I | I | — | S | 0.08 | 0.08 | 0.07 | 896 |
| 841 | | | | | | | | | | | | | | | | | | |
| Phage- | F | D | — | — | — | — | L | — | — | T | I | I | — | P | 0.08 | 0.08 | 0.08 | 897 |
| 842 | | | | | | | | | | | | | | | | | | |
| Phage- | — | D | — | — | S | D | R | G | — | N | — | A | — | H | 0.07 | 0.08 | 0.07 | 898 |
| 843 | | | | | | | | | | | | | | | | | | |
| Phage- | N | I | — | L | A | Q | — | N | — | D | P | T | — | N | 0.07 | 0.08 | 0.08 | 899 |
| 844 | | | | | | | | | | | | | | | | | | |
| Phage- | T | N | — | — | S | K | S | Q | V | — | D | H | — | I | 0.10 | 0.08 | 0.09 | 900 |
| 845 | | | | | | | | | | | | | | | | | | |
| Phage- | N | H | — | H | — | Q | — | W | — | L | T | N | — | N | 0.09 | 0.07 | 0.07 | 901 |
| 846 | | | | | | | | | | | | | | | | | | |
| Phage- | L | L | — | H | — | Q | G | — | L | Y | H | L | — | H | 0.09 | 0.07 | 0.08 | 902 |
| 847 | | | | | | | | | | | | | | | | | | |
| Phage- | T | N | — | D | S | K | L | E | G | D | D | N | — | F | 0.09 | 0.07 | 0.07 | 903 |
| 848 | | | | | | | | | | | | | | | | | | |
| Phage- | N | D | — | — | — | — | M | — | — | — | L | L | — | D | 0.08 | 0.07 | 0.07 | 904 |
| 849 | | | | | | | | | | | | | | | | | | |
| Phage- | F | H | — | — | — | — | V | — | — | — | I | N | — | N | 0.07 | 0.07 | 0.07 | 905 |
| 850 | | | | | | | | | | | | | | | | | | |
| Phage- | D | F | — | — | — | D | G | — | — | T | Y | V | — | S | 0.07 | 0.07 | 0.08 | 906 |
| 851 | | | | | | | | | | | | | | | | | | |
| Phage- | — | S | — | — | — | Q | — | — | — | N | N | T | — | N | 0.07 | 0.07 | 0.08 | 907 |
| 852 | | | | | | | | | | | | | | | | | | |
| Phage- | — | — | — | H | L | — | S | E | Q | F | D | I | — | I | 0.08 | 0.07 | 0.08 | 908 |
| 853 | | | | | | | | | | | | | | | | | | |
| Phage | D | D | — | — | — | — | W | E | F | V | F | F | — | D | 0.08 | 0.07 | 0.08 | 909 |
| 854 | | | | | | | | | | | | | | | | | | |
| Phage- | Y | N | — | E | Q | Q | Q | — | — | D | P | S | — | I | 0.07 | 0.07 | 0.07 | 910 |
| 855 | | | | | | | | | | | | | | | | | | |
| Phage- | N | T | — | — | H | — | Q | H | — | F | N | — | — | L | 0.08 | 0.07 | 0.08 | 911 |
| 856 | | | | | | | | | | | | | | | | | | |
| Phage- | H | P | — | Q | — | G | — | E | — | V | D | Y | — | V | 0.08 | 0.07 | 0.08 | 912 |
| 857 | | | | | | | | | | | | | | | | | | |
| Phage- | — | A | — | S | R | Q | L | G | — | D | A | Y | — | N | 0.07 | 0.07 | 0.07 | 913 |
| 858 | | | | | | | | | | | | | | | | | | |
| Phage- | D | I | — | — | A | Q | E | V | H | V | Y | T | — | P | 0.07 | 0.07 | 0.07 | 914 |
| 859 | | | | | | | | | | | | | | | | | | |
| Phage- | F | F | — | E | G | N | L | — | A | Y | L | L | — | L | 0.08 | 0.07 | 0.08 | 915 |
| 860 | | | | | | | | | | | | | | | | | | |
| Phage- | Y | — | — | — | — | D | G | E | — | N | I | V | — | D | 0.07 | 0.07 | 0.07 | 916 |
| 861 | | | | | | | | | | | | | | | | | | |

TABLE 18
Summary of Peptide Sequences

| | anti-CD3 | | SEQ |
|---|---|---|---|
| Peptide-ID | Panned target | Sequence | ID NO: |
| Peptide-29 | SP34.185 | DDCWPDWEFDFACA | 44 |
| Peptide-30 | SP34.185 | YICGLDFPDFLYCD | 45 |
| Peptide-31 | SP34.185 | FDCWPDWEEYFVCD | 46 |
| Peptide-32 | SP34.185 | YICWPDWEEYFDCD | 47 |
| Peptide-33 | SP34.185 | NICWPDWEDDYFCF | 48 |
| Peptide-34 | SP34.185 | NFCWPDWEYIYPCI | 49 |
| Peptide-35 | SP34.185 | VDCWPDWEEDFLCI | 50 |
| Peptide-36 | SP34.185 | HACWPDWEEYFPCN | 51 |
| Peptide-37 | SP34.185 | YDCGPDVDESYVCV | 52 |
| Peptide-38 | SP34.185 | IDCWPDWEDDTFCY | 53 |
| Peptide-39 | SP34.185 | YLCGPDGDETLACY | 54 |
| Peptide-40 | SP34.185 | VDCGPDGDESILCY | 55 |

TABLE 19
Summary of FIG. 5A

| | Pep- | Pep- | Pep- | Pep- | Pep- | Pep- | Pep- |
|---|---|---|---|---|---|---|---|
| ELISA | tide-29 | tide-30 | tide-31 | tide-32 | tide-33 | tide-34 | tide-2 |
| EC50 | 2.163 | 1.564 | 2.035 | 1.713 | 2.174 | 2.752 | 1.972 |
| nM | | | | | | | |

TABLE 20
Summary of FIG. 5B

| | Pep- | Pep- | Pep- | Pep- | Pep- | Pep- | Pep- |
|---|---|---|---|---|---|---|---|
| ELISA | tide-35 | tide-36 | tide-37 | tide-38 | tide-39 | tide-40 | tide-2 |
| EC50 | 2.804 | 2.695 | 344.3 | 1.652 | 1.286 | 1.895 | 1.623 |
| nM | | | | | | | |

TABLE 21
Summary of FIG. 6A

| | Pep- | Pep- | Pep- | Pep- | Pep- | Pep- | Pep- |
|---|---|---|---|---|---|---|---|
| ELISA | tide-29 | tide-30 | tide-31 | tide-32 | tide-33 | tide-34 | tide-2 |
| IC50 | 7.9 | 3.2 | 8.5 | 6.5 | 17.2 | 11.7 | 0.28 |
| uM | | | | | | | |

TABLE 22
Summary of FIG. 6A

| | Pep- | Pep- | Pep- | Pep- | Pep- | Pep- | Pep- |
|---|---|---|---|---|---|---|---|
| ELISA | tide-35 | tide-36 | tide-37 | tide-38 | tide-39 | tide-40 | tide-2 |
| IC50 | 17.3 | 15.2 | >100 | 2.9 | 0.01 | 0.35 | 0.15 |
| uM | | | | | | | |

What is claimed is:

1. An isolated polypeptide or polypeptide complex comprising a peptide that impairs binding of an anti-CD3 binding domain to cluster of differentiation 3 (CD3) wherein the peptide comprises an amino acid sequence selected from SEQ ID NOs: 1-2 and 19-55 or the peptide comprises 1, 2, or 3, amino acid substitutions, additions, or deletions relative to an amino acid sequence selected from SEQ ID NOs: 1-2 and 19-55.

2. The isolated polypeptide or polypeptide complex of claim 1, wherein the peptide comprises an amino acid sequence selected from SEQ ID NOs: 1-2 and 19-55.

3. The isolated polypeptide or polypeptide complex of claim 2, wherein the peptide comprises the amino acid sequence of SEQ ID NO: 1.

4. The isolated polypeptide or polypeptide complex of claim 2, wherein the peptide comprises the amino acid sequence of SEQ ID NO: 2.

5. The isolated polypeptide or polypeptide complex of claim 2, wherein the peptide comprises the amino acid sequence of SEQ ID NO: 54.

6. An isolated polypeptide or polypeptide complex comprising an anti-CD3 binding domain that is linked to a peptide that impairs binding of the anti-CD3 binding domain to CD3 wherein the peptide comprises an amino acid sequence according to Z₁-Z₂-C-Z₄-P-Z₆-Z₇-Z₈-Z₉-Z₁₀-Z₁₁-Z₁₂-C-Z₁₄ and Z₁ is selected from D, Y, F, I, N, V, H, L, A, T, S, and P; Z₂ is selected from D, Y, L, F, I, N, A, V, H, T, and S; Z₄ is selected from G and W; Z₆ is selected from E, D, V, and P; Z₇ is selected from W, L, F, V, G, M, I, and Y; Z₈ is selected from E, D, P, and Q; Z₉ is selected from E, D, Y, V, F, W, P, L, and Q; Z₁₀ is selected from S, D, Y, T, I, F, V, N, A, P, L, and H; Z₁₁ is selected from I, Y, F, V, L, T, N, S, D, A, and H; Z₁₂ is selected from F, D, Y, L, I, V, A, N, T, P, S, and H; and Z₁₄ is selected from D, Y, N, F, I, P, V, A, T, H, L and S.

7. The isolated polypeptide or polypeptide complex of claim 6, wherein Z₁ is selected from D, Y, F, I, and N; Z₂ is selected from D, Y, L, F, I, and N; Z₄ is selected from G and W; Z₆ is selected from E and D; Z₇ is selected from W, L, F, and V; Z₈ is selected from E and D; Z₉ is selected from E, D, Y, and V; Z₁₀ is selected from S, D, Y, T, and I; Z₁₁ is selected from I, Y, F, V, L, and T; Z₁₂ is selected from F, D, Y, L, I, V, A, and N; and Z₁₄ is selected from D, Y, N, F, I, and P.

8. The isolated polypeptide or polypeptide complex of claim 7, wherein Z₁ is selected from D, Y, and F; Z₂ is selected from D, Y, L, and F; Z₄ is selected from G and W; Z₆ is selected from E and D; Z₇ is selected from W, L, and F; Z₈ is selected from E and D; Z₉ is selected from E and D; Z₁₀ is selected from S, D, and Y; Z₁₁ is selected from I, Y, and F; Z₁₂ is selected from F, D, Y, and L; and Z₁₄ is selected from D, Y, and N.

9. An isolated polypeptide or polypeptide complex comprising an anti-CD3 binding domain that is linked to a peptide that impairs binding of the anti-CD3 binding domain to CD3 wherein the peptide comprises an amino acid sequence according to U₁-U₂-C-U₄-P-U₆-U₇-U₈-U₉-U₁₀-U₁₁-U₁₂-C-U₁₄ and U₁ is selected from D, Y, F, I, N, V, H, L, A, T, S, and P; U₂ is selected from D, Y, L, F, I, N, A, V, H, T, and S; U₄ is selected from G and W; U₆ is selected from E, D, V, and P; U₇ is selected from W, L, F, V, G, M, I, and Y; U₈ is selected from E, D, P, and Q; U₉ is selected from E, D, Y, V, F, W, P, L, and Q; U₁₀ is selected from S, D, Y, T, I, F, V, N, A, P, L, and H; U₁₁ is selected from I, Y, F, V, L, T, N, S, D, A, and H; U₁₂ is selected from F, D, Y, L, I, V, A, N, T, P, S, G, and H; and U₁₄ is selected from D, Y, N, F, I, P, V, A, T, H, L, M, and S.

10. The isolated polypeptide or polypeptide complex of claim 9, wherein U₁ is selected from D, Y, F, I, V, and N; U₂ is selected from D, Y, L, F, I, and N; U₄ is selected from G and W; U₆ is selected from E and D; U₇ is selected from W, L, F, G, and V; U₈ is selected from E and D; U₉ is selected from E, D, Y, and V; U₁₀ is selected from S, D, Y, T, and I; U₁₁ is selected from I, Y, F, V, L, and T; U₁₂ is selected from F, D, Y, L, I, V, A, G, and N; and U₁₄ is selected from D, Y, N, F, I, M, and P.

11. The isolated polypeptide or polypeptide complex of claim 10, wherein U₁ is selected from D, Y, V, and F; U₂ is selected from D, Y, L, and F; U₄ is selected from G and W; U₆ is selected from E and D; U₇ is selected from W, L, G, and F; U₈ is selected from E and D; U₉ is selected from E and D; U₁₀ is selected from S, D, T, and Y; U₁₁ is selected from I, Y, V, L, and F; U₁₂ is selected from F, D, Y, G, A, and L; and U₁₄ is selected from D, Y, M, and N.

12. The isolated polypeptide or polypeptide complex of claim 9, wherein the peptide comprises an amino acid sequence of Table 17.

13. The isolated polypeptide or polypeptide complex of claim 9, wherein the peptide comprises an amino acid sequence according to any one of SEQ ID NOs: 44-55.

14. The isolated polypeptide or polypeptide complex of claim 11, wherein the peptide comprises the amino acid sequence of SEQ ID NO: 2.

15. The isolated polypeptide or polypeptide complex of claim 11, wherein the peptide comprises the amino acid sequence of SEQ ID NO: 54.

16. The isolated polypeptide or polypeptide complex of claim 9, wherein the peptide is connected to the anti-CD3 binding domain in a configuration according to Formula I: A₁-L₁-P₁ wherein A₁ comprises the anti-CD3 binding domain; L₁ comprises a cleavable linker that is a substrate for a tumor specific protease; and P₁ comprises the peptide that impairs binding of the anti-CD3 binding domain to CD3.

17. The isolated polypeptide or polypeptide complex of claim 16, wherein P₁ is connected N-terminal to the cleavable linker and A₁ is connected C-terminal to the cleavable linker.

18. The isolated polypeptide or polypeptide complex of claim 16, wherein P₁ is connected C-terminal to the cleavable linker and A₁ is connected N-terminal to the cleavable linker.

19. The isolated polypeptide or polypeptide complex of claim 16, wherein P₁ is bound to A₁ through ionic interactions, electrostatic interactions, hydrophobic interactions, Pi-stacking interactions, or H-bonding interactions, or a combination thereof.

20. The isolated polypeptide or polypeptide complex of claim 16, wherein A₁ is further linked to a tumor antigen binding domain (A₂).

21. The isolated polypeptide or polypeptide complex of claim 16, wherein the polypeptide or polypeptide complex is according to Formula Ia: P₂-L₂-A₂-A₁-L₁-P₁ wherein P₂ comprises a peptide that impairs binding of A₂ to a tumor antigen; and L₂ comprises a second cleavable linker that connects A₂ to P₂ and is a substrate for a tumor specific protease.

22. The isolated polypeptide or polypeptide complex of claim 21, wherein A₁ comprises an antibody or antibody fragment.

23. The isolated polypeptide or polypeptide complex of claim 21, wherein A₁ comprises an antibody or antibody fragment that is human or humanized.

24. The isolated polypeptide or polypeptide complex of claim 22, wherein L₁ is bound to a N-terminus of the antibody or antibody fragment of A₁.

25. The isolated polypeptide or polypeptide complex of claim 22, wherein A₂ is bound to a N-terminus of the antibody or antibody fragment of A₁.

26. The isolated polypeptide or polypeptide complex of claim 22, wherein L₁ is bound to a C-terminus of the antibody or antibody fragment of A₁.

27. The isolated polypeptide or polypeptide complex of claim 22, wherein A₂ is bound to a C-terminus of the antibody or antibody fragment of A₁.

28. The isolated polypeptide or polypeptide complex of claim 22, wherein the antibody or antibody fragment of A₁ comprises a single chain variable fragment, a single domain antibody, or a Fab fragment.

29. The isolated polypeptide or polypeptide complex of claim 28, wherein A₁ is the single chain variable fragment (scFv).

30. The isolated polypeptide or polypeptide complex of claim 29, wherein the scFv comprises a scFv heavy chain polypeptide and a scFv light chain polypeptide.

31. The isolated polypeptide or polypeptide complex of claim 28, wherein A₁ is the single domain antibody.

32. The isolated polypeptide or polypeptide complex of claim 23, wherein the antibody or antibody fragment thereof comprises a single chain variable fragment (scFv), a heavy chain variable domain (VH domain), a light chain variable domain (VL domain), or a variable domain (VHH) of a camelid derived single domain antibody.

33. The isolated polypeptide or polypeptide complex of claim 32, wherein A₁ comprises an anti-CD3e single chain variable fragment.

34. The isolated polypeptide or polypeptide complex of claim 22, wherein A₁ comprises an anti-CD3e single chain variable fragment that has a KD binding of 1 μM or less to CD3 on CD3 expressing cells.

35. The isolated polypeptide or polypeptide complex of claim 22, wherein A₁ comprises a variable light chain and variable heavy chain each of which is capable of specifically binding to human CD3.

36. The isolated polypeptide or polypeptide complex of claim 30, wherein A₁ comprises the scFv comprising a scFv heavy chain variable domain and an scFv light chain variable domain comprising complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 3; HC-CDR2: SEQ ID NO: 4; HC-CDR3: SEQ ID NO: 5; and the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 6; LC-CDR2: SEQ ID NO: 7; and LC-CDR3: SEQ ID NO: 8.

37. The isolated polypeptide or polypeptide complex of claim 22, wherein A₁ comprises a heavy chain variable domain and a light chain variable domain comprising complementarity determining regions (CDRs): HC-CDR1, HC-CDR2, and HC-CDR3, wherein the HC-CDR1, the HC-CDR2, and the HC-CDR3 of the heavy chain variable domain comprise: HC-CDR1: SEQ ID NO: 3; HC-CDR2: SEQ ID NO: 4; HC-CDR3: SEQ ID NO: 5; and the light chain variable domain comprises complementarity determining regions (CDRs): LC-CDR1, LC-CDR2, and LC-CDR3, wherein the LC-CDR1, the LC-CDR2, and the LC-CDR3 of the light chain variable domain comprise: LC-CDR1: SEQ ID NO: 6; LC-CDR2: SEQ ID NO: 7; and LC-CDR3: SEQ ID NO: 8.

38. The isolated polypeptide or polypeptide complex of claim 16, wherein the polypeptide or polypeptide complex of formula I binds to an effector cell when L₁ is cleaved by the tumor specific protease.

39. The isolated polypeptide or polypeptide complex of claim 16, wherein the polypeptide or polypeptide complex of formula I binds to an effector cell when L₁ is cleaved by the tumor specific protease and A₁ binds to the effector cell.

40. The isolated polypeptide or polypeptide complex of claim 39, wherein the effector cell is a T cell.

41. The isolated polypeptide or polypeptide complex of claim 40, wherein A₁ binds to a polypeptide that is part of a TCR-CD3 complex on the effector cell.

42. The isolated polypeptide or polypeptide complex of claim 41, wherein the polypeptide that is part of the TCR-CD3 complex is human CD3ε.

43. The isolated polypeptide or polypeptide complex of claim 30, wherein A₂ comprises an antibody or antibody fragment.

44. The isolated polypeptide or polypeptide complex of claim 43, wherein the antibody or antibody fragment thereof of A₂ comprises a single chain variable fragment, a single domain antibody, Fab′, or a Fab.

45. The isolated polypeptide or polypeptide complex of claim 43, wherein the antibody or antibody fragment of A₂ comprises a single chain variable fragment (scFv), a heavy chain variable domain (VH domain), a light chain variable domain (VL domain), or a variable domain (VHH) of a camelid derived single domain antibody.

46. The isolated polypeptide or polypeptide complex of claim 43, wherein the antibody or antibody fragment of A₂ is humanized or human.

47. The isolated polypeptide or polypeptide complex of claim 44, wherein A₂ is the Fab or Fab′.

48. The isolated polypeptide or polypeptide complex of claim 47, wherein the Fab or Fab′ comprises (a) a Fab light chain polypeptide and (b) a Fab heavy chain polypeptide.

49. The isolated polypeptide or polypeptide complex of claim 48, wherein A₂ comprises an epidermal growth factor receptor (EGFR) binding domain.

50. The isolated polypeptide or polypeptide complex of claim 48, wherein A₂ comprises a carcinoembryonic antigen-related cell adhesion molecule CEACAM5 binding domain.

51. The isolated polypeptide or polypeptide complex of claim 48, wherein P₁ is further linked to a half-life extending moiety (H₁).

52. The isolated polypeptide or polypeptide complex of claim 51, wherein the half-life extending moiety is a single-domain antibody.

53. The isolated polypeptide or polypeptide complex of claim 51, wherein H₁ comprises a polymer.

54. The isolated polypeptide or polypeptide complex of claim 53, wherein the polymer is polyethylene glycol (PEG).

55. The isolated polypeptide or polypeptide complex of claim 51, wherein H₁ comprises albumin.

56. The isolated polypeptide or polypeptide complex of claim 51, wherein H₁ comprises an Fc domain.

57. The isolated polypeptide or polypeptide complex of claim 55, wherein the albumin is serum albumin.

58. The isolated polypeptide or polypeptide complex of claim 57, wherein the albumin is human serum albumin.

59. The isolated polypeptide or polypeptide complex of claim 51, wherein H₁ comprises a polypeptide, a ligand, or a small molecule.

60. The isolated polypeptide or polypeptide complex of claim 59, wherein the polypeptide, the ligand or the small molecule binds serum protein or a fragment thereof, a circulating immunoglobulin or a fragment thereof, or CD35/CR1.

61. The isolated polypeptide or polypeptide complex of claim 60, wherein the serum protein comprises a thyroxine-binding protein, a transthyretin, a 1-acid glycoprotein, a transferrin, transferrin receptor or a transferrin-binding portion thereof, a fibrinogen, or an albumin.

62. The isolated polypeptide or polypeptide complex of claim 60, wherein the circulating immunoglobulin comprises IgGl, IgG2, IgG3, IgG4, slgA, IgM or IgD.

63. The isolated polypeptide or polypeptide complex of claim 60, wherein the serum protein is albumin.

64. The isolated polypeptide or polypeptide complex of claim 59, wherein the polypeptide is an antibody.

65. The isolated polypeptide or polypeptide complex of claim 64, wherein the antibody comprises a single domain antibody, a single chain variable fragment, or a Fab.

66. The isolated polypeptide or polypeptide complex of claim 65, wherein the single domain antibody comprises a single domain antibody that binds to albumin.

67. The isolated polypeptide or polypeptide complex of claim 65, wherein the single domain antibody is a human or humanized antibody.

68. The isolated polypeptide or polypeptide complex of claim 65, wherein the single domain antibody is 645gH1gL1.

69. The isolated polypeptide or polypeptide complex of claim 65, wherein the single domain antibody is 645dsgH5gL4.

70. The isolated polypeptide or polypeptide complex of claim 65, wherein the single domain antibody is 23-13-A01-sc02.

71. The isolated polypeptide or polypeptide complex of claim 65, wherein the single domain antibody is A10m3 or a fragment thereof.

72. The isolated polypeptide or polypeptide complex of claim 65, wherein the single domain antibody is DOM7r-31.

73. The isolated polypeptide or polypeptide complex of claim 65, wherein the single domain antibody is DOM7h-11-15.

74. The isolated polypeptide or polypeptide complex of claim 65, wherein the single domain antibody is Alb-1, Alb-8, or Alb-23.

75. The isolated polypeptide or polypeptide complex of claim 65, wherein the single domain antibody is 10G or 10GE.

76. The isolated polypeptide or polypeptide complex of claim 65, wherein the single domain antibody is SA21.

77. The isolated polypeptide or polypeptide complex of claim 1, wherein the polypeptide or polypeptide complex comprises a modified amino acid, a non-natural amino acid, a modified non-natural amino acid, or a combination thereof.

78. The isolated polypeptide or polypeptide complex of claim 77, wherein the modified amino acid or modified non-natural amino acid comprises a post-translational modification.

79. The isolated polypeptide or polypeptide complex of claim 51, wherein H₁ comprises a linking moiety (L₃) that connects H₁ to P₁.

80. The isolated polypeptide or polypeptide complex of claim 79, wherein L₃ is a peptide sequence having at least 5 to no more than 50 amino acids.

81. The isolated polypeptide or polypeptide complex of claim 79, wherein L₃ is a peptide sequence having at least 10 to no more than 30 amino acids.

82. The isolated polypeptide or polypeptide complex of claim 79, wherein L₃ is a peptide sequence having at least 10 amino acids.

83. The isolated polypeptide or polypeptide complex of claim 79, wherein L₃ is a peptide sequence having at least 18 amino acids.

84. The isolated polypeptide or polypeptide complex of claim 79, wherein L₃ is a peptide sequence having at least 26 amino acids.

85. The isolated polypeptide or polypeptide complex of claim 79, wherein L₃ has a formula selected from the group consisting of (G₂S)ₙ, (GS)ₙ, (GSGGS)ₙ (SEQ ID NO: 917), (GGGS)ₙ (SEQ ID NO: 918), (GGGGS)ₙ (SEQ ID NO: 919), and (GSSGGS)ₙ (SEQ ID NO: 920), wherein n is an integer of at least 1.

86. The isolated polypeptide or polypeptide complex of claim 79, wherein L₃ comprises an amino acid sequence according to (GGGGSGGGS (SEQ ID NO: 921)).

87. The isolated polypeptide or polypeptide complex of claim 48, wherein the Fab light chain polypeptide of A₂ is bound to a C-terminus of the single chain variable fragment (scFv) of A₁.

88. The isolated polypeptide or polypeptide complex of claim 48, wherein the Fab heavy chain polypeptide of A₂ is bound to a C-terminus of the single chain variable fragment (scFv) A₁.

89. The isolated polypeptide or polypeptide complex of claim 48, wherein the Fab light chain polypeptide of A₂ is bound to a N-terminus of the single chain variable fragment (scFv) of A₁.

90. The isolated polypeptide or polypeptide complex of claim 48, wherein the Fab heavy chain polypeptide of A₂ is bound to a N-terminus of the single chain variable fragment (scFv) A₁.

91. The isolated polypeptide or polypeptide complex of claim 48, wherein the Fab heavy chain polypeptide of A₂ is bound to the scFv heavy chain polypeptide of A₁.

92. The isolated polypeptide or polypeptide complex of claim 48, wherein the Fab light chain polypeptide of A₂ is bound to the scFv heavy chain polypeptide of A₁.

93. The isolated polypeptide or polypeptide complex of claim 48, wherein the Fab heavy chain polypeptide of A₂ is bound to the scFv light chain polypeptide of A₁.

94. The isolated polypeptide or polypeptide complex of claim 48, wherein the Fab light chain polypeptide of A₂ is bound to the scFv light chain polypeptide of A₁.

95. The isolated polypeptide or polypeptide complex of claim 48, wherein the Fab heavy chain polypeptide of A₂ is bound to the scFv heavy chain polypeptide of A₁ and L₂ is bound to the Fab light chain polypeptide of A₂.

96. The isolated polypeptide or polypeptide complex of claim 48, wherein the Fab light chain polypeptide of A₂ is bound to the scFv heavy chain polypeptide of A₁ and L₂ is bound to the Fab heavy chain polypeptide of A₂.

97. The isolated polypeptide or polypeptide complex of claim 48, wherein the Fab heavy chain polypeptide of A₂ is bound to the scFv light chain polypeptide of A₁ and L₂ is bound to the Fab light chain polypeptide of A₂.

98. The isolated polypeptide or polypeptide complex of claim 48, wherein the Fab light chain polypeptide of A₂ is bound to the scFv light chain polypeptide of A₁ and L₂ is bound to the Fab heavy chain polypeptide of A₂.

99. The isolated polypeptide or polypeptide complex of claim 21, wherein P₂ impairs binding of A₂ to the tumor antigen.

100. The isolated polypeptide or polypeptide complex of claim 21, wherein P₂ is bound to A₂ through ionic interactions, electrostatic interactions, hydrophobic interactions, Pi-stacking interactions, or H-bonding interactions, or a combination thereof.

101. The isolated polypeptide or polypeptide complex of claim 21, wherein P₂ is bound to A₂ at or near an antigen binding site.

102. The isolated polypeptide or polypeptide complex of claim 21, wherein P₂ has less than 70% sequence homology to the tumor antigen.

103. The isolated polypeptide or polypeptide complex of claim 21, wherein P₂ comprises a peptide sequence of at least 10 amino acids in length.

104. The isolated polypeptide or polypeptide complex of claim 21, wherein P₂ comprises a peptide sequence of at least 10 amino acids in length and no more than 20 amino acids in length.

105. The isolated polypeptide or polypeptide complex of claim 21, wherein P₂ comprises a peptide sequence of at least 16 amino acids in length.

106. The isolated polypeptide or polypeptide complex of claim 21, wherein P₂ comprises a peptide sequence of no more than 40 amino acids in length.

107. The isolated polypeptide or polypeptide complex of claim 21, wherein P₂ comprises at least two cysteine amino acid residues.

108. The isolated polypeptide or polypeptide complex of claim 21, wherein P₂ comprises a cyclic peptide or a linear peptide.

109. The isolated polypeptide or polypeptide complex of claim 21, wherein P₂ comprises a cyclic peptide.

110. The isolated polypeptide or polypeptide complex of claim 21, wherein P₂ comprises a linear peptide.

111. The isolated polypeptide or polypeptide complex of claim 16, wherein L₁ is bound to a N-terminus of A₁.

112. The isolated polypeptide or polypeptide complex of claim 16, wherein L₁ is bound to a C-terminus of A₁.

113. The isolated polypeptide or polypeptide complex of claim 21, wherein L₂ is bound to a N-terminus of A₂.

114. The isolated polypeptide or polypeptide complex of claim 21, wherein L₂ is bound to a C-terminus of A₂.

115. The isolated polypeptide or polypeptide complex of claim 21, wherein L₁ or L₂ is a peptide sequence having at least 5 to no more than 50 amino acids.

116. The isolated polypeptide or polypeptide complex of claim 21, wherein L₁ or L₂ is a peptide sequence having at least 10 to no more than 30 amino acids.

117. The isolated polypeptide or polypeptide complex of claim 21, wherein L₁ or L₂ is a peptide sequence having at least 10 amino acids.

118. The isolated polypeptide or polypeptide complex of claim 21, wherein L₁ or L₂ is a peptide sequence having at least 18 amino acids.

119. The isolated polypeptide or polypeptide complex of claim 21, wherein L₁ or L₂ is a peptide sequence having at least 26 amino acids.

120. The isolated polypeptide or polypeptide complex of claim 21, wherein L₁ or L₂ has a formula comprising (G₂S)ₙ (SEQ ID NO: 922), wherein n is an integer from 1 to 3.

121. The isolated polypeptide or polypeptide complex of claim 21, wherein L₁ has a formula selected from the group consisting of (G₂S)ₙ, (GS)ₙ, (GSGGS)ₙ (SEQ ID NO: 917), (GGGS)ₙ (SEQ ID NO: 918), (GGGGS)ₙ (SEQ ID NO: 919), and (GSSGGS)ₙ (SEQ ID NO: 920), wherein n is an integer of at least 1.

122. The isolated polypeptide or polypeptide complex of claim 21, wherein P₁ becomes unbound from A₁ when L₁ is cleaved by the tumor specific protease thereby exposing A₁ to CD3.

123. The isolated polypeptide or polypeptide complex of claim 21, wherein P₂ becomes unbound from A₂ when L₂ is cleaved by the tumor specific protease thereby exposing A₂ to the tumor antigen.

124. The isolated polypeptide or polypeptide complex of claim 21, wherein the tumor specific protease is selected from the group consisting of metalloprotease, serine protease, cysteine protease, threonine protease, and aspartic protease.

125. The isolated polypeptide or polypeptide complex of claim 21, wherein L₁ or L₂ comprises a urokinase cleavable amino acid sequence, a matriptase cleavable amino acid sequence, a matrix metalloprotease cleavable amino acid sequence, or a legumain cleavable amino acid sequence.

126. The isolated polypeptide or polypeptide complex of claim 21, wherein L₁ or L₂ comprises an amino acid sequence selected from the group consisting of GGGGSLSGRSDNHGSSGT (SEQ ID NO: 923), GGGGSSGGSGGSGLSGRSDNHGSSGT (SEQ ID NO: 924), ASGRSDNH (SEQ ID NO: 925), LAGRSDNH (SEQ ID NO: 926), ISSGLASGRSDNH (SEQ ID NO: 927), ISSGLLAGRSDNH (SEQ ID NO: 928), LSGRSDNH (SEQ ID NO: 929), ISSGLLSGRSDNP (SEQ ID NO: 930), ISSGLLSGRSDNH (SEQ ID NO: 931), LSGRSDNHSPLGLAGS (SEQ ID NO: 932), SPLGLAGSLSGRSDNH (SEQ ID NO: 933), SPLGLSGRSDNH (SEQ ID NO: 934), LAGRSDNHSPLGLAGS (SEQ ID NO: 935), LSGRSDNHVPLSLKMG (SEQ ID NO: 936), and LSGRSDNHVPLSLSMG (SEQ ID NO: 937).

127. The isolated polypeptide or polypeptide complex of claim 21, wherein L₁ or L₂ comprises an amino acid sequence selected from the group consisting of ASGRSDNH (SEQ ID NO: 925), LAGRSDNH (SEQ ID NO: 926), ISSGLASGRSDNH (SEQ ID NO: 927), and ISSGLLAGRSDNH (SEQ ID NO: 928).

128. A pharmaceutical composition comprising: (i) the polypeptide or polypeptide complex of claim 1; and (ii) a pharmaceutically acceptable excipient.

129. An isolated recombinant nucleic acid molecule encoding the polypeptide or polypeptide complex of claim 1.


SEQUENCE LISTING (943 sequences)

SEQ ID NO: 1 - protein, 14 aa, Artificial Sequence
  Description of Artificial Sequence Synthetic peptide
GSQCLGPEWEVCPY

SEQ ID NO: 2 - protein, 14 aa, Artificial Sequence
  Description of Artificial Sequence Synthetic peptide
VYCGPEFDESVGCM

SEQ ID NO: 3 - protein, 8 aa, Artificial Sequence
  Description of Artificial Sequence Synthetic peptide
GFTFNKYA

SEQ ID NO: 4 - protein, 10 aa, Artificial Sequence
  Description of Artificial Sequence Synthetic peptide
IRSKYNNYAT

SEQ ID NO: 5 - protein, 16 aa, Artificial Sequence
  Description of Artificial Sequence Synthetic peptide
VRHGNFGNSYISYWAY

SEQ ID NO: 6 - protein, 9 aa, Artificial Sequence
  Description of Artificial Sequence Synthetic peptide
TGAVTSGNY

SEQ ID NO: 7 - protein, 3 aa, Artificial Sequence
  Description of Artificial Sequence Synthetic peptide
GTK

SEQ ID NO: 8 - protein, 9 aa, Artificial Sequence
  Description of Artificial Sequence Synthetic peptide
VLWYSNRWV

SEQ ID NO: 9 - protein, 249 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 10 - protein, 214 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 222 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 12 - protein, 115 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSS

SEQ ID NO: 13 - protein, 266 aa, Artificial Sequence
  Description of Artificial Sequence Synthetic polypeptide
QGQSGQLSCEGWAMNREQCRAGSSGGSGGSGGSGISSGLLSGRSDNHGSSGTDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 644 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGGSQCLGPEWEVCPYGGGGSGGGSGGISSGLLSGRSDNHGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 15 - protein, 266 aa, Artificial Sequence; identical to SEQ ID NO: 13
  Description of Artificial Sequence Synthetic polypeptide

SEQ ID NO: 16 - protein, 644 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGGGGSGGGSGGISSGLLSGRSDNHGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 17 - protein, 214 aa, Artificial Sequence
  Description of Artificial Sequence Synthetic polypeptide
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 476 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 19 - protein, 14 aa, Artificial Sequence
  Description of Artificial Sequence Synthetic peptide
ASQCLGPEWEVCPY

SEQ ID NO: 20 - protein, 14 aa, Artificial Sequence
  Description of Artificial Sequence Synthetic peptide
GAQCLGPEWEVCPY

SEQ ID NO: 21 - protein, 14 aa, Artificial Sequence
  Description of Artificial Sequence Synthetic peptide
GSACLGPEWEVCPY

SEQ ID NO: 22 - protein, 14 aa, Artificial Sequence
  Description of Artificial Sequence Synthetic peptide
GSQCAGPEWEVCPY

SEQ ID NO: 23 - protein, 14 aa, Artificial Sequence
  Description of Artificial Sequence Synthetic peptide
GSQCLAPEWEVCPY

SEQ ID NO: 24 - protein, 14 aa, Artificial Sequence
  Description of Artificial Sequence Synthetic peptide
GSQCLGAEWEVCPY

SEQ ID NO: 25 - protein, 14 aa, Artificial Sequence
  Description of Artificial Sequence Synthetic peptide
GSQCLGPAWEVCPY

SEQ ID NO: 26 - protein, 14 aa, Artificial Sequence
  Description of Artificial Sequence Synthetic peptide
GSQCLGPEAEVCPY

SEQ ID NO: 27 - protein, 14 aa, Artificial Sequence
  Description of Artificial Sequence Synthetic peptide
GSQCLGPEWAVCPY

SEQ ID NO: 28 - protein, 14 aa, Artificial Sequence
  Description of Artificial Sequence Synthetic peptide
GSQCLGPEWEACPY

SEQ ID NO: 29 - protein, 14 aa, Artificial Sequence
  Description of Artificial Sequence Synthetic peptide
GSQCLGPEWEVCAY

SEQ ID NO: 30 - protein, 14 aa, Artificial Sequence
  Description of Artificial Sequence Synthetic peptide
GSQCLGPEWEVCPA

SEQ ID NO: 31 - protein, 14 aa, Artificial Sequence
  Description of Artificial Sequence Synthetic peptide
GSQCLGPEWEVCPY

SEQ ID NO: 32 - protein, 14 aa, Artificial Sequence
  Description of Artificial Sequence Synthetic peptide
AYCGPEFDESVGCM

SEQ ID NO: 33 - protein, 14 aa, Artificial Sequence
  Description of Artificial Sequence Synthetic peptide
VACGPEFDESVGCM

SEQ ID NO: 34 - protein, 14 aa, Artificial Sequence
  Description of Artificial Sequence Synthetic peptide
VYCAPEFDESVGCM

SEQ ID NO: 35 - protein, 14 aa, Artificial Sequence
  Description of Artificial Sequence Synthetic peptide
VYCGAEFDESVGCM

SEQ ID NO: 36 - protein, 14 aa, Artificial Sequence
  Description of Artificial Sequence Synthetic peptide
VYCGPAFDESVGCM

SEQ ID NO: 37 - protein, 14 aa, Artificial Sequence
  Description of Artificial Sequence Synthetic peptide
VYCGPEADESVGCM

SEQ ID NO: 38 - protein, 14 aa, Artificial Sequence
  Description of Artificial Sequence Synthetic peptide
VYCGPEFAESVGCM

SEQ ID NO: 39 - protein, 14 aa, Artificial Sequence
  Description of Artificial Sequence Synthetic peptide
VYCGPEFDASVGCM

SEQ ID NO: 40 - protein, 14 aa, Artificial Sequence
  Description of Artificial Sequence Synthetic peptide
VYCGPEFDEAVGCM

SEQ ID NO: 41 - protein, 14 aa, Artificial Sequence
  Description of Artificial Sequence Synthetic peptide
VYCGPEFDESAGCM

SEQ ID NO: 42 - protein, 14 aa, Artificial Sequence
  Description of Artificial Sequence Synthetic peptide
VYCGPEFDESVACM

SEQ ID NO: 43 - protein, 14 aa, Artificial Sequence
  Description of Artificial Sequence Synthetic peptide
VYCGPEFDESVGCA

SEQ ID NO: 44 - protein, 14 aa, Artificial Sequence
  Description of Artificial Sequence Synthetic peptide
DDCWPDWEFDFACA

SEQ ID NO: 45 - protein, 14 aa, Artificial Sequence
  Description of Artificial Sequence Synthetic peptide
YICGLDFPDFLYCD

SEQ ID NO: 46 - protein, 14 aa, Artificial Sequence
  Description of Artificial Sequence Synthetic peptide
FDCWPDWEEYFVCD

SEQ ID NO: 47 - protein, 14 aa, Artificial Sequence
  Description of Artificial Sequence Synthetic peptide
YICWPDWEEYFDCD

SEQ ID NO: 48 - protein, 14 aa, Artificial Sequence
  Description of Artificial Sequence Synthetic peptide
NICWPDWEDDYFCF

SEQ ID NO: 49 - protein, 14 aa, Artificial Sequence
  Description of Artificial Sequence Synthetic peptide
NFCWPDWEYIYPCI

SEQ ID NO: 50 - protein, 14 aa, Artificial Sequence
  Description of Artificial Sequence Synthetic peptide
VDCWPDWEEDFLCI

SEQ ID NO: 51 - protein, 14 aa, Artificial Sequence
  Description of Artificial Sequence Synthetic peptide
HACWPDWEEYFPCN

SEQ ID NO: 52 - protein, 14 aa, Artificial Sequence
  Description of Artificial Sequence Synthetic peptide
YDCGPDVDESYVCV

SEQ ID NO: 53 - protein, 14 aa, Artificial Sequence
  Description of Artificial Sequence Synthetic peptide
IDCWPDWEDDTFCY

SEQ ID NO: 54 - protein, 14 aa, Artificial Sequence
  Description of Artificial Sequence Synthetic peptide
YLCGPDGDETLACY

SEQ ID NO: 55 - protein, 14 aa, Artificial Sequence
  Description of Artificial Sequence Synthetic peptide
VDCGPDGDESILCY

SEQ ID NO: 56 - protein, 14 aa, Artificial Sequence
  Description of Artificial Sequence Synthetic peptide
VYCGPEFDESVGCM

SEQ ID NO: 57 - protein, 14 aa, Artificial Sequence
  Description of Artificial Sequence Synthetic peptide
DDCWPDWEFDFACA

SEQ ID NO: 58 - protein, 14 aa, Artificial Sequence
  Description of Artificial Sequence Synthetic peptide
YICGLDFPDFLYCD

SEQ ID NO: 59 - protein, 14 aa, Artificial Sequence
  Description of Artificial Sequence Synthetic peptide
FDCWPDWEEYFVCD

SEQ ID NO: 60 - protein, 14 aa, Artificial Sequence
  Description of Artificial Sequence Synthetic peptide
YICWPDWEEYFDCD

SEQ ID NO: 61 - protein, 14 aa, Artificial Sequence
  Description of Artificial Sequence Synthetic peptide
NICWPDWEDDYFCF

SEQ ID NO: 62 - protein, 14 aa, Artificial Sequence
  Description of Artificial Sequence Synthetic peptide
NFCWPDWEYIYPCI

SEQ ID NO: 63 - protein, 14 aa, Artificial Sequence
  Description of Artificial Sequence Synthetic peptide
VDCWPDWEEDFLCI

SEQ ID NO: 64 - protein, 14 aa, Artificial Sequence
  Description of Artificial Sequence Synthetic peptide
HACWPDWEEYFPCN

SEQ ID NO: 65 - protein, 14 aa, Artificial Sequence
  Description of Artificial Sequence Synthetic peptide
YDCGPDVDESYVCV

SEQ ID NO: 66 - protein, 14 aa, Artificial Sequence
  Description of Artificial Sequence Synthetic peptide
IDCWPDWEDDTFCY

SEQ ID NO: 67 - protein, 14 aa, Artificial Sequence
  Description of Artificial Sequence Synthetic peptide
YLCGPDGDETLACY

SEQ ID NO: 68 - protein, 14 aa, Artificial Sequence
  Description of Artificial Sequence Synthetic peptide
VDCGPDGDESILCY

SEQ ID NO: 69 - protein, 14 aa, Artificial Sequence
  Description of Artificial Sequence Synthetic peptide
FICWPDWEEDYFCA

SEQ ID NO: 70 - protein, 14 aa, Artificial Sequence
  Description of Artificial Sequence Synthetic peptide
GDCWPDWEWDFYCD

SEQ ID NO: 71 - protein, 14 aa, Artificial Sequence
  Description of Artificial Sequence Synthetic peptide
YLCWPDWEYIDLCD

SEQ ID NO: 72 - protein, 14 aa, Artificial Sequence
  Description of Artificial Sequence Synthetic peptide
SFCWPDWEEYFDCD

SEQ ID NO: 73 - protein, 14 aa, Artificial Sequence
  Description of Artificial Sequence Synthetic peptide
DDCWPDWEEYASCD

SEQ ID NO: 74 - protein, 14 aa, Artificial Sequence
  Description of Artificial Sequence Synthetic peptide
NLCWPDWEYPFFCD

SEQ ID NO: 75 - protein, 14 aa, Artificial Sequence
  Description of Artificial Sequence Synthetic peptide
FDCWPDWEESFVCD

SEQ ID NO: 76 - protein, 14 aa, Artificial Sequence
  Description of Artificial Sequence Synthetic peptide
DICGPDGDETIICD

SEQ ID NO: 77 - protein, 14 aa, Artificial Sequence
  Description of Artificial Sequence Synthetic peptide
DDCWPDWEYYAVCD

SEQ ID NO: 78 - protein, 14 aa, Artificial Sequence
  Description of Artificial Sequence Synthetic peptide
YDCWPDWEEYSNCD

SEQ ID NO: 79 - protein, 14 aa, Artificial Sequence
  Description of Artificial Sequence Synthetic peptide
INCWPDWEDYFFCD

SEQ ID NO: 80 - protein, 14 aa, Artificial Sequence
  Description of Artificial Sequence Synthetic peptide
NICWPDWEDDTFCF

SEQ ID NO: 81 - protein, 14 aa, Artificial Sequence
  Description of Artificial Sequence Synthetic peptide
NICWPDWEPNSFCF

SEQ ID NO: 82 - protein, 14 aa, Artificial Sequence
  Description of Artificial Sequence Synthetic peptide
YDCGPEMDESIDCF

SEQ ID NO: 83 - protein, 14 aa, Artificial Sequence
  Description of Artificial Sequence Synthetic peptide
DFCWPDWEFPFICH

SEQ ID NO: 84 - protein, 14 aa, Artificial Sequence
  Description of Artificial Sequence Synthetic peptide
DFCGPEMDESITCI

SEQ ID NO: 85 - protein, 14 aa, Artificial Sequence
  Description of Artificial Sequence Synthetic peptide
YDCGPEFDESTVCI

SEQ ID NO: 86 - protein, 14 aa, Artificial Sequence
  Description of Artificial Sequence Synthetic peptide
HDCWPDWEWDIFCI

SEQ ID NO: 87 - protein, 14 aa, Artificial Sequence
  Description of Artificial Sequence Synthetic peptide
HACWPDWEEYNPCN

SEQ ID NO: 88 - protein, 14 aa, Artificial Sequence
  Description of Artificial Sequence Synthetic peptide
DVCWPDWEWDFFCN

SEQ ID NO: 89 - protein, 14 aa, Artificial Sequence
  Description of Artificial Sequence Synthetic peptide
NYCWPDWEYYIPCN

SEQ ID NO: 90 - protein, 14 aa, Artificial Sequence
  Description of Artificial Sequence Synthetic peptide
IICWPDWEFIDYCN

SEQ ID NO: 91 - protein, 14 aa, Artificial Sequence
  Description of Artificial Sequence Synthetic peptide
SLCWPDWEYDIACP

SEQ ID NO: 92 - protein, 14 aa, Artificial Sequence
  Description of Artificial Sequence Synthetic peptide
DLCGPELDESIFCP

SEQ ID NO: 93 - protein, 14 aa, Artificial Sequence
  Description of Artificial Sequence Synthetic peptide
TNCWPDWEWVLPCP

SEQ ID NO: 94 - protein, 14 aa, Artificial Sequence
  Description of Artificial Sequence Synthetic peptide
IECWPDWEPNYFCP

SEQ ID NO: 95 - protein, 14 aa, Artificial Sequence
  Description of Artificial Sequence Synthetic peptide
IFCWPDWEDYVDCP

SEQ ID NO: 96 - protein, 14 aa, Artificial Sequence
  Description of Artificial Sequence Synthetic peptide
IDCWPDWEYDFFCP

SEQ ID NO: 97 - protein, 14 aa, Artificial Sequence
  Description of Artificial Sequence Synthetic peptide
LFCWPDWEDSFFCP

SEQ ID NO: 98 - protein, 14 aa, Artificial Sequence
  Description of Artificial Sequence Synthetic peptide
VDCWPDWEDYADCT

SEQ ID NO: 99 - protein, 14 aa, Artificial Sequence
  Description of Artificial Sequence Synthetic peptide
VICWPDWEQYFPCV

SEQ ID NO: 100 - protein, 14 aa, Artificial Sequence
  Description of Artificial Sequence Synthetic peptide
IECWPDWEPIYPCY

SEQ ID NO: 101 - protein, 14 aa, Artificial Sequence
  Description of Artificial Sequence Synthetic peptide
ITCWPDWEVYFPCY

SEQ ID NO: 102 - protein, 14 aa, Artificial Sequence
  Description of Artificial Sequence Synthetic peptide
IDCWPDWEYIHPCY

SEQ ID NO: 103 - protein, 14 aa, Artificial Sequence
  Description of Artificial Sequence Synthetic peptide
IDCWPDWEYINPCY

SEQ ID NO: 104 - protein, 14 aa, Artificial Sequence
  Description of Artificial Sequence Synthetic peptide
ADCWPDWEEAFPCY

SEQ ID NO: 105 - protein, 14 aa, Artificial Sequence
  Description of Artificial Sequence Synthetic peptide
IDCWPDWEYIYPCY

SEQ ID NO: 106 - protein, 14 aa, Artificial Sequence
  Description of Artificial Sequence Synthetic peptide
NICWPDWEDDNFCF

SEQ ID NO: 107 - protein, 14 aa, Artificial Sequence
  Description of Artificial Sequence Synthetic peptide
YDCWPDWEYVDACY

SEQ ID NO: 108 - protein, 14 aa, Artificial Sequence
  Description of Artificial Sequence Synthetic peptide
FYCGPDGDESYVCD

SEQ ID NO: 109 - protein, 14 aa, Artificial Sequence
  Description of Artificial Sequence Synthetic peptide
DICWPDWEYINICS

SEQ ID NO: 110 - protein, 14 aa, Artificial Sequence
  Description of Artificial Sequence Synthetic peptide
FVCWPDWEDFNFCD

SEQ ID NO: 111 - protein, 14 aa, Artificial Sequence
  Description of Artificial Sequence Synthetic peptide
FACWPDWEDYVACD

SEQ ID NO: 112 - protein, 14 aa, Artificial Sequence
  Description of Artificial Sequence Synthetic peptide
DNCWPDWEYDFFCV

SEQ ID NO: 113 - protein, 14 aa, Artificial Sequence
  Description of Artificial Sequence Synthetic peptide
YDCWPDWEEYNDCA

SEQ ID NO: 114 - protein, 14 aa, Artificial Sequence
  Description of Artificial Sequence Synthetic peptide
DDCGPDGDETIICV

SEQ ID NO: 115 - protein, 14 aa, Artificial Sequence
  Description of Artificial Sequence Synthetic peptide
FPCWPDWEEYAICD

SEQ ID NO: 116 - protein, 14 aa, Artificial Sequence
  Description of Artificial Sequence Synthetic peptide
PDCGPDGDESLFCT

SEQ ID NO: 117 - protein, 14 aa, Artificial Sequence
  Description of Artificial Sequence Synthetic peptide
DNCWPDWEYDYFCV

SEQ ID NO: 118 - protein, 14 aa, Artificial Sequence
  Description of Artificial Sequence Synthetic peptide
IFCWPDWEEFYDCY

SEQ ID NO: 119 - protein, 14 aa, Artificial Sequence
  Description of Artificial Sequence Synthetic peptide
ADCWPDWEEYFPCN

SEQ ID NO: 120 - protein, 14 aa, Artificial Sequence
  Description of Artificial Sequence Synthetic peptide
HTCWPDWEDDIFCN

SEQ ID NO: 121 - protein, 14 aa, Artificial Sequence
  Description of Artificial Sequence Synthetic peptide
FACWPDWEEAFLCL

SEQ ID NO: 122 - protein, 14 aa, Artificial Sequence
  Description of Artificial Sequence Synthetic peptide
YDCGPELDESIACD

SEQ ID NO: 123 - protein, 14 aa, Artificial Sequence
  Description of Artificial Sequence Synthetic peptide
NSCWPDWEYDIICD

SEQ ID NO: 124 - protein, 14 aa, Artificial Sequence
  Description of Artificial Sequence Synthetic peptide
FACWPDWEEVAPCY

SEQ ID NO: 125 - protein, 14 aa, Artificial Sequence
  Description of Artificial Sequence Synthetic peptide
LDCGPDGDETLTCY

SEQ ID NO: 126 - protein, 14 aa, Artificial Sequence
  Description of Artificial Sequence Synthetic peptide
VLCWPDWEEFYDCP

SEQ ID NO: 127 - protein, 14 aa, Artificial Sequence
  Description of Artificial Sequence Synthetic peptide
HACWPVWEEYFPCN

SEQ ID NO: 128 - protein, 14 aa, Artificial Sequence
  Description of Artificial Sequence Synthetic peptide
NECWPNGEPTFPCT

SEQ ID NO: 129 - protein, 14 aa, Artificial Sequence
  Description of Artificial Sequence Synthetic peptide
LTCGPDGDETLYCD

SEQ ID NO: 130 - protein, 14 aa, Artificial Sequence
  Description of Artificial Sequence Synthetic peptide
YDCGPEYDESVPCI

SEQ ID NO: 131 - protein, 14 aa, Artificial Sequence
  Description of Artificial Sequence Synthetic peptide
IECWPDWEPNSFCD

SEQ ID NO: 132 - protein, 14 aa, Artificial Sequence
  Description of Artificial Sequence Synthetic peptide
YDCGPELDESIHCY

SEQ ID NO: 133 - protein, 14 aa, Artificial Sequence
  Description of Artificial Sequence Synthetic peptide
IYCGPEFDESTICN

SEQ ID NO: 134 - protein, 14 aa, Artificial Sequence
  Description of Artificial Sequence Synthetic peptide
IYCGPEVEEAYLCY

SEQ ID NO: 135 - protein, 14 aa, Artificial Sequence
  Description of Artificial Sequence Synthetic peptide
FDCGPDGDETVYCD

SEQ ID NO: 136 - protein, 14 aa, Artificial Sequence
  Description of Artificial Sequence Synthetic peptide
IDCGPDGDETISCY

SEQ ID NO: 137 - protein, 14 aa, Artificial Sequence
  Description of Artificial Sequence Synthetic peptide
NYCGPEFDESSTCL

SEQ ID NO: 138 - protein, 14 aa, Artificial Sequence
  Description of Artificial Sequence Synthetic peptide
YDCGPDGDESYFCD

SEQ ID NO: 139 - protein, 14 aa, Artificial Sequence
  Description of Artificial Sequence Synthetic peptide
NFCWPDWEYFNDCN

SEQ ID NO: 140 - protein, 14 aa, Artificial Sequence
  Description of Artificial Sequence Synthetic peptide
VLCWPDWEAFFDCD

SEQ ID NO: 141 - protein, 14 aa, Artificial Sequence
  Description of Artificial Sequence Synthetic peptide
IYCGPEWEWPVACN

SEQ ID NO: 142 - protein, 14 aa, Artificial Sequence
  Description of Artificial Sequence Synthetic peptide
VFCWPDWEDNFFCN

SEQ ID NO: 143 - protein, 14 aa, Artificial Sequence
  Description of Artificial Sequence Synthetic peptide
VVCWPDWETFFPCD

SEQ ID NO: 144 - protein, 14 aa, Artificial Sequence
  Description of Artificial Sequence Synthetic peptide
DNCGPDGDETYICN

SEQ ID NO: 145 - protein, 14 aa, Artificial Sequence
  Description of Artificial Sequence Synthetic peptide
DNCWPDWEYNFFCV

SEQ ID NO: 146 - protein, 14 aa, Artificial Sequence
  Description of Artificial Sequence Synthetic peptide
FYCGPEVEEDYLCI

SEQ ID NO: 147 - protein, 14 aa, Artificial Sequence
  Description of Artificial Sequence Synthetic peptide
DNCWPDWEYDIFCV

SEQ ID NO: 148 - protein, 14 aa, Artificial Sequence
  Description of Artificial Sequence Synthetic peptide
IDCGPEFDESIACP

SEQ ID NO: 149 - protein, 14 aa, Artificial Sequence
  Description of Artificial Sequence Synthetic peptide
YFCGPEVEEYTLCF

SEQ ID NO: 150 - protein, 14 aa, Artificial Sequence
  Description of Artificial Sequence Synthetic peptide
FYCGPEFDESAPCN

SEQ ID NO: 151 - protein, 14 aa, Artificial Sequence
  Description of Artificial Sequence Synthetic peptide
FDCGPEVEEYFYCA

SEQ ID NO: 152 - protein, 14 aa, Artificial Sequence
  Description of Artificial Sequence Synthetic peptide
DFCWPDWEDFFFCA

SEQ ID NO: 153 - protein, 14 aa, Artificial Sequence
  Description of Artificial Sequence Synthetic peptide
FFCGPDGDETLSCN

SEQ ID NO: 154 - protein, 14 aa, Artificial Sequence
  Description of Artificial Sequence Synthetic peptide
FICGPEFDESVACL

SEQ ID NO: 155 - protein, 14 aa, Artificial Sequence
  Description of Artificial Sequence Synthetic peptide
YDCGPEFDEAIGCY

SEQ ID NO: 156 - protein, 14 aa, Artificial Sequence
  Description of Artificial Sequence Synthetic peptide
YICWPDWEEYLYCP

SEQ ID NO: 157 - protein, 14 aa, Artificial Sequence
  Description of Artificial Sequence Synthetic peptide
FDCWPDWEEPTTCH

SEQ ID NO: 158 - protein, 14 aa, Artificial Sequence
  Description of Artificial Sequence Synthetic peptide
YDCWPDWEDFPICD

SEQ ID NO: 159 - protein, 14 aa, Artificial Sequence
  Description of Artificial Sequence Synthetic peptide
VVCWPDWEYIDDCS

SEQ ID NO: 160 - protein, 14 aa, Artificial Sequence
  Description of Artificial Sequence Synthetic peptide
INCWPDWEVISFCD

SEQ ID NO: 161 - protein, 14 aa, Artificial Sequence
  Description of Artificial Sequence Synthetic peptide
LSCWPDWEEVTPCL

SEQ ID NO: 162 - protein, 14 aa, Artificial Sequence
  Description of Artificial Sequence Synthetic peptide
FACWPDWEEVDICY

SEQ ID NO: 163 - protein, 14 aa, Artificial Sequence
  Description of Artificial Sequence Synthetic peptide
YDCGPEMDESIVCD

SEQ ID NO: 164 - protein, 14 aa, Artificial Sequence
  Description of Artificial Sequence Synthetic peptide
YDCWPDWEVFIVCD

SEQ ID NO: 165 - protein, 14 aa, Artificial Sequence
  Description of Artificial Sequence Synthetic peptide
DNCWPDWEHNFFCV

SEQ ID NO: 166 - protein, 14 aa, Artificial Sequence
  Description of Artificial Sequence Synthetic peptide
YDCGPDGDESIYCP

SEQ ID NO: 167 - protein, 14 aa, Artificial Sequence
  Description of Artificial Sequence Synthetic peptide
YDCGPEFEFPYYCF

SEQ ID NO: 168 - protein, 14 aa, Artificial Sequence
  Description of Artificial Sequence Synthetic peptide
ADCGPEYDESVPCV

SEQ ID NO: 169 - protein, 14 aa, Artificial Sequence
  Description of Artificial Sequence Synthetic peptide
FLCGPEVEEVHYCS

SEQ ID NO: 170 - protein, 14 aa, Artificial Sequence
  Description of Artificial Sequence Synthetic peptide
TDCWPDWEYITSCS

SEQ ID NO: 171 - protein, 14 aa, Artificial Sequence
  Description of Artificial Sequence Synthetic peptide
AFCGPELDESITCD

SEQ ID NO: 172 - protein, 14 aa, Artificial Sequence
  Description of Artificial Sequence Synthetic peptide
NDCWPDWEEYFSCY

SEQ ID NO: 173 - protein, 14 aa, Artificial Sequence
  Description of Artificial Sequence Synthetic peptide
FDCGPEWEIVTDCY

SEQ ID NO: 174 - protein, 14 aa, Artificial Sequence
  Description of Artificial Sequence Synthetic peptide
NLCGPEMDESIICP

SEQ ID NO: 175 - protein, 14 aa, Artificial Sequence
  Description of Artificial Sequence Synthetic peptide
DLCGPEMDESIYCD

SEQ ID NO: 176 - protein, 14 aa, Artificial Sequence
  Description of Artificial Sequence Synthetic peptide
FDCGPDGVEDYICD

SEQ ID NO: 177 - protein, 14 aa, Artificial Sequence
  Description of Artificial Sequence Synthetic peptide
YACWPDWEEDFACY

SEQ ID NO: 178 - protein, 14 aa, Artificial Sequence
  Description of Artificial Sequence Synthetic peptide
HDCGPEMDESIVCV

SEQ ID NO: 179 - protein, 14 aa, Artificial Sequence
  Description of Artificial Sequence Synthetic peptide
VFCGPEFEFIFLCA

SEQ ID NO: 180 - protein, 14 aa, Artificial Sequence
  Description of Artificial Sequence Synthetic peptide
YDCGPELDESILCD

SEQ ID NO: 181 - protein, 14 aa, Artificial Sequence
  Description of Artificial Sequence Synthetic peptide
SVCWPDWEEFYSCD

SEQ ID NO: 182 - protein, 14 aa, Artificial Sequence
  Description of Artificial Sequence Synthetic peptide
PYCGPDGDETAICT

SEQ ID NO: 183 - protein, 14 aa, Artificial Sequence
  Description of Artificial Sequence Synthetic peptide
DDCGPELEWYYPCY

SEQ ID NO: 184 - protein, 14 aa, Artificial Sequence
  Description of Artificial Sequence Synthetic peptide
FICGPEFDESLPCN

SEQ ID NO: 185 - protein, 14 aa, Artificial Sequence
  Description of Artificial Sequence Synthetic peptide
IDCGPEFDESLPCD

SEQ ID NO: 186 - protein, 14 aa, Artificial Sequence
  Description of Artificial Sequence Synthetic peptide
FLCGPEFEEDAPCY

SEQ ID NO: 187 - protein, 14 aa, Artificial Sequence
  Description of Artificial Sequence Synthetic peptide
IFCGPDGDETHICH

SEQ ID NO: 188 - protein, 14 aa, Artificial Sequence
  Description of Artificial Sequence Synthetic peptide
VFCWPDWEYIDFCN

SEQ ID NO: 189 - protein, 14 aa, Artificial Sequence
  Description of Artificial Sequence Synthetic peptide
IFCGPEYDESLHCI

SEQ ID NO: 190 - protein, 14 aa, Artificial Sequence
  Description of Artificial Sequence Synthetic peptide
HLCWPDWEWYVDCP

SEQ ID NO: 191 - protein, 14 aa, Artificial Sequence
  Description of Artificial Sequence Synthetic peptide
FICGPEMDESIACN

SEQ ID NO: 192 - protein, 14 aa, Artificial Sequence
  Description of Artificial Sequence Synthetic peptide
IFCGPEVEMIFLCN

SEQ ID NO: 193 - protein, 14 aa, Artificial Sequence
  Description of Artificial Sequence Synthetic peptide
YDCGPEWEFPVDCI

SEQ ID NO: 194 - protein, 14 aa, Artificial Sequence
  Description of Artificial Sequence Synthetic peptide
NLCGPELDESITCF

SEQ ID NO: 195 - protein, 14 aa, Artificial Sequence
  Description of Artificial Sequence Synthetic peptide
FYCGPEVEDFYFCY

SEQ ID NO: 196 - protein, 14 aa, Artificial Sequence
  Description of Artificial Sequence Synthetic peptide
DYCGPEFDESLICN

SEQ ID NO: 197 - protein, 14 aa, Artificial Sequence
  Description of Artificial Sequence Synthetic peptide
DYCGPEFDESLPCD

SEQ ID NO: 198 - protein, 14 aa, Artificial Sequence
  Description of Artificial Sequence Synthetic peptide
AICGPELDESIACP

SEQ ID NO: 199 - protein, 14 aa, Artificial Sequence
  Description of Artificial Sequence Synthetic peptide
VICGPEVEDYNLCY

SEQ ID NO: 200 - protein, 14 aa, Artificial Sequence
  Description of Artificial Sequence Synthetic peptide
HTCWPDWEDYTVCP

SEQ ID NO: 201 - protein, 14 aa, Artificial Sequence
  Description of Artificial Sequence Synthetic peptide
SDCWPDWEYFYDCN

SEQ ID NO: 202 - protein, 14 aa, Artificial Sequence
  Description of Artificial Sequence Synthetic peptide
VFCGPDGDETVHCD

SEQ ID NO: 203 - protein, 14 aa, Artificial Sequence
  Description of Artificial Sequence Synthetic peptide
DYCGPEYDESVHCI

SEQ ID NO: 204 - protein, 14 aa, Artificial Sequence
  Description of Artificial Sequence Synthetic peptide
ADCGPDGDESIICH

SEQ ID NO: 205 - protein, 14 aa, Artificial Sequence
  Description of Artificial Sequence Synthetic peptide
FYCGPELDESLTCV

SEQ ID NO: 206 - protein, 14 aa, Artificial Sequence
  Description of Artificial Sequence Synthetic peptide
ILCGPEVEEDYYCY

SEQ ID NO: 207 - protein, 14 aa, Artificial Sequence
  Description of Artificial Sequence Synthetic peptide
HLCWPDWEEYHSCD

SEQ ID NO: 208 - protein, 14 aa, Artificial Sequence
  Description of Artificial Sequence Synthetic peptide
IFCWPDWEDYNFCT

SEQ ID NO: 209 - protein, 14 aa, Artificial Sequence
  Description of Artificial Sequence Synthetic peptide
IVCGPDGDETLICH

SEQ ID NO: 210 - protein, 14 aa, Artificial Sequence
  Description of Artificial Sequence Synthetic peptide
ADCWPDWEWDYTCD

SEQ ID NO: 211 - protein, 14 aa, Artificial Sequence
  Description of Artificial Sequence Synthetic peptide
ITCGPEFDESTTCN

SEQ ID NO: 212 - protein, 14 aa, Artificial Sequence
  Description of Artificial Sequence Synthetic peptide
YHCWPDWEEYTSCD

SEQ ID NO: 213 - protein, 14 aa, Artificial Sequence
  Description of Artificial Sequence Synthetic peptide
NYCGPEVEEYALCT

SEQ ID NO: 214 - protein, 14 aa, Artificial Sequence
  Description of Artificial Sequence Synthetic peptide
FICGPEMDESIHCD

SEQ ID NO: 215 - protein, 14 aa, Artificial Sequence
  Description of Artificial Sequence Synthetic peptide
DNCWPDWEEFAVCP

SEQ ID NO: 216 - protein, 14 aa, Artificial Sequence
  Description of Artificial Sequence Synthetic peptide
YDCGPELDETVVCD

SEQ ID NO: 217 - protein, 14 aa, Artificial Sequence
  Description of Artificial Sequence Synthetic peptide
YDCGPEFDESIACY

SEQ ID NO: 218 - protein, 14 aa, Artificial Sequence
  Description of Artificial Sequence Synthetic peptide
IDCWPDWEYTVHCD

SEQ ID NO: 219 - protein, 14 aa, Artificial Sequence
  Description of Artificial Sequence Synthetic peptide
DDCGPELDESIICI

SEQ ID NO: 220 - protein, 14 aa, Artificial Sequence
  Description of Artificial Sequence Synthetic peptide
VYCGPEYDESSFCF

SEQ ID NO: 221 - protein, 14 aa, Artificial Sequence
  Description of Artificial Sequence Synthetic peptide
FNCWPDWEDPYFCV

SEQ ID NO: 222 - protein, 14 aa, Artificial Sequence
  Description of Artificial Sequence Synthetic peptide
YDCGPEYDESSYCS

SEQ ID NO: 223 - protein, 14 aa, Artificial Sequence
  Description of Artificial Sequence Synthetic peptide
VACWPDWEYTDSCF

SEQ ID NO: 224 - protein, 14 aa, Artificial Sequence
  Description of Artificial Sequence Synthetic peptide
TDCGPEFDESVACY

SEQ ID NO: 225 - protein, 14 aa, Artificial Sequence
  Description of Artificial Sequence Synthetic peptide
TDCWPDWEFYADCD

SEQ ID NO: 226 - protein, 14 aa, Artificial Sequence
  Description of Artificial Sequence Synthetic peptide
YDCGPELDESVICH

SEQ ID NO: 227 - protein, 14 aa, Artificial Sequence
  Description of Artificial Sequence Synthetic peptide
SDCGPDGDESIICT

SEQ ID NO: 228 - protein, 14 aa, Artificial Sequence
  Description of Artificial Sequence Synthetic peptide
YYCGPEFDESIDCD

SEQ ID NO: 229 - protein, 14 aa, Artificial Sequence
  Description of Artificial Sequence Synthetic peptide
FFCGPEIDESIACV

SEQ ID NO: 230 - protein, 14 aa, Artificial Sequence
  Description of Artificial Sequence Synthetic peptide
DYCGPEFDESTFCD

SEQ ID NO: 231 - protein, 14 aa, Artificial Sequence
  Description of Artificial Sequence Synthetic peptide
YDCGPEWEWPIDCV

SEQ ID NO: 232 - protein, 14 aa, Artificial Sequence
  Description of Artificial Sequence Synthetic peptide
FYCGPEIELFSFCY

SEQ ID NO: 233 - protein, 14 aa, Artificial Sequence
  Description of Artificial Sequence Synthetic peptide
YYCGPEVDESITCP

SEQ ID NO: 234 - protein, 14 aa, Artificial Sequence
  Description of Artificial Sequence Synthetic peptide
ILCGPEFDESINCN

SEQ ID NO: 235 - protein, 14 aa, Artificial Sequence
  Description of Artificial Sequence Synthetic peptide
VVCGPAMGQHYLCD

SEQ ID NO: 236 - protein, 14 aa, Artificial Sequence
  Description of Artificial Sequence Synthetic peptide
VVCGTKMGEHYLCS

SEQ ID NO: 237 - protein, 14 aa, Artificial Sequence
  Description of Artificial Sequence Synthetic peptide
YDCWPDWEYVYACY

SEQ ID NO: 238 - protein, 14 aa, Artificial Sequence
  Description of Artificial Sequence Synthetic peptide
DLCGPELDESVNCD

SEQ ID NO: 239 - protein, 14 aa, Artificial Sequence
  Description of Artificial Sequence Synthetic peptide
YYCGPEFDESTVCY

SEQ ID NO: 240 - protein, 14 aa, Artificial Sequence
  Description of Artificial Sequence Synthetic peptide
LDCWPDWEWPYSCN

SEQ ID NO: 241 - protein, 14 aa, Artificial Sequence
  Description of Artificial Sequence Synthetic peptide
FICWPDWEDDFFCY

SEQ ID NO: 242 - protein, 14 aa, Artificial Sequence
  Description of Artificial Sequence Synthetic peptide
DLCGPEVEWYFFCN

SEQ ID NO: 243 - protein, 14 aa, Artificial Sequence
  Description of Artificial Sequence Synthetic peptide
YDCGPELDESIVCF

SEQ ID NO: 244 - protein, 14 aa, Artificial Sequence
  Description of Artificial Sequence Synthetic peptide
LNCWPVWEDDVFCY

SEQ ID NO: 245 - protein, 14 aa, Artificial Sequence
  Description of Artificial Sequence Synthetic peptide
FNCWPDWEDPNFCV

SEQ ID NO: 246 - protein, 14 aa, Artificial Sequence
  Description of Artificial Sequence Synthetic peptide
VICWPDWEDDYFCP

SEQ ID NO: 247 - protein, 14 aa, Artificial Sequence
  Description of Artificial Sequence Synthetic peptide
FLCGPEFDESSVCY

SEQ ID NO: 248 - protein, 14 aa, Artificial Sequence
  Description of Artificial Sequence Synthetic peptide
YDCGPELDESIFCY

SEQ ID NO: 249 - protein, 14 aa, Artificial Sequence
  Description of Artificial Sequence Synthetic peptide
HLCGPDGDESFTCF

SEQ ID NO: 250 - protein, 14 aa, Artificial Sequence
  Description of Artificial Sequence Synthetic peptide
YFCGPEMDESLYCI

SEQ ID NO: 251 - protein, 14 aa, Artificial Sequence
  Description of Artificial Sequence Synthetic peptide
YYCGPEVEEYANCY

SEQ ID NO: 252 - protein, 14 aa, Artificial Sequence
  Description of Artificial Sequence Synthetic peptide
NTCGPEFDESTACY

SEQ ID NO: 253 - protein, 14 aa, Artificial Sequence
  Description of Artificial Sequence Synthetic peptide
IDCWPDWEEAFNCY

SEQ ID NO: 254 - protein, 14 aa, Artificial Sequence
  Description of Artificial Sequence Synthetic peptide
AYCGPELEEFFLCT

SEQ ID NO: 255 - protein, 14 aa, Artificial Sequence
  Description of Artificial Sequence Synthetic peptide
IYCGPEVEEVHHCY

SEQ ID NO: 256 - protein, 14 aa, Artificial Sequence
  Description of Artificial Sequence Synthetic peptide
FFCGPEFDESVACD

SEQ ID NO: 257 - protein, 14 aa, Artificial Sequence
  Description of Artificial Sequence Synthetic peptide
YDCGPELDETIICA

SEQ ID NO: 258 - protein, 14 aa, Artificial Sequence
  Description of Artificial Sequence Synthetic peptide
ILCGPEWEYPLDCS

SEQ ID NO: 259 - protein, 14 aa, Artificial Sequence
  Description of Artificial Sequence Synthetic peptide
FICGPEFDESTTCN

SEQ ID NO: 260 - protein, 14 aa, Artificial Sequence
  Description of Artificial Sequence Synthetic peptide
FYCGPELDESVSCD

SEQ ID NO: 261 - protein, 14 aa, Artificial Sequence
  Description of Artificial Sequence Synthetic peptide
HLCGPELDESVTCF

SEQ ID NO: 262 - protein, 14 aa, Artificial Sequence
  Description of Artificial Sequence Synthetic peptide
LICGPEVEDYSLCH

SEQ ID NO: 263 - protein, 14 aa, Artificial Sequence
  Description of Artificial Sequence Synthetic peptide
YFCGPEMDESVYCD

SEQ ID NO: 264 - protein, 14 aa, Artificial Sequence
  Description of Artificial Sequence Synthetic peptide
HYCGPEMDESIYCI

SEQ ID NO: 265 - protein, 14 aa, Artificial Sequence
  Description of Artificial Sequence Synthetic peptide
FDCGPELDESINCD

SEQ ID NO: 266 - protein, 14 aa, Artificial Sequence
  Description of Artificial Sequence Synthetic peptide
YYCGPEVEEYIYCT

SEQ ID NO: 267 - protein, 14 aa, Artificial Sequence
  Description of Artificial Sequence Synthetic peptide
LACWPVREEINACI

SEQ ID NO: 268 - protein, 14 aa, Artificial Sequence
  Description of Artificial Sequence Synthetic peptide
IDCWPDWEDITFCD

SEQ ID NO: 269 - protein, 14 aa, Artificial Sequence
  Description of Artificial Sequence Synthetic peptide
IVCGPELDESITCP

SEQ ID NO: 270 - protein, 14 aa, Artificial Sequence
  Description of Artificial Sequence Synthetic peptide
FYCGPEFELPADCD

SEQ ID NO: 271 - protein, 14 aa, Artificial Sequence
  Description of Artificial Sequence Synthetic peptide
FDCGPEFDESNPCF

SEQ ID NO: 272 - protein, 14 aa, Artificial Sequence
  Description of Artificial Sequence Synthetic peptide
DACWPDWEEYSSCD

SEQ ID NO: 273 - protein, 14 aa, Artificial Sequence
  Description of Artificial Sequence Synthetic peptide
DHCWPDWEPNYFCV

SEQ ID NO: 274 - protein, 14 aa, Artificial Sequence
  Description of Artificial Sequence Synthetic peptide
DYCWPDWEINYICF

SEQ ID NO: 275 - protein, 14 aa, Artificial Sequence
  Description of Artificial Sequence Synthetic peptide
IYCWPDWEYVYACN

SEQ ID NO: 276 - protein, 14 aa, Artificial Sequence
  Description of Artificial Sequence Synthetic peptide
DFCGPEVEEDYLCD

SEQ ID NO: 277 - protein, 14 aa, Artificial Sequence
  Description of Artificial Sequence Synthetic peptide
HDCGPDGREDYDCA

SEQ ID NO: 278 - protein, 14 aa, Artificial Sequence
  Description of Artificial Sequence Synthetic peptide
LACWPDWEDDYFCV

SEQ ID NO: 279 - protein, 14 aa, Artificial Sequence
  Description of Artificial Sequence Synthetic peptide
DICWPDWEDYLPCV

SEQ ID NO: 280 - protein, 14 aa, Artificial Sequence
  Description of Artificial Sequence Synthetic peptide
ILCGPEIEVYALCP

SEQ ID NO: 281 - protein, 14 aa, Artificial Sequence
  Description of Artificial Sequence Synthetic peptide
IFCGPEWEFSVLCN

SEQ ID NO: 282 - protein, 14 aa, Artificial Sequence
  Description of Artificial Sequence Synthetic peptide
TYCGPEVEDFSLCV

SEQ ID NO: 283 - protein, 14 aa, Artificial Sequence
  Description of Artificial Sequence Synthetic peptide
FICGPEWEFVDACF

SEQ ID NO: 284 - protein, 14 aa, Artificial Sequence
  Description of Artificial Sequence Synthetic peptide
FACWPDWEEDSPCD

SEQ ID NO: 285 - protein, 14 aa, Artificial Sequence
  Description of Artificial Sequence Synthetic peptide
ILCGPEVEELIFCP

SEQ ID NO: 286 - protein, 14 aa, Artificial Sequence
  Description of Artificial Sequence Synthetic peptide
FYCGPEVEEYIYCY

SEQ ID NO: 287 - protein, 14 aa, Artificial Sequence
  Description of Artificial Sequence Synthetic peptide
DSCGPELDESIICD

SEQ ID NO: 288 - protein, 14 aa, Artificial Sequence
  Description of Artificial Sequence Synthetic peptide
FLCGPDGDETSVCD

SEQ ID NO: 289 - protein, 14 aa, Artificial Sequence
  Description of Artificial Sequence Synthetic peptide
FNCWPNGEPTYFCV

SEQ ID NO: 290 - protein, 14 aa, Artificial Sequence
  Description of Artificial Sequence Synthetic peptide
LACWPVWEYPVTCI

SEQ ID NO: 291 - protein, 14 aa, Artificial Sequence
  Description of Artificial Sequence Synthetic peptide
DYCGPEVEEDVYCY

SEQ ID NO: 292 - protein, 14 aa, Artificial Sequence
  Description of Artificial Sequence Synthetic peptide
ITCWPDWEEYANCT

SEQ ID NO: 293 - protein, 14 aa, Artificial Sequence
  Description of Artificial Sequence Synthetic peptide
FFCGPDGDETYSCI

SEQ ID NO: 294 - protein, 14 aa, Artificial Sequence
  Description of Artificial Sequence Synthetic peptide
TDCWPDWEYATSCD

SEQ ID NO: 295 - protein, 14 aa, Artificial Sequence
  Description of Artificial Sequence Synthetic peptide
FNCGPDGYEDYLCD

SEQ ID NO: 296 - protein, 14 aa, Artificial Sequence
  Description of Artificial Sequence Synthetic peptide
YDCWPDWEVDFHCP

SEQ ID NO: 297 - protein, 14 aa, Artificial Sequence
  Description of Artificial Sequence Synthetic peptide
NICWPDWEDDSFCF

SEQ ID NO: 298 - protein, 14 aa, Artificial Sequence
  Description of Artificial Sequence Synthetic peptide
ATCGPEFDESIGCS

SEQ ID NO: 299 - protein, 14 aa, Artificial Sequence
  Description of Artificial Sequence Synthetic peptide
SYCGPEFDESTFCD

SEQ ID NO: 300 - protein, 14 aa, Artificial Sequence
  Description of Artificial Sequence Synthetic peptide
PICGPEYDESDVCA

SEQ ID NO: 301 - protein, 14 aa, Artificial Sequence
  Description of Artificial Sequence Synthetic peptide
YYCGPDGDEYNSCI

SEQ ID NO: 302 - protein, 14 aa, Artificial Sequence
  Description of Artificial Sequence Synthetic peptide
VDCWPDWEVFIACD

SEQ ID NO: 303 - protein, 14 aa, Artificial Sequence
  Description of Artificial Sequence Synthetic peptide
DLCGPEVEEVNLCL

SEQ ID NO: 304 - protein, 14 aa, Artificial Sequence
  Description of Artificial Sequence Synthetic peptide
FDCGPEMDESTTCF

SEQ ID NO: 305 - protein, 14 aa, Artificial Sequence
  Description of Artificial Sequence Synthetic peptide
NFCWPDWEPIYFCT

SEQ ID NO: 306 - protein, 14 aa, Artificial Sequence
  Description of Artificial Sequence Synthetic peptide
VDCGPDGDESFFCL

SEQ ID NO: 307 - protein, 14 aa, Artificial Sequence
  Description of Artificial Sequence Synthetic peptide
VDCGPDGDETAFCI

SEQ ID NO: 308 - protein, 14 aa, Artificial Sequence
  Description of Artificial Sequence Synthetic peptide
NICGPEMDESLVCI

SEQ ID NO: 309 - protein, 14 aa, Artificial Sequence
  Description of Artificial Sequence Synthetic peptide
IICGPEFDESFFCF

SEQ ID NO: 310 - protein, 14 aa, Artificial Sequence
  Description of Artificial Sequence Synthetic peptide
NFCGPEYDESISCI

SEQ ID NO: 311 - protein, 14 aa, Artificial Sequence
  Description of Artificial Sequence Synthetic peptide
HLCGPEIEEADICN

SEQ ID NO: 312 - protein, 14 aa, Artificial Sequence
  Description of Artificial Sequence Synthetic peptide
DYCGPEVEEDYLCD

SEQ ID NO: 313 - protein, 14 aa, Artificial Sequence
  Description of Artificial Sequence Synthetic peptide
DYCGPELDESINCD

SEQ ID NO: 314 - protein, 14 aa, Artificial Sequence
  Description of Artificial Sequence Synthetic peptide
FYCGPELDESLFCV

SEQ ID NO: 315 - protein, 14 aa, Artificial Sequence
  Description of Artificial Sequence Synthetic peptide
PDCGPEWEFYVTCN

SEQ ID NO: 316 - protein, 14 aa, Artificial Sequence
  Description of Artificial Sequence Synthetic peptide
FDCGPEFEYIYACT

SEQ ID NO: 317 - protein, 14 aa, Artificial Sequence
  Description of Artificial Sequence Synthetic peptide
DYCGPEFDESSICN

SEQ ID NO: 318 - protein, 14 aa, Artificial Sequence
  Description of Artificial Sequence Synthetic peptide
DFCGPEVEEYIFCF

SEQ ID NO: 319 - protein, 14 aa, Artificial Sequence
  Description of Artificial Sequence Synthetic peptide
PVCWPDWEYVSSCD

SEQ ID NO: 320 - protein, 14 aa, Artificial Sequence
  Description of Artificial Sequence Synthetic peptide
YICGPERDESNLCL

SEQ ID NO: 321 - protein, 14 aa, Artificial Sequence
  Description of Artificial Sequence Synthetic peptide
YDCGPELDESIVCD

SEQ ID NO: 322 - protein, 14 aa, Artificial Sequence
  Description of Artificial Sequence Synthetic peptide
HDCWPDWEDFYFCV

SEQ ID NO: 323 - protein, 14 aa, Artificial Sequence
  Description of Artificial Sequence Synthetic peptide
HYCGPEYDESIDCY

SEQ ID NO: 324 - protein, 14 aa, Artificial Sequence
  Description of Artificial Sequence Synthetic peptide
LFCGPEMPEDIFCN

SEQ ID NO: 325 - protein, 14 aa, Artificial Sequence
  Description of Artificial Sequence Synthetic peptide
HDCGPELEFHYACY

SEQ ID NO: 326 - protein, 14 aa, Artificial Sequence
  Description of Artificial Sequence Synthetic peptide
DFCGPELDESINCF

SEQ ID NO: 327 - protein, 14 aa, Artificial Sequence
  Description of Artificial Sequence Synthetic peptide
YFCGPELDESIACN

SEQ ID NO: 328 - protein, 14 aa, Artificial Sequence
  Description of Artificial Sequence Synthetic peptide
TDCWPDWEDDIICD

SEQ ID NO: 329 - protein, 14 aa, Artificial Sequence
  Description of Artificial Sequence Synthetic peptide
YDCGPELDESIYCF

SEQ ID NO: 330 - protein, 14 aa, Artificial Sequence
  Description of Artificial Sequence Synthetic peptide
YYCWPDWWEYVTCD

SEQ ID NO: 331 - protein, 14 aa, Artificial Sequence
  Description of Artificial Sequence Synthetic peptide
PICGPELEESYLCN

SEQ ID NO: 332 - protein, 14 aa, Artificial Sequence
  Description of Artificial Sequence Synthetic peptide
FDCGPEFDESIVCY

SEQ ID NO: 333 - protein, 14 aa, Artificial Sequence
  Description of Artificial Sequence Synthetic peptide
VLCGPDGIEFFDCP

SEQ ID NO: 334 - protein, 14 aa, Artificial Sequence
  Description of Artificial Sequence Synthetic peptide
AYCGPEYDESLTCV

SEQ ID NO: 335 - protein, 14 aa, Artificial Sequence
  Description of Artificial Sequence Synthetic peptide
DFCGPELDESIICA

SEQ ID NO: 336 - protein, 14 aa, Artificial Sequence
  Description of Artificial Sequence Synthetic peptide
YDCGPEFDESLDCN

SEQ ID NO: 337 - protein, 14 aa, Artificial Sequence
  Description of Artificial Sequence Synthetic peptide
AICGPEFDESVACD

SEQ ID NO: 338 - protein, 14 aa, Artificial Sequence
  Description of Artificial Sequence Synthetic peptide
LLCGPDGVEDFFCD

SEQ ID NO: 339 - protein, 14 aa, Artificial Sequence
  Description of Artificial Sequence Synthetic peptide
NFCGPELPEDIFCF

SEQ ID NO: 340 - protein, 14 aa, Artificial Sequence
  Description of Artificial Sequence Synthetic peptide
FYCGPEVEEVSLCN

SEQ ID NO: 341 - protein, 14 aa, Artificial Sequence
  Description of Artificial Sequence Synthetic peptide
YDCGPDGYEAFYCH

SEQ ID NO: 342 - protein, 14 aa, Artificial Sequence
  Description of Artificial Sequence Synthetic peptide
NFCGPEIEFDYLCD

SEQ ID NO: 343 - protein, 14 aa, Artificial Sequence
  Description of Artificial Sequence Synthetic peptide
DYCGPDGVEDFICN

SEQ ID NO: 344 - protein, 14 aa, Artificial Sequence
  Description of Artificial Sequence Synthetic peptide
TDCWPDWEYIYSCS

SEQ ID NO: 345 - protein, 14 aa, Artificial Sequence
  Description of Artificial Sequence Synthetic peptide
FYCGPEFEEITNCI

SEQ ID NO: 346 - protein, 14 aa, Artificial Sequence
  Description of Artificial Sequence Synthetic peptide
FDCWPDWEESFFCH

SEQ ID NO: 347 - protein, 14 aa, Artificial Sequence
  Description of Artificial Sequence Synthetic peptide
DFCGPDGDESVFCP

SEQ ID NO: 348 - protein, 14 aa, Artificial Sequence
  Description of Artificial Sequence Synthetic peptide
HNCGPELDESLVCD

SEQ ID NO: 349 - protein, 14 aa, Artificial Sequence
  Description of Artificial Sequence Synthetic peptide
IYCGPDGAEDYTCD

SEQ ID NO: 350 - protein, 14 aa, Artificial Sequence
  Description of Artificial Sequence Synthetic peptide
FDCGPEFEFPVICF

SEQ ID NO: 351 - protein, 14 aa, Artificial Sequence
  Description of Artificial Sequence Synthetic peptide
YNCGPELDESVTCD

SEQ ID NO: 352 - protein, 14 aa, Artificial Sequence
  Description of Artificial Sequence Synthetic peptide
FDCGPELDESIHCA

SEQ ID NO: 353 - protein, 14 aa, Artificial Sequence
  Description of Artificial Sequence Synthetic peptide
DICGPEVEEYFLCF

SEQ ID NO: 354 - protein, 14 aa, Artificial Sequence
  Description of Artificial Sequence Synthetic peptide
FDCGPEVDESLTCF

SEQ ID NO: 355 - protein, 14 aa, Artificial Sequence
  Description of Artificial Sequence Synthetic peptide
FDCGPEIEEFHLCF

SEQ ID NO: 356 - protein, 14 aa, Artificial Sequence
  Description of Artificial Sequence Synthetic peptide
AYCGPELDESIICD

SEQ ID NO: 357 - protein, 14 aa, Artificial Sequence
  Description of Artificial Sequence Synthetic peptide
YNCGPELDESITCN

SEQ ID NO: 358 - protein, 14 aa, Artificial Sequence
  Description of Artificial Sequence Synthetic peptide
FDCWPDWEEPVDCL

SEQ ID NO: 359 - protein, 14 aa, Artificial Sequence
  Description of Artificial Sequence Synthetic peptide
DVCGPELDESVLCP

SEQ ID NO: 360 - protein, 14 aa, Artificial Sequence
  Description of Artificial Sequence Synthetic peptide
NYCGPELDESLPCP

SEQ ID NO: 361 - protein, 14 aa, Artificial Sequence
  Description of Artificial Sequence Synthetic peptide
YYCWPDWEYDIFCS

SEQ ID NO: 362 - protein, 14 aa, Artificial Sequence
  Description of Artificial Sequence Synthetic peptide
DDCGPEFDESTYCN

SEQ ID NO: 363 - protein, 14 aa, Artificial Sequence
  Description of Artificial Sequence Synthetic peptide
FYCGPEFEEVFHCY

SEQ ID NO: 364 - protein, 14 aa, Artificial Sequence
  Description of Artificial Sequence Synthetic peptide
TDCWPDWEEYFLCD

SEQ ID NO: 365 - protein, 14 aa, Artificial Sequence
  Description of Artificial Sequence Synthetic peptide
VYCGPEWEESYLCP

SEQ ID NO: 366 - protein, 14 aa, Artificial Sequence
  Description of Artificial Sequence Synthetic peptide
DDCGPNGYATFICY

SEQ ID NO: 367 - protein, 14 aa, Artificial Sequence
  Description of Artificial Sequence Synthetic peptide
FLCGPEIEDDTHCY

SEQ ID NO: 368 - protein, 14 aa, Artificial Sequence
  Description of Artificial Sequence Synthetic peptide
FACWPDWEETIPCH

SEQ ID NO: 369 - protein, 14 aa, Artificial Sequence
  Description of Artificial Sequence Synthetic peptide
VLCGPEFDESYNCY

SEQ ID NO: 370 - protein, 14 aa, Artificial Sequence
  Description of Artificial Sequence Synthetic peptide
YDCGPEFDESISCI

SEQ ID NO: 371 - protein, 14 aa, Artificial Sequence
  Description of Artificial Sequence Synthetic peptide
YDCGPEFDESVNCY

SEQ ID NO: 372 - protein, 14 aa, Artificial Sequence
  Description of Artificial Sequence Synthetic peptide
AICWPDWEEFVDCY

SEQ ID NO: 373 - protein, 14 aa, Artificial Sequence
  Description of Artificial Sequence Synthetic peptide
LTCWPVREEIFACD

SEQ ID NO: 374 - protein, 14 aa, Artificial Sequence
  Description of Artificial Sequence Synthetic peptide
VLCGPEFDESYYCN

SEQ ID NO: 375 - protein, 14 aa, Artificial Sequence
  Description of Artificial Sequence Synthetic peptide
NVCGPEYDESAPCN

SEQ ID NO: 376 - protein, 14 aa, Artificial Sequence
  Description of Artificial Sequence Synthetic peptide
HDCGPEFDESISCV

SEQ ID NO: 377 - protein, 14 aa, Artificial Sequence
  Description of Artificial Sequence Synthetic peptide
FDCGPELDETVDCN

SEQ ID NO: 378 - protein, 14 aa, Artificial Sequence
  Description of Artificial Sequence Synthetic peptide
YFCGPEVEEHFYCY

SEQ ID NO: 379 - protein, 14 aa, Artificial Sequence
  Description of Artificial Sequence Synthetic peptide
DYCGPELDESIICH

SEQ ID NO: 380 - protein, 14 aa, Artificial Sequence
  Description of Artificial Sequence Synthetic peptide
DDCGPEVPEDITCY

SEQ ID NO: 381 - protein, 14 aa, Artificial Sequence
  Description of Artificial Sequence Synthetic peptide
DNCGPELDESVVCD

SEQ ID NO: 382 - protein, 14 aa, Artificial Sequence
  Description of Artificial Sequence Synthetic peptide
VHCWPDWEPNYVCD

SEQ ID NO: 383 - protein, 14 aa, Artificial Sequence
  Description of Artificial Sequence Synthetic peptide
DYCGPELDESLFCL

SEQ ID NO: 384 - protein, 14 aa, Artificial Sequence
  Description of Artificial Sequence Synthetic peptide
DDCGPELDESVVCA

SEQ ID NO: 385 - protein, 14 aa, Artificial Sequence
  Description of Artificial Sequence Synthetic peptide
AACGPELDESIVCD

SEQ ID NO: 386 - protein, 14 aa, Artificial Sequence
  Description of Artificial Sequence Synthetic peptide
DFCGPEFEEINNCF

SEQ ID NO: 387 - protein, 14 aa, Artificial Sequence
  Description of Artificial Sequence Synthetic peptide
YYCGPEFDESNACY

SEQ ID NO: 388 - protein, 14 aa, Artificial Sequence
  Description of Artificial Sequence Synthetic peptide
VLCGPEFDESNSCY

SEQ ID NO: 389 - protein, 14 aa, Artificial Sequence
  Description of Artificial Sequence Synthetic peptide
YDCGPEFDESIDCD

SEQ ID NO: 390 - protein, 14 aa, Artificial Sequence
  Description of Artificial Sequence Synthetic peptide
DSCGPEFEFYYVCF

SEQ ID NO: 391 - protein, 14 aa, Artificial Sequence
  Description of Artificial Sequence Synthetic peptide
YICGPELDESLICH

SEQ ID NO: 392 - protein, 14 aa, Artificial Sequence
  Description of Artificial Sequence Synthetic peptide
FDCGPEVEEDYFCY

SEQ ID NO: 393 - protein, 14 aa, Artificial Sequence
  Description of Artificial Sequence Synthetic peptide
FDCGPEYDESLYCF

SEQ ID NO: 394 - protein, 14 aa, Artificial Sequence
  Description of Artificial Sequence Synthetic peptide
VLCGPDGDEYSFCH

SEQ ID NO: 395 - protein, 14 aa, Artificial Sequence
  Description of Artificial Sequence Synthetic peptide
IPCGPEMDESVVCN

SEQ ID NO: 396 - protein, 14 aa, Artificial Sequence
  Description of Artificial Sequence Synthetic peptide
IICGPDGYEDFTCD

SEQ ID NO: 397 - protein, 14 aa, Artificial Sequence
  Description of Artificial Sequence Synthetic peptide
IFCGPELDESIICY

SEQ ID NO: 398 - protein, 14 aa, Artificial Sequence
  Description of Artificial Sequence Synthetic peptide
VDCGPEFDESYDCY

SEQ ID NO: 399 - protein, 14 aa, Artificial Sequence
  Description of Artificial Sequence Synthetic peptide
LSCGPEMDESLYCD

SEQ ID NO: 400 - protein, 14 aa, Artificial Sequence
  Description of Artificial Sequence Synthetic peptide
YDCWPDWEYNIDCT

SEQ ID NO: 401 - protein, 14 aa, Artificial Sequence
  Description of Artificial Sequence Synthetic peptide
NHCGPDGDETIVCF

SEQ ID NO: 402 - protein, 14 aa, Artificial Sequence
  Description of Artificial Sequence Synthetic peptide
NFCGPELDESIPCH

SEQ ID NO: 403 - protein, 14 aa, Artificial Sequence
  Description of Artificial Sequence Synthetic peptide
FHCGPEIEEYALCD

SEQ ID NO: 404 - protein, 14 aa, Artificial Sequence
  Description of Artificial Sequence Synthetic peptide
VYCGPEVEDYNLCY

SEQ ID NO: 405 - protein, 14 aa, Artificial Sequence
  Description of Artificial Sequence Synthetic peptide
VYCGPDGDELANCY

SEQ ID NO: 406 - protein, 14 aa, Artificial Sequence
  Description of Artificial Sequence Synthetic peptide
LICGPVIAEDLPCN

SEQ ID NO: 407 - protein, 14 aa, Artificial Sequence
  Description of Artificial Sequence Synthetic peptide
DICGPEIPEDVSCD

SEQ ID NO: 408 - protein, 14 aa, Artificial Sequence
  Description of Artificial Sequence Synthetic peptide
IYCGPEWEEADYCD

SEQ ID NO: 409 - protein, 14 aa, Artificial Sequence
  Description of Artificial Sequence Synthetic peptide
IDCWPDWEDDSICY

SEQ ID NO: 410 - protein, 14 aa, Artificial Sequence
  Description of Artificial Sequence Synthetic peptide
VLCGPEVEDFTLCD

SEQ ID NO: 411 - protein, 14 aa, Artificial Sequence
  Description of Artificial Sequence Synthetic peptide
LYCGPVIEEIYYCY

SEQ ID NO: 412 - protein, 14 aa, Artificial Sequence
  Description of Artificial Sequence Synthetic peptide
FFCGPEFEVHSDCN

SEQ ID NO: 413 - protein, 14 aa, Artificial Sequence
  Description of Artificial Sequence Synthetic peptide
NDCGPEVELVSDCN

SEQ ID NO: 414 - protein, 14 aa, Artificial Sequence
  Description of Artificial Sequence Synthetic peptide
DLCGPELDESTVCD

SEQ ID NO: 415 - protein, 14 aa, Artificial Sequence
  Description of Artificial Sequence Synthetic peptide
IPCGPEVEDYNLCN

SEQ ID NO: 416 - protein, 14 aa, Artificial Sequence
  Description of Artificial Sequence Synthetic peptide
YYCGPELEWPVVCN

SEQ ID NO: 417 - protein, 14 aa, Artificial Sequence
  Description of Artificial Sequence Synthetic peptide
YDCGPELDESVICN

SEQ ID NO: 418 - protein, 14 aa, Artificial Sequence
  Description of Artificial Sequence Synthetic peptide
VYCGPDGDESFDCA

SEQ ID NO: 419 - protein, 14 aa, Artificial Sequence
  Description of Artificial Sequence Synthetic peptide
NDCGPEWEDTYFCL

SEQ ID NO: 420 - protein, 14 aa, Artificial Sequence
  Description of Artificial Sequence Synthetic peptide
PYCGPEMEELSNCS

SEQ ID NO: 421 - protein, 14 aa, Artificial Sequence
  Description of Artificial Sequence Synthetic peptide
DDCGPEFEVISDCY

SEQ ID NO: 422 - protein, 14 aa, Artificial Sequence
  Description of Artificial Sequence Synthetic peptide
DLCGPEFPEDVPCD

SEQ ID NO: 423 - protein, 14 aa, Artificial Sequence
  Description of Artificial Sequence Synthetic peptide
IYCGPEFDESFVCY

SEQ ID NO: 424 - protein, 14 aa, Artificial Sequence
  Description of Artificial Sequence Synthetic peptide
AYCGPEYEVFADCN

SEQ ID NO: 425 - protein, 14 aa, Artificial Sequence
  Description of Artificial Sequence Synthetic peptide
IDCGPEYDESVDCL

SEQ ID NO: 426 - protein, 14 aa, Artificial Sequence
  Description of Artificial Sequence Synthetic peptide
HICWPDWEEFHDCN

SEQ ID NO: 427 - protein, 14 aa, Artificial Sequence
  Description of Artificial Sequence Synthetic peptide
YDCGPELDETITCL

SEQ ID NO: 428 - protein, 14 aa, Artificial Sequence
  Description of Artificial Sequence Synthetic peptide
YLCGPELDETILCN

SEQ ID NO: 429 - protein, 14 aa, Artificial Sequence
  Description of Artificial Sequence Synthetic peptide
FFCGPEFEEAFLCF

SEQ ID NO: 430 - protein, 14 aa, Artificial Sequence
  Description of Artificial Sequence Synthetic peptide
ILCGPELDESFTCA

SEQ ID NO: 431 - protein, 14 aa, Artificial Sequence
  Description of Artificial Sequence Synthetic peptide
FHCGPEVELYTDCN

SEQ ID NO: 432 - protein, 14 aa, Artificial Sequence
  Description of Artificial Sequence Synthetic peptide
NLCGPEVEEYNFCY

SEQ ID NO: 433 - protein, 14 aa, Artificial Sequence
  Description of Artificial Sequence Synthetic peptide
FDCGPEVEETYYCF

SEQ ID NO: 434 - protein, 14 aa, Artificial Sequence
  Description of Artificial Sequence Synthetic peptide
VFCGPEFEEDHYCY

SEQ ID NO: 435 - protein, 14 aa, Artificial Sequence
  Description of Artificial Sequence Synthetic peptide
AICGPEWEVVADCN

SEQ ID NO: 436 - protein, 14 aa, Artificial Sequence
  Description of Artificial Sequence Synthetic peptide
FICWPDWEEDNYCN

SEQ ID NO: 437 - protein, 14 aa, Artificial Sequence
  Description of Artificial Sequence Synthetic peptide
IYCGPEFDESFICD

SEQ ID NO: 438 - protein, 14 aa, Artificial Sequence
  Description of Artificial Sequence Synthetic peptide
NLCGPEVEDVYDCH

SEQ ID NO: 439 - protein, 14 aa, Artificial Sequence
  Description of Artificial Sequence Synthetic peptide
HYCGPEVEEYHNCN

SEQ ID NO: 440 - protein, 14 aa, Artificial Sequence
  Description of Artificial Sequence Synthetic peptide
DICGPEYDESYSCT

SEQ ID NO: 441 - protein, 14 aa, Artificial Sequence
  Description of Artificial Sequence Synthetic peptide
DYCGPELDETLICA

SEQ ID NO: 442 - protein, 14 aa, Artificial Sequence
  Description of Artificial Sequence Synthetic peptide
IACGPEMPEDIDCY

SEQ ID NO: 443 - protein, 14 aa, Artificial Sequence
  Description of Artificial Sequence Synthetic peptide
IDCGPELDESIFCD

SEQ ID NO: 444 - protein, 14 aa, Artificial Sequence
  Description of Artificial Sequence Synthetic peptide
YFCGPDVEEDFACD

SEQ ID NO: 445 - protein, 14 aa, Artificial Sequence
  Description of Artificial Sequence Synthetic peptide
YNCGPEWEYAILCD

SEQ ID NO: 446 - protein, 14 aa, Artificial Sequence
  Description of Artificial Sequence Synthetic peptide
IYCGPEVEDYIVCN

SEQ ID NO: 447 - protein, 14 aa, Artificial Sequence
  Description of Artificial Sequence Synthetic peptide
YDCGPEIDESTPCA

SEQ ID NO: 448 - protein, 14 aa, Artificial Sequence
  Description of Artificial Sequence Synthetic peptide
DTCWPDWEHIYACD

SEQ ID NO: 449 - protein, 14 aa, Artificial Sequence
  Description of Artificial Sequence Synthetic peptide
DICGPEMDESVTCN

SEQ ID NO: 450 - protein, 14 aa, Artificial Sequence
  Description of Artificial Sequence Synthetic peptide
YDCWPDWERYFPCI

SEQ ID NO: 451 - protein, 14 aa, Artificial Sequence
  Description of Artificial Sequence Synthetic peptide
HLCGPELDESVACS

SEQ ID NO: 452 - protein, 14 aa, Artificial Sequence
  Description of Artificial Sequence Synthetic peptide
YDCGPDGDETTICA

SEQ ID NO: 453 - protein, 14 aa, Artificial Sequence
  Description of Artificial Sequence Synthetic peptide
YYCGPEYEDVLDCF

SEQ ID NO: 454 - protein, 14 aa, Artificial Sequence
  Description of Artificial Sequence Synthetic peptide
DFCGPEMDETISCD

SEQ ID NO: 455 - protein, 14 aa, Artificial Sequence
  Description of Artificial Sequence Synthetic peptide
ILCGPELDESLVCD

SEQ ID NO: 456 - protein, 14 aa, Artificial Sequence
  Description of Artificial Sequence Synthetic peptide
LICGPEWEVITNCD

SEQ ID NO: 457 - protein, 14 aa, Artificial Sequence
  Description of Artificial Sequence Synthetic peptide
YDCGPEFDEYFGCP

SEQ ID NO: 458 - protein, 14 aa, Artificial Sequence
  Description of Artificial Sequence Synthetic peptide
ALCGPEVEVYDVCV

SEQ ID NO: 459 - protein, 14 aa, Artificial Sequence
  Description of Artificial Sequence Synthetic peptide
YHCWPDWEDVNFCY

SEQ ID NO: 460 - protein, 14 aa, Artificial Sequence
  Description of Artificial Sequence Synthetic peptide
FLCGPMGGLTFYCY

SEQ ID NO: 461 - protein, 14 aa, Artificial Sequence
  Description of Artificial Sequence Synthetic peptide
IICGPEFDEYVGCF

SEQ ID NO: 462 - protein, 14 aa, Artificial Sequence
  Description of Artificial Sequence Synthetic peptide
FFCGPEMDESVHCF

SEQ ID NO: 463 - protein, 14 aa, Artificial Sequence
  Description of Artificial Sequence Synthetic peptide
AFCGPEFDESLFCA

SEQ ID NO: 464 - protein, 14 aa, Artificial Sequence
  Description of Artificial Sequence Synthetic peptide
NYCGPDGDETNICD

SEQ ID NO: 465 - protein, 14 aa, Artificial Sequence
  Description of Artificial Sequence Synthetic peptide
YLCGPEWEWVHNCL

SEQ ID NO: 466 - protein, 14 aa, Artificial Sequence
  Description of Artificial Sequence Synthetic peptide
ATCGPDGDESHICA

SEQ ID NO: 467 - protein, 14 aa, Artificial Sequence
  Description of Artificial Sequence Synthetic peptide
VYCGPEVEVLDYCD

SEQ ID NO: 468 - protein, 14 aa, Artificial Sequence
  Description of Artificial Sequence Synthetic peptide
IHCGPEWEFYTDCD

SEQ ID NO: 469 - protein, 14 aa, Artificial Sequence
  Description of Artificial Sequence Synthetic peptide
DDCGPELDETVACD

SEQ ID NO: 470 - protein, 14 aa, Artificial Sequence
  Description of Artificial Sequence Synthetic peptide
YLCGPEFDESIDCN

SEQ ID NO: 471 - protein, 14 aa, Artificial Sequence
  Description of Artificial Sequence Synthetic peptide
LLCGPEVEDVFACY

SEQ ID NO: 472 - protein, 14 aa, Artificial Sequence
  Description of Artificial Sequence Synthetic peptide
YDCGPELDESLTCD

SEQ ID NO: 473 - protein, 14 aa, Artificial Sequence
  Description of Artificial Sequence Synthetic peptide
FDCGPELDETVNCY

SEQ ID NO: 474 - protein, 14 aa, Artificial Sequence
  Description of Artificial Sequence Synthetic peptide
FACWPDWEEINDCH

SEQ ID NO: 475 - protein, 14 aa, Artificial Sequence
  Description of Artificial Sequence Synthetic peptide
YYCGPEYEEDIYCN

SEQ ID NO: 476 - protein, 14 aa, Artificial Sequence
  Description of Artificial Sequence Synthetic peptide
NVCGPEVEDYTFCY

SEQ ID NO: 477 - protein, 14 aa, Artificial Sequence
  Description of Artificial Sequence Synthetic peptide
AYCGPELEEYDFCT

SEQ ID NO: 478 - protein, 14 aa, Artificial Sequence
  Description of Artificial Sequence Synthetic peptide
FDCGPEIDESTICT

SEQ ID NO: 479 - protein, 14 aa, Artificial Sequence
  Description of Artificial Sequence Synthetic peptide
NLCGPELDETLVCA

SEQ ID NO: 480 - protein, 14 aa, Artificial Sequence
  Description of Artificial Sequence Synthetic peptide
YSCWPDWEEYLACN

SEQ ID NO: 481 - protein, 14 aa, Artificial Sequence
  Description of Artificial Sequence Synthetic peptide
GICGPEVEDYNYCD

SEQ ID NO: 482 - protein, 14 aa, Artificial Sequence
  Description of Artificial Sequence Synthetic peptide
FFCGPELDESVNCH

SEQ ID NO: 483 - protein, 14 aa, Artificial Sequence
  Description of Artificial Sequence Synthetic peptide
YYCGPEYEEDFYCF

SEQ ID NO: 484 - protein, 14 aa, Artificial Sequence
  Description of Artificial Sequence Synthetic peptide
LYCGPEYDESTDCY

SEQ ID NO: 485 - protein, 14 aa, Artificial Sequence
  Description of Artificial Sequence Synthetic peptide
DYCGPEVEEDFLCY

SEQ ID NO: 486 - protein, 14 aa, Artificial Sequence
  Description of Artificial Sequence Synthetic peptide
TLCGPEVELYIFCD

SEQ ID NO: 487 - protein, 14 aa, Artificial Sequence
  Description of Artificial Sequence Synthetic peptide
FYCGPEFEQIADCY

SEQ ID NO: 488 - protein, 14 aa, Artificial Sequence
  Description of Artificial Sequence Synthetic peptide
DDCGPEVEEYHLCD

SEQ ID NO: 489 - protein, 14 aa, Artificial Sequence
  Description of Artificial Sequence Synthetic peptide
DYCGPELEDVTLCH

SEQ ID NO: 490 - protein, 14 aa, Artificial Sequence
  Description of Artificial Sequence Synthetic peptide
VLCGPEVEDVNLCY

SEQ ID NO: 491 - protein, 14 aa, Artificial Sequence
  Description of Artificial Sequence Synthetic peptide
DICGPELDETIDCY

SEQ ID NO: 492 - protein, 14 aa, Artificial Sequence
  Description of Artificial Sequence Synthetic peptide
TYCGPEVEEDINCY

SEQ ID NO: 493 - protein, 14 aa, Artificial Sequence
  Description of Artificial Sequence Synthetic peptide
IVCWPDWEEYPNCD

SEQ ID NO: 494 - protein, 14 aa, Artificial Sequence
  Description of Artificial Sequence Synthetic peptide
SDCGPELDESIICT

SEQ ID NO: 495 - protein, 14 aa, Artificial Sequence
  Description of Artificial Sequence Synthetic peptide
YDCGPELPEDYDCN

SEQ ID NO: 496 - protein, 14 aa, Artificial Sequence
  Description of Artificial Sequence Synthetic peptide
NLCWPDWEEYYACD

SEQ ID NO: 497 - protein, 14 aa, Artificial Sequence
  Description of Artificial Sequence Synthetic peptide
DDCGPELDESLPCH

SEQ ID NO: 498 - protein, 14 aa, Artificial Sequence
  Description of Artificial Sequence Synthetic peptide
SLCGPDGQEDYTCF

SEQ ID NO: 499 - protein, 14 aa, Artificial Sequence
  Description of Artificial Sequence Synthetic peptide
LICWPDWEEYNFCT

SEQ ID NO: 500 - protein, 14 aa, Artificial Sequence
  Description of Artificial Sequence Synthetic peptide
FHCGPDGDETVPCI

SEQ ID NO: 501 - protein, 14 aa, Artificial Sequence
  Description of Artificial Sequence Synthetic peptide
FDCGPEWEWIYDCF

SEQ ID NO: 502 - protein, 14 aa, Artificial Sequence
  Description of Artificial Sequence Synthetic peptide
IYCGPEWDESLDCD

SEQ ID NO: 503 - protein, 14 aa, Artificial Sequence
  Description of Artificial Sequence Synthetic peptide
LICGPEIDESASCN

SEQ ID NO: 504 - protein, 14 aa, Artificial Sequence
  Description of Artificial Sequence Synthetic peptide
TSCWVDWEEFSDCI

SEQ ID NO: 505 - protein, 14 aa, Artificial Sequence
  Description of Artificial Sequence Synthetic peptide
YYCGPEVEEDYVCD

SEQ ID NO: 506 - protein, 14 aa, Artificial Sequence
  Description of Artificial Sequence Synthetic peptide
DDCGPEQEFIYACI

SEQ ID NO: 507 - protein, 14 aa, Artificial Sequence
  Description of Artificial Sequence Synthetic peptide
ADCWPDWEEYADCY

SEQ ID NO: 508 - protein, 14 aa, Artificial Sequence
  Description of Artificial Sequence Synthetic peptide
YYCGPEFDESIHCI

SEQ ID NO: 509 - protein, 14 aa, Artificial Sequence
  Description of Artificial Sequence Synthetic peptide
SECWPDWEPFFDCN

SEQ ID NO: 510 - protein, 14 aa, Artificial Sequence
  Description of Artificial Sequence Synthetic peptide
YHCGPEMDESLICT

SEQ ID NO: 511 - protein, 14 aa, Artificial Sequence
  Description of Artificial Sequence Synthetic peptide
SDCWPDWEDAYFCI

SEQ ID NO: 512 - protein, 14 aa, Artificial Sequence
  Description of Artificial Sequence Synthetic peptide
VDCGPEVEEYYHCD

SEQ ID NO: 513 - protein, 14 aa, Artificial Sequence
  Description of Artificial Sequence Synthetic peptide
DNCGPEYDESIACN

SEQ ID NO: 514 - protein, 14 aa, Artificial Sequence
  Description of Artificial Sequence Synthetic peptide
LYCGPVFEFYDYCY

SEQ ID NO: 515 - protein, 14 aa, Artificial Sequence
  Description of Artificial Sequence Synthetic peptide
YTCGPEMDESVTCI

SEQ ID NO: 516 - protein, 14 aa, Artificial Sequence
  Description of Artificial Sequence Synthetic peptide
NFCWPDWEVNSFCD

SEQ ID NO: 517 - protein, 14 aa, Artificial Sequence
  Description of Artificial Sequence Synthetic peptide
NACWPDWEYIDFCN

SEQ ID NO: 518 - protein, 14 aa, Artificial Sequence
  Description of Artificial Sequence Synthetic peptide
YNCGPEMDESIFCS

SEQ ID NO: 519 - protein, 14 aa, Artificial Sequence
  Description of Artificial Sequence Synthetic peptide
LDCGPELDESITCY

SEQ ID NO: 520 - protein, 14 aa, Artificial Sequence
  Description of Artificial Sequence Synthetic peptide
HYCGPEFDESINCD

SEQ ID NO: 521 - protein, 14 aa, Artificial Sequence
  Description of Artificial Sequence Synthetic peptide
IICGPELPEDYVCT

SEQ ID NO: 522 - protein, 14 aa, Artificial Sequence
  Description of Artificial Sequence Synthetic peptide
DICGPEFDESIDCS

SEQ ID NO: 523 - protein, 14 aa, Artificial Sequence
  Description of Artificial Sequence Synthetic peptide
PYCGPEFDESVLCF

SEQ ID NO: 524 - protein, 14 aa, Artificial Sequence
  Description of Artificial Sequence Synthetic peptide
VFCGPEFDESVDCY

SEQ ID NO: 525 - protein, 14 aa, Artificial Sequence
  Description of Artificial Sequence Synthetic peptide
YDCWPDWEEALPCA

SEQ ID NO: 526 - protein, 14 aa, Artificial Sequence
  Description of Artificial Sequence Synthetic peptide
DDCWPDWEDYVFCF

SEQ ID NO: 527 - protein, 14 aa, Artificial Sequence
  Description of Artificial Sequence Synthetic peptide
HFCGPEWELFSDCY

SEQ ID NO: 528 - protein, 14 aa, Artificial Sequence
  Description of Artificial Sequence Synthetic peptide
ITCWPDWEVNFPCY

SEQ ID NO: 529 - protein, 14 aa, Artificial Sequence
  Description of Artificial Sequence Synthetic peptide
PDCGPELDESITCN

SEQ ID NO: 530 - protein, 14 aa, Artificial Sequence
  Description of Artificial Sequence Synthetic peptide
NLCWPDWEAFFPCY

SEQ ID NO: 531 - protein, 14 aa, Artificial Sequence
  Description of Artificial Sequence Synthetic peptide
FYCGPEFEYIRDCY

SEQ ID NO: 532 - protein, 14 aa, Artificial Sequence
  Description of Artificial Sequence Synthetic peptide
FFCGPEFDESIICD

SEQ ID NO: 533 - protein, 14 aa, Artificial Sequence
  Description of Artificial Sequence Synthetic peptide
VLCGPKGGPTYNCS

SEQ ID NO: 534 - protein, 14 aa, Artificial Sequence
  Description of Artificial Sequence Synthetic peptide
LACWPVWEEPGHCD

SEQ ID NO: 535 - protein, 14 aa, Artificial Sequence
  Description of Artificial Sequence Synthetic peptide
DYCGPEVEDVNDCY

SEQ ID NO: 536 - protein, 14 aa, Artificial Sequence
  Description of Artificial Sequence Synthetic peptide
DYCGPEFEEAHYCN

SEQ ID NO: 537 - protein, 14 aa, Artificial Sequence
  Description of Artificial Sequence Synthetic peptide
VLCGPELDETLTCI

SEQ ID NO: 538 - protein, 14 aa, Artificial Sequence
  Description of Artificial Sequence Synthetic peptide
YYCGPEIEDYNLCN

SEQ ID NO: 539 - protein, 14 aa, Artificial Sequence
  Description of Artificial Sequence Synthetic peptide
YICGPEVEEYYNCF

SEQ ID NO: 540 - protein, 14 aa, Artificial Sequence
  Description of Artificial Sequence Synthetic peptide
DICGPELDESIFCF

SEQ ID NO: 541 - protein, 14 aa, Artificial Sequence
  Description of Artificial Sequence Synthetic peptide
DICGPEVEEDYLCY

SEQ ID NO: 542 - protein, 14 aa, Artificial Sequence
  Description of Artificial Sequence Synthetic peptide
TLCGPEFEEDAPCI

SEQ ID NO: 543 - protein, 14 aa, Artificial Sequence
  Description of Artificial Sequence Synthetic peptide
NYCWPDWEYINSCV

SEQ ID NO: 544 - protein, 14 aa, Artificial Sequence
  Description of Artificial Sequence Synthetic peptide
NDCGPEVEEYYYCT

SEQ ID NO: 545 - protein, 14 aa, Artificial Sequence
  Description of Artificial Sequence Synthetic peptide
ITCGPEMDESIDCN

SEQ ID NO: 546 - protein, 14 aa, Artificial Sequence
  Description of Artificial Sequence Synthetic peptide
YYCGPEMAEDLICD

SEQ ID NO: 547 - protein, 14 aa, Artificial Sequence
  Description of Artificial Sequence Synthetic peptide
IDCGPELDESIVCT

SEQ ID NO: 548 - protein, 14 aa, Artificial Sequence
  Description of Artificial Sequence Synthetic peptide
HTCWPDWEWDVYCD

SEQ ID NO: 549 - protein, 14 aa, Artificial Sequence
  Description of Artificial Sequence Synthetic peptide
IHCGPEWELIDDCL

SEQ ID NO: 550 - protein, 14 aa, Artificial Sequence
  Description of Artificial Sequence Synthetic peptide
YTCGPELDESITCT

SEQ ID NO: 551 - protein, 14 aa, Artificial Sequence
  Description of Artificial Sequence Synthetic peptide
FHCGPEVEETVYCF

SEQ ID NO: 552 - protein, 14 aa, Artificial Sequence
  Description of Artificial Sequence Synthetic peptide
DYCGPELDESLICN

SEQ ID NO: 553 - protein, 14 aa, Artificial Sequence
  Description of Artificial Sequence Synthetic peptide
IYCGPEFDESDYCI

SEQ ID NO: 554 - protein, 14 aa, Artificial Sequence
  Description of Artificial Sequence Synthetic peptide
DLCGPEIEEDLVCT

SEQ ID NO: 555 - protein, 14 aa, Artificial Sequence
  Description of Artificial Sequence Synthetic peptide
NICGPELQEDIVCP

SEQ ID NO: 556 - protein, 14 aa, Artificial Sequence
  Description of Artificial Sequence Synthetic peptide
NNCGPEMDESITCY

SEQ ID NO: 557 - protein, 14 aa, Artificial Sequence
  Description of Artificial Sequence Synthetic peptide
HTCGPELDESIVCV

SEQ ID NO: 558 - protein, 14 aa, Artificial Sequence
  Description of Artificial Sequence Synthetic peptide
YYCGPEIEDILVCT

SEQ ID NO: 559 - protein, 14 aa, Artificial Sequence
  Description of Artificial Sequence Synthetic peptide
NTCGPEFEFVHLCP

SEQ ID NO: 560 - protein, 14 aa, Artificial Sequence
  Description of Artificial Sequence Synthetic peptide
DICGPEMDESTVCD

SEQ ID NO: 561 - protein, 14 aa, Artificial Sequence
  Description of Artificial Sequence Synthetic peptide
AICGPEVEIVNYCY

SEQ ID NO: 562 - protein, 14 aa, Artificial Sequence
  Description of Artificial Sequence Synthetic peptide
HLCGPEVEDPTACV

SEQ ID NO: 563 - protein, 14 aa, Artificial Sequence
  Description of Artificial Sequence Synthetic peptide
ADCGPELDESISCT

SEQ ID NO: 564 - protein, 14 aa, Artificial Sequence
  Description of Artificial Sequence Synthetic peptide
FDCGPELDESVICD

SEQ ID NO: 565 - protein, 14 aa, Artificial Sequence
  Description of Artificial Sequence Synthetic peptide
DVCGPEFDESIDCN

SEQ ID NO: 566 - protein, 14 aa, Artificial Sequence
  Description of Artificial Sequence Synthetic peptide
YLCGPEVEEISICF

SEQ ID NO: 567 - protein, 14 aa, Artificial Sequence
  Description of Artificial Sequence Synthetic peptide
SACGPEFDESLHCV

SEQ ID NO: 568 - protein, 14 aa, Artificial Sequence
  Description of Artificial Sequence Synthetic peptide
HLCWPDWEEDSACN

SEQ ID NO: 569 - protein, 14 aa, Artificial Sequence
  Description of Artificial Sequence Synthetic peptide
HTCWPDWEYDYDCF

SEQ ID NO: 570 - protein, 14 aa, Artificial Sequence
  Description of Artificial Sequence Synthetic peptide
YDCGPEWEEVALCN

SEQ ID NO: 571 - protein, 14 aa, Artificial Sequence
  Description of Artificial Sequence Synthetic peptide
TLCGPEIEEYIVCY

SEQ ID NO: 572 - protein, 14 aa, Artificial Sequence
  Description of Artificial Sequence Synthetic peptide
SYCGPEFDESIFCT

SEQ ID NO: 573 - protein, 14 aa, Artificial Sequence
  Description of Artificial Sequence Synthetic peptide
DICGPEFDESLHCY

SEQ ID NO: 574 - protein, 14 aa, Artificial Sequence
  Description of Artificial Sequence Synthetic peptide
LFCGPEFEEAYLCI

SEQ ID NO: 575 - protein, 14 aa, Artificial Sequence
  Description of Artificial Sequence Synthetic peptide
IFCGPEIEEDFVCT

SEQ ID NO: 576 - protein, 14 aa, Artificial Sequence
  Description of Artificial Sequence Synthetic peptide
DDCGPEWEYYVACV

SEQ ID NO: 577 - protein, 14 aa, Artificial Sequence
  Description of Artificial Sequence Synthetic peptide
DDCGPELDETTICY

SEQ ID NO: 578 - protein, 14 aa, Artificial Sequence
  Description of Artificial Sequence Synthetic peptide
ASCGPELDESIACD

SEQ ID NO: 579 - protein, 14 aa, Artificial Sequence
  Description of Artificial Sequence Synthetic peptide
YLCGPEVEDYDYCY

SEQ ID NO: 580 - protein, 14 aa, Artificial Sequence
  Description of Artificial Sequence Synthetic peptide
DLCGPEWEETIFCA

SEQ ID NO: 581 - protein, 14 aa, Artificial Sequence
  Description of Artificial Sequence Synthetic peptide
DFCGPDGEEFYICP

SEQ ID NO: 582 - protein, 14 aa, Artificial Sequence
  Description of Artificial Sequence Synthetic peptide
VYCGPEVEENILCH

SEQ ID NO: 583 - protein, 14 aa, Artificial Sequence
  Description of Artificial Sequence Synthetic peptide
DYCGPEVEENYFCF

SEQ ID NO: 584 - protein, 14 aa, Artificial Sequence
  Description of Artificial Sequence Synthetic peptide
NDCWPDWYEFLSCD

SEQ ID NO: 585 - protein, 14 aa, Artificial Sequence
  Description of Artificial Sequence Synthetic peptide
RDCWPDWEVPYFCD

SEQ ID NO: 586 - protein, 14 aa, Artificial Sequence
  Description of Artificial Sequence Synthetic peptide
NLCGPEVEEAVYCY

SEQ ID NO: 587 - protein, 14 aa, Artificial Sequence
  Description of Artificial Sequence Synthetic peptide
FDCWPDGELNYLCT

SEQ ID NO: 588 - protein, 14 aa, Artificial Sequence
  Description of Artificial Sequence Synthetic peptide
YYCGPEVEDVNLCI

SEQ ID NO: 589 - protein, 14 aa, Artificial Sequence
  Description of Artificial Sequence Synthetic peptide
DNCGPEYDESITCL

SEQ ID NO: 590 - protein, 14 aa, Artificial Sequence
  Description of Artificial Sequence Synthetic peptide
LNCWPDWEEDYSCN

SEQ ID NO: 591 - protein, 14 aa, Artificial Sequence
  Description of Artificial Sequence Synthetic peptide
PTCGPEVEELLSCN

SEQ ID NO: 592 - protein, 14 aa, Artificial Sequence
  Description of Artificial Sequence Synthetic peptide
DHCGPEVELIFYCH

SEQ ID NO: 593 - protein, 14 aa, Artificial Sequence
  Description of Artificial Sequence Synthetic peptide
FHCGPELDESIFCY

SEQ ID NO: 594 - protein, 14 aa, Artificial Sequence
  Description of Artificial Sequence Synthetic peptide
FDCGPELEETVVCP

SEQ ID NO: 595 - protein, 14 aa, Artificial Sequence
  Description of Artificial Sequence Synthetic peptide
DFCGPELDESLPCA

SEQ ID NO: 596 - protein, 14 aa, Artificial Sequence
  Description of Artificial Sequence Synthetic peptide
DSCGPELDESIYCD

SEQ ID NO: 597 - protein, 14 aa, Artificial Sequence
  Description of Artificial Sequence Synthetic peptide
ADCWPDWEEFLLCF

SEQ ID NO: 598 - protein, 14 aa, Artificial Sequence
  Description of Artificial Sequence Synthetic peptide
ILCGPEVEELDFCN

SEQ ID NO: 599 - protein, 14 aa, Artificial Sequence
  Description of Artificial Sequence Synthetic peptide
FFCGPEFEEIFLCY

SEQ ID NO: 600 - protein, 14 aa, Artificial Sequence
  Description of Artificial Sequence Synthetic peptide
YNCGPDGDESYDCH

SEQ ID NO: 601 - protein, 14 aa, Artificial Sequence
  Description of Artificial Sequence Synthetic peptide
DNCGPELDETITCF

SEQ ID NO: 602 - protein, 14 aa, Artificial Sequence
  Description of Artificial Sequence Synthetic peptide
HNCGPDGDEAFICN

SEQ ID NO: 603 - protein, 14 aa, Artificial Sequence
  Description of Artificial Sequence Synthetic peptide
DYCGPEVEEDLVCP

SEQ ID NO: 604 - protein, 14 aa, Artificial Sequence
  Description of Artificial Sequence Synthetic peptide
DYCGPELDESLNCF

SEQ ID NO: 605 - protein, 14 aa, Artificial Sequence
  Description of Artificial Sequence Synthetic peptide
DYCGPEFQEDFHCH

SEQ ID NO: 606 - protein, 14 aa, Artificial Sequence
  Description of Artificial Sequence Synthetic peptide
YDCGPEWEFTDDCI

SEQ ID NO: 607 - protein, 14 aa, Artificial Sequence
  Description of Artificial Sequence Synthetic peptide
DICGPEYEEDIICY

SEQ ID NO: 608 - protein, 14 aa, Artificial Sequence
  Description of Artificial Sequence Synthetic peptide
FDCGPELDETVPCP

SEQ ID NO: 609 - protein, 14 aa, Artificial Sequence
  Description of Artificial Sequence Synthetic peptide
NYCGPELDETSVCD

SEQ ID NO: 610 - protein, 14 aa, Artificial Sequence
  Description of Artificial Sequence Synthetic peptide
YYCGPEWEFSFDCD

SEQ ID NO: 611 - protein, 14 aa, Artificial Sequence
  Description of Artificial Sequence Synthetic peptide
YACGPELDESVTCD

SEQ ID NO: 612 - protein, 14 aa, Artificial Sequence
  Description of Artificial Sequence Synthetic peptide
FLCGPEVEQDYFCV

SEQ ID NO: 613 - protein, 14 aa, Artificial Sequence
  Description of Artificial Sequence Synthetic peptide
DNCGPEFDESVRCD

SEQ ID NO: 614 - protein, 14 aa, Artificial Sequence
  Description of Artificial Sequence Synthetic peptide
NDCGPDGIETVDCY

SEQ ID NO: 615 - protein, 14 aa, Artificial Sequence
  Description of Artificial Sequence Synthetic peptide
YFCGPEVEDYNDCF

SEQ ID NO: 616 - protein, 14 aa, Artificial Sequence
  Description of Artificial Sequence Synthetic peptide
NLCGPEFDESIFCY

SEQ ID NO: 617 - protein, 14 aa, Artificial Sequence
  Description of Artificial Sequence Synthetic peptide
IDCWPDWEEYIPCT

SEQ ID NO: 618 - protein, 14 aa, Artificial Sequence
  Description of Artificial Sequence Synthetic peptide
IYCGPDGDESFICA

SEQ ID NO: 619 - protein, 14 aa, Artificial Sequence
  Description of Artificial Sequence Synthetic peptide
DYCGPEFDESVVCY

SEQ ID NO: 620 - protein, 14 aa, Artificial Sequence
  Description of Artificial Sequence Synthetic peptide
VFCGPEWEDITDCD

SEQ ID NO: 621 - protein, 14 aa, Artificial Sequence
  Description of Artificial Sequence Synthetic peptide
LDCGPEVDETFTCH

SEQ ID NO: 622 - protein, 14 aa, Artificial Sequence
  Description of Artificial Sequence Synthetic peptide
DDCGPEYDESFACH

SEQ ID NO: 623 - protein, 14 aa, Artificial Sequence
  Description of Artificial Sequence Synthetic peptide
IYCGPEYQEDLPCN

SEQ ID NO: 624 - protein, 14 aa, Artificial Sequence
  Description of Artificial Sequence Synthetic peptide
LDCGPEVEEYNYCV

SEQ ID NO: 625 - protein, 14 aa, Artificial Sequence
  Description of Artificial Sequence Synthetic peptide
YVCGPEFDESSACN

SEQ ID NO: 626 - protein, 14 aa, Artificial Sequence
  Description of Artificial Sequence Synthetic peptide
TPCGPELEEAIGCY

SEQ ID NO: 627 - protein, 14 aa, Artificial Sequence
  Description of Artificial Sequence Synthetic peptide
YFCGPEFDESADCN

SEQ ID NO: 628 - protein, 14 aa, Artificial Sequence
  Description of Artificial Sequence Synthetic peptide
DDCGPEFEEDIICD

SEQ ID NO: 629 - protein, 14 aa, Artificial Sequence
  Description of Artificial Sequence Synthetic peptide
LYCGPVVEELNHCN

SEQ ID NO: 630 - protein, 14 aa, Artificial Sequence
  Description of Artificial Sequence Synthetic peptide
VICGPDGEELIACA

SEQ ID NO: 631 - protein, 14 aa, Artificial Sequence
  Description of Artificial Sequence Synthetic peptide
FACWPDWQETYVCN

SEQ ID NO: 632 - protein, 14 aa, Artificial Sequence
  Description of Artificial Sequence Synthetic peptide
YICGPEVEFLFFCN

SEQ ID NO: 633 - protein, 14 aa, Artificial Sequence
  Description of Artificial Sequence Synthetic peptide
TQCGPKGEPTYHCY

SEQ ID NO: 634 - protein, 14 aa, Artificial Sequence
  Description of Artificial Sequence Synthetic peptide
NYCGPEVEEYHNCD

SEQ ID NO: 635 - protein, 14 aa, Artificial Sequence
  Description of Artificial Sequence Synthetic peptide
VYCGPEWEFFSDCA

SEQ ID NO: 636 - protein, 14 aa, Artificial Sequence
  Description of Artificial Sequence Synthetic peptide
FYCGPELEESFFCY

SEQ ID NO: 637 - protein, 14 aa, Artificial Sequence
  Description of Artificial Sequence Synthetic peptide
DYCGPEIEENFYCY

SEQ ID NO: 638 - protein, 14 aa, Artificial Sequence
  Description of Artificial Sequence Synthetic peptide
YACGPEVEEYVYCA

SEQ ID NO: 639 - protein, 14 aa, Artificial Sequence
  Description of Artificial Sequence Synthetic peptide
VDCGPELDESIICD

SEQ ID NO: 640 - protein, 14 aa, Artificial Sequence
  Description of Artificial Sequence Synthetic peptide
NDCGPEMDESIACY

SEQ ID NO: 641 - protein, 14 aa, Artificial Sequence
  Description of Artificial Sequence Synthetic peptide
VYCGPEFDEYLACA

SEQ ID NO: 642 - protein, 14 aa, Artificial Sequence
  Description of Artificial Sequence Synthetic peptide
VICGPEFDESANCD

SEQ ID NO: 643 - protein, 14 aa, Artificial Sequence
  Description of Artificial Sequence Synthetic peptide
LTCWPDWEEDFFCN

SEQ ID NO: 644 - protein, 14 aa, Artificial Sequence
  Description of Artificial Sequence Synthetic peptide
FYCGPEQEEINYCY

SEQ ID NO: 645 - protein, 14 aa, Artificial Sequence
  Description of Artificial Sequence Synthetic peptide
TYCGPELEEFFLCY

SEQ ID NO: 646 - protein, 14 aa, Artificial Sequence
  Description of Artificial Sequence Synthetic peptide
PDCGPELDESVACH

SEQ ID NO: 647 - protein, 14 aa, Artificial Sequence
  Description of Artificial Sequence Synthetic peptide
NDCGPEFEEIIFCV

SEQ ID NO: 648 - protein, 14 aa, Artificial Sequence
  Description of Artificial Sequence Synthetic peptide
YICGPEFDESFYCN

SEQ ID NO: 649 - protein, 14 aa, Artificial Sequence
  Description of Artificial Sequence Synthetic peptide
HACGPELDESLLCN

SEQ ID NO: 650 - protein, 14 aa, Artificial Sequence
  Description of Artificial Sequence Synthetic peptide
YYCGPEWEEAVLCA

SEQ ID NO: 651 - protein, 14 aa, Artificial Sequence
  Description of Artificial Sequence Synthetic peptide
AFCGPEVEEYDLCN

SEQ ID NO: 652 - protein, 14 aa, Artificial Sequence
  Description of Artificial Sequence Synthetic peptide
YNCGPEFDESVACS

SEQ ID NO: 653 - protein, 14 aa, Artificial Sequence
  Description of Artificial Sequence Synthetic peptide
YLCGPEVEDDTLCA

SEQ ID NO: 654 - protein, 14 aa, Artificial Sequence
  Description of Artificial Sequence Synthetic peptide
AVCGPEFDESVNCD

SEQ ID NO: 655 - protein, 14 aa, Artificial Sequence
  Description of Artificial Sequence Synthetic peptide
NYCGPEWEVYSLCP

SEQ ID NO: 656 - protein, 14 aa, Artificial Sequence
  Description of Artificial Sequence Synthetic peptide
DFCGPEVEEDTYCH

SEQ ID NO: 657 - protein, 14 aa, Artificial Sequence
  Description of Artificial Sequence Synthetic peptide
ISCGPEYEWDYACN

SEQ ID NO: 658 - protein, 14 aa, Artificial Sequence
  Description of Artificial Sequence Synthetic peptide
VNCGPELDESIICY

SEQ ID NO: 659 - protein, 14 aa, Artificial Sequence
  Description of Artificial Sequence Synthetic peptide
YDCGPEFDETAPCY

SEQ ID NO: 660 - protein, 14 aa, Artificial Sequence
  Description of Artificial Sequence Synthetic peptide
YLCGPEFEENFLCT

SEQ ID NO: 661 - protein, 14 aa, Artificial Sequence
  Description of Artificial Sequence Synthetic peptide
FDCGPEVDESVDCA

SEQ ID NO: 662 - protein, 14 aa, Artificial Sequence
  Description of Artificial Sequence Synthetic peptide
YDCGPEQEEISFCN

SEQ ID NO: 663 - protein, 14 aa, Artificial Sequence
  Description of Artificial Sequence Synthetic peptide
IDCGPEIELYDDCF

SEQ ID NO: 664 - protein, 14 aa, Artificial Sequence
  Description of Artificial Sequence Synthetic peptide
TFCGPELDESVYCY

SEQ ID NO: 665 - protein, 14 aa, Artificial Sequence
  Description of Artificial Sequence Synthetic peptide
FFCGPEIDESNACV

SEQ ID NO: 666 - protein, 14 aa, Artificial Sequence
  Description of Artificial Sequence Synthetic peptide
YHCWPDWEPIYICI

SEQ ID NO: 667 - protein, 14 aa, Artificial Sequence
  Description of Artificial Sequence Synthetic peptide
AICGPEYEEDHYCY

SEQ ID NO: 668 - protein, 14 aa, Artificial Sequence
  Description of Artificial Sequence Synthetic peptide
PLCGPDGFENYNCF

SEQ ID NO: 669 - protein, 14 aa, Artificial Sequence
  Description of Artificial Sequence Synthetic peptide
FPCWPDWEWDNNCH

SEQ ID NO: 670 - protein, 14 aa, Artificial Sequence
  Description of Artificial Sequence Synthetic peptide
VDCGPDGDELAACH

SEQ ID NO: 671 - protein, 14 aa, Artificial Sequence
  Description of Artificial Sequence Synthetic peptide
VDCWPDWEEYYSCD

SEQ ID NO: 672 - protein, 14 aa, Artificial Sequence
  Description of Artificial Sequence Synthetic peptide
VYCGPEYDESYDCT

SEQ ID NO: 673 - protein, 14 aa, Artificial Sequence
  Description of Artificial Sequence Synthetic peptide
NLCGPEWENFADCF

SEQ ID NO: 674 - protein, 14 aa, Artificial Sequence
  Description of Artificial Sequence Synthetic peptide
YLCGPELEVFFVCD

SEQ ID NO: 675 - protein, 14 aa, Artificial Sequence
  Description of Artificial Sequence Synthetic peptide
IFCGPELEDYSICF

SEQ ID NO: 676 - protein, 14 aa, Artificial Sequence
  Description of Artificial Sequence Synthetic peptide
DYCGPELEQYDLCF

SEQ ID NO: 677 - protein, 14 aa, Artificial Sequence
  Description of Artificial Sequence Synthetic peptide
LLCGPVNEDPLDCY

SEQ ID NO: 678 - protein, 14 aa, Artificial Sequence
  Description of Artificial Sequence Synthetic peptide
IDCGPEFDESVFCY

SEQ ID NO: 679 - protein, 14 aa, Artificial Sequence
  Description of Artificial Sequence Synthetic peptide
IICGPEVEEIDICS

SEQ ID NO: 680 - protein, 14 aa, Artificial Sequence
  Description of Artificial Sequence Synthetic peptide
IACWPDWEDYSSCP

SEQ ID NO: 681 - protein, 14 aa, Artificial Sequence
  Description of Artificial Sequence Synthetic peptide
YYCGPEVEDINDCI

SEQ ID NO: 682 - protein, 14 aa, Artificial Sequence
  Description of Artificial Sequence Synthetic peptide
NICGPEMDESIDCI

SEQ ID NO: 683 - protein, 14 aa, Artificial Sequence
  Description of Artificial Sequence Synthetic peptide
FDCWPDWEELVSCY

SEQ ID NO: 684 - protein, 14 aa, Artificial Sequence
  Description of Artificial Sequence Synthetic peptide
YFCGPEWEDHFFCD

SEQ ID NO: 685 - protein, 14 aa, Artificial Sequence
  Description of Artificial Sequence Synthetic peptide
TYCGPEFEEDSYCD

SEQ ID NO: 686 - protein, 14 aa, Artificial Sequence
  Description of Artificial Sequence Synthetic peptide
NLCGPEVELIDICS

SEQ ID NO: 687 - protein, 14 aa, Artificial Sequence
  Description of Artificial Sequence Synthetic peptide
DNCGPEWEEVYLCN

SEQ ID NO: 688 - protein, 14 aa, Artificial Sequence
  Description of Artificial Sequence Synthetic peptide
FLCGPEFDESDLCF

SEQ ID NO: 689 - protein, 14 aa, Artificial Sequence
  Description of Artificial Sequence Synthetic peptide
HICGPEQDESIGCT

SEQ ID NO: 690 - protein, 14 aa, Artificial Sequence
  Description of Artificial Sequence Synthetic peptide
FDCWPDWEDNSYCD

SEQ ID NO: 691 - protein, 14 aa, Artificial Sequence
  Description of Artificial Sequence Synthetic peptide
TACGPEWEFDFNCD

SEQ ID NO: 692 - protein, 14 aa, Artificial Sequence
  Description of Artificial Sequence Synthetic peptide
HHCWPDWEDYSTCP

SEQ ID NO: 693 - protein, 14 aa, Artificial Sequence
  Description of Artificial Sequence Synthetic peptide
YYCGPEFDESVNCF

SEQ ID NO: 694 - protein, 14 aa, Artificial Sequence
  Description of Artificial Sequence Synthetic peptide
LHCWPDWEEIDICD

SEQ ID NO: 695 - protein, 14 aa, Artificial Sequence
  Description of Artificial Sequence Synthetic peptide
DICGPDGQEDFVCS

SEQ ID NO: 696 - protein, 14 aa, Artificial Sequence
  Description of Artificial Sequence Synthetic peptide
DVCWPDWEVNYFCD

SEQ ID NO: 697 - protein, 14 aa, Artificial Sequence
  Description of Artificial Sequence Synthetic peptide
VNCGPEMDESIDCA

SEQ ID NO: 698 - protein, 14 aa, Artificial Sequence
  Description of Artificial Sequence Synthetic peptide
DNCGPEFDEATVCN

SEQ ID NO: 699 - protein, 14 aa, Artificial Sequence
  Description of Artificial Sequence Synthetic peptide
DLCGPEFEEVHNCN

SEQ ID NO: 700 - protein, 14 aa, Artificial Sequence
  Description of Artificial Sequence Synthetic peptide
VNCGPEFDESSYCF

SEQ ID NO: 701 - protein, 14 aa, Artificial Sequence
  Description of Artificial Sequence Synthetic peptide
NICWPDWEEDNFCS

SEQ ID NO: 702 - protein, 14 aa, Artificial Sequence
  Description of Artificial Sequence Synthetic peptide
FVCGPEWEVYDDCD

SEQ ID NO: 703 - protein, 14 aa, Artificial Sequence
  Description of Artificial Sequence Synthetic peptide
AYCGPELEVVHLCV

SEQ ID NO: 704 - protein, 14 aa, Artificial Sequence
  Description of Artificial Sequence Synthetic peptide
PFCGPEMDETIDCY

SEQ ID NO: 705 - protein, 14 aa, Artificial Sequence
  Description of Artificial Sequence Synthetic peptide
LLCGPVMEDVFACY

SEQ ID NO: 706 - protein, 14 aa, Artificial Sequence
  Description of Artificial Sequence Synthetic peptide
DLCGPEFDESTNCY

SEQ ID NO: 707 - protein, 14 aa, Artificial Sequence
  Description of Artificial Sequence Synthetic peptide
HDCGPEMEEYYLCP

SEQ ID NO: 708 - protein, 14 aa, Artificial Sequence
  Description of Artificial Sequence Synthetic peptide
TDCGPEYDESIICP

SEQ ID NO: 709 - protein, 14 aa, Artificial Sequence
  Description of Artificial Sequence Synthetic peptide
VLCWPDWEDYADCN

SEQ ID NO: 710 - protein, 14 aa, Artificial Sequence
  Description of Artificial Sequence Synthetic peptide
NDCGPELDESLTCD

SEQ ID NO: 711 - protein, 14 aa, Artificial Sequence
  Description of Artificial Sequence Synthetic peptide
IYCGPELDESIACY

SEQ ID NO: 712 - protein, 14 aa, Artificial Sequence
  Description of Artificial Sequence Synthetic peptide
NYCGPEVEEFNFCH

SEQ ID NO: 713 - protein, 14 aa, Artificial Sequence
  Description of Artificial Sequence Synthetic peptide
DVCGPEIEEYSFCI

SEQ ID NO: 714 - protein, 14 aa, Artificial Sequence
  Description of Artificial Sequence Synthetic peptide
DLCGPEVEEITDCA

SEQ ID NO: 715 - protein, 14 aa, Artificial Sequence
  Description of Artificial Sequence Synthetic peptide
HDCGPEFDESVFCI

SEQ ID NO: 716 - protein, 14 aa, Artificial Sequence
  Description of Artificial Sequence Synthetic peptide
PLCGPVLEEDIYCY

SEQ ID NO: 717 - protein, 14 aa, Artificial Sequence
  Description of Artificial Sequence Synthetic peptide
DLCGPEFEDIIDCN

SEQ ID NO: 718 - protein, 14 aa, Artificial Sequence
  Description of Artificial Sequence Synthetic peptide
DYCGPEVEVPSNCN

SEQ ID NO: 719 - protein, 14 aa, Artificial Sequence
  Description of Artificial Sequence Synthetic peptide
IICGPELDESTACD

SEQ ID NO: 720 - protein, 14 aa, Artificial Sequence
  Description of Artificial Sequence Synthetic peptide
DHCGPEFDESVNCD

SEQ ID NO: 721 - protein, 14 aa, Artificial Sequence
  Description of Artificial Sequence Synthetic peptide
FDCGPEFDESLYCS

SEQ ID NO: 722 - protein, 14 aa, Artificial Sequence
  Description of Artificial Sequence Synthetic peptide
FACWPDWEEVYICY

SEQ ID NO: 723 - protein, 14 aa, Artificial Sequence
  Description of Artificial Sequence Synthetic peptide
LYCGPEFDESLDCS

SEQ ID NO: 724 - protein, 14 aa, Artificial Sequence
  Description of Artificial Sequence Synthetic peptide
DLCGPELEEAFLCA

SEQ ID NO: 725 - protein, 14 aa, Artificial Sequence
  Description of Artificial Sequence Synthetic peptide
FACGPELDETLTCL

SEQ ID NO: 726 - protein, 14 aa, Artificial Sequence
  Description of Artificial Sequence Synthetic peptide
FDCGPEVEEISNCD

SEQ ID NO: 727 - protein, 14 aa, Artificial Sequence
  Description of Artificial Sequence Synthetic peptide
VHCGPELEYPFDCN

SEQ ID NO: 728 - protein, 14 aa, Artificial Sequence
  Description of Artificial Sequence Synthetic peptide
AYCGPEFEEHTTCN

SEQ ID NO: 729 - protein, 14 aa, Artificial Sequence
  Description of Artificial Sequence Synthetic peptide
LYCGPVSEQFTFCI

SEQ ID NO: 730 - protein, 14 aa, Artificial Sequence
  Description of Artificial Sequence Synthetic peptide
DYCGPELDESYDCN

SEQ ID NO: 731 - protein, 14 aa, Artificial Sequence
  Description of Artificial Sequence Synthetic peptide
FYCGPEWEEFDVCI

SEQ ID NO: 732 - protein, 14 aa, Artificial Sequence
  Description of Artificial Sequence Synthetic peptide
FTCGPEVEEYDHCI

SEQ ID NO: 733 - protein, 14 aa, Artificial Sequence
  Description of Artificial Sequence Synthetic peptide
YNCGPEFDESVTCF

SEQ ID NO: 734 - protein, 14 aa, Artificial Sequence
  Description of Artificial Sequence Synthetic peptide
AVCGPENDESNSCA

SEQ ID NO: 735 - protein, 14 aa, Artificial Sequence
  Description of Artificial Sequence Synthetic peptide
IYCWPDWEVPNDCA

SEQ ID NO: 736 - protein, 14 aa, Artificial Sequence
  Description of Artificial Sequence Synthetic peptide
YFCGPEFEEFFHCY

SEQ ID NO: 737 - protein, 14 aa, Artificial Sequence
  Description of Artificial Sequence Synthetic peptide
YVCGPDGDESSFCD

SEQ ID NO: 738 - protein, 14 aa, Artificial Sequence
  Description of Artificial Sequence Synthetic peptide
ISCGPEVEEFFYCY

SEQ ID NO: 739 - protein, 14 aa, Artificial Sequence
  Description of Artificial Sequence Synthetic peptide
LICGPVFEEDVYCD

SEQ ID NO: 740 - protein, 14 aa, Artificial Sequence
  Description of Artificial Sequence Synthetic peptide
VYCGPEVEDHNYCA

SEQ ID NO: 741 - protein, 14 aa, Artificial Sequence
  Description of Artificial Sequence Synthetic peptide
LDCGPEFEFVYICA

SEQ ID NO: 742 - protein, 14 aa, Artificial Sequence
  Description of Artificial Sequence Synthetic peptide
YDCGPEFEEDLPCI

SEQ ID NO: 743 - protein, 14 aa, Artificial Sequence
  Description of Artificial Sequence Synthetic peptide
DVCGPEVEEDYYCD

SEQ ID NO: 744 - protein, 14 aa, Artificial Sequence
  Description of Artificial Sequence Synthetic peptide
NDCWPDWEYDNVCV

SEQ ID NO: 745 - protein, 14 aa, Artificial Sequence
  Description of Artificial Sequence Synthetic peptide
DLCGPEFEVANDCN

SEQ ID NO: 746 - protein, 14 aa, Artificial Sequence
  Description of Artificial Sequence Synthetic peptide
HDCGPELDESISCN

SEQ ID NO: 747 - protein, 14 aa, Artificial Sequence
  Description of Artificial Sequence Synthetic peptide
LDCWPDWEETTHCD

SEQ ID NO: 748 - protein, 14 aa, Artificial Sequence
  Description of Artificial Sequence Synthetic peptide
IICGPEVEEDDYCL

SEQ ID NO: 749 - protein, 14 aa, Artificial Sequence
  Description of Artificial Sequence Synthetic peptide
YYCWPDWEEVIICD

SEQ ID NO: 750 - protein, 14 aa, Artificial Sequence
  Description of Artificial Sequence Synthetic peptide
FDCGPEIDEYTNCN

SEQ ID NO: 751 - protein, 14 aa, Artificial Sequence
  Description of Artificial Sequence Synthetic peptide
ITCGPELDETINCD

SEQ ID NO: 752 - protein, 14 aa, Artificial Sequence
  Description of Artificial Sequence Synthetic peptide
DSCGPEVEEDIYCI

SEQ ID NO: 753 - protein, 14 aa, Artificial Sequence
  Description of Artificial Sequence Synthetic peptide
YLCGPEFDESGNCH

SEQ ID NO: 754 - protein, 14 aa, Artificial Sequence
  Description of Artificial Sequence Synthetic peptide
DNCGPELPEDYFCD

SEQ ID NO: 755 - protein, 14 aa, Artificial Sequence
  Description of Artificial Sequence Synthetic peptide
VLCGPEFEEVSNCN

SEQ ID NO: 756 - protein, 14 aa, Artificial Sequence
  Description of Artificial Sequence Synthetic peptide
VDCWPDWEEDIVCD

SEQ ID NO: 757 - protein, 14 aa, Artificial Sequence
  Description of Artificial Sequence Synthetic peptide
VYCWPDWEDNFPCY

SEQ ID NO: 758 - protein, 14 aa, Artificial Sequence
  Description of Artificial Sequence Synthetic peptide
DYCGPEVEEHFNCH

SEQ ID NO: 759 - protein, 14 aa, Artificial Sequence
  Description of Artificial Sequence Synthetic peptide
ADCGPEIEEDAYCY

SEQ ID NO: 760 - protein, 14 aa, Artificial Sequence
  Description of Artificial Sequence Synthetic peptide
ILCWPDWEDATFCY

SEQ ID NO: 761 - protein, 14 aa, Artificial Sequence
  Description of Artificial Sequence Synthetic peptide
IHCWPDWEDFNICP

SEQ ID NO: 762 - protein, 14 aa, Artificial Sequence
  Description of Artificial Sequence Synthetic peptide
TICGPEVEDYNDCI

SEQ ID NO: 763 - protein, 14 aa, Artificial Sequence
  Description of Artificial Sequence Synthetic peptide
DDCGPELDESVACI

SEQ ID NO: 764 - protein, 14 aa, Artificial Sequence
  Description of Artificial Sequence Synthetic peptide
DDCWPDWEDHIFCF

SEQ ID NO: 765 - protein, 14 aa, Artificial Sequence
  Description of Artificial Sequence Synthetic peptide
VNCGPEVEEIIFCD

SEQ ID NO: 766 - protein, 14 aa, Artificial Sequence
  Description of Artificial Sequence Synthetic peptide
IFCWPDWEDDTVCI

SEQ ID NO: 767 - protein, 14 aa, Artificial Sequence
  Description of Artificial Sequence Synthetic peptide
IICGPEFEEISDCL

SEQ ID NO: 768 - protein, 14 aa, Artificial Sequence
  Description of Artificial Sequence Synthetic peptide
FDCGPEVEEYNDCD

SEQ ID NO: 769 - protein, 14 aa, Artificial Sequence
  Description of Artificial Sequence Synthetic peptide
NDCGPELDETLYCI

SEQ ID NO: 770 - protein, 14 aa, Artificial Sequence
  Description of Artificial Sequence Synthetic peptide
AICGPELEEDISCN

SEQ ID NO: 771 - protein, 14 aa, Artificial Sequence
  Description of Artificial Sequence Synthetic peptide
HLCGPEFDESTNCY

SEQ ID NO: 772 - protein, 14 aa, Artificial Sequence
  Description of Artificial Sequence Synthetic peptide
SYCGPELDESVACI

SEQ ID NO: 773 - protein, 14 aa, Artificial Sequence
  Description of Artificial Sequence Synthetic peptide
LYCGPEFEQLADCT

SEQ ID NO: 774 - protein, 14 aa, Artificial Sequence
  Description of Artificial Sequence Synthetic peptide
IDCGPELDESIACN

SEQ ID NO: 775 - protein, 14 aa, Artificial Sequence
  Description of Artificial Sequence Synthetic peptide
FDCGPDGQEDLVCN

SEQ ID NO: 776 - protein, 14 aa, Artificial Sequence
  Description of Artificial Sequence Synthetic peptide
NLCGPEFEEFFDCY

SEQ ID NO: 777 - protein, 14 aa, Artificial Sequence
  Description of Artificial Sequence Synthetic peptide
SICGPELQEDIVCP

SEQ ID NO: 778 - protein, 14 aa, Artificial Sequence
  Description of Artificial Sequence Synthetic peptide
NPCGPEYDESAHCD

SEQ ID NO: 779 - protein, 14 aa, Artificial Sequence
  Description of Artificial Sequence Synthetic peptide
YDCGPELDEYYNCN

SEQ ID NO: 780 - protein, 14 aa, Artificial Sequence
  Description of Artificial Sequence Synthetic peptide
VDCGPELDETIFCD

SEQ ID NO: 781 - protein, 14 aa, Artificial Sequence
  Description of Artificial Sequence Synthetic peptide
DNCGPELDESVTCT

SEQ ID NO: 782 - protein, 14 aa, Artificial Sequence
  Description of Artificial Sequence Synthetic peptide
VDCGPELDESSYCD

SEQ ID NO: 783 - protein, 14 aa, Artificial Sequence
  Description of Artificial Sequence Synthetic peptide
YYCGPEFEFIDFCF

SEQ ID NO: 784 - protein, 14 aa, Artificial Sequence
  Description of Artificial Sequence Synthetic peptide
YDCWPDWEVITYCN

SEQ ID NO: 785 - protein, 14 aa, Artificial Sequence
  Description of Artificial Sequence Synthetic peptide
FDCGPEIEEDFFCV

SEQ ID NO: 786 - protein, 14 aa, Artificial Sequence
  Description of Artificial Sequence Synthetic peptide
IFCWPDWDDINFCD

SEQ ID NO: 787 - protein, 14 aa, Artificial Sequence
  Description of Artificial Sequence Synthetic peptide
NFCGPELPEDITCY

SEQ ID NO: 788 - protein, 14 aa, Artificial Sequence
  Description of Artificial Sequence Synthetic peptide
SLCGPEFEEYYHCL

SEQ ID NO: 789 - protein, 14 aa, Artificial Sequence
  Description of Artificial Sequence Synthetic peptide
ASCGPELDESLDCL

SEQ ID NO: 790 - protein, 14 aa, Artificial Sequence
  Description of Artificial Sequence Synthetic peptide
SFCGPEREWDLACY

SEQ ID NO: 791 - protein, 14 aa, Artificial Sequence
  Description of Artificial Sequence Synthetic peptide
HLCGPEFEDVLDCI

SEQ ID NO: 792 - protein, 14 aa, Artificial Sequence
  Description of Artificial Sequence Synthetic peptide
LDCGPDGDEFYYCL

SEQ ID NO: 793 - protein, 14 aa, Artificial Sequence
  Description of Artificial Sequence Synthetic peptide
DNCWPDWEEDIACT

SEQ ID NO: 794 - protein, 14 aa, Artificial Sequence
  Description of Artificial Sequence Synthetic peptide
SDCGPELDETIHCI

SEQ ID NO: 795 - protein, 14 aa, Artificial Sequence
  Description of Artificial Sequence Synthetic peptide
NSCGPDGDESVDCL

SEQ ID NO: 796 - protein, 14 aa, Artificial Sequence
  Description of Artificial Sequence Synthetic peptide
DLCGPELDESTLCI

SEQ ID NO: 797 - protein, 14 aa, Artificial Sequence
  Description of Artificial Sequence Synthetic peptide
FDCGPESDESFNCY

SEQ ID NO: 798 - protein, 14 aa, Artificial Sequence
  Description of Artificial Sequence Synthetic peptide
DLCGPEFEEDDICY

SEQ ID NO: 799 - protein, 14 aa, Artificial Sequence
  Description of Artificial Sequence Synthetic peptide
HACGPEFEEDTYCF

SEQ ID NO: 800 - protein, 14 aa, Artificial Sequence
  Description of Artificial Sequence Synthetic peptide
SDCGPELDESVACI

SEQ ID NO: 801 - protein, 14 aa, Artificial Sequence
  Description of Artificial Sequence Synthetic peptide
IVCGPELPEDYNCY

SEQ ID NO: 802 - protein, 14 aa, Artificial Sequence
  Description of Artificial Sequence Synthetic peptide
DLCGPEFDESFICF

SEQ ID NO: 803 - protein, 14 aa, Artificial Sequence
  Description of Artificial Sequence Synthetic peptide
YDCGPEFDETLTCN

SEQ ID NO: 804 - protein, 14 aa, Artificial Sequence
  Description of Artificial Sequence Synthetic peptide
YDCGPEVEEIVNCD

SEQ ID NO: 805 - protein, 14 aa, Artificial Sequence
  Description of Artificial Sequence Synthetic peptide
VFCGPEIEDDHVCI

SEQ ID NO: 806 - protein, 14 aa, Artificial Sequence
  Description of Artificial Sequence Synthetic peptide
FICGPEWEDDYACS

SEQ ID NO: 807 - protein, 14 aa, Artificial Sequence
  Description of Artificial Sequence Synthetic peptide
NFCGPEFDESVGCN

SEQ ID NO: 808 - protein, 14 aa, Artificial Sequence
  Description of Artificial Sequence Synthetic peptide
NLCGPEVEEILICD

SEQ ID NO: 809 - protein, 14 aa, Artificial Sequence
  Description of Artificial Sequence Synthetic peptide
DSCGPEVEEYDLCN

SEQ ID NO: 810 - protein, 14 aa, Artificial Sequence
  Description of Artificial Sequence Synthetic peptide
TLCGPEFEEITDCN

SEQ ID NO: 811 - protein, 14 aa, Artificial Sequence
  Description of Artificial Sequence Synthetic peptide
TVCGPKMEMNSTCD

SEQ ID NO: 812 - protein, 14 aa, Artificial Sequence
  Description of Artificial Sequence Synthetic peptide
VHCWPDWEDAVSCN

SEQ ID NO: 813 - protein, 14 aa, Artificial Sequence
  Description of Artificial Sequence Synthetic peptide
DLCGPDGNELDFCF

SEQ ID NO: 814 - protein, 14 aa, Artificial Sequence
  Description of Artificial Sequence Synthetic peptide
DLCGPDGEEHYYCD

SEQ ID NO: 815 - protein, 14 aa, Artificial Sequence
  Description of Artificial Sequence Synthetic peptide
FNCGPEVEEILLCT

SEQ ID NO: 816 - protein, 14 aa, Artificial Sequence
  Description of Artificial Sequence Synthetic peptide
HYCGPEVENINDCI

SEQ ID NO: 817 - protein, 14 aa, Artificial Sequence
  Description of Artificial Sequence Synthetic peptide
IDCGPELDESVICD

SEQ ID NO: 818 - protein, 14 aa, Artificial Sequence
  Description of Artificial Sequence Synthetic peptide
IFCGPEIEQPALCY

SEQ ID NO: 819 - protein, 14 aa, Artificial Sequence
  Description of Artificial Sequence Synthetic peptide
YDCGPEFQEDLVCP

SEQ ID NO: 820 - protein, 14 aa, Artificial Sequence
  Description of Artificial Sequence Synthetic peptide
HACWPDWEEPNYCD

SEQ ID NO: 821 - protein, 14 aa, Artificial Sequence
  Description of Artificial Sequence Synthetic peptide
NVCWPDWEEDYNCY

SEQ ID NO: 822 - protein, 14 aa, Artificial Sequence
  Description of Artificial Sequence Synthetic peptide
SFCGQELGDNYDCI

SEQ ID NO: 823 - protein, 14 aa, Artificial Sequence
  Description of Artificial Sequence Synthetic peptide
DDCGPELDETTVCY

SEQ ID NO: 824 - protein, 14 aa, Artificial Sequence
  Description of Artificial Sequence Synthetic peptide
NFCGPEWEVATLCL

SEQ ID NO: 825 - protein, 14 aa, Artificial Sequence
  Description of Artificial Sequence Synthetic peptide
DLCGPEVEEDTYCN

SEQ ID NO: 826 - protein, 14 aa, Artificial Sequence
  Description of Artificial Sequence Synthetic peptide
ALCGPEVEQVDLCT

SEQ ID NO: 827 - protein, 14 aa, Artificial Sequence
  Description of Artificial Sequence Synthetic peptide
DDCGPELDESVNCN

SEQ ID NO: 828 - protein, 14 aa, Artificial Sequence
  Description of Artificial Sequence Synthetic peptide
IFCGPEFEQIIYCD

SEQ ID NO: 829 - protein, 14 aa, Artificial Sequence
  Description of Artificial Sequence Synthetic peptide
SDCWPDWEEVYYCS

SEQ ID NO: 830 - protein, 14 aa, Artificial Sequence
  Description of Artificial Sequence Synthetic peptide
FFCGPDGDEVAICD

SEQ ID NO: 831 - protein, 14 aa, Artificial Sequence
  Description of Artificial Sequence Synthetic peptide
DDCWPDWEDDVYCY

SEQ ID NO: 832 - protein, 14 aa, Artificial Sequence
  Description of Artificial Sequence Synthetic peptide
YDCGPEFDETVVCP

SEQ ID NO: 833 - protein, 14 aa, Artificial Sequence
  Description of Artificial Sequence Synthetic peptide
SYCGPELDESVNCV

SEQ ID NO: 834 - protein, 14 aa, Artificial Sequence
  Description of Artificial Sequence Synthetic peptide
AFCVSFQQSLPHCD

SEQ ID NO: 835 - protein, 14 aa, Artificial Sequence
  Description of Artificial Sequence Synthetic peptide
VNCGPEVEEYFVCF

SEQ ID NO: 836 - protein, 14 aa, Artificial Sequence
  Description of Artificial Sequence Synthetic peptide
TNCWPDWEEDFACV

SEQ ID NO: 837 - protein, 14 aa, Artificial Sequence
  Description of Artificial Sequence Synthetic peptide
DDCGPEFEEIILCF

SEQ ID NO: 838 - protein, 14 aa, Artificial Sequence
  Description of Artificial Sequence Synthetic peptide
NSCGPELEDYHLCP

SEQ ID NO: 839 - protein, 14 aa, Artificial Sequence
  Description of Artificial Sequence Synthetic peptide
IHCGPDSDGFDFCD

SEQ ID NO: 840 - protein, 14 aa, Artificial Sequence
  Description of Artificial Sequence Synthetic peptide
FDCGPELEEDHLCF

SEQ ID NO: 841 - protein, 14 aa, Artificial Sequence
  Description of Artificial Sequence Synthetic peptide
TVCWPDFEEYADCD

SEQ ID NO: 842 - protein, 14 aa, Artificial Sequence
  Description of Artificial Sequence Synthetic peptide
YFCWPDWEEAADCL

SEQ ID NO: 843 - protein, 14 aa, Artificial Sequence
  Description of Artificial Sequence Synthetic peptide
VSCGPEFDESVDCI

SEQ ID NO: 844 - protein, 14 aa, Artificial Sequence
  Description of Artificial Sequence Synthetic peptide
AVCGPELPEDIVCY

SEQ ID NO: 845 - protein, 14 aa, Artificial Sequence
  Description of Artificial Sequence Synthetic peptide
VLCGPEVEEYHLCA

SEQ ID NO: 846 - protein, 14 aa, Artificial Sequence
  Description of Artificial Sequence Synthetic peptide
SLCWPDWEEVDNCF

SEQ ID NO: 847 - protein, 14 aa, Artificial Sequence
  Description of Artificial Sequence Synthetic peptide
TICGPDGQEDYNCH

SEQ ID NO: 848 - protein, 14 aa, Artificial Sequence
  Description of Artificial Sequence Synthetic peptide
FLCGPEGEPTYLCT

SEQ ID NO: 849 - protein, 14 aa, Artificial Sequence
  Description of Artificial Sequence Synthetic peptide
DDCWLEQHDIYVCA

SEQ ID NO: 850 - protein, 14 aa, Artificial Sequence
  Description of Artificial Sequence Synthetic peptide
IFCGPEVEEVAFCF

SEQ ID NO: 851 - protein, 14 aa, Artificial Sequence
  Description of Artificial Sequence Synthetic peptide
ALCGPEVEDDYDCL

SEQ ID NO: 852 - protein, 14 aa, Artificial Sequence
  Description of Artificial Sequence Synthetic peptide
DDCGPEIELYLTCA

SEQ ID NO: 853 - protein, 14 aa, Artificial Sequence
  Description of Artificial Sequence Synthetic peptide
TDCWPDWEDDSICD

SEQ ID NO: 854 - protein, 14 aa, Artificial Sequence
  Description of Artificial Sequence Synthetic peptide
SICGPELEEIFLCN

SEQ ID NO: 855 - protein, 14 aa, Artificial Sequence
  Description of Artificial Sequence Synthetic peptide
NDCGPELEEDILCF

SEQ ID NO: 856 - protein, 14 aa, Artificial Sequence
  Description of Artificial Sequence Synthetic peptide
DDCGPELDETYSCY

SEQ ID NO: 857 - protein, 14 aa, Artificial Sequence
  Description of Artificial Sequence Synthetic peptide
FVCGPEWEEIDLCI

SEQ ID NO: 858 - protein, 14 aa, Artificial Sequence
  Description of Artificial Sequence Synthetic peptide
YDCGPELDESLSCP

SEQ ID NO: 859 - protein, 14 aa, Artificial Sequence
  Description of Artificial Sequence Synthetic peptide
NLCGPELDESIICP

SEQ ID NO: 860 - protein, 14 aa, Artificial Sequence
  Description of Artificial Sequence Synthetic peptide
IDCWPDWEEFNNCF

SEQ ID NO: 861 - protein, 14 aa, Artificial Sequence
  Description of Artificial Sequence Synthetic peptide
AYCGPELEEHDYCY

SEQ ID NO: 862 - protein, 14 aa, Artificial Sequence
  Description of Artificial Sequence Synthetic peptide
DDCSPQFDQIDLCD

SEQ ID NO: 863 - protein, 14 aa, Artificial Sequence
  Description of Artificial Sequence Synthetic peptide
SYCGPESDESIYCY

SEQ ID NO: 864 - protein, 14 aa, Artificial Sequence
  Description of Artificial Sequence Synthetic peptide
SLCGPEFQEDAPCN

SEQ ID NO: 865 - protein, 14 aa, Artificial Sequence
  Description of Artificial Sequence Synthetic peptide
DACGPELDETTHCD

SEQ ID NO: 866 - protein, 14 aa, Artificial Sequence
  Description of Artificial Sequence Synthetic peptide
NDCGPEVEEVADCF

SEQ ID NO: 867 - protein, 14 aa, Artificial Sequence
  Description of Artificial Sequence Synthetic peptide
YICGPEFEQDYFCF

SEQ ID NO: 868 - protein, 14 aa, Artificial Sequence
  Description of Artificial Sequence Synthetic peptide
TDCGPDGDETNYCF

SEQ ID NO: 869 - protein, 14 aa, Artificial Sequence
  Description of Artificial Sequence Synthetic peptide
YDCGPEVDESVLCP

SEQ ID NO: 870 - protein, 14 aa, Artificial Sequence
  Description of Artificial Sequence Synthetic peptide
IDCGPEFDESAYCT

SEQ ID NO: 871 - protein, 14 aa, Artificial Sequence
  Description of Artificial Sequence Synthetic peptide
FDCGPEFEEFFHCY

SEQ ID NO: 872 - protein, 14 aa, Artificial Sequence
  Description of Artificial Sequence Synthetic peptide
FPCGPEIEEYDYCV

SEQ ID NO: 873 - protein, 14 aa, Artificial Sequence
  Description of Artificial Sequence Synthetic peptide
ADCGPIFESIDICV

SEQ ID NO: 874 - protein, 14 aa, Artificial Sequence
  Description of Artificial Sequence Synthetic peptide
ISCGPDLWPTDICT

SEQ ID NO: 875 - protein, 14 aa, Artificial Sequence
  Description of Artificial Sequence Synthetic peptide
DDCGPDGDEVHTCN

SEQ ID NO: 876 - protein, 14 aa, Artificial Sequence
  Description of Artificial Sequence Synthetic peptide
IDCWPDWEGSFACN

SEQ ID NO: 877 - protein, 14 aa, Artificial Sequence
  Description of Artificial Sequence Synthetic peptide
LNCGPDGDETFYCD

SEQ ID NO: 878 - protein, 14 aa, Artificial Sequence
  Description of Artificial Sequence Synthetic peptide
IHCGPELGAYISCS

SEQ ID NO: 879 - protein, 14 aa, Artificial Sequence
  Description of Artificial Sequence Synthetic peptide
TICWPDWEEDYFCY

SEQ ID NO: 880 - protein, 14 aa, Artificial Sequence
  Description of Artificial Sequence Synthetic peptide
HSCLAQFDQDLVCI

SEQ ID NO: 881 - protein, 14 aa, Artificial Sequence
  Description of Artificial Sequence Synthetic peptide
NNCASDLSEDNSCI

SEQ ID NO: 882 - protein, 14 aa, Artificial Sequence
  Description of Artificial Sequence Synthetic peptide
VNCWPDWEEDVACN

SEQ ID NO: 883 - protein, 14 aa, Artificial Sequence
  Description of Artificial Sequence Synthetic peptide
VYCGPELDESFYCF

SEQ ID NO: 884 - protein, 14 aa, Artificial Sequence
  Description of Artificial Sequence Synthetic peptide
LFCGTVLDEFFDCD

SEQ ID NO: 885 - protein, 14 aa, Artificial Sequence
  Description of Artificial Sequence Synthetic peptide
FDCGPELDESVSCA

SEQ ID NO: 886 - protein, 14 aa, Artificial Sequence
  Description of Artificial Sequence Synthetic peptide
YYCGPEVEENVYCI

SEQ ID NO: 887 - protein, 14 aa, Artificial Sequence
  Description of Artificial Sequence Synthetic peptide
DYCGPELDDSTICH

SEQ ID NO: 888 - protein, 14 aa, Artificial Sequence
  Description of Artificial Sequence Synthetic peptide
VFCGPQWAEANACF

SEQ ID NO: 889 - protein, 14 aa, Artificial Sequence
  Description of Artificial Sequence Synthetic peptide
FTCGPEYDESITCP

SEQ ID NO: 890 - protein, 14 aa, Artificial Sequence
  Description of Artificial Sequence Synthetic peptide
LNCGPVNDESSVCI

SEQ ID NO: 891 - protein, 14 aa, Artificial Sequence
  Description of Artificial Sequence Synthetic peptide
AICWPDWEEFSDCH

SEQ ID NO: 892 - protein, 14 aa, Artificial Sequence
  Description of Artificial Sequence Synthetic peptide
YYCGVDLGANVYCY

SEQ ID NO: 893 - protein, 14 aa, Artificial Sequence
  Description of Artificial Sequence Synthetic peptide
HYCGPEVEEDYHCD

SEQ ID NO: 894 - protein, 14 aa, Artificial Sequence
  Description of Artificial Sequence Synthetic peptide
NDCGSLQYDIPTCV

SEQ ID NO: 895 - protein, 14 aa, Artificial Sequence
  Description of Artificial Sequence Synthetic peptide
YVCRPQLDVYHYCN

SEQ ID NO: 896 - protein, 14 aa, Artificial Sequence
  Description of Artificial Sequence Synthetic peptide
HDCGPDGDESIICS

SEQ ID NO: 897 - protein, 14 aa, Artificial Sequence
  Description of Artificial Sequence Synthetic peptide
FDCGPELDETIICP

SEQ ID NO: 898 - protein, 14 aa, Artificial Sequence
  Description of Artificial Sequence Synthetic peptide
VDCGSDRGENAACH

SEQ ID NO: 899 - protein, 14 aa, Artificial Sequence
  Description of Artificial Sequence Synthetic peptide
NICLAQFNEDPTCN

SEQ ID NO: 900 - protein, 14 aa, Artificial Sequence
  Description of Artificial Sequence Synthetic peptide
TNCGSKSQVSDHCI

SEQ ID NO: 901 - protein, 14 aa, Artificial Sequence
  Description of Artificial Sequence Synthetic peptide
NHCHPQFWELTNCN

SEQ ID NO: 902 - protein, 14 aa, Artificial Sequence
  Description of Artificial Sequence Synthetic peptide
LLCHPQGDLYHLCH

SEQ ID NO: 903 - protein, 14 aa, Artificial Sequence
  Description of Artificial Sequence Synthetic peptide
TNCDSKLEGDDNCF

SEQ ID NO: 904 - protein, 14 aa, Artificial Sequence
  Description of Artificial Sequence Synthetic peptide
NDCGPEMDESLLCD

SEQ ID NO: 905 - protein, 14 aa, Artificial Sequence
  Description of Artificial Sequence Synthetic peptide
FHCGPEVDESINCN

SEQ ID NO: 906 - protein, 14 aa, Artificial Sequence
  Description of Artificial Sequence Synthetic peptide
DFCGPDGDETYVCS

SEQ ID NO: 907 - protein, 14 aa, Artificial Sequence
  Description of Artificial Sequence Synthetic peptide
VSCGPQFDENNTCN

SEQ ID NO: 908 - protein, 14 aa, Artificial Sequence
  Description of Artificial Sequence Synthetic peptide
VYCHLESEQFDICI

SEQ ID NO: 909 - protein, 14 aa, Artificial Sequence
  Description of Artificial Sequence Synthetic peptide
DDCGPEWEFVFFCD

SEQ ID NO: 910 - protein, 14 aa, Artificial Sequence
  Description of Artificial Sequence Synthetic peptide
YNCEQQQDEDPSCI

SEQ ID NO: 911 - protein, 14 aa, Artificial Sequence
  Description of Artificial Sequence Synthetic peptide
NTCGTEQHEFNGCL

SEQ ID NO: 912 - protein, 14 aa, Artificial Sequence
  Description of Artificial Sequence Synthetic peptide
HPCQPGFEEVDYCV

SEQ ID NO: 913 - protein, 14 aa, Artificial Sequence
  Description of Artificial Sequence Synthetic peptide
VACSRQLGEDAYCN

SEQ ID NO: 914 - protein, 14 aa, Artificial Sequence
  Description of Artificial Sequence Synthetic peptide
DICGAQEVHVYTCP

SEQ ID NO: 915 - protein, 14 aa, Artificial Sequence
  Description of Artificial Sequence Synthetic peptide
FFCEGNLDAYLLCL

SEQ ID NO: 916 - protein, 14 aa, Artificial Sequence
  Description of Artificial Sequence Synthetic peptide
YYCGPDGEENIVCD

SEQ ID NO: 917 - protein, 5 aa, Artificial Sequence
  Description of Artificial Sequence Synthetic peptide
GSGGS

SEQ ID NO: 918 - protein, 4 aa, Artificial Sequence
  Description of Artificial Sequence Synthetic peptide
GGGS

SEQ ID NO: 919 - protein, 5 aa, Artificial Sequence
  Description of Artificial Sequence Synthetic peptide
GGGGS

SEQ ID NO: 920 - protein, 6 aa, Artificial Sequence
  Description of Artificial Sequence Synthetic peptide
GSSGGS

SEQ ID NO: 921 - protein, 9 aa, Artificial Sequence
  Description of Artificial Sequence Synthetic peptide
GGGGSGGGS

SEQ ID NO: 922 - protein, 9 aa, Artificial Sequence
  Description of Artificial Sequence Synthetic peptide
  SITE (1)..(9): This sequence may encompass 1-3 “Gly Gly Ser” repeating units
GGSGGSGGS

SEQ ID NO: 923 - protein, 18 aa, Artificial Sequence
  Description of Artificial Sequence Synthetic peptide
GGGGSLSGRSDNHGSSGT

SEQ ID NO: 924 - protein, 26 aa, Artificial Sequence
  Description of Artificial Sequence Synthetic peptide
GGGGSSGGSGGSGLSGRSDNHGSSGT

SEQ ID NO: 925 - protein, 8 aa, Artificial Sequence
  Description of Artificial Sequence Synthetic peptide
ASGRSDNH

SEQ ID NO: 926 - protein, 8 aa, Artificial Sequence
  Description of Artificial Sequence Synthetic peptide
LAGRSDNH

SEQ ID NO: 927 - protein, 13 aa, Artificial Sequence
  Description of Artificial Sequence Synthetic peptide
ISSGLASGRSDNH

SEQ ID NO: 928 - protein, 13 aa, Artificial Sequence
  Description of Artificial Sequence Synthetic peptide
ISSGLLAGRSDNH

SEQ ID NO: 929 - protein, 8 aa, Artificial Sequence
  Description of Artificial Sequence Synthetic peptide
LSGRSDNH

SEQ ID NO: 930 - protein, 13 aa, Artificial Sequence
  Description of Artificial Sequence Synthetic peptide
ISSGLLSGRSDNP

SEQ ID NO: 931 - protein, 13 aa, Artificial Sequence
  Description of Artificial Sequence Synthetic peptide
ISSGLLSGRSDNH

SEQ ID NO: 932 - protein, 16 aa, Artificial Sequence
  Description of Artificial Sequence Synthetic peptide
LSGRSDNHSPLGLAGS

SEQ ID NO: 933 - protein, 16 aa, Artificial Sequence
  Description of Artificial Sequence Synthetic peptide
SPLGLAGSLSGRSDNH

SEQ ID NO: 934 - protein, 12 aa, Artificial Sequence
  Description of Artificial Sequence Synthetic peptide
SPLGLSGRSDNH

SEQ ID NO: 935 - protein, 16 aa, Artificial Sequence
  Description of Artificial Sequence Synthetic peptide
LAGRSDNHSPLGLAGS

SEQ ID NO: 936 - protein, 16 aa, Artificial Sequence
  Description of Artificial Sequence Synthetic peptide
LSGRSDNHVPLSLKMG

SEQ ID NO: 937 - protein, 16 aa, Artificial Sequence
  Description of Artificial Sequence Synthetic peptide
LSGRSDNHVPLSLSMG

SEQ ID NO: 938 - protein, 6 aa, Artificial Sequence
  Description of Artificial Sequence Synthetic peptide
  SITE (1)..(6): This sequence may encompass 1-3 “Gly Ser” repeating units
GSGSGS

SEQ ID NO: 939 - protein, 15 aa, Artificial Sequence
  Description of Artificial Sequence Synthetic peptide
  SITE (1)..(15): This sequence may encompass 1-3 “Gly Ser Gly Gly Ser” repeating units
GSGGSGSGGSGSGGS

SEQ ID NO: 940 - protein, 12 aa, Artificial Sequence
  Description of Artificial Sequence Synthetic peptide
  SITE (1)..(12): This sequence may encompass 1-3 “Gly Gly Gly Ser” repeating units
GGGSGGGSGGGS

SEQ ID NO: 941 - protein, 15 aa, Artificial Sequence
  Description of Artificial Sequence Synthetic peptide
  SITE (1)..(15): This sequence may encompass 1-3 “Gly Gly Gly Gly Ser” repeating units
GGGGSGGGGSGGGGS

SEQ ID NO: 942 - protein, 18 aa, Artificial Sequence
  Description of Artificial Sequence Synthetic peptide
  SITE (1)..(18): This sequence may encompass 1-3 “Gly Ser Ser Gly Gly Ser” repeating units
GSSGGSGSSGGSGSSGGS

SEQ ID NO: 943 - protein, 28 aa, Artificial Sequence
  Description of Artificial Sequence Synthetic peptide
GGGGSGGGSGGISSGLLSGRSDNHGGGS